(19) United States
(12) Patent Application Publication
(10) Pub. No.: US 2022/0220536 A1
(43) Pub. Date: Jul. 14, 2022

(54) COMPOSITIONS AND METHODS OF USING A DNA NANOSWITCH FOR THE DETECTION OF RNA

(71) Applicant: The Research Foundation for the State University of New York, Albany, NY (US)

(72) Inventors: Kenneth A. Halvorsen, Glenmont, NY (US); Lifeng Zhou, Albany, NY (US); Arun Richard Chandrasekaran, Albany, NY (US)

(21) Appl. No.: 17/573,529

(22) Filed: Jan. 11, 2022

Related U.S. Application Data
(60) Provisional application No. 63/136,183, filed on Jan. 11, 2021.

(51) Int. Cl. C12Q 1/6809; C12Q 1/6837

(52) U.S. Cl. CPC C12Q 1/6809; C12Q 1/6837

(57) ABSTRACT

The present disclosure is directed to a nucleic acid, including: a DNA nanoswitch-nucleic acid complex including a deoxyribonucleic acid (DNA) nanoswitch and a ribonucleic acid binding site, wherein the DNA nanoswitch has a first conformation characterized as open, and a second conformation characterized as closed when in a presence of ribonucleic acid-of-interest. DNA nanoswitches that hybridize to preselected viral RNA are also disclosed, as well as methods of detecting or identifying an RNA virus, and kits related thereto.

### CROSS-REFERENCE TO RELATED APPLICATIONS

The present disclosure claims priority or the benefit under 35 U.S.C. § 119 of U.S. provisional application No. 63/136,183 filed 11 Jan. 2021, which is herein entirely incorporated by reference.

### STATEMENT OF GOVERNMENTAL SUPPORT

This invention was made with governmental support under grant no. GM124720 awarded by the National Institutes of Health and grant no. CBET2030279 awarded by the National Science Foundation. The government has certain rights in this invention.

### REFERENCE TO A SEQUENCE LISTING

The instant application contains a sequence listing which has been submitted electronically in ASCII format and is hereby incorporated by reference in its entirety. Said ASCII copy, created on 24 Mar. 2022, is named sequences_rna_finalmwk1a_ST25.txt and is 791,000 bytes in size.

### FIELD OF THE INVENTION

The present disclosure relates to nucleic acid compositions and methods of use thereof for detecting RNA such as viral RNA and fragments thereof. More specifically, the present disclosure relates to methods of altering a nucleic acid shape or conformation such as a DNA nanoswitch with one or more ribonucleic acids and/or detecting or identifying the one or more ribonucleic acids.

### BACKGROUND

Newly emerging or re-emerging viruses pose significant challenges to health care systems, particularly as globalization has contributed to the rampant spread of these viruses (See e.g., K. F. Smith, et al., Globalization of human infectious disease. Ecology. 88, 1903-1910 (2007)). RNA viruses are frequently the cause of sweeping outbreaks as these viruses have high mutation rates and thus evolve rapidly (See e.g., R. Sanjuán, et al., Viral Mutation Rates. Journal of Virology. 84, 9733-9748 (2010); and D. A. Steinhauer, J. J. Holland, Rapid Evolution of RNA Viruses. Annual Review of Microbiology. 41, 409-431 (1987)). Examples of this include the annual influenza outbreak, Ebola virus, Zika virus (ZIKV) and the SARS-CoV-2 virus responsible for the COVID-19 pandemic. Technological advancements in structural biology and genomics have been important for identifying viruses, and for advancing fundamental viral research and antiviral therapeutics (See e.g., H. D. Marston, et al., Emerging Viral Diseases: Confronting Threats with New Technologies. Science Translational Medicine. 6, 253ps10-253ps10 (2014)). However, the inventors have observed that clinical methods for robust, low-cost and rapid detection of viral infections remain a major challenge for emergent viruses, especially in resource limited areas.

Detection of RNA viruses in the clinical setting is typically performed using either immunological detection based on enzyme-linked immunosorbent assay (ELISA) to detect IgM antibodies or nucleic acid testing (NAT) based on a reverse transcription polymerase chain reaction (RT-PCR) assay to detect viral RNA. (See e.g., K. R. Jerome, Lennette's Laboratory Diagnosis of Viral Infections (CRC Press, 2016); V. M. Corman, et al., Assay optimization for molecular detection of Zika virus. Bull World Health Organ. 94, 880-892 (2016); and D. Musso, D. J. Gubler, Zika Virus. Clinical Microbiology Reviews. 29, 487-524)(2016); D. J. Clark, et al., The current landscape of nucleic acid tests for filovirus detection. Journal of Clinical Virology. 103, 27-36 (2018)). Diagnosing RNA viruses, is made challenging by several factors including a limited time window for detection, low or varying viral load, cross-reactive IgM antibodies, and laboratory resources. The detection time windows can vary widely from as short as a few days to as long as several months (See e.g., K. R. Jerome, Lennette's Laboratory Diagnosis of Viral Infections (CRC Press, 2016)), and molecular detection techniques are usually most reliable if performed within the first two weeks of the disease (See e.g., K. James, Immunoserology of infectious diseases. Clinical Microbiology Reviews. 3, 132-152 (1990); and L. R. Petersen, D. J. Jamieson, A. M. Powers, M. A. Honein, Zika Virus. New England Journal of Medicine. 374, 1552-1563 (2016)). Depending on the timing of testing relative to infection, even highly sensitive NAT assays may still produce false negative or false positive results (See e.g., V. M. Corman, et al., Assay optimization for molecular detection of Zika virus. Bull World Health Organ. 94, 880-892 (2016)). On the other hand, results from IgM serology tests often cannot distinguish related viruses or different strains of the same virus due to cross-reactivity of IgM antibodies, thus leading to false positive results (See W. Dejnirattisai, et al., Dengue virus sero-cross-reactivity drives antibody-dependent enhancement of infection with zika virus. Nature Immunology. 17, 1102-1108 (2016); and C. R. Woods, False-Positive Results for Immunoglobulin M Serologic Results: Explanations and Examples. J Pediatric Infect Dis Soc. 2, 87-90 (2013)). These detection challenges are further exacerbated when outbreaks occur in low resource settings where infrastructure for these lab-intensive tests can be lacking, accelerating the spread of disease (See e.g., D. Musso, D. J. Gubler, Zika Virus. Clinical Microbiology Reviews. 29, 487-524 (2016), and J. V. Lazarus, et al., Too many people with viral hepatitis are diagnosed late—with dire consequences. Nat Rev Gastroenterol Hepatol. 16, 451-452 (2019)). In response to some of these challenges, new techniques are being developed to detect emerging viruses. Among these are methods that adopt nanoparticles (M. S. Draz, H. Shafiee, Applications of gold nanoparticles in virus detection. Theranostics. 8, 1985-2017 (2018)), graphene-based biosensors (See e.g., S. Afsahi, et al., Novel graphene-based biosensor for early detection of Zika virus infection. Biosensors and Bioelectronics. 100, 85-88 (2018)), and CRISPR-based methods (See J. S. Gootenberg, et al., Nucleic acid detection with CRISPR-Cas13a/C2c2. Science, eaam9321 (2017); and K. Pardee, et al., Rapid, Low-Cost Detection of Zika Virus Using Programmable Biomolecular Components. Cell. 165, 1255-1266 (2016)), to name a few. Many of these proposed strategies, although based on cutting-edge technology, require multiple reactions or signal transformation steps.

DNA nanoswitches are versatile nucleic acid complexes typically including a nucleic acid, either single- or double-stranded, which is modified to contain preselected segments of nucleic acids and designed to assume a linear (or open) conformation or a looped (or closed) formation depending upon the presence of an oligonucleotide such as an oligonucleotide-of-interest. DNA nanoswitches have been described in U.S. Patent Publication No. 2018/0223344 (herein entirely incorporated by reference) however it has not been heretofore contemplated to alter the conformation of a DNA nanoswitch as described herein such that when the DNA nanoswitch is combined or contacted with a ribonucleic nucleic acid-of-interest, it forms a DNA nanoswitch-ribonucleic acid complex suitable for providing a signal for RNA viral detection/identification as described herein, such as when contacted with viral RNA or one or more portions thereof.

There is a continuing need for robust methods of detecting RNA and identifying viral RNA.

### SUMMARY

The present disclosure relates to a method of reconfiguring or changing the shape of a nucleic acid in accordance with the present disclosure, which is useful for, inter alia, detecting ribonucleic acid-of-interest such as viral ribonucleic acids or portions thereof. In embodiments, the present disclosure is directed towards a method of detecting an RNA virus by reconfiguring or changing the shape of a nucleic acid including: contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal.

In some embodiments, the present disclosure relates to a method of reconfiguring a nucleic acid including: contacting a deoxyribonucleic acid (DNA) nanoswitch having a first conformation, with a ribonucleic acid to form a DNA nanoswitch-nucleic acid complex having a second conformation, wherein the second conformation is characterized as locked; processing a mixture under conditions sufficient to separate the first conformation and the second conformation; and contacting the first conformation and second conformation with an indicator under conditions sufficient to form a signal. In some embodiments, the signal is predetermined to show a presence or absence of ribonucleic acid-of-interest.

In some embodiments, the present disclosure relates to a nucleic acid, including: a DNA nanoswitch-nucleic acid complex including a deoxyribonucleic acid (DNA) nanoswitch and one or more ribonucleic acid binding sites, wherein the DNA nanoswitch has a first conformation characterized as open, and a second conformation characterized as closed when in a presence of one or more ribonucleic acids-of-interest.

In embodiments, the present disclosure includes a DNA nanoswitch suitable for forming a DNA nanoswitch-ribonucleic acid complex, including: a scaffold including a plurality of nucleotides or a polynucleotide sequence; a plurality of backbone oligonucleotides hybridized to the scaffold to form a backbone polynucleotide; a first detector strand including a nucleic acid sequence having a first segment hybridized to the scaffold or the backbone polynucleotide, and a second segment characterized as an overhang, wherein the second segment is hybridizable to, when present a first segment of a preselected target RNA nucleotide sequence or target RNA polynucleotide-of-interest; and a second detector strand comprising a nucleic acid sequence having a first segment hybridized to the scaffold or the backbone polynucleotide, and a second segment characterized as an overhang, wherein the second segment is hybridizable to, when present, a second segment of the preselected target RNA nucleotide sequence or the target RNA polynucleotide-of-interest.

In embodiments, the present disclosure includes a kit, including: one or more DNA nanoswitches of the present disclosure, wherein, when present, the DNA nanoswitch hybridizes a viral RNA or a fragment thereof; and a separation medium, and optionally a buffer solution.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A to 1C depict a DNA nanoswitch process flow embodiment for viral RNA sensing. FIG. 1A depicts a schematic of the DNA nanoswitch and detection of a viral RNA sequence. FIG. 1B depicts fast development cycle of nanoswitches for RNA viruses. FIG. 1C depicts a nanoswitch-based assay that allows direct detection using a non-enzymatic approach (top panel) and can optionally be combined with an isothermal amplification step like NASBA: nucleic acid sequence-based amplification (bottom panel).

FIGS. 2A-2F depict detection of viral RNA using DNA nanoswitches and a mixture thereof in accordance with the present disclosure. FIG. 2A depicts a schematic of the fragmentation of viral RNA and subsequent detection by the DNA nanoswitch in accordance with the present disclosure. FIG. 2B depicts fragmentation analysis of ZIKV RNA that was fragmented at 94° C. for 1, 3, 6, and 9 minutes. FIG. 2C depicts proof-of-concept showing detection of a preselected target region chosen from the literature (See e.g., R. S. Lanciotti, et al., Genetic and Serologic Properties of Zika Virus Associated with an Epidemic, Yap State, Micronesia, 2007. Emerg Infect Dis. 14, 1232-1239 (2008) (herein entirely incorporated by reference)) (0.8% agarose gel in 0.5×TBE buffer). FIG. 2D depicts a schematic of multiple nanoswitches or a nanoswitch mixture for detection with a signal multiplication strategy of the present disclosure. FIG. 2E depicts validation of a signal multiplication strategy of the present disclosure: the detection signal was increased for a fixed pool of DNA targets when using multiple targeting nanoswitches. FIG. 2F depicts detection sensitivity of the pooled nanoswitches for ZIKV RNA in 10 μl reaction. Error bars represent standard deviation from triplicate experiments.

FIGS. 3A-3D depict DNA nanoswitches specifically and differentially detecting RNA from two different flaviviruses and between two highly similar ZIKV isolates. FIG. 3A depicts ZIKV nanoswitches specifically detecting ZIKV RNA but not DENV RNA, and vice versa. FIG. 3B depicts multiplexed detection of ZIKV and DENV RNA. FIG. 3C depicts an illustration showing culture and RNA extraction of two different lineages of zika virus, namely, ZIKV Cambodia (Asian lineage) and Uganda (African lineage) strains. The mismatches in a representative target sequence between the two strains are shown. FIG. 3D depicts a specificity test of Cambodia and Uganda strains of ZIKV RNA. * denotes a band of contaminating cellular DNA following RNA isolation.

FIGS. 4A-4C depict DNA nanoswitches directly detecting ZIKV RNA extracted from infected human liver cells. FIG. 4A depicts RNA isolated from mock-infected Huh7 cells at 1, 2, and 3 days post infection shows no ZIKV detection. FIG. 4B depicts RNA isolated from Zika-infected Huh7 cells at 1, 2, and 3 days post infection shows increasing detection of ZIKV RNA over time, with red arrows denoting detection bands. * denotes a band of contaminating cellular DNA following RNA extraction. FIG. 4C depicts quantification of nanoswitch detection signal, with error bars representing standard deviation from triplicate experiments.

FIGS. 5A-5C depict prior extraction or pre-amplification of target RNA facilitates detection of ZIKV and SARS-CoV-2 RNA at clinically relevant levels in biofluids. FIG. 5A depicts positive identification of ZIKV RNA in spiked urine by first isolating in vitro transcribed target RNA using a commercially available viral RNA extraction kit, followed by direct, non-enzymatic detection using DNA nanoswitches. FIG. 5B depicts positive identification of ZIKV RNA from virus particles spiked into urine based on NASBA. FIG. 5C depicts positive detection of in vitro transcribed SARS-CoV-2 RNA in human saliva based on NASBA. Error bars represent standard deviation from triplicate experiments.

FIGS. 6A-6C depict a DNA nanoswitch construction and in vitro transcription (IVT) of viral RNA. FIG. 6A depicts an illustration of M13 scaffold linearization and assembly of DNA nanoswitch with backbone oligos and detectors. FIG. 6B depicts a schematic of in vitro transcription reaction. Plasmids containing the full-length infectious cDNA clone of either the ZIKV or DENV genomes were linearized, in vitro transcribed, followed by purification of the RNA product. FIG. 6C depicts the integrity of in vitro transcribed (IVT) ZIKV RNA was analyzed by electrophoresis in a native 0.8% agarose/TBE gel. Red arrow indicates the band corresponding to ZIKV RNA. Note: IVT and purification were performed using MEGAscript™ T7 Transcription Kit and MEGAclear™ Transcription Clean-Up Kit from Thermo Fisher Scientific. Protocols of these two kits except that no heat was applied during the purification column in the elution step of the viral RNA as high temperature could result in degradation of the viral RNA.

FIGS. 7A-7E depict a fragmentation analysis of ZIKV RNA. FIGS. 7A, 7B, and 7C depict triplicate results of the ZIKV RNA fragmentation. In vitro transcribed ZIKV RNA was fragmented at 94° C. using the RNA fragmentation buffer from New England Biolabs for 1, 3, 6 and 9 minutes. FIG. 7D depicts an example of fragmentation gel image from the RNA fragmentation analyzer showing optimal fragmentation and size following 9 minutes of fragmentation. FIG. 7E depicts detection of fragmented ZIKV RNA with different fragmentation times by using 18 nanoswitches mix. Here, 5 ng (˜8.5×10⁸ copies) of fragmented in vitro transcribed ZIKV RNA was used for each lane.

FIGS. 8A and 8B depict optimization of detection arm length. FIG. 8A depicts a schematic of the DNA nanoswitch. FIG. 8B depicts nanoswitches with detector oligonucleotides of different lengths (10-15 nucleotides long, or 10 nt, 11 nt, 12 nt, 13 nt, 14 nt or 15 nt (nt=number of nucleotides)) were incubated with in vitro transcribed ZIKV RNA that was fragmented at 94° C. with the NEB fragmentation buffer for 3, 6 and 9 minutes. An example 0.8% agarose/TBE gel image showing detection of ZIKV RNA is shown for each fragmentation time. These results revealed optimal detection of ZIKV RNA following 9 minutes of RNA fragmentation and with a nanoswitch containing a 15-nucleotide detector arm length. The nanoswitch used in this experiment is the third nanoswitch in Table 3.

FIGS. 9A and 9B depict considerations for choosing target sequences of viral RNA. FIG. 9A depicts a schematic of self-binding and formation of a stable secondary structure that should be excluded as a target sequence. FIG. 9B depicts an example of two comparable targets when G-U base pairing is taken into consideration.

FIGS. 10A and 10B depict a schematic showing assembly of DNA nanoswitch and interference by excess backbone oligos. FIG. 10A depicts a single stranded M13 DNA is annealed with backbone oligonucleotides and detector strands specific to the RNA target. Normal assembly results in the detection oligonucleotides have free detection arms. FIG. 10B depicts, in contrast, abnormal assembly of the DNA nanoswitch may result when excess backbone oligonucleotides interact with the detector oligonucleotides and occlude the detection arms thus blocking recognition of target RNA.

FIG. 11 depicts a graphical user interface (GUI) for obtaining potential viral RNA targets. More selections could be added to the procedure and the Matlab code can be easily customized to obtain the desired target regions of viral RNAs.

FIG. 12 depicts an analysis of the 18 DNA nanoswitches designed for ZIKV RNA detection. Top panel shows the negative control test of just the different DNA nanoswitches. The middle panel shows the positive control of complementary ssDNA (2 nM) annealed with the corresponding nanoswitch. The bottom panel shows detection of ZIKV RNA by individual DNA nanoswitches. 5 ng (˜8.5×10⁸ copies) of fragmented in vitro transcribed ZIKV RNA was used to test the nanoswitches in 10 μl reaction. * represents dimers formed by DNA nanoswitches.

FIG. 13 depicts an example of gel image of the 18 mixed nanoswitches detection sensitivity test. This is the representative gel shown as inset in FIG. 2F (45 second exposure is shown at the top and 30 second exposure at the bottom).

FIGS. 14A and 14B depict a detection sensitivity test of single nanoswitch. FIG. 14A depicts a sensitivity test of a high-performing single nanoswitch (third nanoswitch listed in Table 3). An example of gel image with detection bands is presented as an inset within the graph and the profiles of the detection bands are shown on the left as an inset. FIG. 14B depicts the entire gel image presented at the bottom of FIG. 14A: A visible band can be seen to at least the 8.5×10⁵ copies/μl (1.4 pM) lane. Experiment was performed in triplicates and error bars represent the standard deviation.

FIG. 15 depicts an analysis of the 12 DNA nanoswitches designed for DENV RNA detection. Top panel shows the negative control test of just the different DNA nanoswitches. The middle panel shows the positive control of complementary ssDNA (2 nM) annealed with the corresponding nanoswitch. The bottom panel shows detection of DENV RNA by individual DNA nanoswitches. 10 ng (˜1.7×10⁹ copies) of fragmented in vitro transcribed DENV RNA was used to test the nanoswitches in 10 μl reaction.

FIG. 16 depicts tuning the loop size of a DNA nanoswitch of the present disclosure. The size of v4-v8 loop is about 2580 bp and the size of v4-v6 loop is about 1260 bp. Note in the table of oligos below, all detection ssDNA oligos are named with prefix v4- or v8- or v6-.

FIGS. 17A and 17B depict targets and a gel image of specificity test with Cambodia and Uganda strains of ZIKV. FIG. 17A depicts the five targets for the specificity test of Cambodia and Uganda strains of ZIKV. Strain-specific nucleotides are colored in red.

FIG. 17B depicts a representative gel image from the assay demonstrating nanoswitch specificity for detecting and differentiating between ZIKV Cambodia and Uganda strains used in FIGS. 3C-3D in the main text. * indicates contaminating cellular DNA left in the total RNA and the area in the red frame at the bottom indicates the unbound fragmented pieces of cellular and viral RNA isolated from mock- and ZIKV-infected Huh7 cells. The oligos of corresponding nanoswitches are listed in Table 7.

FIGS. 18A-18C depict detection of ZIKV RNA in total RNA extracted from human liver cells. FIG. 18A depicts detection of ZIKV RNA in total RNA of infected human liver cells, NS: nanoswitch. FIG. 18B depicts control experiment using total RNA from mock-infected human liver cells. FIG. 18C depicts fragmented total RNA only. Note: the red arrows indicate the detection bands that contain looped DNA nanoswitches and asterisks indicate the genomic DNA in the total RNA.

FIGS. 19A-19C depict gel images of the ZIKV RNA detection in samples mimicking the urine of patients. Triplicate experiments of detecting ZIKV RNA extracted from human urine at FIG. 19A 8.5×10⁵ copies/μl (1.4 pM), FIG. 19B depicts 1.7×10⁵ copies/μl (0.28 pM), and FIG. 19C depicts the negative control. The quantified detection results are presented in FIG. 5A.

FIGS. 20A to 20E depict detection of ZIKV RNA based on pre-amplification with NASBA. FIG. 20A depicts basic process of Nucleic Acid Sequence Based Amplification (NASBA), RT: reverse transcription. FIG. 20B depicts a test of detection based on NASBA amplification. Two targets were chosen on the amplified region of the in vitro transcribed ZIKV RNA (targets A and B in Table 8). FIG. 20C depicts a schematic of viral RNA detection based on NASBA. FIG. 20D depicts a positive detection of ZIKV RNA from infectious virus in PBS. FIG. 20E depicts an example gel images of the ZIKV RNA detection based on NASBA by spiking virus particles into PBS and urine (final concentration is 10%), the nanoswitch used here is the nanoswitch for target A in Table 8.

FIGS. 21A-21C depicts a portable e-gel system for detection of ZIKV RNA based on pre-amplification with NASBA. FIG. 21A depicts a commercially available E-gel system; FIG. 21B depicts an image capture of an E-gel cartridge testing viral nanoswitch detection (run at 48 volts for 1 hour). FIG. 21C depicts a gel image of the detection of ZIKV RNA based on pre-amplification with NASBA. The concentrations of ZIKV particle in the human urine (10%) are 897, 200 and 20 pfu/μl for lane 3, 4 and 5 respectively. The nanoswitch used here is the one for target A in Table 8.

FIGS. 22A-22D depict detection of a SARS-CoV-2 RNA fragment. FIG. 22A depicts a schematic of producing SARS-CoV-2 RNA fragment. FIG. 22B depicts an RT-PCR detection of SARS-CoV-2 RNA in 10% human saliva. Based on the Cq value shown on the right, the detection limitation of RT-PCR in this scenario is about 0.22 fM. FIG. 22C depicts a detection test of SARS-CoV-2 RNA with different concentration in buffer. FIG. 22D depicts detection of SARS-CoV-2 RNA fragment based on NASBA.

FIGS. 23A-23D depicts detection of SARS-CoV-2 full genome RNA in human saliva. FIG. 23A depicts a sketch of two targets selected on the amplified region by using NASBA. FIG. 23B depicts detection of target RNA pieces using a mixture of the two designed nanoswitches. FIG. 23C depicts a demonstration of the detection ability of the SARS-CoV-2 full genome RNA by using NASBA sample. The gel was run at 75 V for 45 min. FIG. 23D depicts detection of different concentrations of SARS-CoV-2 RNA in human saliva. Here, gels were run at 90 V for 25 min.

FIG. 24 depicts development cycle for DNA nanoswitch based detection of viral RNAs. Direct detection can be accomplished in ˜1-13 hours and in only 2-5 hours with pre-amplification. Bottom left shows the minimum equipment (heating block and E-gel system, pipettes, tips, and tubes are not shown here) needed for the methods of the present disclosure.

FIG. 25 is a process sequence of a method 100 of detecting viral nucleic acid in accordance with the present disclosure.

FIG. 26 depicts sample collection, a nanoswitch assay, and a portable reader in accordance with the present disclosure, and further depicts the process flow of a COVID-19 test, readout, shelf-life, and portable read-out. FIG. 26 also depicts a kit including one or more of a swab, nanoswitch, separation medium (gel-based readout), and portable reader.

FIGS. 27A-27D depict a process flow for a method of detecting SARS-CoV-2 RNA in accordance with the present disclosure.

FIGS. 28A and 28B depict a multiple-detection strategy to amplify signal in accordance with the present disclosure. A plurality of coupled detector strands are shown nested and affixed to backbone oligos of a single nanoswitch of the present disclosure.

FIG. 29 depicts RNA detection with in vitro transcribed RNA and sensitivity. Here, in vitro transcribed RNA from Microbiologics (˜1 knt fragment containing N gene) was subjected to a 30 minute reaction with nanoswitches (40 C), followed by 20 minute gel and 5 minute imaging (Bio-Rad Gel Doc). This detection uses 8 targets.

FIG. 30A-30H depicts DNA nanoswitch detection of SARS-CoV-2 RNA.

FIG. 31A-31G depicts a process sequence for improving the sensitivity with mult-targeting nanoswitches.

FIG. 32A-32G depicts a process sequence for the detection of SARS-CoV-2 RNA.

FIG. 33A-D depict detection of clinical SARS-CoV-2 in accordance with the present disclosure. Boxes 33B, 33C, 33D are prophetic examples of suitable process sequences for use herein.

FIG. 34 depicts an embodiment of the present disclosure where a DNA nanoswitch includes two detector strands hybridizable to scaffold and target viral RNA of interest, a DNA nanoswitch assembly, and detection of a target viral RNA.

### DETAILED DESCRIPTION

The present disclosure is directed towards compositions, kits, and methods of detecting a virus by reconfiguring a nucleic acid such as a DNA nanoswitch including: contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. Advantages of the various embodiments of the present disclosure include overcoming biosensing challenges by using programmable DNA nanoswitches for detection of viral RNA at clinically relevant levels.

Embodiments of the compositions, kits, and methods of the present disclosure are validated in the examples below where, e.g., a viral RNA detection strategy was performed using ZIKV as a model RNA virus. Zika has high global health relevance and is a continued threat due to its re-emerging mosquito-borne nature. Although ZIKV infections are typically associated with mild symptoms, they have been linked to devastating birth defects associated with intrauterine infections, development of Guillian-Barré syndrome in adults, and the possibility of sexual transmission (see e.g., D. Musso, D. J. Gubler, Zika Virus. Clinical Microbiology Reviews. 29, 487-524 (2016); L. R. Petersen, et al., Zika Virus. New England Journal of Medicine. 374, 1552-1563 (2016)). Moreover, despite significant advances in understanding the molecular biology of ZIKV, there is still a lack of antiviral drugs and vaccines, making robust detection of ZIKV vital to controlling the spread of the disease and implementing early treatments (See e.g., A. D. T. Barrett, Current status of Zika vaccine development: Zika vaccines advance into clinical evaluation. npj Vaccines. 3, 24 (2018)). Non-limiting example of ribonucleic acid-of-interest for identification or detection herein include RNA from one or more riboviruses, or RNA from one or more RNA viruses. Non-limiting examples of RNA viruses include: virus that causes the common cold, influenza virus, SARS virus, SARS-CoV-2, Dengue virus, hepatitis C virus, hepatitis E virus, West Nile fever virus, Ebola virus, rabies virus, polio virus, measles virus, Zika virus, as well as variants and strains of these viruses. In some embodiments, RNA viruses include those in which The International Committee on Taxonomy of Viruses (ICTV) classifies as RNA viruses such as those that belong to Group III, Group IV or Group V of the Baltimore classification system of classifying viruses and does not consider viruses with DNA intermediates in their life cycle as RNA viruses. In embodiments, viruses with RNA as their genetic material which also include DNA intermediates in their replication cycle, Retrovirus, and include Group VI of the Baltimore classification such as HIV-1 and HIV-2 may also be identified in accordance with the methods of the present disclosure. In some embodiments; the ribonucleic acid of interest may include ribonucleic acid from double stranded RNA viruses, single stranded plus sense RNA viruses or single stranded negative sense RNA viruses. In embodiments, Zika virus is an example of a single-stranded RNA virus of the Flaviviridae family, genus Flavivirus suitable for identification and or detection in accordance with the present disclosure. The complete genome of the Zika virus is known. (See e.g., NCIB accession no. NC_012532).

In embodiments, positive-strand RNA virus, such as sense-strand RNA virus is suitable for detection and/or identification in accordance with the present disclosure. Examples of positive strand RNA viruses include polio virus, Coxsackie virus, echovirus, and the like. In embodiments, negative-strand RNA virus, such as antisense-strand RNA virus (e.g., -ssRNA viruses) is suitable for detection and/or identification in accordance with the present disclosure. Non-limiting examples of ssRNA viruses include Ebola virus, hantaviruses, influenza viruses, Lassa fever virus, rabies virus, family members of these, variants of these, and combinations thereof.

In embodiments, the compositions and methods of the present disclosure are suitable for detecting the presence of viral RNA based on using DNA nanoswitches designed to undergo a conformational change (from linear to looped) upon binding a target viral RNA. In embodiments, the presence of the viral RNA would be indicated by shifted migration of the looped nanoswitch by gel electrophoresis. In some embodiments, the methods include a common nucleic acid staining of the nanoswitch itself that can intercalate thousands of dye molecules to provide an inherently strong signal. In embodiments, as applied here to viral RNA detection, challenges of detecting a long viral RNA (>10,000 nucleotides) in clinically relevant samples have been overcome.

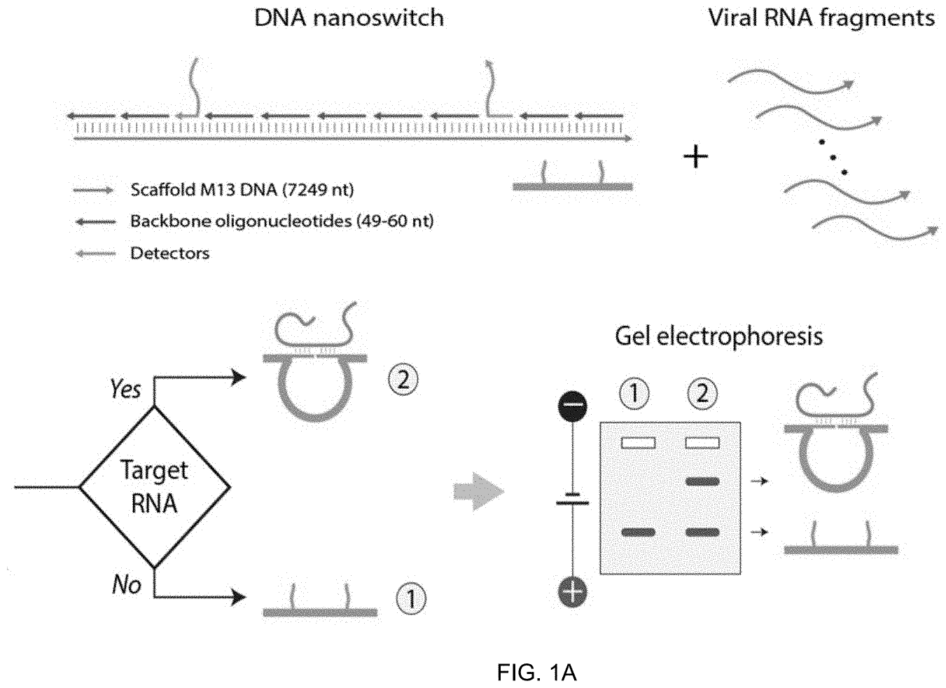

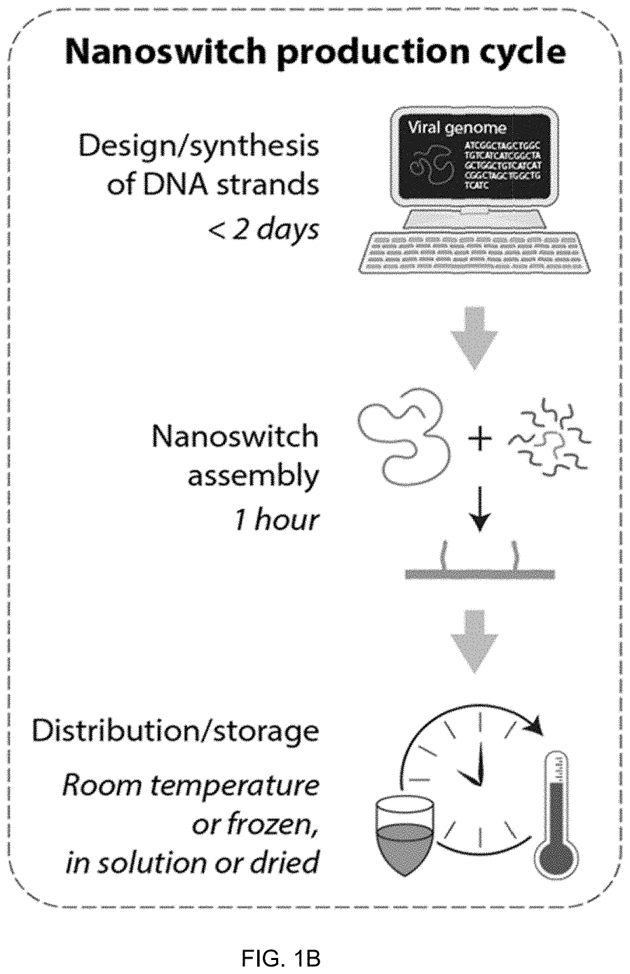

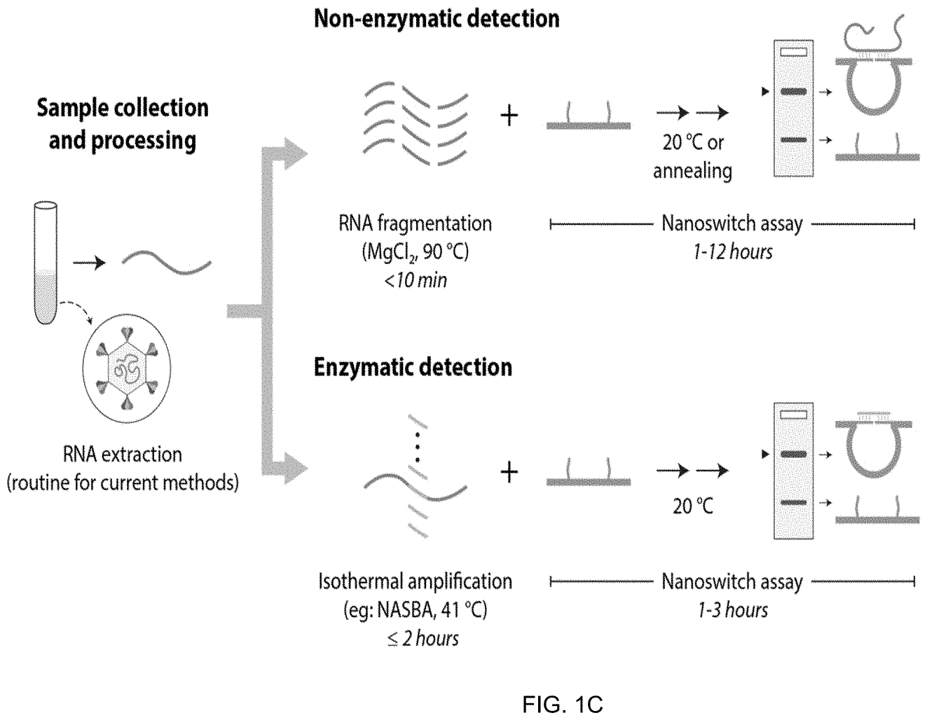

Embodiments of the present disclosure include an RNA fragmentation process, wherein e.g., large RNA strands are separated into small RNA segments, a signal multiplication feature, use of an algorithm for choosing target sequences, and workflows for measuring viral loads in biological and mock clinical samples with or without RNA pre-amplification. In some embodiments, multiplexing is used to detect multiple viruses simultaneously from a single sample and demonstrate high specificity even between closely related strains of virus, such as Zika. In some embodiments, DNA nanoswitches (FIG. 1B) for the detection of SARS-CoV-2 RNA spiked into human saliva are provided. While embodiments, of the present disclosure may be non-enzymatic, embodiments can optionally be combined with an isothermal amplification step, allowing use in low resource areas (FIG. 1C). In embodiments, the direct detection of viral RNA is obtained without amplification advantageously paving the way toward a low-cost assay for detection of RNA viruses.

In embodiments, the compositions and methods of the present disclosure herein relate to altering the conformation of one or more DNA nanoswitches for signaling depending upon signal needs. In some embodiments, the present disclosure relates to a method of detecting a virus by reconfiguring a nucleic acid including: contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed. In embodiments, a process flow subsequently includes processing the mixture under conditions sufficient to separate the first conformation, and when present, the second conformation; and reacting the first conformation, and when present, the second conformation with an indicator under conditions sufficient to form a signal. In embodiments, reacting may be as simple as adding a dye to make a first conformation and/or second confirmation detectable. In embodiments, the ribonucleic acid-of-interest is derived from an RNA virus in a biological specimen or sample.

The embodiments of the present disclosure may advantageously provide nucleic acid complexes configured to provide one or more useful signals such as the presence or absence of viral RNA. In embodiments, the present disclosure advantageously provides improved methods, compositions, and assays for the detection, or identification of one or more RNA targets-of-interest such as one or more viral RNAs, or species or fragments thereof, and combinations thereof.

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the terms “bind” and “binding” generally refer to a non-covalent interaction between a pair of partner molecules or portions thereof that exhibit mutual affinity or binding capacity. In embodiments, binding can occur such that the partners are able to interact with each other to a substantially higher degree than with other, similar substances. This specificity can result in stable complexes that remain bound during handling steps such as chromatography, centrifugation, filtration, and other techniques typically used for separations and other processes.

The terms “deoxyribonucleotide” and “DNA” refer to a nucleotide or polynucleotide including at least one ribosyl moiety that has an H at the 2′ position of a ribosyl moiety. In embodiments, a deoxyribonucleotide is a nucleotide having an H at its 2′ position.

By “hybridizable” or “complementary” or “substantially complementary” a nucleic acid (e.g. RNA, DNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (e.g., a nucleic acid specifically binds to a complementary nucleic acid) under the appropriate in vitro and/or in vivo conditions of temperature and solution ionic strength. Standard Watson-Crick base-pairing includes: adenine/adenosine) (A) pairing with thymidine/thymidine (T), A pairing with uracil/uridine (U), and guanine/guanosine) (G) pairing with cytosine/cytidine (C). In addition, for hybridization between two RNA molecules, and for hybridization of a DNA molecule with an RNA molecule (e.g., when a DNA nanoswitch base pairs with a target RNA, etc.): G can also base pair with U. For example, G/U base-pairing is partially responsible for the degeneracy (i.e., redundancy) of the genetic code in the context of tRNA anti-codon base-pairing with codons in mRNA. In embodiments, hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). It is understood that the sequence of a polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure, a ‘bulge’, and the like). A polynucleotide can include 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. The remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “elute” and “eluting” refer to the disruption of non-covalent interactions between partner molecules such that the partners become unbound from one another. In embodiments, the disruption can be effected via introduction of a competitive binding species, introduction of RNase, or via a change in environmental conditions (e.g., ionic strength, pH, or other conditions).

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

An “isolated nucleic acid molecule” is a polymer of RNA or DNA that is single- or double-stranded, optionally containing synthetic, non-natural or altered nucleotide bases. An isolated nucleic acid molecule in the form of a polymer of DNA may be one or more segments of cDNA, genomic DNA or synthetic DNA.

The term “nucleotide” refers to a ribonucleotide or a deoxyribonucleotide or modified form thereof, as well as an analog thereof.

The term “nanoswitch” refers to a nucleic acid molecule, either single- or double-stranded, which is modified to contain segments of nucleic acids in a manner that would not otherwise exist in nature, that assumes a linear (or open) conformation in the absence of a predetermined nucleic acid or a looped (or closed) formation in the presence of the same predetermined nucleic acid.

Nucleic acid construct: The term “nucleic acid construct” as used herein refers to a nucleic acid molecule, either single- or double-stranded, which is modified to contain segments of nucleic acids in a manner that would not otherwise exist in nature.

As used herein, the term “nucleic acid molecule” refers to any molecule containing multiple nucleotides (e.g., molecules including a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As described further below, bases include C, T, U, C, and G, as well as variants thereof. As used herein, the term refers to ribonucleotides (including oligoribonucleotides (ORN)) as well as deoxyribonucleotides (including oligodeoxynucleotides (ODN)). The term shall also include polynucleosides (e.g., a polynucleotide minus the phosphate) and any other organic base containing polymer. Nucleic acid molecules can be obtained from existing nucleic acid sources (e.g., genomic or cDNA), but include synthetic (e.g., produced by oligonucleotide synthesis). In embodiments, the terms “nucleic acid” “nucleic acid molecule” and “polynucleotide” may be used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

In embodiments, the term “oligonucleotide” refers to a polynucleotide of between 4 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized.

The term “isolated” means a substance in a form or environment that does not occur in nature. Non-limiting examples of isolated substances include (1) any non-naturally occurring substance, (2) any substance such as a variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

The term “polynucleotide” refers to polymers of nucleotides. In embodiments, the term polynucleotide includes but is not limited to DNA, RNA, DNA/RNA hybrids including polynucleotide chains of regularly and irregularly alternating deoxyribosyl moieties and ribosyl moieties (e.g., wherein alternate nucleotide units have an —OH, then and —H, then an —OH, then an —H, and so on at the 2′ position of a sugar moiety), and modifications of these kinds of polynucleotides wherein the attachment of various entities or moieties to the nucleotide units at any position are included.

The term “polyribonucleotide” refers to a polynucleotide including two or more modified or unmodified ribonucleotides and/or their analogs.

The term “ribonucleotide” and the phrase “ribonucleic acid” (RNA), refer to a modified or unmodified nucleotide or polynucleotide including at least one ribonucleotide unit. A ribonucleotide unit includes an oxygen attached to the 2′ position of a ribosyl moiety having a nitrogenous base attached in N-glycosidic linkage at the 1′ position of a ribosyl moiety, and a moiety that either allows for linkage to another nucleotide or precludes linkage.

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may include additions or deletions (e.g., gaps) as compared to the reference sequence (which does not include additions or deletions) for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity.

It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

In embodiments, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid or nucleotide in the two aligned sequences on a given position in the alignment; and 3) dividing the number of exact matches with the length of the reference sequence. In one embodiment, the degree of sequence identity between a query sequence and a reference sequence is determined by: 1) aligning the two sequences by any suitable alignment program using the default scoring matrix and default gap penalty; 2) identifying the number of exact matches, where an exact match is where the alignment program has identified an identical amino acid; or nucleotide in the two aligned sequences on a given position in the alignment; and 3) dividing the number of exact matches with the length of the longest of the two sequences. In some embodiments, the degree of sequence identity refers to and may be calculated as described under “Degree of Identity” in U.S. Pat. No. 10,531,672 starting at Column 11, line 56. U.S. Pat. No. 10,531,672 is incorporated by reference in its entirety. In embodiments, an alignment program suitable for calculating percent identity performs a global alignment program, which optimizes the alignment over the full-length of the sequences. In embodiments, the global alignment program is based on the Needleman-Wunsch algorithm (Needleman, Saul B.; and Wunsch, Christian D. (1970), “A general method applicable to the search for similarities in the amino acid sequence of two proteins”, Journal of Molecular Biology 48 (3): 443-53). Examples of current programs performing global alignments using the Needleman-Wunsch algorithm are EMBOSS Needle and EMBOSS Stretcher programs, which are both available on the world wide web at www.ebi.ac.uk/Tools/psa/. In some embodiments a global alignment program uses the Needleman-Wunsch algorithm, and the sequence identity is calculated by identifying the number of exact matches identified by the program divided by the “alignment length”, where the alignment length is the length of the entire alignment including gaps and overhanging parts of the sequences. In embodiments, the mafft alignment program is suitable for use herein.

The term “recombinant” when used herein to characterize a nucleic acid sequence such as a plasmid, vector, construct, or complex refers to an artificial combination of two otherwise separated segments of sequence, e.g., by chemical synthesis and/or by manipulation of isolated segments of nucleic acids by genetic engineering techniques.

The term “substantially purified,” as used herein, refers to a component of interest that may be substantially or essentially free of other components which normally accompany or interact with the component of interest prior to purification. By way of example only, a component of interest may be “substantially purified” when the preparation of the component of interest contains less than about 30%, less than about 25%, less than about 20%, less than about 15%, less than about 10%, less than about 5%, less than about 4%, less than about 3%, less than about 2%, or less than about 1 (by dry weight) of contaminating components. Thus, a “substantially purified” component of interest may have a purity level of about 70%, about 75%, about 80%, about 85%, about 90%, about 95%, about 96%, about 97%, about 98%, about 99% or greater.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Before embodiments are further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DESCRIPTION OF CERTAIN EMBODIMENTS OF THE PRESENT DISCLOSURE

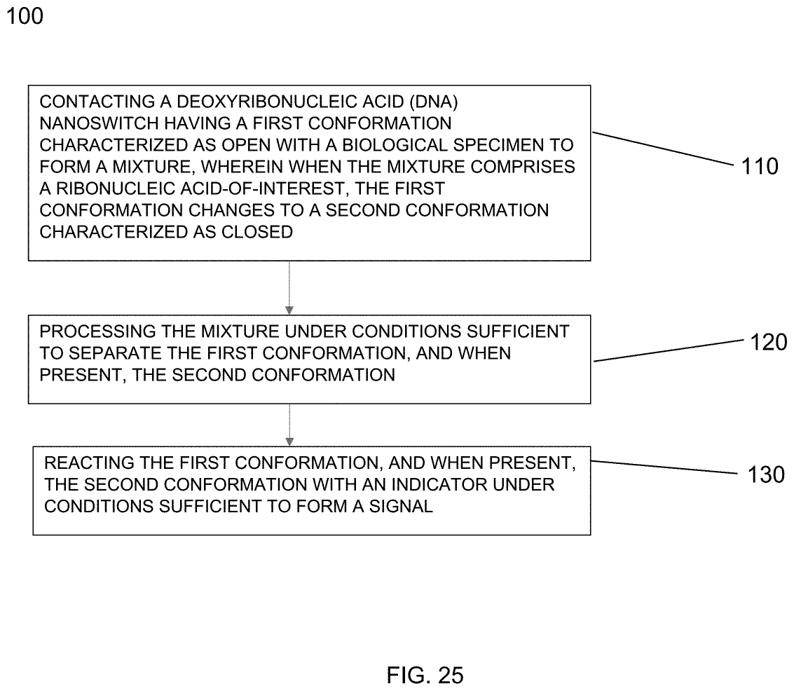

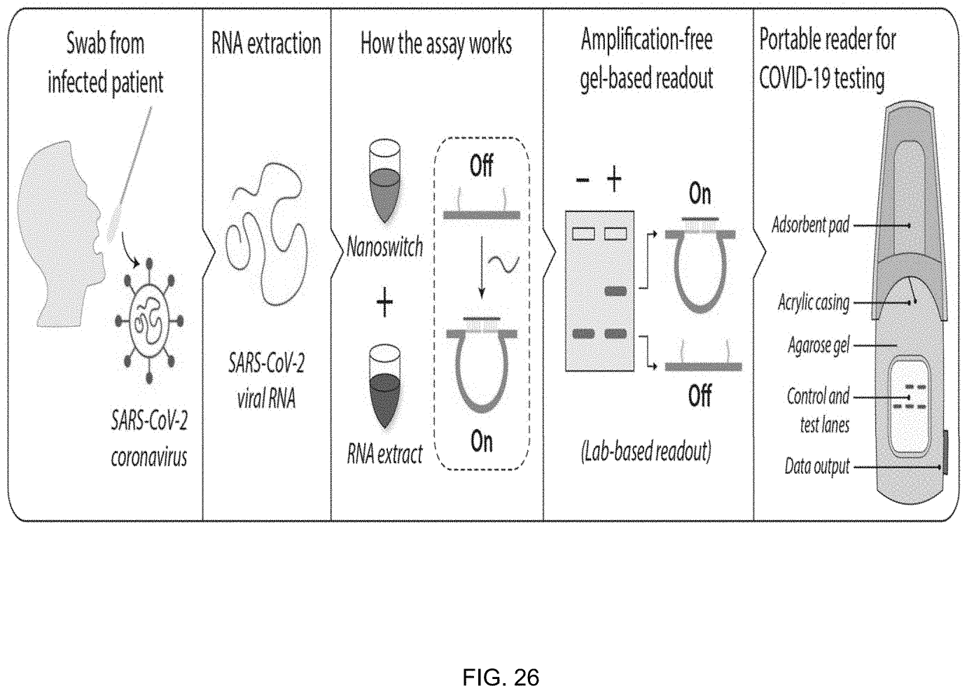

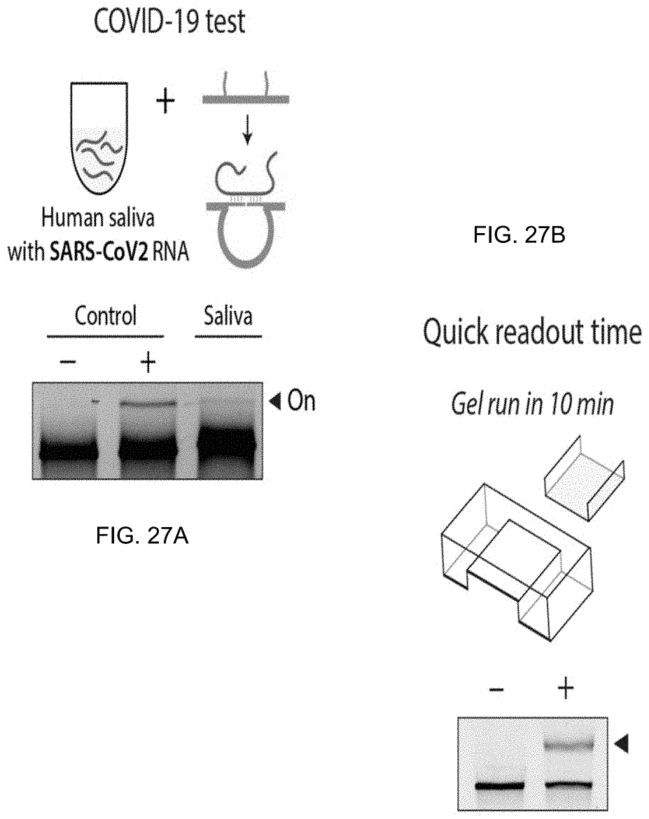

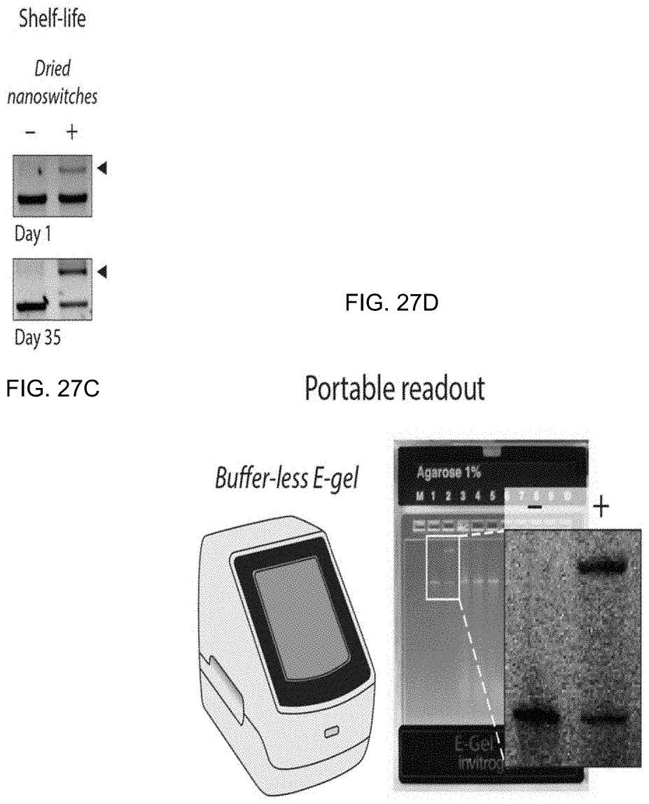

In embodiments, the present disclosure relates to kits, compositions, or one or more methods of detecting an RNA virus by reconfiguring a nucleic acid such as a polynucleotide including: contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. FIG. 25 is a flow diagram of a method 100 for reconfiguring a nucleic acid or polynucleotide in accordance with some embodiments of the present disclosure. The method 100 is described below with respect to the stages of processing as depicted in, e.g., FIGS. 1A-1C and may be performed, for example, in a suitable labware or electrophoresis gel medium as shown below.

In embodiments, one or more DNA nanoswitches are preformed or preselected to combine with a preselected ribonucleotide-of-interest such as a preselected RNA, preselected RNA oligonucleotide, preselected RNA polynucleotide, viral RNA, fragments thereof, and combinations thereof. In embodiments, a DNA nanoswitch-nucleic acid complex suitable for use herein is a nucleic acid complex for use in detecting targets such as RNA virus. Targets are detected based on their interactions with the DNA nanoswitch-nucleic acid complex and the conformational changes that are induced in the nanoswitches and/or DNA nanoswitch-nucleic acid complex as result of such interactions. In embodiments, the DNA nanoswitch-nucleic acid complexes are designed so that in the absence of the target they typically maintain a linear (or open) conformation or shape and assume a looped (or closed) conformation or shape in the presence of a target such as viral RNA. In embodiments, conformation(s) refer to one or more structures of a DNA nanoswitch, including but not limited to any of the spatial arrangement which the atoms in the nanoswitch molecule may adopt and freely convert between, such as by rotation about individual single bonds therein. In embodiments, a linear or substantially linear conformation and a looped or closed conformation are detectable and physically separable from each other using various techniques including but not limited to gel electrophoresis. In the context of gel electrophoresis, the open and closed conformations migrate to different extents through a gel, and they can emit signal (such as when stained with a dye) from the gel in order to, in some embodiments, further inform that the target has altered the conformation of the nanoswitch. In embodiments, linear conformations migrate more faster through a gel medium under the same electrophoretic conditions, in comparison to looped or closed conformations formed of the linear conformations.

In embodiments, nanoswitches and DNA nanoswitch-nucleic acid complexes of the present disclosure are designed to detect and/or identify one or more preselected viral RNA targets. For example, in embodiments, a nanoswitch of the present disclosure is configured to bind to a binding partner to alter the shape of the nanoswitch, e.g., viral RNA. In embodiments, a predetermined viral RNA or fragment thereof is a binding partner with a DNA nanoswitch of the present disclosure. In embodiments, the binding partner may be one or more preselected ribonucleic acids, e.g. viral RNA, or fragments thereof that bind(s) to the nanoswitch and lock(s) the nanoswitch into a closed conformation. In embodiments, the preselected nucleic acid is RNA based on sequence complementarity. In embodiments, and as shown in FIG. 1C, nanoswitches of the present disclosure bind to a pre-selected ribonucleic acid such as viral RNA fragments and change the conformation of the nanoswitch to a closed conformation. The closed conformation presence is indicated by gel electrophoresis and the presence of a corresponding band.

Non-limiting examples of viral RNA targets, and fragments thereof include targets derived from the viral RNA disposed within e.g., the virus that causes the common cold, influenza virus, SARS virus, SARS-CoV-2, Dengue virus, hepatitis C virus, hepatitis E virus, West Nile fever virus, Ebola virus, rabies virus, polio virus, measles virus, as well as variants and strains of these viruses. In embodiments, the viral RNA targets include one or more segments of RNA having a first length, such as a length between 20 nucleotides to 1000 nucleotides, which may be fragmented into a shorter length. In embodiments, the target RNA has a length of 20-200 nucleotides, 20-100 nucleotides, 20-60 nucleotides, 20-50 nucleotides, 20-40 nucleotides, 20-30 nucleotides, and the like.

In embodiments, detection of one or more viral RNA targets-of-interest is important for a variety of applications including for example in the fields of medicine and forensics. In some embodiments, the present disclosure provides a programmable nucleic acid-based nanoswitch that undergoes a pre-defined conformational change upon contact with a target ribonucleic acid such as viral RNA, converting a nanoswitch from a linear “open” state to a looped “closed” state (or conformation) within a DNA-nanoswitch-nucleic acid complex.

In embodiments, the looped “locked” state relates to a DNA complex or conformation that includes a combination of the DNA nanoswitch and a preselected ribonucleic acid combined to form a DNA nanoswitch-nucleic acid complex having a second conformation, wherein the second conformation is characterized as locked.

In embodiments, a DNA nanoswitch and/or DNA nanoswitch ribonucleic acid complex can be detected using separation techniques such as standard gel electrophoresis, which are capable of physically separating the open and locked conformations from each other and from other components in a mixture, and in some instances also are capable of facilitating isolation of DNA nanoswitch nucleic acid complex having a first conformation and/or nanoswitch having a second conformation different than the first conformation. In embodiments, other separation medium suitable for use herein may include liquid chromatography medium such as those used HPLC columns, or other medium such as those used in capillary electrophoresis.

In embodiments, the present disclosure demonstrates successful detection of one or more viral RNAs. In embodiments, the detection method can be accomplished quickly, including as demonstrated herein within 15 minutes from sample mixture to readout. The approach is a low cost and technically accessible, and thus well-suited for point-of-use detection.

In addition, the compositions of the present disclosure may also be used to simultaneously detect more than one viral RNA targets-of-interest. For example, the nanoswitch nucleic acid complex may be designed to include one or more nucleic acids configured to form the close looped shaped, wherein the one on more nucleic acids have different lengths and/or discernable structure. Variation in loop size may facilitate varying reaction conditions among a plurality of targets-of-interest such as different types of viral RNAs and facilitate detection between various viral RNAs.

In embodiments, a nucleic acid such as a DNA nanoswitch and/or DNA nanoswitch-ribonucleic acid complex, as described herein includes a scaffold nucleic acid hybridized in a sequence specific manner to a plurality of oligonucleotides. The scaffold and the oligonucleotides may be referred to herein as being single-stranded. In embodiments, prior to hybridization to each other, both nucleic acid species are single-stranded. In embodiments, upon hybridization, such as when subjected to conditions suitable for hybridization, a double-stranded nucleic acid is formed. Typically, the oligonucleotides hybridize to the scaffold nucleic acid in a consecutive, non-overlapping, manner. FIG. 1A depicts the formation of a DNA nanoswitch including scaffold M13 DNA, backbone oligonucleotides such as those having a length of 49-60 nucleotides, and two detector strands. In embodiments, each detector strand includes a first segment and a second segment. In embodiments, a first segment of a detector strand is hybridizable, complementary, or substantially complementary to a segment of a backbone oligonucleotide, and a second segment of the detector strand is hybridizable, complementary, or substantially complementary to a segment of a preselected target RNA segment on e.g., an RNA-of-interest in accordance with the present disclosure. In embodiments, a nanoswitch includes a pair of detector strands, wherein each pair of detector strands includes a first segment of a detector strand which is hybridizable, complementary, or substantially complementary to a segment of a backbone oligonucleotide, and a second segment of the detector strand which is hybridizable, complementary, or substantially complementary to a segment of a preselected target RNA segment on e.g., an RNA-of-interest in accordance with the present disclosure. In embodiments, pairs of detector strands include a second segment of the detector strand which is hybridizable, complementary, or substantially complementary to a first segment of a preselected target RNA segment on e.g., an RNA-of-interest in accordance with the present disclosure, and a second segment of a preselected target RNA segment on e.g., an RNA-of-interest in accordance with the present disclosure, wherein the first segment of a preselected target RNA is adjacent, or immediately adjacent, the second segment of preselected target RNA. In embodiments, adjacent or immediately adjacent may include a segment that is immediately upstream or downstream a nucleotide molecule such as in a 5′ to 3′ orientation.

Still referring to FIG. 1A, in some non-limiting embodiments, the nucleic acid such as nanoswitches are formed by hybridizing a scaffold nucleic acid to one or more oligonucleotides. Accordingly, in embodiments, scaffold nucleic acids of the present disclosure are hybridizable, complementary, or substantially complementary to a one or more backbone oligonucleotides of the present disclosure. The disclosure contemplates any variety of means and methods for generating the nanoswitches and nanoswitch-nucleic acid complexes described herein. It is also to be understood that while for the sake of brevity the disclosure refers to oligonucleotides that are hybridized or substantially complementary to a scaffold nucleic acid, such a complex may have been formed by hybridizing single stranded scaffold to single stranded oligonucleotides, but it is not intended that it was exclusively formed in this manner. In embodiments, the nucleic acid complexes such as nanoswitches and nanoswitch-ribonucleic acid may include double-stranded and single-stranded regions. As used herein, a double-stranded region is a region in which all nucleotides on a scaffold are hybridized to their complementary nucleotides on the oligonucleotide. Double-stranded regions may include “single-stranded nicks” as the hybridized oligonucleotides typically are not ligated to each other. The single-stranded regions are scaffold sequences that are not hybridized to oligonucleotides. Certain complexes may include one or more single-stranded regions in between double-stranded regions (typically as a result of unhybridized nucleotides in between adjacent hybridized oligonucleotides). The complexes may be at least 50%, at least 60%, at least 70%, at least 80%, at least 90%, at least 95%, at least 96%, at least 97%, at least 98%, at least 99%, or 100% double-stranded. In some embodiments, they are at least 80% double stranded.

In embodiments, the DNA nanoswitch are modular complexes to which can be attached one or more nucleic acids of interest such as one or more oligonucleotides, one or more binding pairs of interest, and the like. The terms attach, link and conjugate are used interchangeably throughout this disclosure unless otherwise stated. In embodiments, as shown in FIG. 1A, the combination of a DNA nanoswitch and ribonucleic acid-of-interest such as viral RNA forms a DNA nanoswitch-ribonucleic acid complex of the present disclosure, in a locked looped formation. In embodiments, removal of the ribonucleic acid-of-interest forms a DNA nanoswitch of the present disclosure, in an open, non-looped, or linear conformation.

In embodiments, the nanoswitches of the present disclosure provided herein are stable in complex fluids such as but not limited to serum-containing samples, including up to 30% FBS. In some embodiments, nanoswitches for use herein are configured to convert from unbound to bound forms in the presence of complex fluids (e.g., 30% FBS). Moreover, the nanoswitches are also stable for an extended period of time. Once synthesized, the nanoswitches may be dried and stored for days, weeks or months. Similarly, DNA nanoswitch-nucleic acid complexes of the present disclosure may be stable for an extended period of time. Once synthesized, the DNA nanoswitch-nucleic acid complexes may be dried and stored for days, weeks or months.

In some embodiments, the nanoswitches of the present disclosure and/or DNA nanoswitch-ribonucleic acid complexes of the present disclosure can be made using nucleic acid nanostructure techniques such as but not limited to DNA origami. (See e.g., Rothemund P. W. K. (2006) Nature 440: 297-302; Douglas S. M. et al. (2009) Nature 459: 414-8). In embodiments, the nanoswitches of the present disclosure may be formed as described in U.S. Patent Publication No. 2018/0223344 entitled Compositions and Methods for Analyte Detection Using Nanoswitches published on 9 Aug. 2018 to Chandrasekaren et al. (herein entirely incorporated by reference).

### Scaffolds

In embodiments, scaffold nucleic acid suitable for use herein may be of any length sufficient to allow association (i.e., binding) and dissociation (i.e., unbinding) of binding partners to occur and to be distinguished from other association and/or dissociation events using the read out methods provided herein, including gel electrophoresis.

In embodiments, the scaffold nucleic acid is at least 500 nucleotides in length, and it may be as long as 50,000 nucleotides in length (or it may be longer). The scaffold nucleic acid may therefore be 1000-20,000 nucleotides in length, 1000-15,000 nucleotides in length, 1000-10,000 in length, or any range therebetween. In some embodiments, the scaffold ranges in length from about 5,000-10,000 nucleotides, and may be about 7000-7500 nucleotides in length or about 7250 nucleotides in length.

FIG. 1A depicts scaffold M13 DNA (7249 nt). In some embodiments, the scaffold may be a naturally occurring nucleic acid (e.g., M13 scaffolds such as M13mp18). M13 scaffolds are disclosed by Rothemund 2006 Nature 440:297-302, the teachings of which are incorporated by reference herein. Such scaffolds are about 7249 nucleotides in length.

In some embodiments, the scaffold nucleic acid may also be non-naturally occurring nucleic acids such as polymerase chain reaction (PCR)-generated nucleic acids, rolling circle amplification (RCA)-generated nucleic acids, etc. In some embodiments, the scaffold nucleic acid is rendered single-stranded either during or post synthesis. Methods for generating a single-stranded scaffold include asymmetric PCR. Alternatively, double-stranded nucleic acids may be subjected to strand separation techniques in order to obtain the single-stranded scaffold nucleic acids. The scaffold nucleic acid may comprise DNA, RNA, DNA analogs, RNA analogs, or a combination thereof, provided it is able to hybridize in a sequence-specific and non-overlapping manner to the oligonucleotides or backbone oligonucleotides of the present disclosure. In some instances, the scaffold nucleic acid is a DNA.

### Oligonucleotides

In embodiments, the scaffold nucleic acid is hybridized to a plurality of oligonucleotides, such as the backbone oligonucleotides depicted in FIG. 1A. Each of the plurality of oligonucleotides is able to hybridize, thus is hybridizable, complementary, or substantially complementary to a scaffold nucleic acid in a sequence-specific and non-overlapping manner (i.e., each oligonucleotide hybridizes to a distinct sequence in the scaffold). The length and the number of oligonucleotides used may vary. In some instances, the length and sequence of the oligonucleotides is chosen so that each oligonucleotide is bound to the scaffold nucleic acid at a similar strength. This is important if a single condition is used to hybridize a plurality of oligonucleotides to the scaffold nucleic acid, such as for example in a one-pot synthesis scheme.

In embodiments, the number of oligonucleotides will depend in part on the application, the length of the scaffold, and the length of the oligonucleotides themselves. In embodiments, the oligonucleotides are designed to be of approximately equal length. In some embodiments, the oligonucleotides may be about 20-100 nucleotides in length. The oligonucleotides may be, without limitation, about 20, about 30, about 40, about 50, about 60, about 70, about 80, about 90 or about 100 nucleotides in length. In some embodiments, the oligonucleotides may be about 40-80 nucleotides in length. In some embodiments, the oligonucleotides may be about 60 nucleotides in length.

The number of oligonucleotides in the plurality may be about 70, about 80, about 90, about 100, about 110, about 120, about 130, about 140, about 150, about 160, about 170, about 180, about 190, about 200, about 300, about 400, about 400, about 500, about 600, about 700, about 800, about 900, or about 1000, without limitation.

In some embodiments, the nucleic acid complex may include the M13 ssDNA as the scaffold and about 120 oligonucleotides each equal to or about 60 nucleotides in length.

In embodiments, the oligonucleotides may be characterized as modified or unmodified or variable oligonucleotides. In embodiments, the variable oligonucleotides may be conjugated to reactive groups that are not normally present in a nucleic acid sequence, such as for example click chemistry reactive groups, or they may be conjugated to target-specific binding partners such as antibodies or antibody fragments, or they may include other moieties which are not typically present in an unmodified oligonucleotide. An example is a variable oligonucleotide including a phosphate at their 5′ end (referred to herein as a 5′ phosphate). Oligonucleotides having this latter modification are used herein in the detection of target nucleic acids, and in this context such oligonucleotides are referred to as “detector” strands since they are preselected to bind to nucleic acids of the interest via hybridization to form a DNA Nanoswitch-nucleic acid complex having a first conformation, wherein the first conformation is characterized as locked.

In some embodiments, the first and last oligonucleotides as well as “internal” oligonucleotides, typically at pre-defined positions along the length of the scaffold, may be modified oligonucleotides. The position of the variable oligonucleotides may be, but are not necessarily, evenly distributed along the length of the scaffold

### Binding Interactions and Looped Conformations

Binding interactions and looped conformations are shown in FIG. 1A. In embodiments, the location of the variable oligonucleotides dictates the location of the various substituents in the complex, such as the two or more detector strands, nucleic acid binding partners, etc. It also dictates the size of the loops that are formed once the various substituents bind to each other. This will in turn dictate the migration distance of the looped (closed) complex (such as through a gel), and thus the ability of the end user to physically separate and thus distinguish between complexes of interest (e.g., locked complexes such as nanoswitch-ribonucleic acid complexes having a second conformation, wherein the first conformation is characterized as open and/or linear) and nanoswitch-ribonucleic acid complexes having a second conformation (e.g., closed complexes such as where the ribonucleic acid-of-interest binds to the DNA nanoswitch to form a looped or closed conformation).

In embodiments, a nanoswitch may include a first and a second oligonucleotide such as a first and second detector strand that together hybridize to one or more preselected ribonucleic acids-of-interest such as preselected viral RNA. In these embodiments, the hybridization of the nanoswitch to the ribonucleic acid is considered a first binding interaction. Alternatively, a second binding interaction may be an additional binding interaction that occurs upon hybridization of a second nucleic acid such as to a second pair of detector strands. In embodiments, a single nanoswitch of the present disclosure may include 1-75 pairs of predetermined detector strands of the present disclosure, such as 2-50 pairs of detector strands, 5-30 pairs of detectors strands, 10-25 pairs of detector strands, or 20, 21, 22, 23, 24, 25, 26, 27 pairs of detector strands.

In embodiments, the nanoswitch is designed to detect one target ribonucleotide by hybridization of a first oligonucleotide and a second oligonucleotide, each having an overhang (i.e., a single-stranded region that is available for hybridization to the nucleic acid such as an oligo-ribonucleotide of interest). The first and second oligonucleotides, in this example, may be referred to as partially hybridized to the scaffold since each has a single-stranded overhang region and a region that is hybridized to the scaffold. The first and second oligonucleotides may be denoted as “detector 1” and “detector 2”. The overhangs may be referred to herein as 3′ overhangs and 5′ overhangs, referring to the directionality of the single-stranded region. The distance between the first and the second oligonucleotides, when bound to the scaffold, dictates the size of the loop and ultimately the migration distance of the nanoswitch when it is bound to the target (or when it is stabilized) via a latch binding interaction. In embodiments, the detector length may have a length of 5 to 30 or 7-20 nucleotides. In embodiments, the overhang segment of the detector may be about half the length of the total detector strand length.

In some embodiments, the first oligonucleotide and the second oligonucleotide, e.g., detector strands, are separated from each other by 100-6,000 nucleotides. In some embodiments, the first oligonucleotide and the second oligonucleotide are separated from each other by 500 to 5,000 nucleotides, 600-5,000 nucleotides, 1,000-5,000 nucleotides, or 1,000-3,000 nucleotides. In some embodiments, the first oligonucleotide and the second oligonucleotide are separated from each other by at least 500, 600, 700, 800, 900, 1,000, 1,500, 2,000, 2,500, 3,000, 3,500, 4,000, 4,500, 5,000, 5,500, 6,000, or more nucleotides. In some embodiments, the first oligonucleotide and the second oligonucleotide are located about equidistant about the center of the scaffold nucleic acid. In some embodiments, the first and second oligonucleotides bind to regions of the scaffold nucleic acid that are internal to the scaffold (i.e., such regions exclude the most 5′ and the most 3′ nucleotides of the scaffold).

### Gel Electrophoresis

In embodiments, such as when measured using gel electrophoresis, the open and closed nanoswitch conformations migrate differentially through a medium such as a gel medium. In embodiments, a circular scaffold such as circular M13 migrates the slowest, a linearized double-stranded version of M13 (without internal binding interactions) migrates fastest, and nanoswitches in looped conformations migrate in between. In embodiments, the migration distance differs based on the length of the loop. As an example, loops that are on the order of about 2590 base pairs are clearly distinguishable from loops that are on the order of about 600 base pairs. Loops of other sizes can also be distinguished from each other. The ability to distinguish between loops of different sizes means that the presence (or absence) of multiple targets (each detected by a complex having a loop of a particular size) can be determined simultaneously in a multiplexed assay. Such methods may be used to detect the presence of a single or multiple target and may form the basis of a diagnostic assay. Moreover, it should also be understood that nanoswitches having one loop can also be distinguished from nanoswitches having more than one loop, including those that have 2, 3 or more loops. In embodiments, a single type of nanoswitch can be used to detect two different targets and depending on the conformation of the nanoswitch (as determined by its migration distance in a gel), an end user can determine whether either or both targets are present in a sample. These nanoswitches can then also be extracted from the gel and the bound targets can be isolated.

In embodiments, electrophoresis is performed wherein a gel is run at 4 degrees Celsius to maintain the interaction of the targets to their binding partners (e.g., the binding of a protein target to target-specific antibodies) or to maintain latch binding interactions. It is contemplated that other separation medium is suitable for use herein such those used in capillary electrophoresis and liquid chromatography. In embodiments, reacting a first conformation of a nanoswitch, and when present, the second conformation of a nanoswitch with an indicator under conditions sufficient to form a signal refers to staining the one or more nanoswitches with a dye suitable for gel electrophoresis sufficient to fluoresce, glow or emit light as a signal.

### Nanoswitches

In some embodiments, nanoswitches designed for DNA nanoswitch-ribonucleic acid complex formation are provided, such as those shown in FIG. 1A. In some embodiments, nanoswitches of the present disclosure include a scaffold nucleic acid hybridized to a plurality of oligonucleotides such as detectors, as described herein. In embodiments, the nanoswitch includes a first and a second oligonucleotide such as detector strands that are partially hybridized to the scaffold nucleic acid (i.e., each of these oligonucleotides is partially hybridized to the scaffold and thus each is partially single-stranded). The first oligonucleotide includes a 3′ overhang and the second oligonucleotide includes a 5′ overhang.

In embodiments, the 3′ overhang is not complementary to the 5′ overhang, and rather both the 3′ and the 5′ overhangs are complementary to a ribonucleic acid-of-interest such as viral RNA and suitable for forming a DNA nanoswitch-ribonucleic acid complex of the present disclosure. In embodiments, the entire ribonucleic acid-of-interest such as viral RNA (referred to as “Target “Key” or “oligonucleotide”) hybridizes to a combination of the 3′ and 5′ overhang. However, in embodiments, the method can also be performed in which the 3′ and 5′ overhangs are designed to hybridize only the 5′ and 3′ regions of a ribonucleic acid-of-interest, with the internal or middle region of the target ribonucleic acid remaining unhybridized. In this latter instance, the nanoswitch is designed to include a plurality of ribonucleic acids-of-interest differing sequences provided that they are at least complementary to the 3′ and 5′ overhangs. In embodiments, the nanoswitch detects non-adjacent sequences on the target. Such non-adjacent sequences may be separated by 1 or 2 nucleotides or by 10's or 100's of nucleotides, without limitation. In embodiments, ribonucleic acid-of-interest hybridized to the nanoswitch. In embodiments, the nanoswitch detects adjacent sequences on the target, depending upon the predetermined selection of overhang segments of the detector strands.

In embodiments, a nucleic acid structure of the present disclosure includes a DNA nanoswitch-ribonucleic acid complex including a deoxyribonucleic acid (DNA) nanoswitch and an oligonucleotide, wherein the DNA nanoswitch and oligonucleotide are configured to form a DNA-nanoswitch-ribonucleic acid complex having a second conformation characterized as locked, and a first conformation characterized as open in the absence of ribonucleotide such as viral RNA. In embodiments, the nanoswitch is configured such that the 3′ and 5′ overhangs come into sufficient proximity to each other in the presence of the ribonucleic acid-of-interest, and that it is only once the ribonucleic acid-of-interest hybridizes to the 3′ and 5′ overhangs that a looped conformation of a DNA-nanoswitch-ribonucleic acid complex is formed.

In some embodiments, the overhangs may be of different or identical lengths, relative to each other. The overhang length may range from 5-20 nucleotides in length, without limitation. The overhangs may have a length of 5 or more, or 6 or more, or 7 or more nucleotides. One or both overhangs may have a length of 7, 8, 9, 10, 11, 12, 13, 14, 15, 16, 17, 18, 19 or 20 nucleotides. The combined length of the overhangs may vary and may depend on their sequence and the length of the target nucleic acid. Their combined length may be 14 nucleotides or longer, without limitation. In some instances, the 3′ overhang and the 5′ overhang are of different lengths and their combined length is at least about 22 nucleotides.

In some embodiments, the combined length of the overhangs may be the same length as the ribonucleic acid-of-interest. Alternatively, the combined length of the overhangs may be longer or shorter than the length of the nucleic acid-of-interest. In some embodiments, the ribonucleic acid-of-interest may not bind to both overhangs to the same extent. In other words, one overhang may share more sequence complementarity with the ribonucleic acid-of-interest than the other overhang. In some embodiments, the overhangs will be referred to herein as the 3′ and 5′ overhangs intending the directionality of the overhangs. In some embodiments, the overhangs will be ligated to each other, as described herein, and thus the 3′ overhang may comprise a 3′ hydroxyl and the 5′ overhang may comprise a 5′ phosphate.

In some embodiments, the overhangs may be designed such that they include secondary structure such as but not limited to hairpin conformations. Such secondary structures may be melted during hybridization to the target, or they may be melted as a result of a change in condition or contact with an extrinsic trigger. Thus, also provided herein are compositions comprising any of the foregoing nucleic acid complexes. The composition may comprise a plurality of nucleic acid complexes. The nucleic acid complexes in the plurality may be identical to each other.

Alternatively, the nucleic acid complexes in the plurality may be different from each other. The nanoswitches may differ from each other with respect to their ribonucleic acid-of-interest specificity (e.g., the nucleotide sequence of their 3′ overhangs and/or the sequence of the 5′ overhangs). Nanoswitches may also differ from each other with respect to the distance between the 3′ overhang and the 5′ overhang along the length of the scaffold nucleic acid. Alternative embodiments for increasing signal are also contemplated herein and described below.

### Ribonucleic Acid-of-Interest

In embodiments, and as described above, one or more ribonucleic acids-of-interest may be pre-selected to hybridize to the one or more detectors and form a DNA-nanoswitch-ribonucleic acid complex of the present disclosure. In the embodiments, the one or more ribonucleic acids-of-interest may be an RNA, viral RNA, or a combination thereof. In embodiments, the ribonucleic acid-of-interest may be a naturally occurring ribonucleic acid, or one or more fragments thereof.

In embodiments, the ribonucleic acid-of-interest, as used herein, refers to the ribonucleic acid that is hybridized to the nanoswitch. It is to be understood that the ribonucleic acid-of-interest may derive from and thus be a fragment of a much larger ribonucleic acid such as for example viral RNA. Thus, a binding portion of the ribonucleic acid-of-interest (i.e., the ribonucleic acid bound to the nanoswitch) may range from about 7-50 nucleotides, or e.g., 10 to 35 nucleotides, or 5 to 30 nucleotides in some instances, while its parent ribonucleic acid may be much longer (for example on the order to kbs or more).

In some embodiments, the conditions that allow ribonucleic acid-of-interest such as viral RNA to hybridize to the 3′ overhang and the 5′ overhang may be standard hybridization conditions as known in the art. Such conditions may include a suitable concentration of salt(s) and optionally a buffer. The condition may also include EDTA in order to preserve the DNA nanoswitch-ribonucleic acid complex.

In some embodiments, the hybridization may be accomplished using a constant annealing temperature. Such constant temperature may range from about 4° C. to 55° C., 15° C. to 30° C., or 20° C. to 30° C., or may be about 25° C. The temperature may be regarded as room temperature (RT). The hybridization may be carried out over a period of hours such as 1, 2, 3, 4, 5 hours or more.

In some embodiments, the hybridization may be accomplished by decreasing the temperature from a temperature at which the ribonucleic acid-of-interest and the overhangs are not hybridized to each other to a temperature at which they are hybridized to each other. This is referred to herein as a temperature ramp or a decreasing annealing temperature. The starting temperature may be about 40-60° C. without limitation. The ending temperature may be about 4-25° C. without limitation. Thus, the temperature ramp may be from about 50° C. to about 4° C. or about 40° C. to about 4° C. In embodiments, a temperature ramp from about 46° C. to about 4° C. The change in temperature is typically carried out over 1-12 hours. Thus, the change in temperature may decrease by about 0.1-1° C. per minute.

Regardless of whether a constant or decreasing annealing temperature is used, the hybridization may also be carried out for much shorter periods of time, for example on the order of 10-30 minutes, provided readout can be achieved. Thus, in some instances, if the method determines if the nucleic acid-of-interest is present and has formed a DNA nanoswitch-ribonucleic acid complex of the present disclosure, then the hybridization period can be short, particularly if the ribonucleic acid-of-interest such as viral RNA is present in abundance. In some embodiments, longer hybridization times may be required. Similarly, if the ribonucleic acid-of-interest is present in low abundance, longer hybridization times may be required, particularly if an amplifying latch mechanism is used.

In some embodiments, only a portion or preselected portion of the ribonucleic acid-of-interest hybridizes to the nanoswitch of the present disclosure.

### Test Sample(s)

In embodiments, the biological sample is a sample that is being tested for the presence of one or more targets-of-interest such as viral RNA as described above. In embodiments, a target-of-interest may be present and thus contacted with a DNA nanoswitch of the present disclosure within in a mixture such as a biological sample or specimen. In embodiments, a target-of-interest may be disposed within a sample that may contain the target(s) or it may be suspected of containing the target(s). In embodiments, a sample may comprise non-target nucleic acid and be in the form of a mixture. Non-target nucleic acid, as used herein, refers to nucleic acids that are not the targets-of-interest or do not include a binding portion to the nanoswitch. In embodiments, the non-targets may be a peptide, protein or the like that does not react with or alter the conformation of the DNA nanoswitch or form a DNA nanoswitch-nucleic acid complex. The methods provided herein allow for the detection of a target such as a predetermined target even if such target is present in a molar excess of non-target nucleic acid. Thus, the sample may include on the order of micromolar quantities of non-target nucleic acid or protein and only nanomolar or picomolar quantities of target and still be able to detect the target. The target and non-targets may be present in the sample at a molar ratio of 1:10², 1:10³, 1:10⁴, 1:10⁵, or up to 1:10⁹.

In some embodiments, the sample may be derived from a biological sample such as a bodily fluid (e.g., a blood sample, a urine sample, a saliva sample, a sputum sample, a stool sample, a biopsy, and the like). The disclosure contemplates that such samples may be manipulated prior to contact or mixing with the nanoswitches of the present disclosure. For example, the samples may be treated to lyse cells, degrade or remove protein components, fragment nucleic acids such as genomic DNA, and the like.

In some instances, the target is or is derived from or is a fragment of viral RNA.

In embodiments, the methods of the present disclosure include detection of viral RNA. Such methods may be used to diagnose a condition, and thus may be referred to herein as diagnostic methods.

In embodiments, a method of the present disclosure includes contacting any of the foregoing DNA nanoswitch with viral RNA to form one or more DNA nanoswitch-ribonucleic acid complexes under conditions that allow a target, if present in the sample, to bind to the DNA nanoswitch and form a DNA nanoswitch-ribonucleic acid complex. In embodiments, the methods include detecting a conformation change between a DNA nanoswitch and a DNA nanoswitch-ribonucleic acid complex by moving the structures through a separation medium such as a gel medium. In embodiments, wherein a looped conformation is present within a medium such as an electrophoresis gel medium, the conformation is indicative of the presence of a specific target-of-interest such as a target viral RNA in the biological sample. In embodiments, in the presence of a target RNA, the DNA-nanoswitch-ribonucleic acid complex adopts a looped conformation as the DNA nanoswitch-target complex eliminates the linear conformation of the DNA nanoswitch.

In some embodiments, the conformation of the ribonucleic acid complex, e.g., DNA nanoswitch with viral RNA may be determined (or detected) using gel electrophoresis or liquid chromatography, or other separation technique. The gel electrophoresis may be a bufferless gel electrophoresis such as the E-Gel®. Agarose Gel Electrophoresis System (Life Technologies). In embodiments, methods may include detection of the target ribonucleic acid and detection and optionally purification or substantial purification of the target nucleic acid. In some embodiments, the method may also include measuring an absolute or relative amount of target ribonucleic acid. This can be done for example by measuring the intensity of bands on a gel or of fractions from a liquid chromatography separation.

Referring back to FIG. 25, in some embodiments, method 100 may start at process sequence 110 by contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed. In embodiments, the second conformation is characterized as a DNA nanoswitch-ribonucleic acid complex including a DNA nanoswitch component and a ribonucleic acid-of-interest component hybridized to the DNA nanoswitch as described above. For example, the DNA nanoswitch-ribonucleic acid complex includes a DNA nanoswitch component and a ribonucleic acid-of-interest component hybridized to the DNA nanoswitch in the form of a first conformation wherein the DNA nanoswitch-ribonucleic acid target complex is in a closed loop configuration. In embodiments, DNA nanoswitch-ribonucleic acid complex can be used to determine the presence of one or more targets-of-interest such as viral RNA, long viral RNA, or fragments thereof. The looped conformation DNA nanoswitch-ribonucleic acid target complex can be physically separated using gel electrophoresis from linear conformation nanoswitches. In embodiments, the looped conformation DNA nanoswitch-ribonucleic acid complex therefore may be physically separated from a complex mixture. In embodiments, the non-looped DNA nanoswitch can also be separated from the sample or mixture.

Referring back to FIG. 25, some embodiments of method 100 may start at process sequence 110 by contacting a deoxyribonucleic acid (DNA) nanoswitch having a first conformation characterized as open with a biological specimen to form a mixture, wherein when the mixture includes a ribonucleic acid-of-interest, the first conformation changes to a second conformation characterized as closed. In embodiments, the second conformation is characterized as a DNA nanoswitch-nucleic acid complex including a DNA nanoswitch component and a ribonucleic acid-of-interest component hybridized to the DNA nanoswitch as described above. For example, the DNA nanoswitch-nucleic acid complex includes a DNA nanoswitch component and a ribonucleic acid-of-interest component hybridized to the DNA nanoswitch in the form of a first conformation wherein the DNA nanoswitch-nucleic acid target complex is in a closed loop configuration. In embodiments, DNA nanoswitch-nucleic acid complex can be used to determine the presence of one or more RNA targets-of-interest such as viral RNA, or one or more fragments thereof. The looped conformation DNA nanoswitch-ribonucleic acid target complex can be physically separated using gel electrophoresis from linear conformation nanoswitches. In embodiments, the looped conformation DNA nanoswitch-nucleic acid complex therefore may be physically separated from a complex mixture. In embodiments, the non-looped DNA nanoswitch can also be separated from the sample or mixture.

Still referring to FIG. 25 once the first conformation changes to a second conformation characterized as closed, the process continues at process sequence 120 including processing the mixture under conditions sufficient to separate the first conformation, and when present, the second conformation. For example, in embodiments, the locked and open conformations can be separated using gel electrophoresis. In some embodiments, specific RNA nucleic acids-of-interest will be present in the looped conformation nanoswitches, and these looped conformation nanoswitches, e.g., a DNA nanoswitch-target complex can be isolated by gel extraction from electrophoresis forming a DNA nanoswitch-target complex within a gel medium.

Still referring to FIG. 25, once processing the mixture under conditions sufficient to separate the first conformation, and when present, the second conformation is complete, process sequence 130 includes reacting the first conformation, and when present, the second conformation with an indicator under conditions sufficient to form a signal. In embodiments, the reacting may include contacting the separation medium with a dye. In some embodiments, reacting may be performed during or after gel electrophoresis, a visible signal within the gel medium.

In embodiments, the present disclosure relates to a method of detecting a virus by reconfiguring a nucleic acid including: contacting a deoxyribonucleic acid (DNA) nanoswitch having a first conformation characterized as open with a biological specimen to form a mixture, wherein when the mixture comprises a ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. In some embodiments, the ribonucleic acid of interest is viral ribonucleic acid (viral RNA), long viral RNA, or one or more fragments thereof. In some embodiments, the ribonucleic acid-of-interest binds to the deoxyribonucleic acid (DNA) nanoswitch to form a second conformation including a loop. In some embodiments, the biological specimen comprises one or more ribonucleic acids-of-interest. In some embodiments, the one or more ribonucleic acid-of-interest include RNA or a fragment thereof from ZIKA, EBOLA, SARS, or SARS-CoV-2, or variants thereof. In some embodiments, the ribonucleic acid-of-interest is SEQ ID NO:1, or a fragment thereof. In some embodiments, the ribonucleic acid-of-interest is SARA-CoV-2 RNA, or a fragment thereof as described in U.S. Pat. No. 10,815,539 (herein entirely incorporated by reference). In some embodiments, processing the mixture includes electrophoresing the mixture under conditions sufficient to separate the first conformation and the second conformation. In some embodiments, a formation of the second conformation signals a presence of one or more ribonucleic acids. In some embodiments, the second conformation has an altered functionality compared to the first conformation. In some embodiments, the first conformation is configured to change to a second conformation when contacted with ribonucleic acid-of-interest, and wherein the second conformation is configured to report a ribonucleic acid-of-interest.

In some embodiments, the present disclosure relates to a method of reconfiguring a nucleic acid including: contacting a deoxyribonucleic acid (DNA) nanoswitch having a first conformation, with a ribonucleic acid to form a DNA nanoswitch-nucleic acid complex having a second conformation, wherein the second conformation is characterized as locked; processing a mixture under conditions sufficient to separate the first conformation and the second conformation; and contacting the first conformation and second conformation with an indicator under conditions sufficient to form a signal. In some embodiments, the signal is predetermined to show a presence or absence of ribonucleic acid.

In some embodiments, the present disclosure relates to a nucleic acid structure or molecule, including: a DNA nanoswitch-nucleic acid complex including a deoxyribonucleic acid (DNA) nanoswitch and a ribonucleic acid binding site, wherein the DNA nanoswitch has a first conformation characterized as open, and a second conformation characterized as closed when in a presence of ribonucleic acid.

In some embodiments, the present disclosure relates to a nucleic acid structure or molecule, including: a DNA nanoswitch-nucleic acid complex including a deoxyribonucleic acid (DNA) nanoswitch and an RNA binding site, wherein the DNA nanoswitch has a first conformation characterized as open, and a second conformation characterized as closed when in a presence of RNA bound to the RNA binding site.

### Additional Embodiments

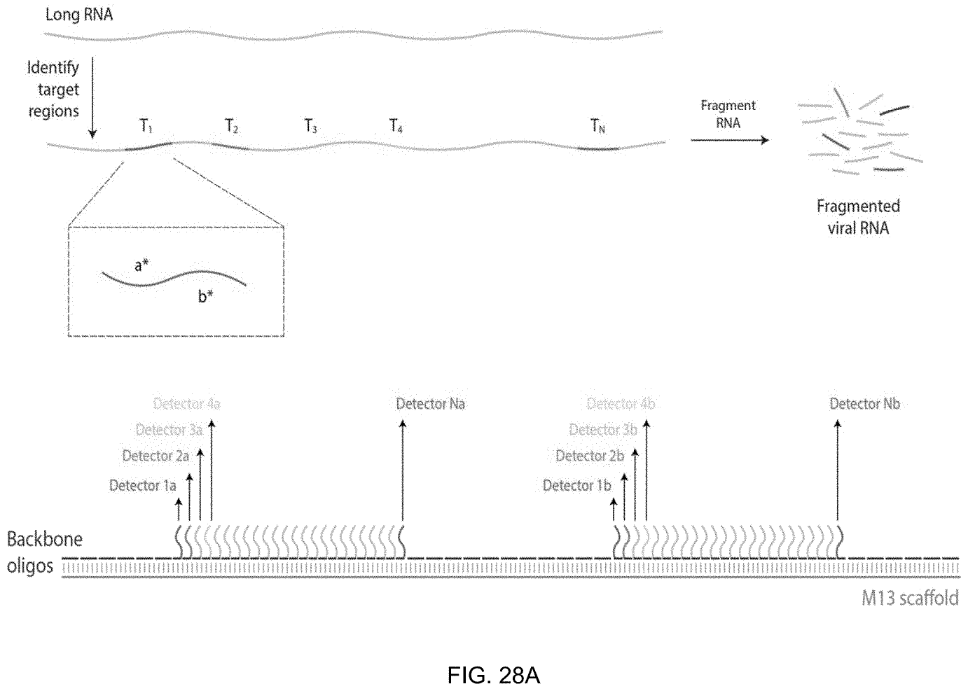

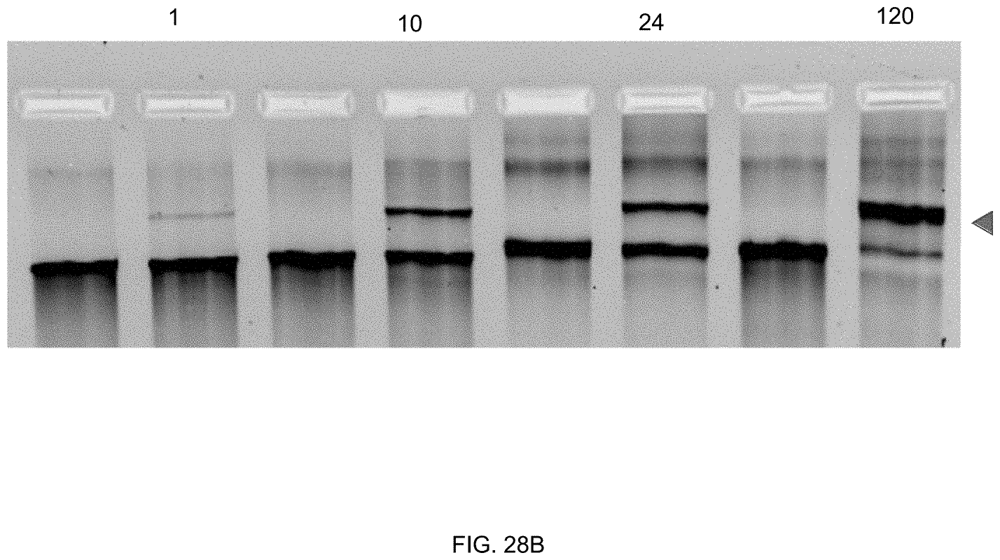

Referring to FIGS. 28A and 28B, embodiments, of the present disclosure include multi-detection strategies to amplify signal. In embodiments, the present disclosure relates to one or more (such as a plurality) of single target nanoswitches produced and/or provided in a mixture to target multiple regions of a long (viral) RNA, wherein the long (viral RNA) is fragmented. In embodiments, a multi-targeting approach has the advantage of increasing the signal of a DNA nanoswitch subjected to gel electrophoresis. For example, embodiments, of the present disclosure may include a method of detecting an RNA virus by reconfiguring a plurality of nucleic acid including: contacting a plurality of deoxyribonucleic acid (DNA) nanoswitches having a first conformation characterized as open with a biological specimen to form a mixture, wherein the mixture includes a plurality of RNA segments from a single long ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. In embodiments, the single long viral nucleic acid-of-interest is viral ribonucleic acid (viral RNA) characterized as long such as e.g., a viral RNA including nucleotides in an amount greater than 5,000 nucleotides, 6,000 nucleotides, 7,000 nucleotides, 8,000 nucleotides, 9,000 nucleotides, or greater than 10,000 nucleotides. In embodiments, the single long ribonucleic acid-of-interest is fragmented into a plurality of RNA segments. In embodiments, the plurality of deoxyribonucleic acid (DNA) nanoswitches include DNA nanoswitches including a predetermined RNA binding region, configured to bind to different predetermined segments of a fragmented long viral RNA. For example, a first DNA nanoswitch may be configured to change conformation when contacted with a predetermined RNA sequence or segment in a first segment of a fragmented long viral RNA, and a second DNA nanoswitch may be configured to change conformation when contacted with a predetermined RNA sequence or segment in a second segment of the fragmented long viral RNA, and a third DNA nanoswitch may be configured to change conformation when contacted with a predetermined RNA sequence or segment in a third segment or sequence of the fragmented long viral RNA. In embodiments, there may be 3-1000 segments of fragmented long viral RNA that are detectable in accordance with the present disclosure. In embodiments, there may be 3-1000 DNA nanoswitches configured to change conformation (such as to a loop) when contacted with 3-1000 different sequences or segments of a fragmented long viral RNA. In embodiments, a multi-targeting approach to a plurality of predetermined RNA sequences derived from a long RNA may increase the signal of the present disclosure. By increasing the signal, the methods of the present disclosure are highly sensitive and robust.

In embodiments, the present disclosure relates to one or more (such as a plurality) of DNA nanoswitches which individually include a plurality of RNA binding sites configured to target different segments of a long (viral RNA). In embodiments, such as where a DNA nanoswitch is configured to have a multi-targeting approach by the inclusion of a plurality of RNA binding sites within the DNA nanoswitch, wherein each RNA binding site may be preselected to bind to a separate region or segment of viral RNA-of-interest, such embodiments, have the advantage of increasing the signal of the DNA nanoswitches subjected to gel electrophoresis. For example, embodiments, of the present disclosure may include a method of detecting an RNA virus by reconfiguring a plurality of nucleic acid including: contacting a plurality of deoxyribonucleic acid (DNA) nanoswitches having a first conformation characterized as open with a biological specimen to form a mixture, wherein the mixture includes a plurality of RNA segments from a single long ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. In embodiments, the single long viral ribonucleic acid-of-interest is viral ribonucleic acid (viral RNA) characterized as long such as e.g., a viral RNA including nucleotides in an amount greater than 5,000 nucleotides, 6,000 nucleotides, 7,000 nucleotides, 8,000 nucleotides, 9,000 nucleotides, or greater than 10,000 nucleotides. In embodiments, the single long ribonucleic acid-of-interest is not fragmented and/or is fragmented into a plurality of RNA segments. In embodiments, the plurality of deoxyribonucleic acid (DNA) nanoswitches include DNA nanoswitches including a plurality of different predetermined RNA binding sites, configured to bind to different predetermined segments of a fragmented long viral RNA or unfragmented long viral RNA. For example, a first DNA nanoswitch may be configured to change conformation when contacted with a first, second, or third predetermined RNA sequence or segment at a first, second, or third RNA binding site of the DNA nanoswitch. In embodiments, a single DNA nanoswitch of the present disclosure may be configured to change conformation (such as to a loop) when contacted with 3-100, or 10-50 different sequences or segments of unfragmented or fragmented long viral RNA. In embodiments, a multi-targeting approach where a single DNA nanoswitch may react with a plurality of predetermined RNA sequences such as those of a long RNA, or those derived from a long RNA may increase the signal of the present disclosure. By increasing the signal, the methods of the present disclosure are highly sensitive and robust.

In embodiments, the present disclosure relates to one or more (such as a plurality) of DNA nanoswitches which individually or collectively include a plurality of variant RNA binding sites. Here the DNA nanoswitches may be configured to target the same segment of a long (viral RNA). In embodiments, such as where a DNA nanoswitch is configured to include a plurality of variant RNA binding sites a single-targeting approach by the inclusion of a plurality of variant target regions within the DNA nanoswitch may have the advantage of improving binding kinetics between the variant RNA binding sites of the DNA nanoswitch and the RNA-of-interest. In embodiments, signal formation may be promoted for such DNA nanoswitches subjected to gel electrophoresis. For example, embodiments of the present disclosure may include a method of detecting an RNA virus by reconfiguring a plurality of nucleic acid structures including: contacting a plurality of deoxyribonucleic acid (DNA) nanoswitches having a first conformation characterized as open with a biological specimen to form a mixture, wherein the mixture includes an RNA of interest such as from a single long ribonucleic acid-of-interest, the first conformation changes to a second conformation characterized as closed; processing the mixture under conditions sufficient to separate the first conformation, and when present, the second conformation; and reacting the first conformation, and when present, the second conformation with an indicator under conditions sufficient to form a signal. In embodiments, the DNA nanoswitch may be include one or more RNA binding sites characterized as a variant binding sites. As used herein the variant target region may vary between different DNA nanoswitches. The RNA binding sites variants may be highly related sequences, or highly homologous sequences having 90%, 95%, 97%, 99% sequence identity. The variant RNA binding sites may also bind to the same region of target RNA under substantially similar hybridization conditions. The variants may have different lengths, such that one variant appears truncated upon comparison to a highly related variant.

Embodiments, of the present disclosure further include embodiments as described herein alone, or in combination. For example, it is possible to mix and match the embodiments described herein. In some embodiments, the methods of the present disclosure are preselected to obtain a maximum signal. For example, it is possible to use a plurality of different multi-targeting DNA nanoswitches to target a plurality of regions on a viral RNA-of-interest. In embodiments, 1-5 different multiple (1-25) target nanoswitches may be used to target over 100 regions on SARA-CoV-2 viral RNA, which can give a 120 fold benefit in limit of detection. In embodiments, the techniques and DNA nanoswitches are expandable up to about 50 targets per nanoswitch, and about 10-20 nanoswitches targeting about 1,000 regions with about 1000-fold increase in sensitivity.

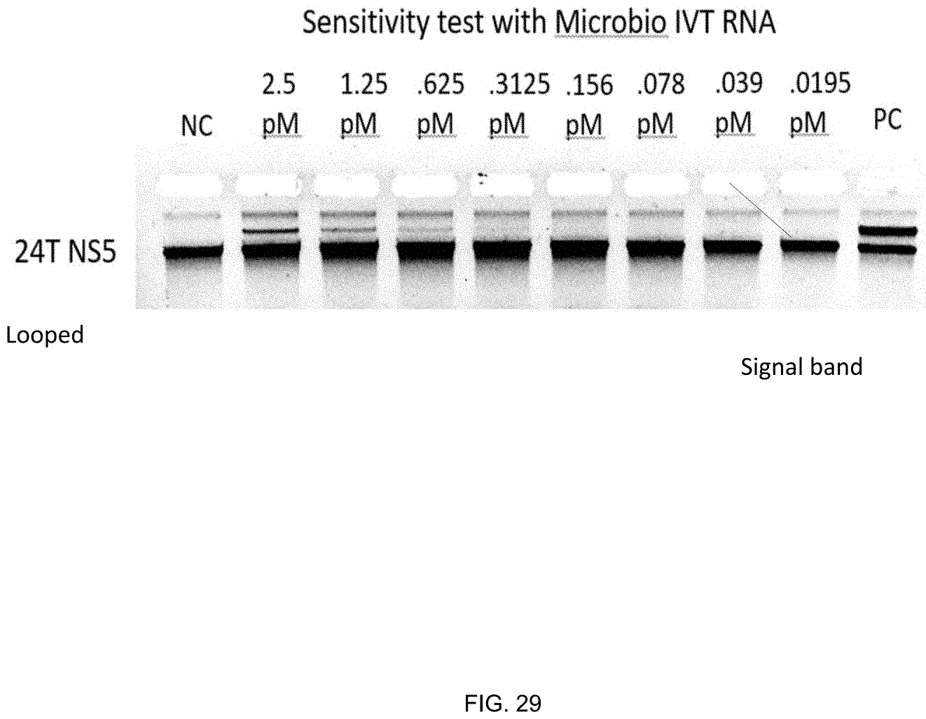

Referring now to FIG. 29, embodiments of the present disclosure are suitable for 1-hour detection with in vitro transcribed RNA.

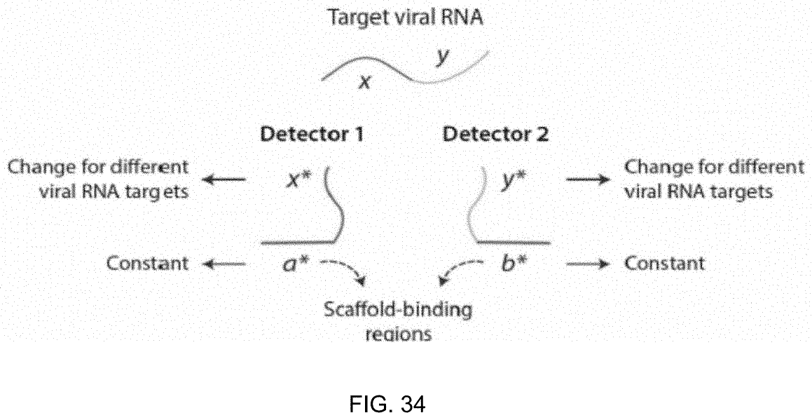

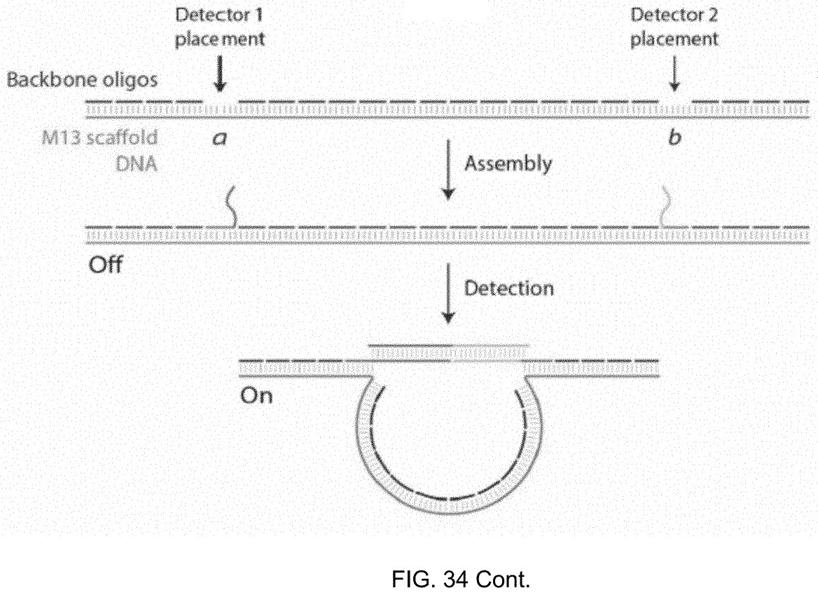

FIG. 34 depicts an embodiment of the present disclosure where a DNA nanoswitch includes two detector strands hybridizable to scaffold and target viral RNA of interest, a DNA nanoswitch assembly, and detection of a target viral RNA. Referring to FIG. 34, a DNA nanoswitch suitable for forming a DNA nanoswitch-ribonucleic acid complex is shown including: a scaffold, such as M13 scaffold DNA including plurality of nucleotides or a polynucleotide sequence(s). Referring to FIG. 34 a plurality of backbone oligonucleotides are shown hybridized to the scaffold to form a backbone polynucleotide. A first detector strand “Detector 1” is shown including a nucleic acid sequence having a first segment (α*) hybridized to a scaffold or scaffold-binding region, and a second segment (x*) characterized as an overhang, wherein the second segment is hybridizable to, when present a first segment (X) of a preselected target RNA nucleotide sequence or target RNA polynucleotide-of-interest. Still referring to FIG. 34, a second detector strand “Detector 2” including a nucleic acid sequence having a first segment (b*) hybridized to the scaffold or a scaffold binding region, and a second segment (y*) characterized as an overhang, wherein the second segment is hybridizable to, when present, a second segment (Y) of the preselected target RNA nucleotide sequence or the target RNA polynucleotide-of-interest. In embodiments, the first detector strand includes a nucleic acid sequence having a first segment hybridized to the scaffold, and a second segment characterized as an overhang, wherein the length of the first segment is about 5 to 30 nucleotides, and the length of the second segment is about 5 to 30 nucleotides. In embodiments, the second detector strand includes a nucleic acid sequence having a first segment hybridized to the scaffold, and a second segment characterized as an overhang, wherein the length of the first segment is about 5 to 30 nucleotides, and the length of the second segment is about 5-30 nucleotides. In embodiments, first detector such as “Detector 1” and the second detector such as “Detector 2” form a pair of detectors. Although not shown in FIG. 34, in embodiments, the DNA nanoswitch further includes one or more additional pair of detectors, wherein the additional pair of detectors are hybridizable to one or more different segments of the preselected target RNA nucleotide sequence or the target RNA polynucleotide-of-interest. In embodiments, the DNA nanoswitch further includes 20-50 additional pair of detectors, wherein the additional pair of detectors are hybridizable to one or more different segments of the preselected target RNA nucleotide sequence or the target RNA polynucleotide-of-interest.

In embodiments, the present disclosure includes a kit, including: one or more DNA nanoswitches of the present disclosure, wherein, when present, the DNA nanoswitch hybridizes a viral RNA or a fragment thereof; and a separation medium such as a gel strip. In embodiments, the kit further includes a buffer.

### EXAMPLES

### Example I

Detection of viruses is critical for controlling disease spread and for informing clinical interventions. Recent emerging viral threats including Zika virus, Ebola virus, and SARS-Cov-2 (responsible for the COVID-19 outbreak) highlight the cost and difficulty in responding rapidly. To address these challenges, the present disclosure provides a platform for low-cost and rapid detection of viral RNA with DNA nanoswitches designed to mechanically reconfigure in response to specific viruses. Using Zika virus as a model system, non-enzymatic detection of viral RNA is shown to the attomole level, with selective and multiplexed detection between related viruses and viral strains. For clinical-level sensitivity in biological fluids, the assay was paired with a sample preparation step using either RNA extraction or isothermal pre-amplification. The assays of the present disclosure can be performed with minimal or no lab infrastructure, and are readily adaptable to detect other RNA viruses. The adaptability of the methods of the present disclosure was demonstrated by quickly developing and testing DNA nanoswitches for detecting a fragment of SARS-CoV-2 RNA in human saliva. Given this versatility, field implementation will improve the ability to detect emergent viral threats and ultimately limit their impact.

### Materials and Methods

### Construction and Purification of Nanoswitches

Oligonucleotides were purchased from Integrated DNA Technologies (IDT) with standard desalting, and the full sequences of all strands is listed in the tables below (Tables 1 to 10).

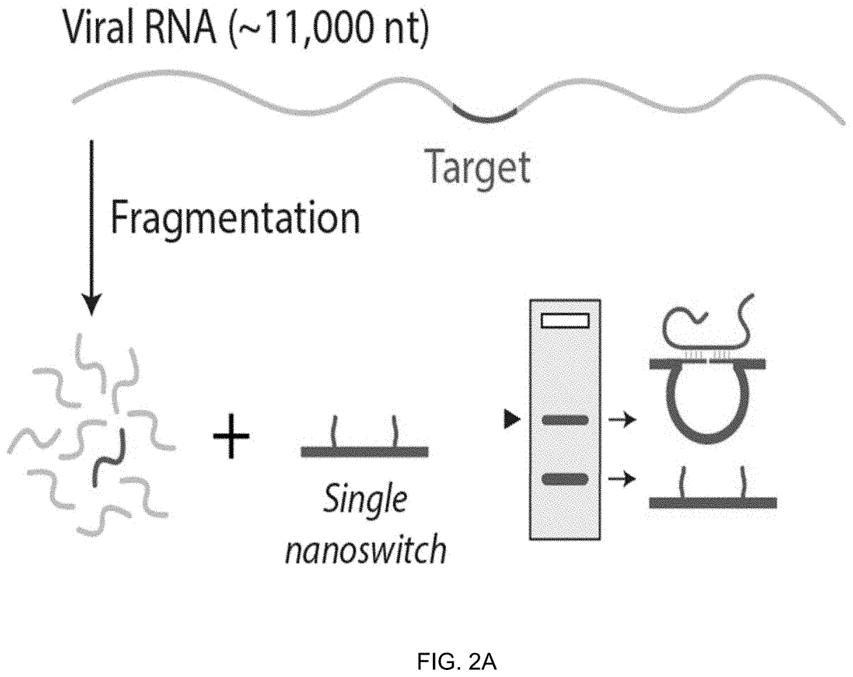

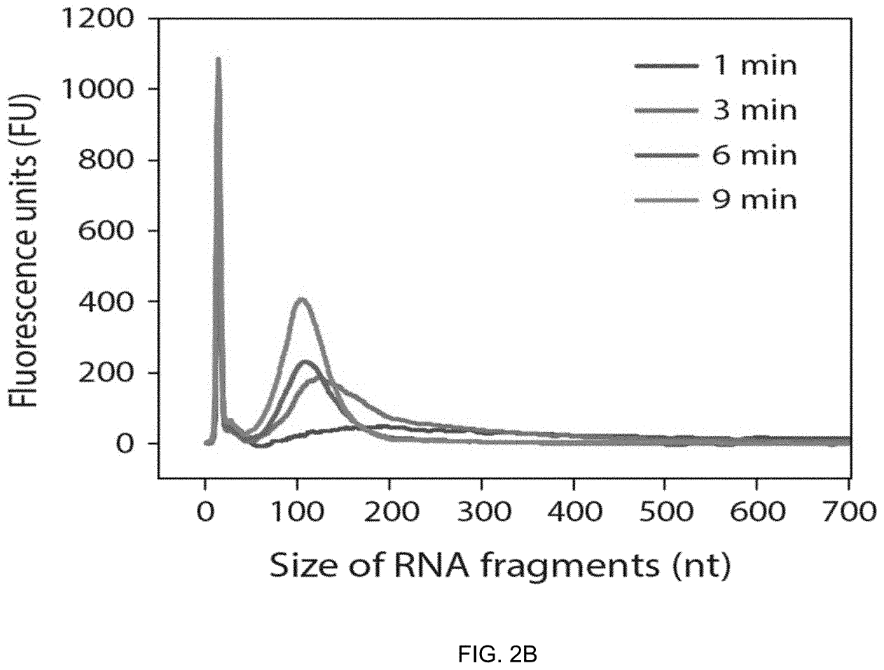

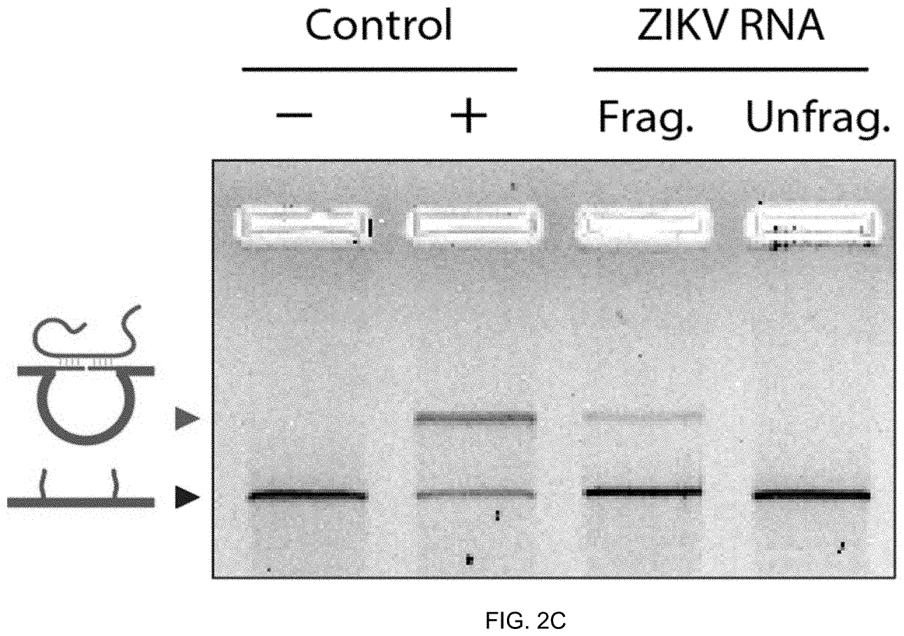

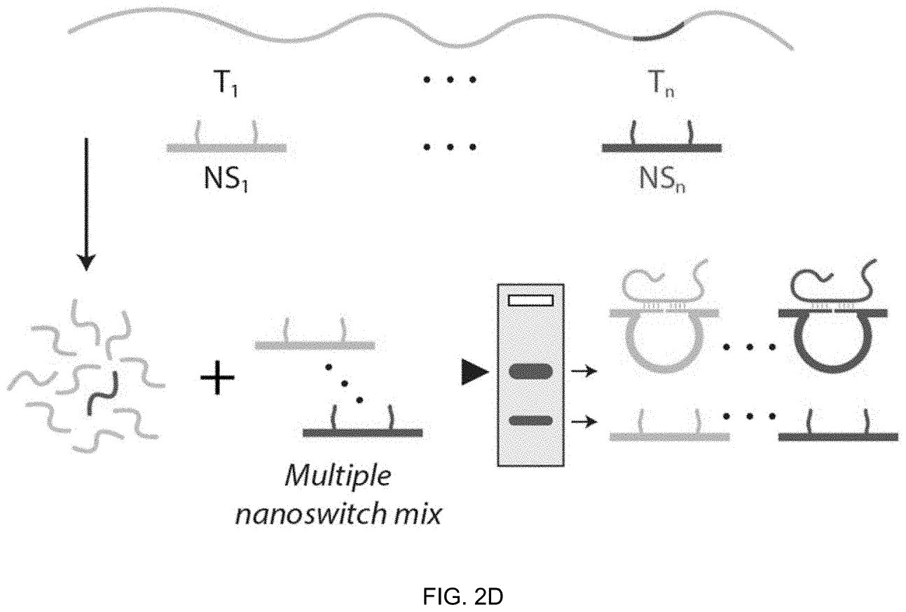

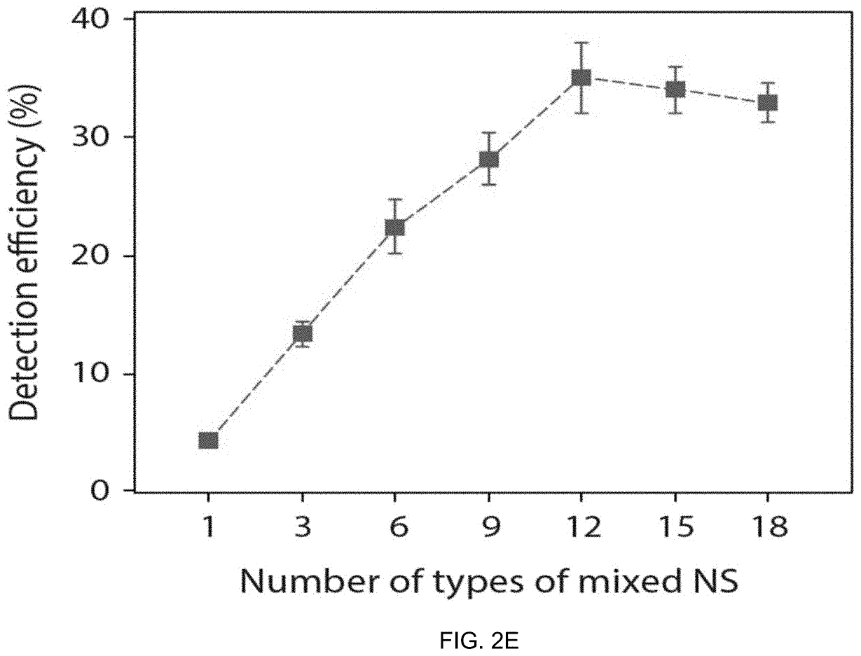

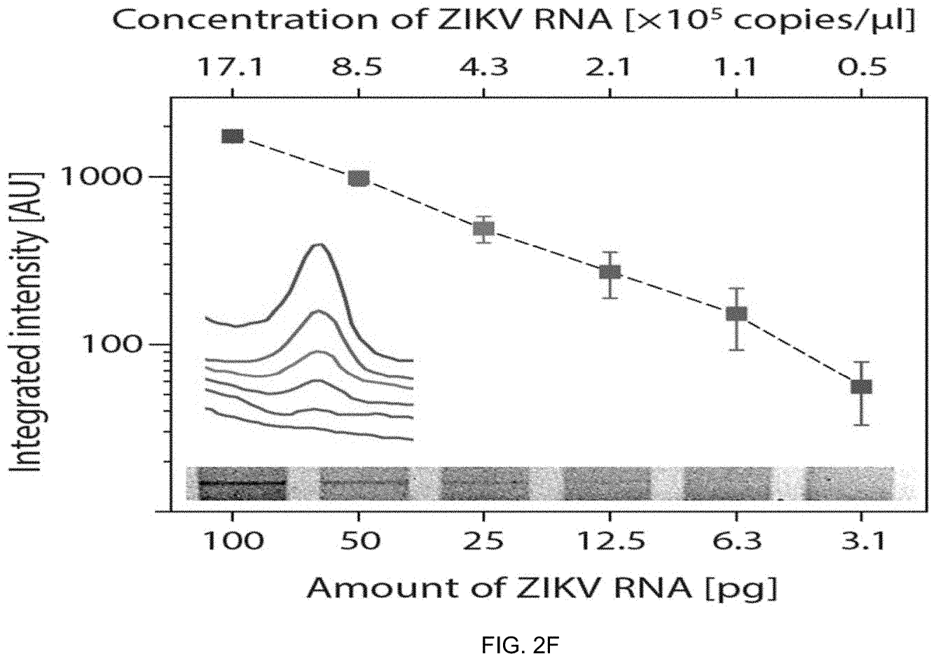

TABLE 1
The ZIKV RNA target sequence and its corresponding detector
ssDNA used in the experiment of FIG. 2C. In embodiments, suitable
targets include those sequences having at least 80%, 90%, 95%, 97%,
99% sequence identity to SEQ ID NOS: 1-3 as set forth in table 1.

| Name | Sequence (5′-3′) | length |
|---|---|---|
| ZIKV_s1_ | AGCCTACCTTGACAAGCAATCAGACACTCA (SEQ ID NO: 1) | 30 |
| Target | | |
| v4- | ACCGTTGTAGCAATACTTCTTTGATTAGTAATAACATCACTG | 55 |
| ZIKV_s1- | AGTGTCTGATTGC (SEQ ID NO: 2) | |
| 40-15 | | |
| v8- | TTGTCAAGGTAGGCTTCAACCGATTGAGGGAGGGAAGGTA | 55 |
| ZIKV_s1_ | AATATTGACGGAAAT (SEQ ID NO: 3) | |
| 15-40 | | |

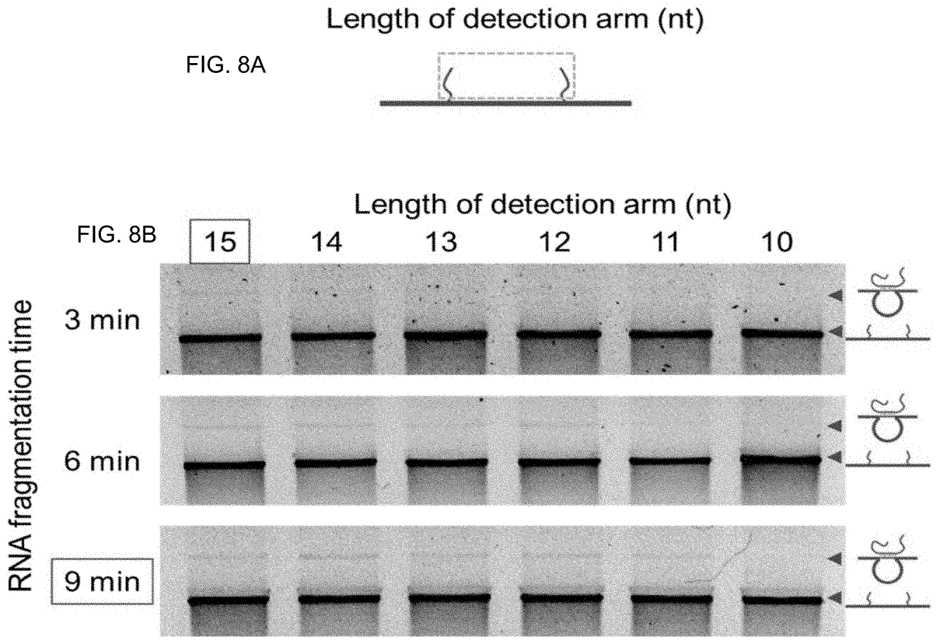

TABLE 2
Target sequence and different lengths of detector ssDNA (15, 14,
13, 12, 11, 10 nt) for optimization the design of nanoswitch (FIGS. 8A
and 8B). In embodiments, suitable polynucleotides include those having a
nucleotide sequence having at least 80%, 90%, 95%, 97%, 99% sequence identity
to SEQ ID NOS: 4-16 as set forth in the table below.

| Name | Sequence (5′ to 3′) | lenght |
|---|---|---|
| ZIKV_arm length | AACGCCCAATTCACCAAGAGCCGAAGCCAC (SEQ ID | 30 |
| test_Target | NO: 4) | |
| v4-ZIKV arm | CAATACTTCTTTGATTAGTAATAACATCAC [symbol] | 45 |
| length test 30-15 | [symbol] (SEQ ID NO: 5) | |
| v8-ZIKV arm | [symbol] TCAACCGATTGAGGGAGGGA | 45 |
| length test 15-30 | AGGTAAATAT (SEQ ID NO: 6) | |
| v4-ZIKV arm | CAATACTTCTTTGATTAGTAATAACATCAC [symbol] | 44 |
| length test 30-14 | [symbol] (SEQ ID NO: 7) | |
| v8-ZIKV arm | [symbol] TCAACCGATTGAGGGAGGGAA | 44 |
| length test 14-30 | GGTAAATAT (SEQ ID NO: 8) | |
| v4-ZIKV arm | CAATACTTCTTTGATTAGTAATAACATCAC [symbol] | 43 |
| length test 30-13 | [symbol] (SEQ ID NO: 9) | |
| v8-ZIKV arm | [symbol] TCAACCGATTGAGGGAGGGAAG | 43 |
| length test 13-30 | GTAAATAT (SEQ ID NO: 10) | |
| v4-ZIKV arm | CAATACTTCTTTGATTAGTAATAACATCAC [symbol] | 42 |
| length test 30-12 | [symbol] (SEQ ID NO: 11) | |
| v8-ZIKV arm | [symbol] TCAACCGATTGAGGGAGGGAAGG | 42 |
| length test 12-30 | TAAATAT (SEQ ID NO: 12) | |
| v4-ZIKV arm | CAATACTTCTTTGATTAGTAATAACATCAC [symbol] | 41 |
| length test 30-11 | [symbol] (SEQ ID NO: 13) | |
| v8-ZIKV arm | [symbol] TCAACCGATTGAGGGAGGGAAGGT | 41 |
| length test 11-30 | AAATAT (SEQ ID NO: 14) | |
| v4-ZIKV arm | CAATACTTCTTTGATTAGTAATAACATCAC [symbol] | 40 |
| length test 30-10 | [symbol] (SEQ ID NO: 15) | |
| v8-ZIKV arm | [symbol] TCAACCGATTGAGGGAGGGAAGGTA | 40 |
| length test 10-30 | AATAT (SEQ ID NO: 16) | |

TABLE 3
The eighteen target sequences and corresponding detector
ssDNA oligos for the detection of ZIKV RNA (FIGS. 2E, 2F, 3A, 3B, 3D, 5A, 12,
13, 14). Note: the position of each target sequence on the ZIKV RNA is shown
in the far-right column. In embodiments, suitable polynucleotides include those
having a nucleotide sequence having at least 80%, 90%, 95%, 97%, 99% sequence
identity to SEQ ID NOS: 17-70 as set forth in the table below.

| Nanoswitch | Name | Sequence (5′-3′) | Len. | Pos. |
|---|---|---|---|---|
| 1 | ZIKV_ | GTGTGATGCCACCATGAGCTATGAATG | 30 | 605 |
| | Target1 | CCC (SEQ ID NO: 17) | | |
| | v4-ZIKV T1 | ACCGTTGTAGCAATACTTCTTTGATTAG | | |
| | 40-15 | TAATAACATCACGGGCATTCATAGCTC | 55 | |
| | | (SEQ ID NO: 18) | | |
| | v8-ZIKV T1 | ATGGTGGCATCACACTCAACCGATTGA | | |
| | 15-40 | GGGAGGGAAGGTAAATATTGACGGAA | 55 | |
| | | AT (SEQ ID NO: 19) | | |
| 2 | ZIKV_ | AGTGGACAGAGGCTGGGGAAATGGAT | 30 | 1265 |
| | Target2 | GTGG (SEQ ID NO: 20) | | |
| | v4-ZIKV T2 | ACCGTTGTAGCAATACTTCTTTGATTAG | 55 | |
| | 40-15 | TAATAACATCACCCACATCCATTTCCC | | |
| | | (SEQ ID NO: 21) | | |
| | v8-ZIKV T2 | CAGCCTCTGTCCACTTCAACCGATTGA | 55 | |
| | 15-40 | GGGAGGGAAGGTAAATATTGACGGAA | | |
| | | AT (SEQ ID NO: 22) | | |
| 3 | ZIKV_ | AACGCCCAATTCACCAAGAGCCGAAG | 30 | 1484 |
| | Target3 | CCAC (SEQ ID NO: 23) | | |
| | v4-ZIKV T3 | ACCGTTGTAGCAATACTTCTTTGATTAG | 55 | |
| | 40-15 | TAATAACATCACGTGGCTTCGGCTCTT | | |
| | | (SEQ ID NO: 24) | | |
| | v8-ZIKV T3 | GGTGAATTGGGCGTTTCAACCGATTGA | 55 | |
| | 15-40 | GGGAGGGAAGGTAAATATTGACGGAA | | |
| | | AT (SEQ ID NO: 25) | | |
| 4 | ZIKV_Target4 | AGGGAGTCAAGAAGGAGCAGTTCACA | 30 | 1751 |
| | | CGGC (SEQ ID NO: 26) | | |
| | v4-ZIKV T4 | ACCGTTGTAGCAATACTTCTTTGATTAG | 55 | |
| | 40-15 | TAATAACATCACGCCGTGTGAACTGCT | | |
| | | (SEQ ID NO: 27) | | |
| | v8-ZIKV T4 | CCTTCTTGACTCCCTTCAACCGATTGA | 55 | |
| | 15-40 | GGGAGGGAAGGTAAATATTGACGGAA | | |
| | | AT (SEQ ID NO: 28) | | |
| 5 | ZIKV_ | GTACCATCCTGACTCCCCTCGTAGATT | 30 | 2588 |
| | Target5 | GGC (SEQ ID NO: 29) | | |
| | v4-ZIKV T5 | ACCGTTGTAGCAATACTTCTTTGATTAG | 55 | |
| | 40-15 | TAATAACATCACGCCAATCTACGAGGG | | |
| | | (SEQ ID NO: 30) | | |
| | v8-ZIKV T5 | GAGTCAGGATGGTACTCAACCGATTG | 55 | |
| | 15-40 | AGGGAGGGAAGGTAAATATTGACGGA | | |
| | | AAT (SEQ ID NO: 31) | | |
| 6 | ZIKV_ | ACATCATGTGGAGATCAGTAGAAGGG | 30 | 2683 |
| | Target6 | GAGC (SEQ ID NO: 32) | | |
| | v4-ZIKV T6 | ACCGTTGTAGCAATACTTCTTTGATTAG | 55 | |
| | 40-15 | TAATAACATCACGCTCCCCTTCTACTG | | |
| | | (SEQ ID NO: 33) | | |
| | v8-ZIKV T6 | ATCTCCACATGATGTTCAACCGATTGA | 55 | |
| | 15-40 | GGGAGGGAAGGTAAATATTGACGGAA | | |
| | | AT (SEQ ID NO: 34) | | |
| 7 | ZIKV_ | GAAGAACGACACATGGAGGCTGAAGA | 30 | 3104 |
| | Target7 | GGGC (SEQ ID NO: 35) | | |
| | v4-ZIKV T7 | ACCGTTGTAGCAATACTTCTTTGATTAG | 55 | |
| | 40-15 | TAATAACATCACGCCCTCTTCAGCCTC | | |
| | | (SEQ ID NO: 36) | | |
| | v8-ZIKV T7 | CATGTGTCGTTCTTCTCAACCGATTGA | 55 | |
| | 15-40 | GGGAGGGAAGGTAAATATTGACGGAA | | |
| | | AT (SEQ ID NO: 37) | | |
| 8 | ZIKV | CTAATTGGACACCCCGTGAGAGCATGC | 30 | 3835 |
| | Target 8 | TGC (SEQ ID NO: 38) | | |
| | v4-ZIKV T8 | ACCGTTGTAGCAATACTTCTTTGATTAG | 55 | |
| | 40-15 | TAATAACATCACGCAGCATGCTCTCAC | | |
| | | (SEQ ID NO: 39) | | |
| | v8-ZIKV T8 | GGGGTGTCCAATTAGTCAACCGATTGA | 55 | |
| | 15-40 | GGGAGGGAAGGTAAATATTGACGGAA | | |
| | | AT (SEQ ID NO: 40) | | |
| 9 | ZIKV_ | AAACAGTCCCCGGCTCGATGTGGCAC | 30 | 4430 |
| | Target9 | TAGA (SEQ ID NO: 41) | | |
| | v4-ZIKV T9 | ACCGTTGTAGCAATACTTCTTTGATTAG | 55 | |
| | 40-15 | TAATAACATCACTCTAGTGCCACATCG | | |
| | | (SEQ ID NO: 42) | | |
| | v8-ZIKV T9 | AGCCGGGGACTGTTTTCAACCGATTGA | 55 | |
| | 15-40 | GGGAGGGAAGGTAAATATTGACGGAA | | |
| | | AT (SEQ ID NO: 43) | | |
| 10 | ZIKV_ | CCCGGAGAGAGAGCGAGGAACATCCA | 30 | 4917 |
| | Target10 | GACT (SEQ ID NO: 44) | | |
| | v4-ZIKV | ACCGTTGTAGCAATACTTCTTTGATTAG | | |
| | T10 40-15 | TAATAACATCACAGTCTGGATGTTCCT | 55 | |
| | | (SEQ ID NO: 45) | | |
| | v8-ZIKV | CGCTCTCTCTCCGGGTCAACCGATTGA | 55 | |
| | T10 15-40 | GGGAGGGAAGGTAAATATTGACGGAA | | |
| | | AT (SEQ ID NO: 46) | | |
| 11 | ZIKV_ | GGACTACCCAGCAGGAACTTCAGGAT | 30 | 4997 |
| | Target11 | CTCC (SEQ ID NO: 47) | | |
| | v4-ZIKV | ACCGTTGTAGCAATACTTCTTTGATTAG | 55 | |
| | T11 40-15 | TAATAACATCACGGAGATCCTGAAGTT | | |
| | | (SEQ ID NO: 48) | | |
| | v8-ZIKV | CCTGCTGGGTAGTCCTCAACCGATTGA | 55 | |
| | T11 15-40 | GGGAGGGAAGGTAAATATTGACGGAA | | |
| | | AT (SEQ ID NO: 49) | | |
| 12 | ZIKV_ | GTGACGCATTCCCGGACTCCAACTCAC | 30 | 5581 |
| | Target12 | CAA (SEQ ID NO: 50) | | |
| | v4-ZIKV | ACCGTTGTAGCAATACTTCTTTGATTAG | 55 | |
| | T12 40-15 | TAATAACATCACTTGGTGAGTTGGAGT | | |
| | | (SEQ ID NO: 51) | | |
| | v8-ZIKV | CCGGGAATGCGTCACTCAACCGATTG | 55 | |
| | T12 15-40 | AGGGAGGGAAGGTAAATATTGACGGA | | |
| | | AAT (SEQ ID NO: 52) | | |
| 13 | ZIKV_ | GAGTTCCAGAAAACAAAACATCAAGAG | 30 | 5793 |
| | Target13 | TGG (SEQ ID NO: 53) | | |
| | v4-ZIKV | ACCGTTGTAGCAATACTTCTTTGATTAG | 55 | |
| | T13 40-15 | TAATAACATCACCCACTCTTGATGTTT | | |
| | | (SEQ ID NO: 54) | | |
| | v8-ZIKV | TGTTTTCTGGAACTCTCAACCGATTGA | 55 | |
| | T13 15-40 | GGGAGGGAAGGTAAATATTGACGGAA | | |
| | | AT (SEQ ID NO: 55) | | |
| 14 | ZIKV_ | CATCTAATGGGAAGGAGAGAGGAGGG | 30 | 6957 |
| | Target14 | GGCA (SEQ ID NO: 56) | | |
| | v4-ZIKV | ACCGTTGTAGCAATACTTCTTTGATTAG | 55 | |
| | T1440-15 | TAATAACATCACTGCCCCCTCCTCTCT | | |
| | | (SEQ ID NO: 57) | | |
| | v8-ZIKV | CCTTCCCATTAGATGTCAACCGATTGA | 55 | |
| | T14 15-40 | GGGAGGGAAGGTAAATATTGACGGAA | | |
| | | AT (SEQ ID NO: 58) | | |
| 15 | ZIKV_ | CACAGGAATAGCCATGACCGACACCA | 30 | 8684 |
| | Target15 | CACC (SEQ ID NO: 59) | | |
| | v4-ZIKV | ACCGTTGTAGCAATACTTCTTTGATTAG | 55 | |
| | T15 40-15 | TAATAACATCACGGTGTGGTGTCGGTC | | |
| | | (SEQ ID NO: 60) | | |
| | v8-ZIKV | ATGGCTATTCCTGTGTCAACCGATTGA | 55 | |
| | T15 15-40 | GGGAGGGAAGGTAAATATTGACGGAA | | |
| | | AT (SEQ ID NO: 61) | | |
| 16 | ZIKV_ | GGATGGGGAGAGAGAATTCAGGAGGT | 30 | 9160 |
| | Target16 | GGTG (SEQ ID NO: 62) | | |
| | v4-ZIKV | ACCGTTGTAGCAATACTTCTTTGATTAG | 55 | |
| | T16 40-15 | TAATAACATCACCACCACCTCCTGAAT | | |
| | | (SEQ ID NO: 63) | | |
| | v8-ZIKV | TCTCTCTCCCCATCCTCAACCGATTGA | 55 | |
| | T16 15-40 | GGGAGGGAAGGTAAATATTGACGGAA | | |
| | | AT (SEQ ID NO: 64) | | |
| 17 | ZIKV_ | GAGGAAGTTCTAGAGATGCAAGACTTG | 30 | 9549 |
| | Target17 | TGG (SEQ ID NO: 65) | | |
| | v4-ZIKV | ACCGTTGTAGCAATACTTCTTTGATTAG | 55 | |
| | T17 40-15 | TAATAACATCACCCACAAGTCTTGCAT | | |
| | | (SEQ ID NO: 66) | | |
| | v8-ZIKV | CTCTAGAACTTCCTCTCAACCGATTGA | 55 | |
| | T17 15-40 | GGGAGGGAAGGTAAATATTGACGGAA | | |
| | | AT (SEQ ID NO: 67) | | |
| 18 | ZIKV_ | CTGAGTCAAAAAACCCCACGCGCTTGG | 30 | 10543 |
| | Target18 | AGG (SEQ ID NO: 68) | | |
| | v4-ZIKV | ACCGTTGTAGCAATACTTCTTTGATTAG | 55 | |
| | T18 40-15 | TAATAACATCACCCTCCAAGCGCGTGG | | |
| | | (SEQ ID NO: 69) | | |
| | v8-ZIKV | GGTTTTTTGACTCAGTCAACCGATTGA | 55 | |
| | T18 15-40 | GGGAGGGAAGGTAAATATTGACGGAA | | |
| | | AT (SEQ ID NO: 70) | | |

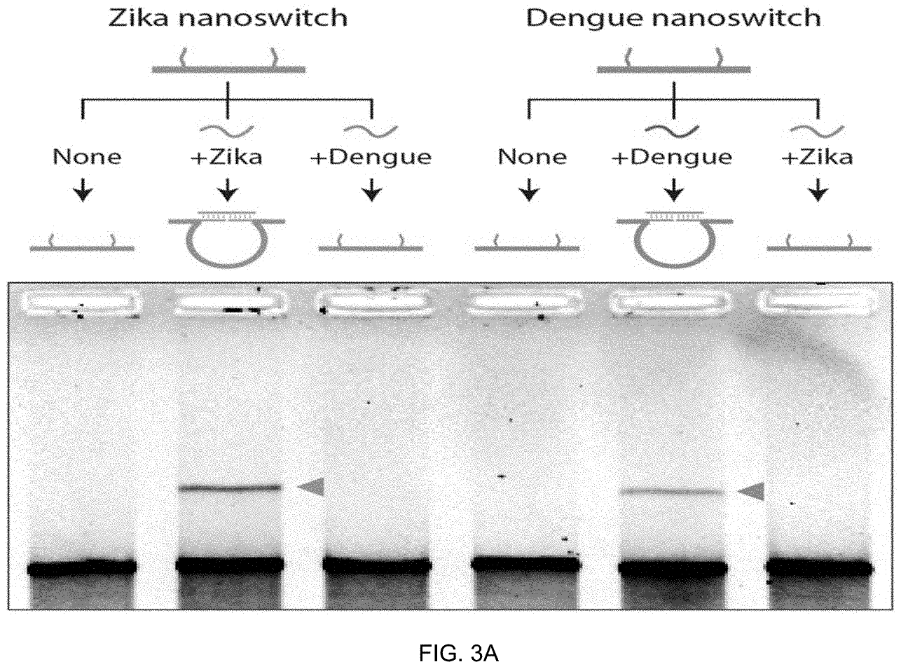

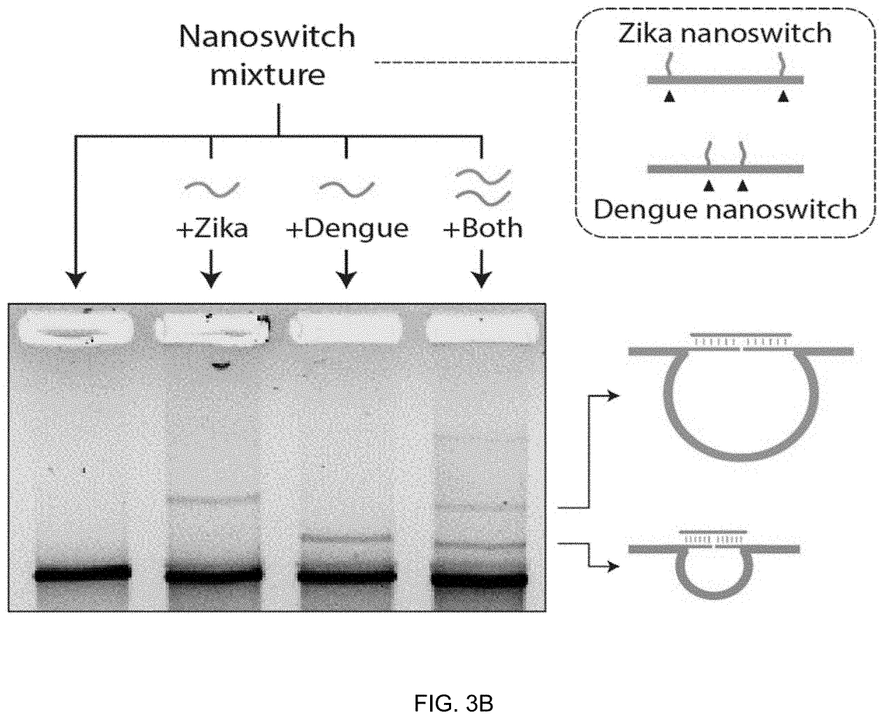

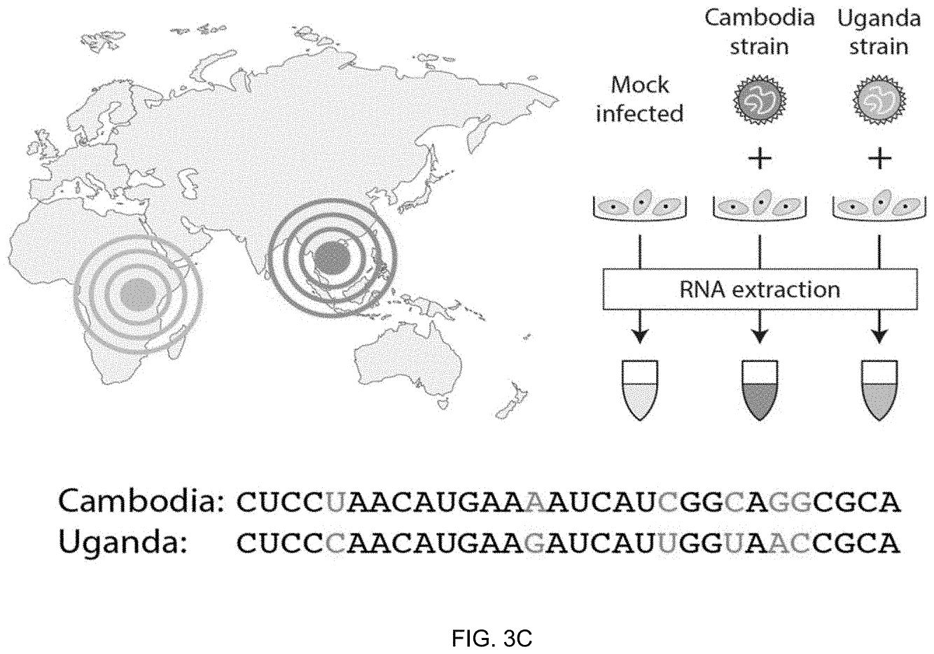

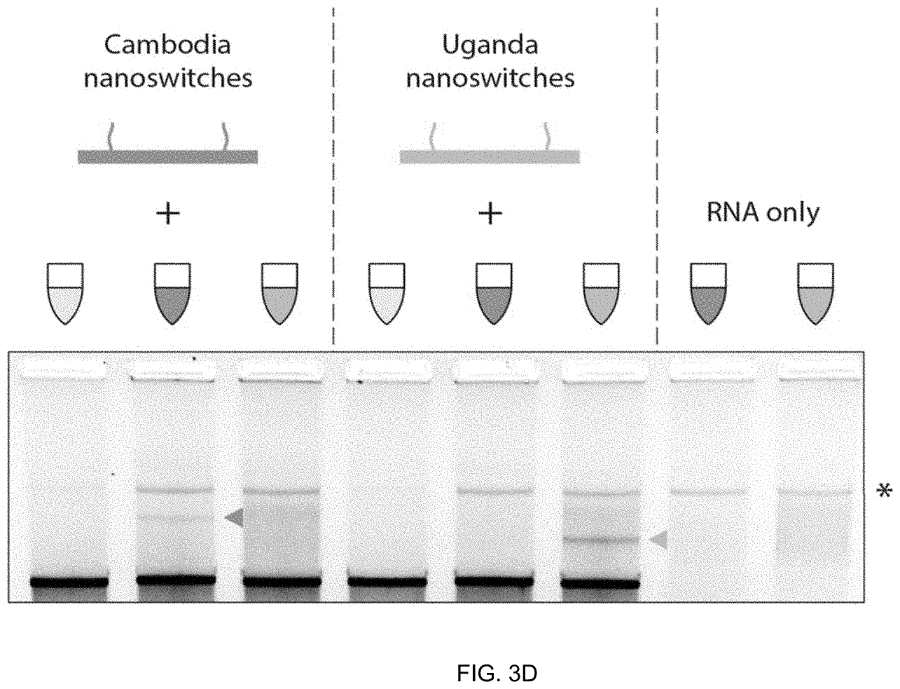

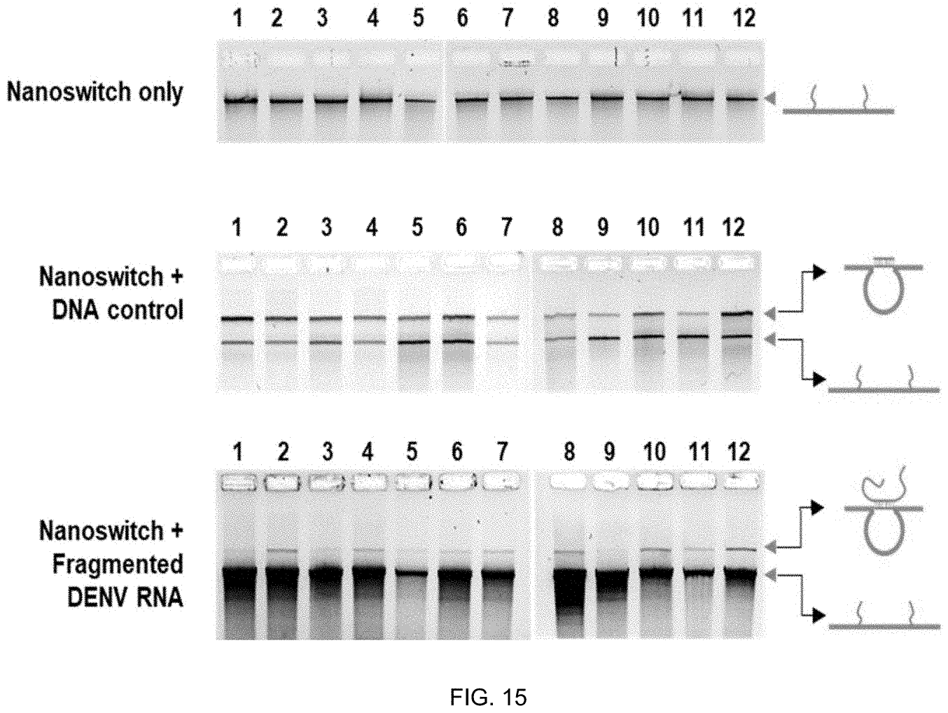

TABLE 4
The twelve target sequences and corresponding detector ssDNA
oligos for the detection of DENV RNA (FIG. 3A, FIG. 15). Note: the position of
each target sequence on the DENV RNA is shown in the far-right column. In
embodiments, suitable polynucleotides include those having a nucleotide sequence
having at least 80%, 90%, 95%, 97%, 99% sequence identity to SEQ ID NOS: 71-106 as
set forth in the table below.

| Nanoswitch | Name | Sequence (5′-3′) | Len. | Pos |
|---|---|---|---|---|
| 1 | DENV Target | GTGACTGAGGACTGCGGAAATAGAGG | 30 | 2823 |
| | 1 | ACCC (SEQ ID NO: 71) | | |
| | v4-DENV T1 | ACCGTTGTAGCAATACTTCTTTGATTAG | | |
| | 40-15 | TAATAACATCACGGGTCCTCTATTTCC | 55 | |
| | | (SEQ ID NO: 72) | | |
| | v8-DENV T1 | GCAGTCCTCAGTCACTCAACCGATTGA | | |
| | 15-40 | GGGAGGGAAGGTAAATATTGACGGAA | 55 | |
| | | AT (SEQ ID NO: 73) | | |
| 2 | DENV Target | CTCTCCTCCCAGAGCACTATACCAGAG | 30 | 3280 |
| | 2 | ACC (SEQ ID NO: 74) | | |
| | v4-DENV T2 | ACCGTTGTAGCAATACTTCTTTGATTAG | 55 | |
| | 40-15 | TAATAACATCACGGTCTCTGGTATAGT | | |
| | | (SEQ ID NO: 75) | | |
| | v8-DENV T2 | GCTCTGGGAGGAGAGTCAACCGATTG | 55 | |
| | 15-40 | AGGGAGGGAAGGTAAATATTGACGGA | | |
| | | AAT (SEQ ID NO: 76) | | |
| 3 | DENV Target | TGCTCACTGGACGATCGGCCGATTTG | 30 | 3805 |
| | 3 | GAAC (SEQ ID NO: 77) | | |
| | v4-DENV T3 | ACCGTTGTAGCAATACTTCTTTGATTAG | 55 | |
| | 40-15 | TAATAACATCACGTTCCAAATCGGCCG | | |
| | | (SEQ ID NO: 78) | | |
| | v8-DENV T3 | ATCGTCCAGTGAGCATCAACCGATTGA | 55 | |
| | 15-40 | GGGAGGGAAGGTAAATATTGACGGAA | | |
| | | AT (SEQ ID NO: 79) | | |
| 4 | DENV Target | GGCCAGCACTCCAAGCAAAAGCATCC | 30 | 4259 |
| | 4 | AGAG (SEQ ID NO: 80) | | |
| | v4-DENV T4 | ACCGTTGTAGCAATACTTCTTTGATTAG | 55 | |
| | 40-15 | TAATAACATCACCTCTGGATGCTTTTG | | |
| | | (SEQ ID NO: 81) | | |
| | v8-DENV T4 | CTTGGAGTGCTGGCCTCAACCGATTGA | 55 | |
| | 15-40 | GGGAGGGAAGGTAAATATTGACGGAA | | |
| | | AT (SEQ ID NO: 82) | | |
| 5 | DENV Target | CACACCAGAAGGGAAAGTAGTGGACC | 30 | 5488 |
| | 5 | TCGG (SEQ ID NO: 83) | | |
| | v4-DENV T5 | ACCGTTGTAGCAATACTTCTTTGATTAG | 55 | |
| | 40-15 | TAATAACATCACCCGAGGTCCACTACT | | |
| | | (SEQ ID NO: 84) | | |
| | v8-DENV T5 | TTCCCTTCTGGTGTGTCAACCGATTGA | 55 | |
| | 15-40 | GGGAGGGAAGGTAAATATTGACGGAA | | |
| | | AT (SEQ ID NO: 85) | | |
| 6 | DENV Target | AAGCCACTTACGAGCCGGATGTTGACC | 30 | 6432 |
| | 6 | TCG (SEQ ID NO: 86) | | |
| | v4-DENV T6 | ACCGTTGTAGCAATACTTCTTTGATTAG | 55 | |
| | 40-15 | TAATAACATCACCGAGGTCAACATCCG | | |
| | | (SEQ ID NO: 87) | | |
| | v8-DENV T6 | GCTCGTAAGTGGCTTTCAACCGATTGA | 55 | |
| | 15-40 | GGGAGGGAAGGTAAATATTGACGGAA | | |
| | | AT (SEQ ID NO: 88) | | |
| 7 | DENV Target | GCATGGCGTAGTGGACTAGCGGTTAG | 30 | 7190 |
| | 7 | AGGA (SEQ ID NO: 89) | | |
| | v4-DENV T7 | ACCGTTGTAGCAATACTTCTTTGATTAG | 55 | |
| | 40-15 | TAATAACATCACTCCTCTAACCGCTAG | | |
| | | (SEQ ID NO: 90) | | |
| | v8-DENV T7 | TCCACTACGCCATGCTCAACCGATTGA | 55 | |
| | 15-40 | GGGAGGGAAGGTAAATATTGACGGAA | | |
| | | AT (SEQ ID NO: 91) | | |
| 8 | DENV Target | CAAGCTACAGCTCAAAGGAATGTCATA | 30 | 7782 |
| | 8 | CTC (SEQ ID NO: 92) | | |
| | v4-DENV T8 | ACCGTTGTAGCAATACTTCTTTGATTAG | 55 | |
| | 40-15 | TAATAACATCACGAGTATGACATTCCT | | |
| | | (SEQ ID NO: 93) | | |
| | v8-DEN T8 | TTGAGCTGTAGCTTGTCAACCGATTGA | 55 | |
| | 15-40 | GGGAGGGAAGGTAAATATTGACGGAA | | |
| | | AT (SEQ ID NO: 94) | | |
| 9 | DENV Target | GACCCATTTCCTCAGAGCAATGCACCA | 30 | 8315 |
| | 9 | ATC (SEQ ID NO: 95) | | |
| | v4-DENV T9 | ACCGTTGTAGCAATACTTCTTTGATTAG | 55 | |
| | 40-15 | TAATAACATCACGATTGGTGCATTGCT | | |
| | | (SEQ ID NO: 96) | | |
| | v8-DENV T9 | CTGAGGAAATGGGTCTCAACCGATTG | 55 | |
| | 15-40 | AGGGAGGGAAGGTAAATATTGACGGA | | |
| | | AAT (SEQ ID NO: 97) | | |
| 10 | DENV Target | GAAGGCAAGAAACGCACTGGACAACTT | 30 | 8653 |
| | 13 | AGC (SEQ ID NO: 98) | | |
| | v4-DENV T10 | ACCGTTGTAGCAATACTTCTTTGATTAG | 55 | |
| | 40-15 | TAATAACATCACGCTAAGTTGTCCAGT | | |
| | | (SEQ ID NO: 99) | | |
| | v8-DENV T10 | GCGTTTCTTGCCTTCTCAACCGATTGA | 55 | |
| | 15-40 | GGGAGGGAAGGTAAATATTGACGGAA | | |
| | | AT (SEQ ID NO: 100) | | |
| 11 | DENV Target | AGAACCCAAGAACCGAAAGAAGGCAC | 30 | 9313 |
| | 11 | GAAG (SEQ ID NO: 101) | | |
| | v4-DENV T11 | ACCGTTGTAGCAATACTTCTTTGATTAG | 55 | |
| | 40-15 | TAATAACATCACCTTCGTGCCTTCTTT | | |
| | | (SEQ ID NO: 102) | | |
| | v8-DENV T11 | CGGTTCTTGGGTTCTTCAACCGATTGA | 55 | |
| | 15-40 | GGGAGGGAAGGTAAATATTGACGGAA | | |
| | | AT (SEQ ID NO: 103) | | |
| 12 | DENV Target | AGACCAACACCAAGAGGCACAGTAATG | 30 | 10481 |
| | 12 | GAC (SEQ ID NO: 104) | | |
| | v4-DENV T12 | ACCGTTGTAGCAATACTTCTTTGATTAG | 55 | |
| | 40-15 | TAATAACATCACGTCCATTACTGTGCC | | |
| | | (SEQ ID NO: 105) | | |
| | v8-DENV T12 | TCTTGGTGTTGGTCTTCAACCGATTGA | 55 | |
| | 15-40 | GGGAGGGAAGGTAAATATTGACGGAA | | |
| | | AT (SEQ ID NO: 106) | | |

TABLE 5
Variable oligos for constructing nanoswitches with different loop
sizes used in FIG. 3B, and FIG. 3D. In embodiments, suitable polynucleotides
include those having a nucleotide sequence having at least 80%, 90%, 95%, 97%,
99% sequence identity to SEQ ID NOS: 107 to 112 as set forth in the table below.

| | Name | Sequence (5′-3′) | Length |
|---|---|---|---|
| For v4-v8 | v4 oligo | Oligos with prefix ‘v4-’ | |
| loop | v8 oligo | Oligos with prefix ‘v8-’ | |
| nanoswitch | Var 4 filler | TCTGTCCATCACGCAAATTA | 20 |
| | | (SEQ ID NO: 107) | |
| | Var 8 filler | TATTCATTAAAGGTGAATTA | 20 |
| | | (SEQ ID NO: 108) | |
| For v4-v6 | v4 oligo | Oligos with prefix ‘v4-’ | |
| loop | v8 oligo | Oligos with prefix ‘v6-’ | |
| nanoswitch | Var 4 filler | TCTGTCCATCACGCAAATTA | 20 |
| | | (SEQ ID NO: 109) | |
| | Var 6 filler | TCGCAAGACAAAGAACGCGA | 20 |
| | | (SEQ ID NO: 110) | |
| For v4-v7 | v4 oligo | Oligos with prefix ‘v4-’ | |
| loop | v7 oligo | Oligos with prefix ‘v7-’ | 20 |
| nanoswitch | Var 4 filler | TCTGTCCATCACGCAAATTA | 20 |
| | | (SEQ ID NO: 111) | |
| | Var 7 filler | TCGCAAGACAAAGAACGCGA | |
| | | (SEQ ID NO: 112) | |

TABLE 6
Target sequences and the corresponding detector ssDNA for the
ZIKV and DENV multiplexing test (FIG. 3B). In embodiments, suitable
polynucleotides include those having a nucleotide sequence having at least
80%, 90%, 95%, 97%, 99% sequence identity to SEQ ID NOS: 113 to 118
as set forth in the table below.

| Name | Sequence (5′-3′) | Len. |
|---|---|---|
| ZIKV_Target | AACGCCCAATTCACCAAGAGCCGAAGCCAC (SEQ ID | 30 |
| 3 | NO: 113) | |
| v4-ZIKV T3 | ACCGTTGTAGCAATACTTCTTTGATTAGTAATAACATCACGT | 55 |
| 40-15 | GGCTTCGGCTCTT (SEQ ID NO: 114) | |
| v8-ZIKV T3 | GGTGAATTGGGCGTTTCAACCGATTGAGGGAGGGAAGGTA | 55 |
| 15-40 | AATATTGACGGAAAT (SEQ ID NO: 115) | |
| DENV_ | GCATGGCGTAGTGGACTAGCGGTTAGAGGA (SEQ ID | 30 |
| Target 10 | NO: 116) | |
| v4-DENV | ACCGTTGTAGCAATACTTCTTTGATTAGTAATAACATCACTC | 55 |
| T10 40-15 | CTCTAACCGCTAG (SEQ ID NO: 117) | |
| v6-DENV | TCCACTACGCCATGCTGGGTTATATAACTATATGTAAATGC | 55 |
| T10 15-40 | TGATGCAAATCCAA (SEQ ID NO: 118) | |

TABLE 7
Target sequences and the corresponding detection arm ssDNA for
the ZIKV Cambodia and Uganda specificity test (FIG. 3D). In embodiments,
suitable polynucleotides include those having a nucleotide sequence having at
least 80%, 90%, 95%, 97%, 99% sequence identity to SEQ ID NOS: 119 to 148
as set forth in the table below.

| Name | Sequence (5′-3′) | Len. |
|---|---|---|
| Cambodia_1st | AGACTATCATGCTTTTGGGGTTGCTGGGAA (SEQ ID | 30 |
| | NO: 119) | |
| v4- | ACCGTTGTAGCAATACTTCTTTGATTAGTAATAACATCAC | 55 |
| Cambodia_1st_ | TTCCCAGCAACCCCA (SEQ ID NO: 120) | |
| 40_15 | | |
| v8- | AAAGCATGATAGTCTTCAACCGATTGAGGGAGGGAAGG | 55 |
| Cambodia_1st_ | TAAATATTGACGGAAAT (SEQ ID NO: 121) | |
| 15_40 | | |
| Cambodia_2nd | TTGTTCGGTATGGGTAAAGGGATGCCATTC (SEQ ID | 30 |
| | NO: 122) | |
| v4- | ACCGTTGTAGCAATACTTCTTTGATTAGTAATAACATCAC | 55 |
| Cambodia_2nd_ | GAATGGCATCCCTTT (SEQ ID NO: 123) | |
| 40_15 | | |
| v8- | ACCCATACCGAACAATCAACCGATTGAGGGAGGGAAGG | 55 |
| Cambodia_2nd_ | TAAATATTGACGGAAAT (SEQ ID NO: 124) | |
| 15_40 | | |
| Cambodia_3rd | GCGAAGGTTGAGATAACGCCCAATTCACCA (SEQ ID | 30 |
| | NO: 125) | |
| v4- | ACCGTTGTAGCAATACTTCTTTGATTAGTAATAACATCAC | 55 |
| Cambodia_3rd_ | TGGTGAATTGGGCGT (SEQ ID NO: 126) | |
| 40_15 | | |
| v8- | TATCTCAACCTTCGCTCAACCGATTGAGGGAGGGAAGG | 55 |
| Cambodia_3rd_ | TAAATATTGACGGAAAT (SEQ ID NO: 127) | |
| 15_40 | | |
| Cambodia_4th | GTACCGCAGCGTTCACATTCACTAAGATCC (SEQ ID | 30 |
| | NO: 128) | |
| v4- | ACCGTTGTAGCAATACTTCTTTGATTAGTAATAACATCAC | 55 |
| Cambodia_4th_ | GGATCTTAGTGAATG (SEQ ID NO: 129) | |
| 40_15 | | |
| v8- | TGAACGCTGCGGTACTCAACCGATTGAGGGAGGGAAGG | 55 |
| Cambodia_4th_ | TAAATATTGACGGAAAT (SEQ ID NO: 130) | |
| 15_40 | | |
| Cambodia_5th | CTGCTCTGACAACTTTCATTACCCCAGCCG (SEQ ID | 30 |
| | NO: 131) | |
| v4- | ACCGTTGTAGCAATACTTCTTTGATTAGTAATAACATCAC | 55 |
| Cambodia_5th_ | CGGCTGGGGTAATGA (SEQ ID NO: 132) | |
| 40_15 | | |
| v8- | AAGTTGTCAGAGCAGTCAACCGATTGAGGGAGGGAAGG | 55 |
| Cambodia_5th_ | TAAATATTGACGGAAAT (SEQ ID NO: 133) | |
| 15_40 | | |
| Uganda_1st | AGACCATTATGCTCTTAGGTTTGCTGGGAA (SEQ ID | 30 |
| | NO: 134) | |
| v4- | ACCGTTGTAGCAATACTTCTTTGATTAGTAATAACATCAC | 55 |
| Uganda_1st_ | TTCCCAGCAAACCTA (SEQ ID NO: 135) | |
| 40_15 | | |
| v7- | AGAGCATAATGGTCTGTTTTAGCGAACCTCCCGACTTGC | 55 |
| Uganda_1st_ | GGGAGGTTTTGAAGCC (SEQ ID NO: 136) | |
| 15_40 | | |
| Uganda_2nd | CTGTTTGGCATGGGCAAAGGGATGCCATTT (SEQ ID | 30 |
| | NO: 137) | |
| v4- | ACCGTTGTAGCAATACTTCTTTGATTAGTAATAACATCAC | 55 |
| Uganda_2nd_ | AAATGGCATCCCTTT (SEQ ID NO: 138) | |
| 40_15 | | |
| v7- | GCCCATGCCAAACAGGTTTTAGCGAACCTCCCGACTTG | 55 |
| Uganda_2nd_ | CGGGAGGTTTTGAAGCC (SEQ ID NO: 139) | |
| 15_40 | | |
| Uganda_3rd | GCGAAAGTCGAGGTTACGCCTAATTCACCA (SEQ ID | 30 |
| | NO: 140) | |
| v4- | ACCGTTGTAGCAATACTTCTTTGATTAGTAATAACATCAC | 55 |
| Uganda_3rd_ | TGGTGAATTAGGCGT (SEQ ID NO: 141) | |
| 40_15 | | |
| v7- | AACCTCGACTTTCGCGTTTTAGCGAACCTCCCGACTTGC | 55 |
| Uganda_3rd_ | GGGAGGTTTTGAAGCC (SEQ ID NO: 142) | |
| 15_40 | | |
| Uganda_4th | GCACTGCGGCATTCACATTCACCAAGGTCC (SEQ ID | 30 |
| | NO: 143) | |
| v4- | ACCGTTGTAGCAATACTTCTTTGATTAGTAATAACATCAC | 55 |
| Uganda_4th_ | GGACCTTGGTGAATG (SEQ ID NO: 144) | |
| 40_15 | | |
| v7- | TGAATGCCGCAGTGCGTTTTAGCGAACCTCCCGACTTG | 55 |
| Uganda_4th_ | CGGGAGGTTTTGAAGCC (SEQ ID NO: 145) | |
| 15_40 | | |
| Uganda_5th | CCGCATTGACAACTCTCATCACCCCAGCTG (SEQ ID | 30 |
| | NO: 146) | |
| v4- | ACCGTTGTAGCAATACTTCTTTGATTAGTAATAACATCAC | 55 |
| Uganda_5th_ | CAGCTGGGGTGATGA (SEQ ID NO: 147) | |
| 40_15 | | |
| v7- | GAGTTGTCAATGCGGGTTTTAGCGAACCTCCCGACTTG | 55 |
| Uganda_5th_ | CGGGAGGTTTTGAAGCC (SEQ ID NO: 148) | |
| 15_40 | | |

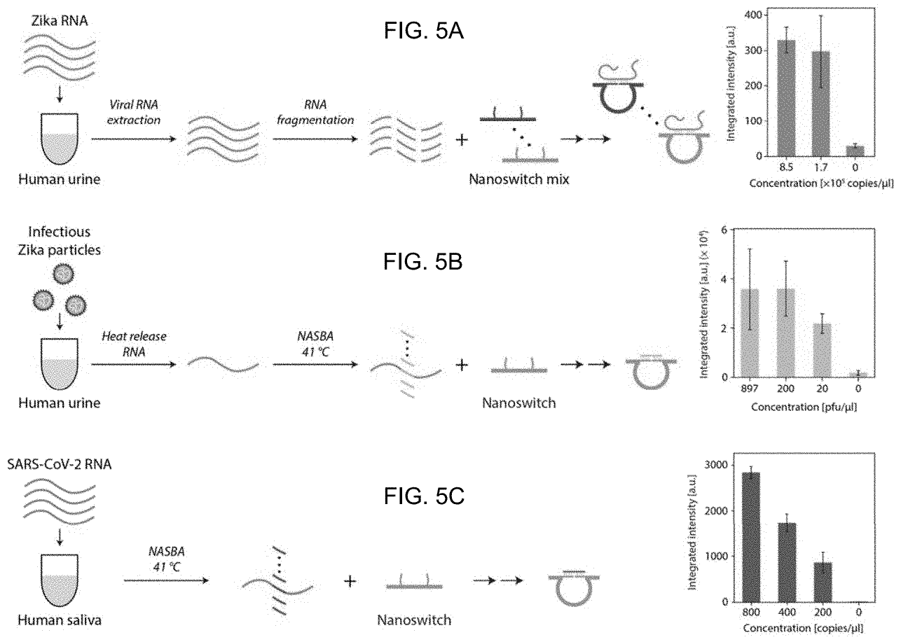

TABLE 8
Amplified region of ZIKV RNA, primers, targets and corresponding
detector ssDNA used in NASBA related experiments in FIGS. 5B, 20, and 21.
In embodiments, suitable polynucleotides include those having a nucleotide
sequence having at least 80%, 90%, 95%, 97%, 99% sequence identity to SEQ ID
NOS: 150 to 158 as set forth in the table below.
The forward primer has a T7 promoter:
AATTCTAATACGACTCACTATAGGGAGAAGG. (SEQ ID NO: 149).

| Name | Sequence (5′-3′) | Len. |
|---|---|---|
| Amplified region on | AATGCTGTCAGTTCATGGCTCCCAGCACAGTGG | 167 |
| ZIKV RNA (1394-1560) | GATGATCGTTAATGATACAGGACATGAAACTGAT | |
| | GAGAATAGAGCGAAGGTTGAGATAACGCCCAAT | |
| | TCACCAAGAGCCGAAGCCACCCTGGGGGGTTTT | |
| | GGAAGCCTAGGACTTGATTGTGAACCGAGGAC | |
| | AG (SEQ ID NO: 150) | |
| ZIKV NASBA_Reverse | CTGTCCTCGGTTCACAATCA (SEQ ID NO: 151) | 20 |
| primer | | |
| ZIKV NASBA_Forward | AATTCTAATACGACTCACTATAGGGAGAAGGAA | 55 |
| primer | TGCTGTCAGTTCATGGCTCCCA (SEQ ID NO: 152) | |
| ZIKV_NASBA_Target A | AACGCCCAATTCACCAAGAGCCGAAGCCAC | 30 |
| | (SEQ ID NO: 153) | |
| v4-ZIKV NASBA_Target | ACCGTTGTAGCAATACTTCTTTGATTAGTAATAA | 55 |
| A 40-15 | CATCACGTGGCTTCGGCTCTT (SEQ ID NO: 154) | |
| v8-ZIKV NASBA_Target | GGTGAATTGGGCGTTTCAACCGATTGAGGGAGG | 55 |
| A 15-40 | GAAGGTAAATATTGACGGAAAT (SEQ ID NO: 155) | |
| ZIKV_NASBA_Target B | GATGATCGTTAATGATACAGGACATGAAAC (SEQ | 30 |
| | ID NO: 156) | |
| v4-ZIKV NASBA_Target | ACCGTTGTAGCAATACTTCTTTGATTAGTAATAA | 55 |
| B 40-15 | CATCACGTTTCATGTCCTGTA (SEQ ID NO: 157) | |
| v8-ZIKV NASBA_Target | TCATTAACGATCATCTCAACCGATTGAGGGAGG | 55 |
| B 15-40 | GAAGGTAAATATTGACGGAAAT (SEQ ID NO: 158) | |

TABLE 9
DNA template, primers, targets and the corresponding detector
ssDNA for SARS-CoV-2 RNA detection. In embodiments, suitable polynucleotides
include those having a nucleotide sequence having at least 80%, 90%, 95%, 97%,
99% sequence identity to SEQ ID NOS: 159 to 168 as set forth in the table below.

| Name | Sequence (5′-3′) | Len. |
|---|---|---|
| DNA template | TGGGGTTTTACAGGTAACCTACAAAGCAACCAT | 132 |
| | GATCTGTATTGTCAAGTCCATGGTAATGCACATG | |
| | TAGCTAGTTGTGATGCAATCATGACTAGGTGTCT | |
| | AGCTGTCCACGAGTGCTTTGTTAAGCGTGTT | |
| | (SEQ ID NO: 159) | |
| SARS-CoV-2 RNA | UGGGGUUUUACAGGUAACCUACAAAGCAACCA | 132 |
| fragment | UGAUCUGUAUUGUCAAGUCCAUGGUAAUGCAC | |
| | AUGUAGCUAGUUGUGAUGCAAUCAUGACUAGG | |
| | UGUCUAGCUGUCCACGAGUGCUUUGUUAAGCG | |
| | UGUU (SEQ ID NO: 160) | |
| Forward primer | AATTCTAATACGACTCACTATAGGGAGAAGGTG | 55 |
| | GGGTTTTACRGGTAACCT (SEQ ID NO: 161) | |
| Reverse primer | AACACGCTTAACAAAGCACTC (SEQ ID NO: 162) | 30 |
| Target1 | CCATGATCTGTATTGTCAAGTCCATGGTAA (SEQ | |
| | ID NO: 163) | |
| T1-v4-COVID19 40-15 | ACCGTTGTAGCAATACTTCTTTGATTAGTAATAA | 55 |
| | CATCACTTACCATGGACTTGA (SEQ ID NO: 164) | |
| T1-V8-COVID19 15-40 | CAATACAGATCATGGTCAACCGATTGAGGGAGG | 55 |
| | GAAGGTAAATATTGACGGAAAT (SEQ ID NO: 165) | |
| Target2 | ATGCAATCATGACTAGGTGTCTAGCTGTCC (SEQ | |
| | ID NO: 166) | |
| T2-v4-COVID19 40-15 | ACCGTTGTAGCAATACTTCTTTGATTAGTAATAA | 55 |
| | CATCACGGACAGCTAGACACC (SEQ ID NO: 167) | |
| T2-V8-COVID19 15-40 | TAGTCATGATTGCATTCAACCGATTGAGGGAGG | 55 |
| | GAAGGTAAATATTGACGGAAAT (SEQ ID NO: 168) | |

TABLE 10
Backbone and basic variable oligos for the construction of
nanoswitches and other oligos. In embodiments, suitable
polynucleotides include those having a nucleotide sequence
having at least 80%, 90%, 95%, 97%, 99% sequence identity
to SEQ ID NOS: 169 to 277 as set forth in the table below.
Backbone oligos

| # | Sequence (5′-3′) | Length |
|---|---|---|
| 1 | AGAGCATAAAGCTAAATCGGTTGTACCAAAAACATTATGACCCTGTA | 60 |
| | ATACTTTTGCGGG (SEQ ID NO: 169) | |
| 2 | AGAAGCCTTTATTTCAACGCAAGGATAAAAATTTTTAGAACCCTCATA | 60 |
| | TATTTTAAATGC (SEQ ID NO: 170) | |
| 3 | AATGCCTGAGTAATGTGTAGGTAAAGATTCAAAAGGGTGAGAAAGG | 60 |
| | CCGGAGACAGTCAA (SEQ ID NO: 171) | |
| 4 | ATCACCATCAATATGATATTCAACCGTTCTAGCTGATAAATTAATGCC | 60 |
| | GGAGAGGGTAGC (SEQ ID NO: 172) | |
| 5 | TATTTTTGAGAGATCTACAAAGGCTATCAGGTCATTGCCTGAGAGTC | 60 |
| | TGGAGCAAACAAG (SEQ ID NO: 173) | |
| 6 | AGAATCGATGAACGGTAATCGTAAAACTAGCATGTCAATCATATGTA | 60 |
| | CCCCGGTTGATAA (SEQ ID NO: 174) | |
| 7 | TCAGAAAAGCCCCAAAAACAGGAAGATTGTATAAGCAAATATTTAAA | 60 |
| | TTGTAAACGTTAA (SEQ ID NO: 175) | |
| 8 | TATTTTGTTAAAATTCGCATTAAATTTTTGTTAAATCAGCTCATTTTTT | 60 |
| | AACCAATAGGA (SEQ ID NO: 176) | |
| 9 | ACGCCATCAAAAATAATTCGCGTCTGGCCTTCCTGTAGCCAGCTTTC | 60 |
| | ATCAACATTAAAT (SEQ ID NO: 177) | |
| 10 | GGATAGGTCACGTTGGTGTAGATGGGCGCATCGTAACCGTGCATCT | 60 |
| | GCCAGTTTGAGGGG (SEQ ID NO: 178) | |
| 11 | ACGACGACAGTATCGGCCTCAGGAAGATCGCACTCCAGCCAGCTTT | 60 |
| | CCGGCACCGCTTCT (SEQ ID NO: 179) | |
| 12 | GGTGCCGGAAACCAGGCAAAGCGCCATTCGCCATTCAGGCTGCGC | 60 |
| | AACTGTTGGGAAGGG (SEQ ID NO: 180) | |
| 13 | CGATCGGTGCGGGCCTCTTCGCTATTACGCCAGCTGGCGAAAGGG | 60 |
| | GGATGTGCTGCAAGG (SEQ ID NO: 181) | |
| 14 | CGATTAAGTTGGGTAACGCCAGGGTTTTCCCAGTCACGACGTTGTA | 60 |
| | AAACGACGGCCAGT (SEQ ID NO: 182) | |
| 15 | GCCAAGCTTGCATGCCTGCAGGTCGACTCTAGAGGATCCCCGGGT | 60 |
| | ACCGAGCTCGAATTC (SEQ ID NO: 183) | |
| 16 | GTAATCATGGTCATAGCTGTTTCCTGTGTGAAATTGTTATCCGCTCA | 60 |
| | CAATTCCACACAA (SEQ ID NO: 184) | |
| 17 | CATACGAGCCGGAAGCATAAAGTGTAAAGCCTGGGGTGCCTAATGA | 60 |
| | GTGAGCTAACTCAC (SEQ ID NO: 185) | |
| 18 | ATTAATTGCGTTGCGCTCACTGCCCGCTTTCCAGTCGGGAAACCTG | 60 |
| | TCGTGCCAGCTGCA (SEQ ID NO: 186) | |
| 19 | TTAATGAATCGGCCAACGCGCGGGGAGAGGCGGTTTGCGTATTGG | 60 |
| | GCGCCAGGGTGGTTT (SEQ ID NO: 187) | |
| 20 | GTTGCAGCAAGCGGTCCACGCTGGTTTGCCCCAGCAGGCGAAAAT | 60 |
| | CCTGTTTGATGGTGG (SEQ ID NO: 188) | |
| 21 | TTCCGAAATCGGCAAAATCCCTTATAAATCAAAAGAATAGCCCGAGA | 60 |
| | TAGGGTTGAGTGT (SEQ ID NO: 189) | |
| 22 | TGTTCCAGTTTGGAACAAGAGTCCACTATTAAAGAACGTGGACTCCA | 60 |
| | ACGTCAAAGGGCG (SEQ ID NO: 190) | |
| 23 | AAAAACCGTCTATCAGGGCGATGGCCCACTACGTGAACCATCACCC | 60 |
| | AAATCAAGTTTTTT (SEQ ID NO: 191) | |
| 24 | GGGGTCGAGGTGCCGTAAAGCACTAAATCGGAACCCTAAAGGGAG | 60 |
| | CCCCCGATTTAGAGC (SEQ ID NO: 192) | |
| 25 | TTGACGGGGAAAGCCGGCGAACGTGGCGAGAAAGGAAGGGAAGAA | 60 |
| | AGCGAAAGGAGCGGG (SEQ ID NO: 193) | |
| 26 | CGCTAGGGCGCTGGCAAGTGTAGCGGTCACGCTGCGCGTAACCAC | 60 |
| | CACACCCGCCGCGCT (SEQ ID NO: 194) | |
| 27 | TAATGCGCCGCTACAGGGCGCGTACTATGGTTGCTTTGACGAGCAC | 60 |
| | GTATAACGTGCTTT (SEQ ID NO: 195) | |
| 28 | CCTCGTTAGAATCAGAGCGGGAGCTAAACAGGAGGCCGATTAAAGG | 60 |
| | GATTTTAGACAGGA (SEQ ID NO: 196) | |
| 29 | ACGGTACGCCAGAATCCTGAGAAGTGTTTTTATAATCAGTGAGGCC | 60 |
| | ACCGAGTAAAAGAG (SEQ ID NO: 197) | |
| 30 | TTGCCTGAGTAGAAGAACTCAAACTATCGGCCTTGCTGGTAATATCC | 60 |
| | AGAACAATATTAC (SEQ ID NO: 198) | |
| 31 | CGCCAGCCATTGCAACAGGAAAAACGCTCATGGAAATACCTACATTT | 60 |
| | TGACGCTCAATCG (SEQ ID NO: 199) | |
| 32 | TCTGAAATGGATTATTTACATTGGCAGATTCACCAGTCACACGACCA | 60 |
| | GTAATAAAAGGGA (SEQ ID NO: 200) | |
| 33 | CATTCTGGCCAACAGAGATAGAACCCTTCTGACCTGAAAGCGTAAG | 60 |
| | AATACGTGGCACAG (SEQ ID NO: 201) | |
| 34 | ACAATATTTTTGAATGGCTATTAGTCTTTAATGCGCGAACTGATAGC | 60 |
| | CCTAAAACATCGC (SEQ ID NO: 202) | |
| 35 | CATTAAAAATACCGAACGAACCACCAGCAGAAGATAAAACAGAGGT | 60 |
| | GAGGCGGTCAGTAT (SEQ ID NO: 203) | |
| 36 | TAACACCGCCTGCAACAGTGCCACGCTGAGAGCCAGCAGCAAATGA | 60 |
| | AAAATCTAAAGCAT (SEQ ID NO: 204) | |
| 37 | CACCTTGCTGAACCTCAAATATCAAACCCTCAATCAATATCTGGTCA | 60 |
| | GTTGGCAAATCAA (SEQ ID NO: 205) | |
| 38 | CAGTTGAAAGGAATTGAGGAAGGTTATCTAAAATATCTTTAGGAGCA | 60 |
| | CTAACAACTAATA (SEQ ID NO: 206) | |
| 39 | GATTAGAGCCGTCAATAGATAATACATTTGAGGATTTAGAAGTATTA | 60 |
| | GACTTTACAAACA (SEQ ID NO: 207) | |
| 40 | CATTATCATTTTGCGGAACAAAGAAACCACCAGAAGGAGCGGAATTA | 60 |
| | TCATCATATTCCT (SEQ ID NO: 208) | |
| 41 | GATTATCAGATGATGGCAATTCATCAATATAATCCTGATTGTTTGGAT | 60 |
| | TATACTTCTGAA (SEQ ID NO: 209) | |
| 42 | TAATGGAAGGGTTAGAACCTACCATATCAAAATTATTTGCACGTAAA | 60 |
| | ACAGAAATAAAGA (SEQ ID NO: 210) | |
| 43 | AATTGCGTAGATTTTCAGGTTTAACGTCAGATGAATATACAGTAACA | 60 |
| | GTACCTTTTACAT (SEQ ID NO: 211) | |
| 44 | CGGGAGAAACAATAACGGATTCGCCTGATTGCTTTGAATACCAAGTT | 60 |
| | ACAAAATCGCGCA (SEQ ID NO: 212) | |
| 45 | GAGGCGAATTATTCATTTCAATTACCTGAGCAAAAGAAGATGATGAA | 60 |
| | ACAAACATCAAGA (SEQ ID NO: 213) | |
| 46 | AAACAAAATTAATTACATTTAACAATTTCATTTGAATTACCTTTTTTAA | 60 |
| | TGGAAACAGTA (SEQ ID NO: 214) | |
| 47 | CATAAATCAATATATGTGAGTGAATAACCTTGCTTCTGTAAATCGTCG | 60 |
| | CTATTAATTAAT (SEQ ID NO: 215) | |
| 48 | TTTCCCTTAGAATCCTTGAAAACATAGCGATAGCTTAGATTAAGACG | 60 |
| | CTGAGAAGAGTCA (SEQ ID NO: 216) | |
| 49 | ATAGTGAATTTATCAAAATCATAGGTCTGAGAGACTACCTTTTTAACC | 60 |
| | TCCGGCTTAGGT (SEQ ID NO: 217) | |
| 50 | GAAAACTTTTTCAAATATATTTTAGTTAATTTCATCTTCTGACCTAAAT | 60 |
| | TTAATGGTTTG (SEQ ID NO: 218) | |
| 51 | AAATACCGACCGTGTGATAAATAAGGCGTTAAATAAGAATAAACACC | 60 |
| | GGAATCATAATTA (SEQ ID NO: 219) | |
| 52 | CTAGAAAAAGCCTGTTTAGTATCATATGCGTTATACAAATTCTTACCA | 60 |
| | GTATAAAGCCAA (SEQ ID NO: 220) | |
| 53 | CGCTCAACAGTAGGGCTTAATTGAGAATCGCCATATTTAACAACGCC | 60 |
| | AACATGTAATTTA (SEQ ID NO: 221) | |
| 54 | GGCAGAGGCATTTTCGAGCCAGTAATAAGAGAATATAAAGTACCGA | 60 |
| | CAAAAGGTAAAGTA (SEQ ID NO: 222) | |
| 55 | ATTCTGTCCAGACGACGACAATAAACAACATGTTCAGCTAATGCAGA | 60 |
| | ACGCGCCTGTTTA (SEQ ID NO: 223) | |
| 56 | TCAACAATAGATAAGTCCTGAACAAGAAAAATAATATCCCATCCTAAT | 60 |
| | TTACGAGCATGT (SEQ ID NO: 224) | |
| 57 | AGAAACCAATCAATAATCGGCTGTCTTTCCTTATCATTCCAAGAACG | 60 |
| | GGTATTAAACCAA (SEQ ID NO: 225) | |
| 58 | GTACCGCACTCATCGAGAACAAGCAAGCCGTTTTTATTTTCATCGTA | 60 |
| | GGAATCATTACCG (SEQ ID NO: 226) | |
| 59 | CGCCCAATAGCAAGCAAATCAGATATAGAAGGCTTATCCGGTATTCT | 60 |
| | AAGAACGCGAGGC (SEQ ID NO: 227) | |
| 60 | ATTTTGCACCCAGCTACAATTTTATCCTGAATCTTACCAACGCTAAC | 60 |
| | GAGCGTCTTTCCA (SEQ ID NO: 228) | |
| 61 | GAGCCTAATTTGCCAGTTACAAAATAAACAGCCATATTATTTATCCCA | 60 |
| | ATCCAAATAAGA (SEQ ID NO: 229) | |
| 62 | AACGATTTTTTGTTTAACGTCAAAAATGAAAATAGCAGCCTTTACAGA | 60 |
| | GAGAATAACATA (SEQ ID NO: 230) | |
| 63 | AAAACAGGGAAGCGCATTAGACGGGAGAATTAACTGAACACCCTGA | 60 |
| | ACAAAGTCAGAGGG (SEQ ID NO: 231) | |
| 64 | TAATTGAGCGCTAATATCAGAGAGATAACCCACAAGAATTGAGTTAA | 60 |
| | GCCCAATAATAAG (SEQ ID NO: 232) | |
| 65 | AGCAAGAAACAATGAAATAGCAATAGCTATCTTACCGAAGCCCTTTT | 60 |
| | TAAGAAAAGTAAG (SEQ ID NO: 233) | |
| 66 | CAGATAGCCGAACAAAGTTACCAGAAGGAAACCGAGGAAACGCAAT | 60 |
| | AATAACGGAATACC (SEQ ID NO: 234) | |
| 67 | CAAAAGAACTGGCATGATTAAGACTCCTTATTACGCAGTATGTTAGC | 60 |
| | AAACGTAGAAAAT (SEQ ID NO: 235) | |
| 68 | ACATACATAAAGGTGGCAACATATAAAAGAAACGCAAAGACACCAC | 60 |
| | GGAATAAGTTTATT (SEQ ID NO: 236) | |
| 69 | TTGTCACAATCAATAGAAAATTCATATGGTTTACCAGCGCCAAAGAC | 60 |
| | AAAAGGGCGACAT (SEQ ID NO: 237) | |
| 70 | TCACCGTCACCGACTTGAGCCATTTGGGAATTAGAGCCAGCAAAAT | 60 |
| | CACCAGTAGCACCA (SEQ ID NO: 238) | |
| 71 | TTACCATTAGCAAGGCCGGAAACGTCACCAATGAAACCATCGATAG | 60 |
| | CAGCACCGTAATCA (SEQ ID NO: 239) | |
| 72 | GTAGCGACAGAATCAAGTTTGCCTTTAGCGTCAGACTGTAGCGCGT | 60 |
| | TTTCATCGGCATTT (SEQ ID NO: 240) | |
| 73 | TCGGTCATAGCCCCCTTATTAGCGTTTGCCATCTTTTCATAATCAAAA | 60 |
| | TCACCGGAACCA (SEQ ID NO: 241) | |
| 74 | GAGCCACCACCGGAACCGCCTCCCTCAGAGCCGCCACCCTCAGAA | 60 |
| | CCGCCACCCTCAGAG (SEQ ID NO: 242) | |
| 75 | CCACCACCCTCAGAGCCGCCACCAGAACCACCACCAGAGCCGCCG | 60 |
| | CCAGCATTGACAGGA (SEQ ID NO: 243) | |
| 76 | GGTTGAGGCAGGTCAGACGATTGGCCTTGATATTCACAAACAAATA | 60 |
| | AATCCTCATTAAAG (SEQ ID NO: 244) | |
| 77 | CCAGAATGGAAAGCGCAGTCTCTGAATTTACCGTTCCAGTAAGCGT | 60 |
| | CATACATGGCTTTT (SEQ ID NO: 245) | |
| 78 | GATGATACAGGAGTGTACTGGTAATAAGTTTTAACGGGGTCAGTGC | 60 |
| | CTTGAGTAACAGTG (SEQ ID NO: 246) | |
| 79 | CCCGTATAAACAGTTAATGCCCCCTGCCTATTTCGGAACCTATTATT | 60 |
| | CTGAAACATGAAA (SEQ ID NO: 247) | |
| 80 | CCAGGCGGATAAGTGCCGTCGAGAGGGTTGATATAAGTATAGCCCG | 60 |
| | GAATAGGTGTATCA (SEQ ID NO: 248) | |
| 81 | CCGTACTCAGGAGGTTTAGTACCGCCACCCTCAGAACCGCCACCCT | 60 |
| | CAGAACCGCCACCC (SEQ ID NO: 249) | |
| 82 | TCAGAGCCACCACCCTCATTTTCAGGGATAGCAAGCCCAATAGGAA | 60 |
| | CCCATGTACCGTAA (SEQ ID NO: 250) | |
| 83 | CACTGAGTTTCGTCACCAGTACAAACTACAACGCCTGTAGCATTCCA | 60 |
| | CAGACAGCCCTCA (SEQ ID NO: 251) | |
| 84 | TAGTTAGCGTAACGATCTAAAGTTTTGTCGTCTTTCCAGACGTTAGT | 60 |
| | AAATGAATTTTCT (SEQ ID NO: 252) | |
| 85 | GTATGGGATTTTGCTAAACAACTTTCAACAGTTTCAGCGGAGTGAGA | 60 |
| | ATAGAAAGGAACA (SEQ ID NO: 253) | |
| 86 | ACTAAAGGAATTGCGAATAATAATTTTTTCACGTTGAAAATCTCCAAA | 60 |
| | AAAAAGGCTCCA (SEQ ID NO: 254) | |
| 87 | AAAGGAGCCTTTAATTGTATCGGTTTATCAGCTTGCTTTCGAGGTGA | 60 |
| | ATTTCTTAAACAG (SEQ ID NO: 255) | |
| 88 | CTTGATACCGATAGTTGCGCCGACAATGACAACAACCATCGCCCAC | 60 |
| | GCATAACCGATATA (SEQ ID NO: 256) | |
| 89 | TTCGGTCGCTGAGGCTTGCAGGGAGTTAAAGGCCGCTTTTGCGGG | 60 |
| | ATCGTCACCCTCAGC (SEQ ID NO: 257) | |
| 90 | CTTTTTCATGAGGAAGTTTCCATTAAACGGGTAAAATACGTAATGCC | 60 |
| | ACTACGAAGGCAC (SEQ ID NO: 258) | |
| 91 | CAACCTAAAACGAAAGAGGCAAAAGAATACACTAAAACACTCATCTT | 60 |
| | TGACCCCCAGCGA (SEQ ID NO: 259) | |
| 92 | TTATACCAAGCGCGAAACAAAGTACAACGGAGATTTGTATCATCGCC | 60 |
| | TGATAAATTGTGT (SEQ ID NO: 260) | |
| 93 | CGAAATCCGCGACCTGCTCCATGTTACTTAGCCGGAACGAGGCGCA | 60 |
| | GACGGTCAATCATA (SEQ ID NO: 261) | |
| 94 | AGGGAACCGAACTGACCAACTTTGAAAGAGGACAGATGAACGGTGT | 60 |
| | ACAGACCAGGCGCA (SEQ ID NO: 262) | |
| 95 | TAGGCTGGCTGACCTTCATCAAGAGTAATCTTGACAAGAACCGGAT | 60 |
| | ATTCATTACCCAAA (SEQ ID NO: 263) | |
| 96 | TCAACGTAACAAAGCTGCTCATTCAGTGAATAAGGCTTGCCCTGAC | 60 |
| | GAGAAACACCAGAA (SEQ ID NO: 264) | |
| 97 | CGAGTAGTAAATTGGGCTTGAGATGGTTTAATTTCAACTTTAATCATT | 60 |
| | GTGAATTACCTT (SEQ ID NO: 265) | |
| 98 | ATGCGATTTTAAGAACTGGCTCATTATACCAGTCAGGACGTTGGGAA | 60 |
| | GAAAAATCTACGT (SEQ ID NO: 266) | |
| 99 | TAATAAAACGAACTAACGGAACAACATTATTACAGGTAGAAAGATTC | 60 |
| | ATCAGTTGAGATT (SEQ ID NO: 267) | |
| 100 | TAAGAGCAACACTATCATAACCCTCGTTTACCAGACGACGATAAAAA | 60 |
| | CCAAAATAGCGAG (SEQ ID NO: 268) | |
| 101 | AGGCTTTTGCAAAAGAAGTTTTGCCAGAGGGGGTAATAGTAAAATGT | 60 |
| | TTAGACTGGATAG (SEQ ID NO: 269) | |
| 102 | CGTCCAATACTGCGGAATCGTCATAAATATTCATTGAATCCCCCTCA | 60 |
| | AATGCTTTAAACA (SEQ ID NO: 270) | |
| 103 | GTTCAGAAAACGAGAATGACCATAAATCAAAAATCAGGTCTTTACCC | 60 |
| | TGACTATTATAGT (SEQ ID NO: 271) | |
| 104 | CAGAAGCAAAGCGGATTGCATCAAAAAGATTAAGAGGAAGCCCGAA | 60 |
| | AGACTTCAAATATC (SEQ ID NO: 272) | |
| 105 | GCGTTTTAATTCGAGCTTCAAAGCGAACCAGACCGGAAGCAAACTC | 60 |
| | CAACAGGTCAGGAT (SEQ ID NO: 273) | |
| 106 | TAGAGAGTACCTTTAATTGCTCCTTTTGATAAGAGGTCATTTTTGCG | 60 |
| | GATGGCTTAGAGC (SEQ ID NO: 274) | |
| 107 | TTAATTGCTGAATATAATGCTGTAGCTCAACATGTTTTAAATATGCAA | 60 |
| | CTAAAGTACGGT (SEQ ID NO: 275) | |
| 108 | GTCTGGAAGTTTCATTCCATATAACAGTTGATTCCCAATTCTGCGAA | 60 |
| | CGAGTAGATTTAG (SEQ ID NO: 276) | |
| 109 | TTTGACCATTAGATACATTTCGCAAATGGTCAATAACCTGTTTAGCTA | 49 |
| | T (SEQ ID NO: 277) | |

Table 11 (variable oligos) In embodiments, suitable polynucleotides include those having a nucleotide sequence having at least 80%, 90%, 95%, 97%, 99% sequence identity to SEQ ID NOS: 278 to 289 as set forth in the table below.

TABLE 11
Variable oligos

| Name | Sequence (5′-3′) | Length |
|---|---|---|
| Var 1 | AACATCCAATAAATCATACAGGCAAGGCAAAGAATTAGCAAAATT | 60 |
| | AAGCAATAAAGCCTC (SEQ ID NO: 278) | |
| Var 2 | GTGAGCGAGTAACAACCCGTCGGATTCTCCGTGGGAACAAACGG | 60 |
| | CGGATTGACCGTAATG (SEQ ID NO: 279) | |
| Var 3 | TTCTTTTCACCAGTGAGACGGGCAACAGCTGATTGCCCTTCACCG | 60 |
| | CCTGGCCCTGAGAGA (SEQ ID NO: 280) | |
| Var 4 | TCTGTCCATCACGCAAATTAACCGTTGTAGCAATACTTCTTTGATT | 60 |
| | AGTAATAACATCAC (SEQ ID NO: 281) | |
| Var 5 | ATTCGACAACTCGTATTAAATCCTTTGCCCGAACGTTATTAATTT | 60 |
| | TAAAAGTTTGAGTAA (SEQ ID NO: 282) | |
| Var 6 | TGGGTTATATAACTATATGTAAATGCTGATGCAAATCCAATCGCA | 60 |
| | AGACAAAGAACGCGA (SEQ ID NO: 283) | |
| Var 7 | GTTTTAGCGAACCTCCCGACTTGCGGGAGGTTTTGAAGCCTTAA | 60 |
| | ATCAAGATTAGTTGCT (SEQ ID NO: 284) | |
| Var 8 | TCAACCGATTGAGGGAGGGAAGGTAAATATTGACGGAAATTATT | 60 |
| | CATTAAAGGTGAATTA (SEQ ID NO: 285) | |
| Var 9 | GTATTAAGAGGCTGAGACTCCTCAAGAGAAGGATTAGGATTAGCG | 60 |
| | GGGTTTTGCTCAGTA (SEQ ID NO: 286) | |
| Var 10 | AGCGAAAGACAGCATCGGAACGAGGGTAGCAACGGCTACAGAG | 60 |
| | GCTTTGAGGACTAAAGA (SEQ ID NO: 287) | |
| Var 11 | TAGGAATACCACATTCAACTAATGCAGATACATAACGCCAAAAGG | 60 |
| | AATTACGAGGCATAG (SEQ ID NO: 288) | |
| Var 12 | ATTTTCATTTGGGGCGCGAGCTGAAAAGGTGGCATCAATTCTACT | 60 |
| | AATAGTAGTAGCATT (SEQ ID NO: 289) | |

Table 12 depicts filler oligonucleotides as described herein.

TABLE 12
Filler oligos

| Name | Sequence (5′-3′) | Length |
|---|---|---|
| Var 4 filler | TCTGTCCATCACGCAAATTA | 20 |
| | (SEQ ID NO: 290) | |
| Var 5 filler | AATTTTAAAAGTTTGAGTAA | 20 |
| | (SEQ ID NO: 291) | |
| Var 6 filler | TCGCAAGACAAAGAACGCGA | 20 |
| | (SEQ ID NO: 292) | |
| Var 7 filler | TCGCAAGACAAAGAACGCGA | 20 |
| | (SEQ ID NO: 293) | |
| Var 8 filler | TATTCATTAAAGGTGAATTA | 20 |
| | (SEQ ID NO: 294) | |
| Var 9 filler | TAGCGGGGTTTTGCTCAGTA | 20 |
| | (SEQ ID NO: 295) | |

TABLE 13
Other oligos

| | | |
|---|---|---|
| Blocking | TCTCATGGCCCTTC (SEQ ID NO: 296) | 14 |
| BtsCI cut | CTACTAATAGTAGTAGCATTAACATCCAATAA | 40 |
| site oligo | ATCATACA (SEQ ID NO: 297) | |

In embodiments, suitable polynucleotides include those having a nucleotide sequence having at least 80%, 90%, 95%, 97%, 99% sequence identity to SEQ ID NOS: 290 to 297 as set forth in the tables above.

Nanoswitches were constructed as described previously (See e.g, M. A. Koussa, et al., DNA nanoswitches: a quantitative platform for gel-based biomolecular interaction analysis. Nature Methods. 12, 123 (2015); A. R. Chandrasekaran, et al., Cellular microRNA detection with miRacles: microRNA-activated conditional looping of engineered switches. Science Advances. 5, eaau9443 (2019)), and/or as described in U.S. Patent Publication No. 2018/0223344 (all of which are herein entirely incorporated by reference). In an embodiment, a genomic single-stranded DNA (New England Biolabs M13mp18) was linearized using targeted cleavage with BtsCl restriction enzyme. The linearized single-stranded DNA was then mixed with a molar excess of an oligonucleotide mixture containing backbone oligos, detectors, and annealed from 90° C. to 20° C. at 1° C. min⁻¹ in a T100™ Thermal Cycler (Bio-Rad, USA). Following construction, the nanoswitches were purified using liquid chromatography (LC) purification (See e.g., K. Halvorsen, et al., Shear Dependent LC Purification of an Engineered DNA Nanoswitch and Implications for DNA Origami. Anal. Chem. 89, 5673-5677 (2017)) to remove excess oligonucleotides. The concentration of purified nanoswitches were determined by measuring A260 absorbance with a Thermo Scientific NanoDrop 2000.

### In Vitro Transcription (IVT) of Viral RNA

Plasmids containing the full-length ZIKV (Cambodia FSS13025 strain; pFLZIKV) and DENV-2 (strain 16681, pD2/IC-30P) cDNAs were provided. pFLZIKV was linearized with ClaI (New England Biolabs, NEB), and pD2/IC-30P was linearized with XbaI (NEB). Digested plasmids were extracted with phenol:chloroform:isoamyl alcohol and then precipitated. Linearized plasmids were in vitro transcribed (Thermo Fisher Scientific) and the resulting viral RNA cleaned by MEGAclear Transcription Clean-Up Kit (Thermo Fisher Scientific). The protocols of these two kits were followed except that the purification column was not heated in the elution step of the viral RNA because high temperature can result in degradation of the viral RNA.

### Viral RNA Fragmentation Test

Viral RNA was fragmented by using 10× Fragmentation buffer (NEB) and the recommended protocol. Briefly, the ZIKV RNA obtained from in vitro transcription was mixed with fragmentation buffer (1× final) and then incubated at 94° C. in a thermal cycler for 1, 3, 6, or 9 min. RNA fragmentation analyzer (Agilent, model 5003) was used to quantify the length distribution of RNA fragments by using the DNF-471 Standard Sensitivity RNA Analysis Kit (FIG. 2B).

### Cell Culture, ZIKV Infections and Extraction of Total RNA and Virus Particles

Human hepatocarcinoma (Huh7) cells were maintained in Dulbecco's modified Eagle's medium (DMEM) (Life Technologies) supplemented with 10% fetal bovine serum (FBS, VWR Life Science Seradigm), 10 mM non-essential amino acids (NEAA; Life Technologies), and 5 mM L-glutamine (Life Technologies). Cells were passaged once every three days and maintained at 37° C. with 5% CO₂. Twenty-four hours prior to infection Huh7 cells were seeded into tissue culture plates. The following day, one plate was counted. The other two plates were used for mock- and ZIKV-infection, where cells were infected at a multiplicity of infection of one. The original Cambodia and Uganda (MR766) ZIKV stocks were provided. To isolate RNA from mock- and ZIKV-infected cells, media from the cells was aspirated and then the cell monolayer was washed once with ice-cold PBS. Hereafter, the cells in each tissue culture plate were lysed in 1 mL TRIzol (Invitrogen) and total RNA extracted per the manufacturer's instructions.

For experiments using ZIKV infectious particles in PBS/urine (FIG. 5B), Huh7 cells were infected as described above. At 24 hours post-infection, the cell culture media from ZIKV-infected cells, which contained newly assembled and released virions, was collected and concentrated using Amicon Ultra 15 centrifuge filters. The concentrated virus was then stored at −80° C. Plaque assays, as described previously (See e.g., G. Bonenfant, et al., Zika Virus Subverts Stress Granules to Promote and Restrict Viral Gene Expression. Journal of Virology, 93, e00520-19 (2019)) were used to determine the number of infectious particles.

### DNA Nanoswitch Detection

The total detection sample volume was 10 μl with 10 mM MgCl₂, 1×PBS, nanoswitch at 100 pM final concentration. Samples were incubated in a thermal cycler with thermal annealing from 40° C. to 25° C. at 1° C. min⁻¹ or room temperature (e.g. the NASBA related detections). Before loading into the gel, the samples were stained by GelRed (Biotium Inc.) at 1× concentration (or 3.3× for total RNA detection) and mixed with 2 μl 6× loading dye (15% Ficoll with 6.6% of a saturated bromophenol blue solution in water).

### Viral RNA Detection

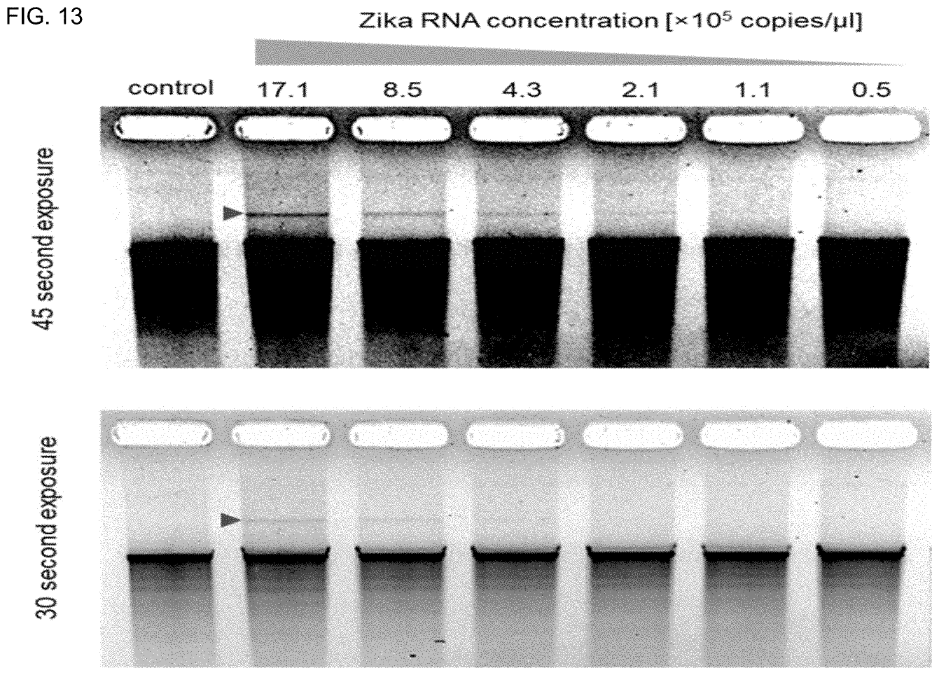

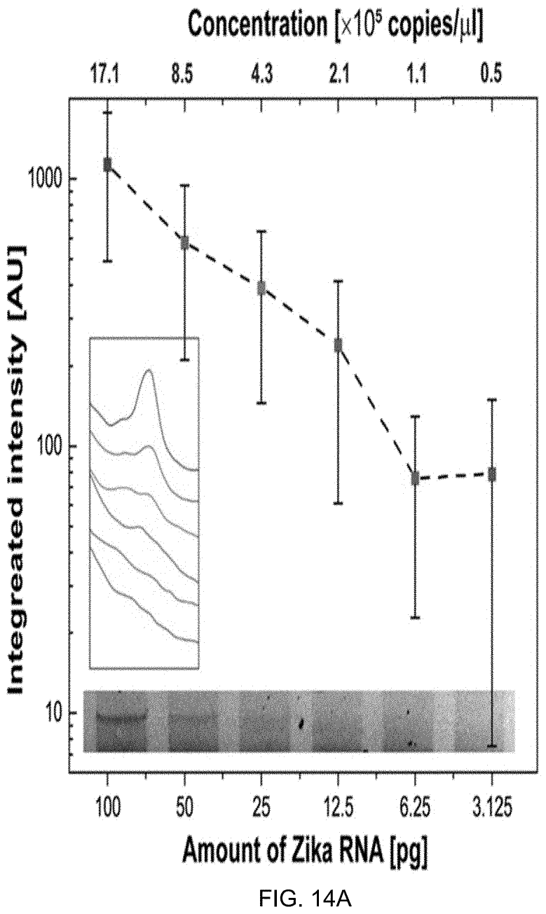

For the experiment in FIG. 2C, 5 ng (˜8.5×10⁸ copies) ZIKV RNA was used in 10 μl detection assay. Samples were run in 25 ml 0.8% agarose gels, cast from molecular biology grade agarose (Fisher BioReagents) dissolved in 0.5× Tris-Borate-EDTA (TBE) buffer. For the experiments in FIG. 2F and FIG. 13, first, all nanoswitches were purified by LC and then their concentrations were determined by measuring A260 absorbance with a Thermo Scientific NanoDrop 2000. Nanoswitch mixtures were made by mixing nanoswitches in equimolar concentrations. The detection reaction volume is 10 μl with nanoswitch (100 pM final concentration), MgCl₂ (10 mM), 1×PBS and blocking oligos (200 nM). The blocking oligos are short oligos (14 nucleotides) that can prevent the binding of target RNA to the inner surface of plastic tubes (See A. R. Chandrasekaran, et al., Cellular microRNA detection with miRacles: microRNA-activated conditional looping of engineered switches. Science Advances. 5, eaau9443 (2019)). Samples were incubated in a thermal cycler with thermal annealing from 40° C. to 25° C. over ˜12 hours (at −0.1° C./cycle and 5 min for each cycle, for a total of 150 cycles).

Detection of Viral RNA from Total RNA

First, 500 ng total RNA extracted from uninfected/infected cells was fragmented at 94° C. for 9 minutes in 1× fragmentation buffer. Fragmented total RNA was then mixed with nanoswitches (100 pM, MgCl₂ (10 mM) and PBS (1×), and the mixture was made up to 10 μl with nuclease-free water. Samples were then incubated in a thermal annealing ramp from 40° C. to 25° C. over ˜12 hours (at −0.1° C./cycle and 5 min for each cycle, for a total of 150 cycles). After the incubation, samples were stained with GelRed at 3.3× concentration and incubated at room temperature for 30 min. Before loading the gel, 2 μl of 6× blue loading dye was mixed with each sample, and 10 μl sample was loaded to each well. Samples were run in a 0.8% agarose gel at 65-75 V for about 70-90 minutes in the cold room.

Detection of Viral RNA Extracted from Urine

For the detection of viral RNA extracted from urine, DNA/RNA shield buffer was added (included with the Quick-RNA Viral Kit from ZYMO research) into urine and then mixed in the RNA with blocking oligos (200 nM) into 200 μl human urine (purchased from Innovative Research, Inc.) to mimic a clinical sample, and performed the RNA extraction immediately. Then, Quick-RNA Viral Kit (Zymo research) was used to extract the viral RNA from the urine. After RNA extraction, RNase Inhibitor was added (final concentration, 1 U/μl) to the solution. Different amounts of ZIKV RNA were tested (FIG. 5A). Here, the amount of human urine can be scaled up as needed according to the protocol of the kit. Finally, the viral RNA was eluted from the filter column by using 15 μl nuclease-free water. Then 5 μl extracted RNA was fragmented at 94° C. for 9 minutes by using 0.2× fragmentation buffer (NEB) before conducting the nanoswitch detection. Here, the use of fragmentation buffer was lowered in the consideration of the small amount of RNA in the extracted sample as too much fragmentation buffer could destroy the DNA nanoswitches.

### Isothermal Amplification by NASBA

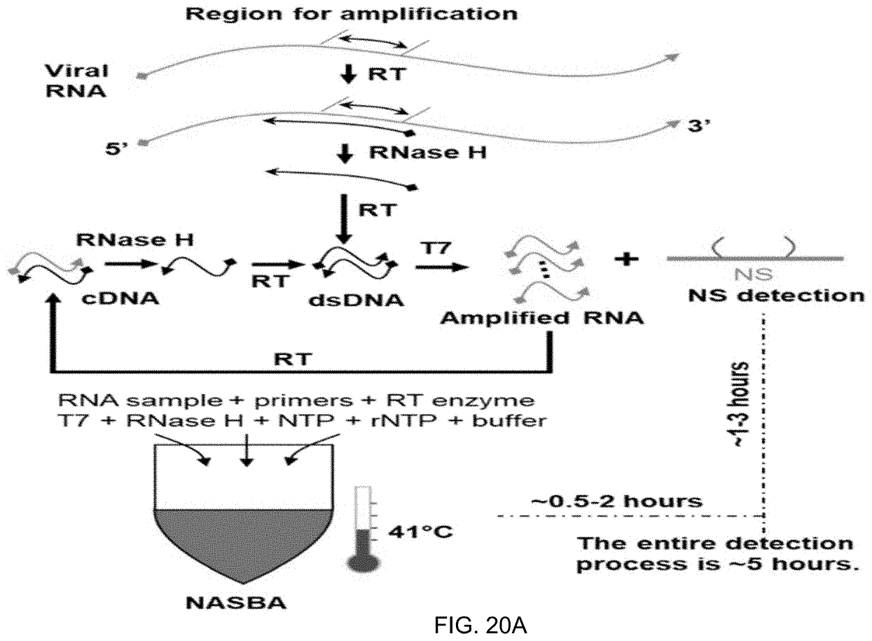

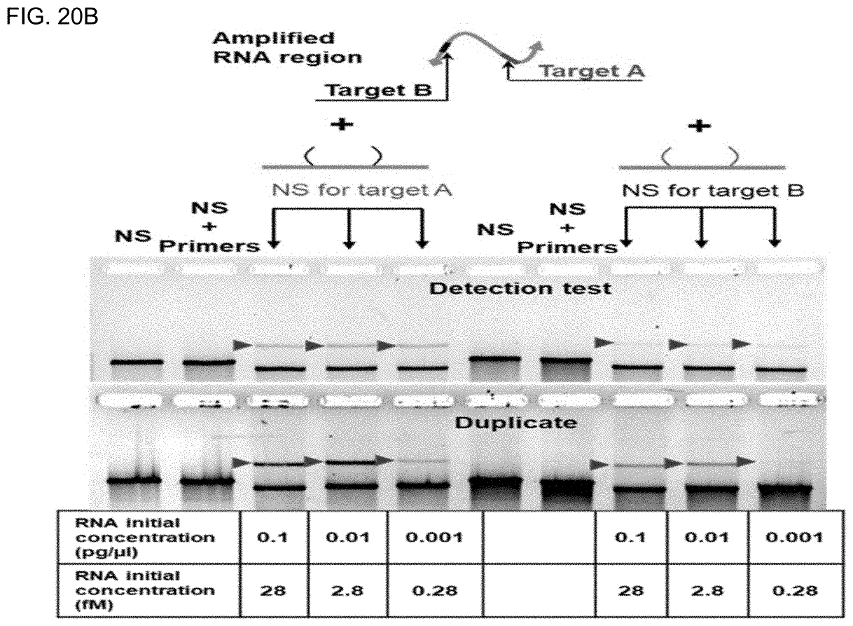

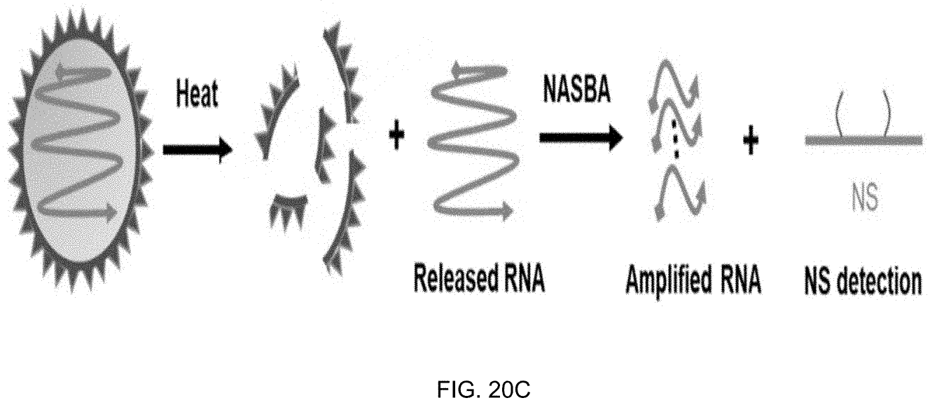

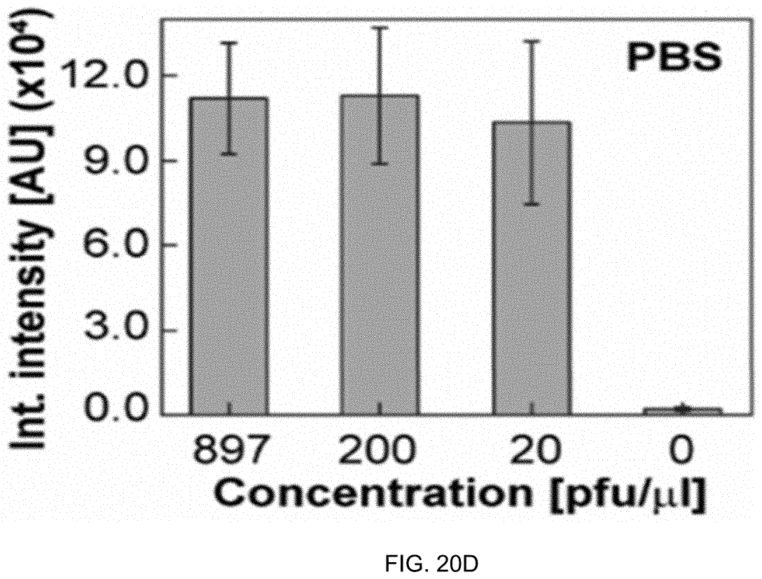

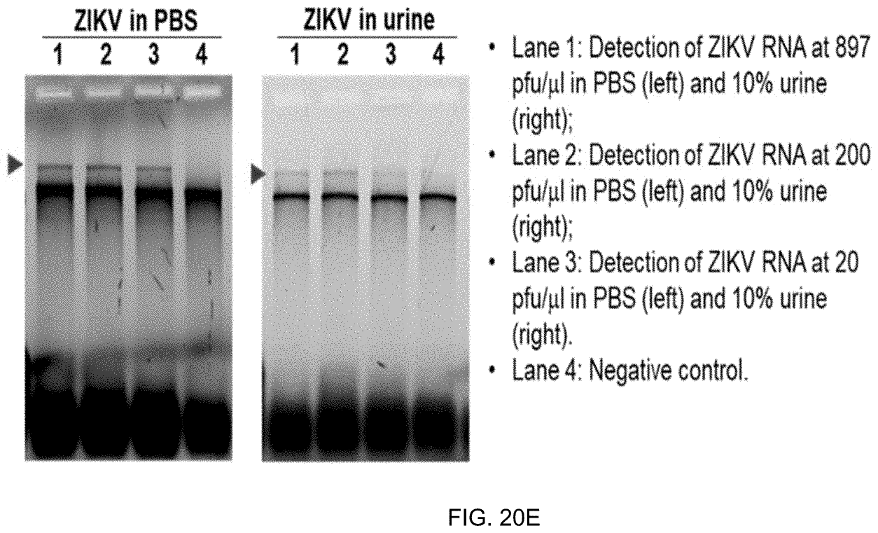

First, the classic NASBA protocol (34) was employed to prove the concept (FIGS. 20A-20D). The 25 μl one-pot reaction contained 3 μl RNA sample at various concentrations, 0.4 μM forward and reverse primers, 8 U AMV Reverse Transcriptase, 50 U T7 RNA Polymerase, 0.1 U RNase H, 40 U RNase Inhibitor (NEB, Murine), 2 mM NTP mix, 1 mM rNTP mix, 12 mM MgCl₂, 40 mM Tris-HCl, 42 mM KCl, 5 mM Dithiotreitol (DTT), 15% (v/v) dimethyl sulfoxide. The primers were chosen from reference (17). The sample was incubated at 41° C. for 2 hours in the thermal cycler followed by heating at 94° C. for 10 minutes to deactivate all enzymes. 3 μl of the NASBA sample was used in the following DNA nanoswitch detection assay in PCR tubes with 10 μl final volumes. After mixing with the DNA nanoswitch and reaction buffer, the mixture was incubated at room temperature for two hours. GelRed (Biotium Inc.) at 1× concentration was added to the detection samples before loading to the 0.8% agarose gel. The gel was run at room temperature for 45 minutes at 75 volts.

For the ZIKV related NASBA experiments, the ZIKV infectious particles were spiked (starting at 1180 pfu/μl) into 1×PBS or 10% human urine (purchased from Innovative Research, Inc.) to concentrations of 897, 200, and 20 pfu/μl. Subsequently, blocking oligo (200 nM) was added and the viral RNA was released by heating the samples at 94° C. for 3 minutes within 10 ul volume. For the human urine samples, RNase Inhibitor (NEB, Murine) was also added at a concentration of 2 U/μl before heating. After cooling down to room temperature, 0.5 μl RNase Inhibitor at 40 U/μl (NEB, Murine) was added to 10 μl human urine sample to protect the viral RNA. Total volume of each NASBA reaction was scaled down to 6 μl which contains 1.25 μl Enzyme COCKTAIL (NEC 1-24), 2 μl 3× buffer (NECB-24), 0.48 μl NTPs mix at 25 mM, 0.3 μl dNTPs mix at 20 mM, 0.2 μl two primers mix at 10 pM, 0.2 μl RNase Inhibitor with 40 U/μl (NEB, Murine) and 1.57 μl of viral RNA. The sample was incubated at 41° C. for 2 hours in the thermal cycler and followed by heating at 94° C. for 5 minutes to deactivate all enzymes. Then 1 μl of the NASBA sample was used in the following DNA nanoswitch detection assay in PCR tubes with 10 μl final volumes. The assay was finished by incubating at room temperature for two hours. After mixing with GelRed at 1× concentration and 2 μl 6× blue loading dye, the detection samples were loaded to the 25 ml 0.8% agarose gel which was run in 0.5×TBE buffer at 75 V at room temperature for 45 minutes.

### Detection of SARS-CoV-2 RNA

A gBlock gene fragment of the SARS-CoV-2 RNA segment (56) was purchased from IDT (Table 9). Then, PCR amplification (Qiagen, Taq PCR Core Kit) was used to create more copies with a T7 promoter that was added to the 5′ end of the forward primer. Afterword, dsDNA template was cleaned by the QIAquick PCR Purification Kit (Qiagen). Finally, the SARS-CoV-2 RNA was obtained by in vitro transcription (NEB, HiScribe™ T7 High Yield RNA Synthesis Kit) and cleaned by MEGAclear Transcription Clean-Up Kit (Thermo Fisher Scientific). RT-PCR detection was performed using the Luna Universal One-Step RT-qPCR kit from NEB and following its protocol. 1.5 μl RNA sample was used in 20 μl reaction mix.

For preparation of human saliva sample, pooled human saliva (purchased from Lee Biosolutions, Inc) was heated at 94° C. for 3 min to mimic the process of destroying viral capsid to release the RNA. Then, the SARS-CoV-2 RNA was spiked into diluted saliva (10%) with blocking oligos (˜200 nM) and RNase Inhibitor (2 U/μl). For the NASBA-based detection of SARS-CoV-2 RNA purchased from Twist Biosciences, NASBA kits purchased from Life Sciences Advanced Technologies Inc. were used. Briefly, 3.3 μl 3× buffer (NECB-24), 1.7 μl 6× Nucleotide Mix, 0.4 μl two primers mix at 10 μM, 0.25 μl RNase Inhibitor with 40 U/μl (NEB, Murine) and 2 μl viral RNA sample with different concentrations were mixed first and heated at 65° C. for 2 min and then the samples were incubated at 41° C. and 2.5 μl Enzyme COCKTAIL (NEC 1-24) was mixed. The samples were incubated at 41° C. for 40 min in the thermal cycler followed by heating at 94° C. for 5 minutes to deactivate all enzymes.

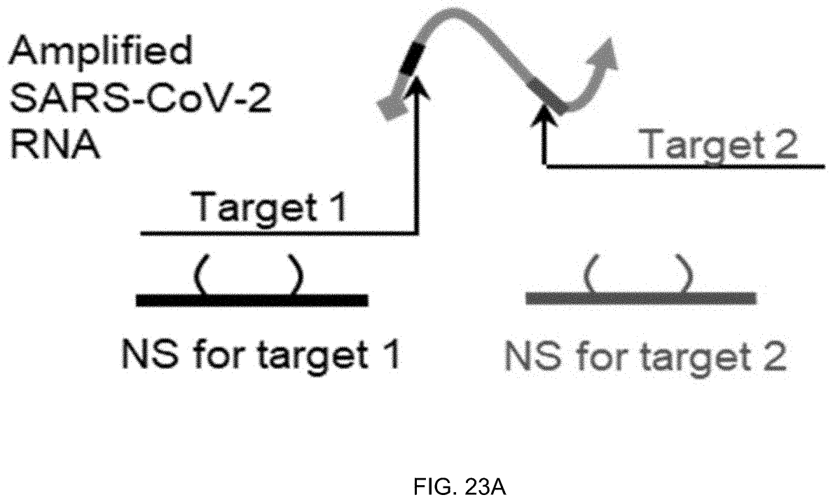

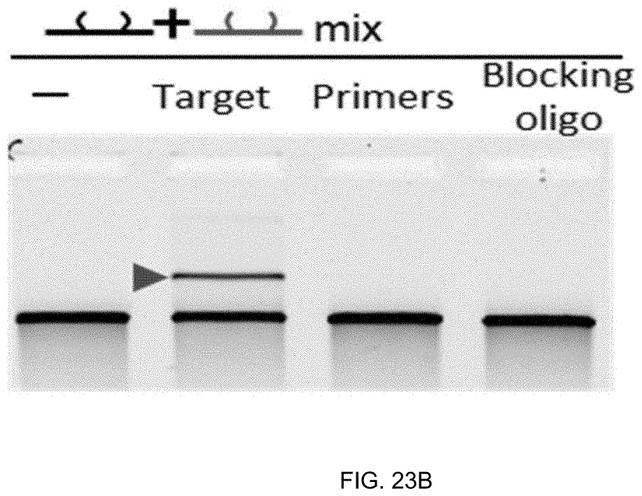

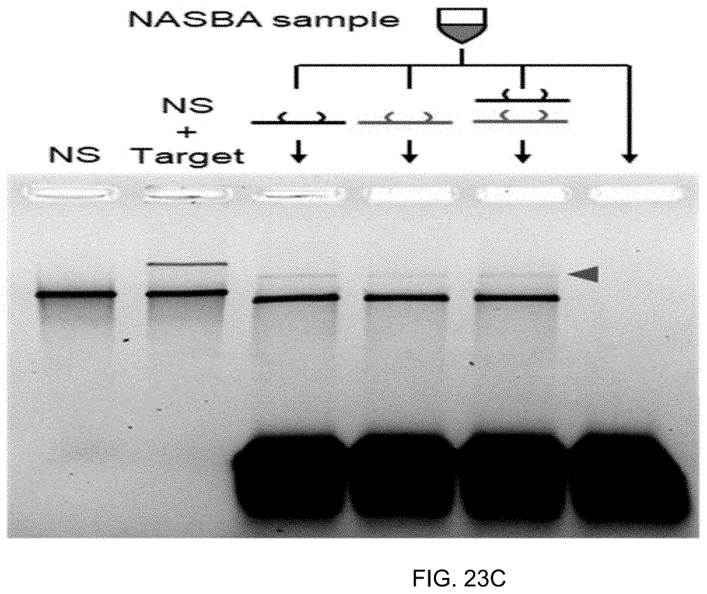

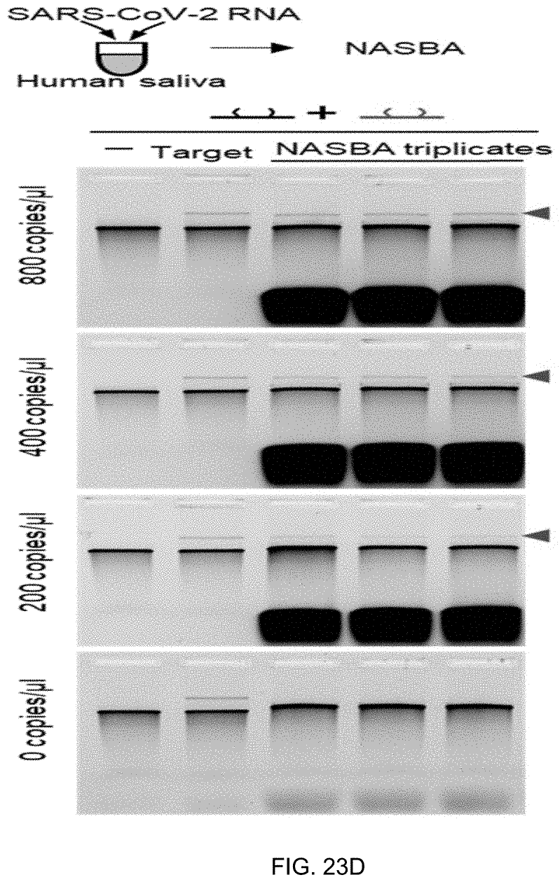

Then 1 μl of the NASBA amplified RNA sample was used in the following DNA nanoswitch detection assay in PCR tubes with 10 μl final volumes. Two nanoswitches were developed and used to target the amplified RNA pieces on two different regions (FIGS. 23A-23-D). The assay was finished by incubating at room temperature for 40 min. After mixing with GelRed at 1× concentration and 2 μl 6× blue loading dye, the detection samples were loaded to the 25 ml 0.8% agarose gel which was run in 0.5×TBE buffer at 90 V at room temperature for 25 minutes.

### Gel Imaging and Analysis

The detection samples were run in 25 ml 0.8% agarose gels unless otherwise noted, cast from molecular biology grade agarose (Fisher BioReagents) dissolved in 0.5×TBE buffer. Typical running conditions were 75 V for 45 to 70 minutes at room temperature or cold room. Samples were mixed with a Ficoll-based blue loading dye prior to loading. Imaging was completed on a Bio-Rad Gel Doc XR+ imager with different exposure times based on the brightness of the detection bands. The detection efficiency was analyzed using included Image Lab software (FIG. 2E). The profiles of detection bands were obtained in ImageJ (See e.g., C. A. Schneider, et al., NIH Image to ImageJ: 25 years of image analysis. Nat Methods. 9, 671-675 (2012)) and then their integrated intensities were obtained by using the peak analysis function in Origin (OriginLab Corporation), such as the data presented in FIGS. 2F, 4C and FIGS. 5A-B. Detailed analysis procedure can be found in a previous publication (See e.g., A. R. Chandrasekaran, et al., Cellular microRNA detection with miRacles: microRNA-activated conditional looping of engineered switches. Science Advances. 5, eaau9443 (2019)). For the E-gel related experiments, Invitrogen E-gel agarose system (Thermo Fisher Scientific) was used and its precast agarose gel (1.0%, SYBR stained). 10 μl of nanoswitch detection sample was loaded to each lane and the gel was run at 48 V for 1 h at room temperature. Since the E-gel system does not allow user control of the voltage, an external power supply was used, connected with the negative and positive electrodes of the precast agarose gel to supply 48 V.

Embodiments for detecting the presence of viral RNA are based on using DNA nanoswitches that have been designed to undergo a conformational change (from linear to looped) upon binding a target viral RNA (FIG. 1A). The presence of the viral RNA would be indicated by shifted migration of the looped nanoswitch by gel electrophoresis. Importantly, the system is designed to use common nucleic acid staining of the nanoswitch itself that can intercalate thousands of dye molecules to provide an inherently strong signal. Previously, sensitive and specific detection of DNA oligonucleotides (See e.g., A. R. Chandrasekaran, J. Zavala, K. Halvorsen, Programmable DNA Nanoswitches for Detection of Nucleic Acid Sequences. ACS Sens. 1, 120-123 (2016) and microRNAs (˜22 nucleotides long) (See A. R. Chandrasekaran, M. MacIsaac, P. Dey, O. Levchenko, L. Zhou, M. Andres, B. K. Dey, K. Halvorsen, Cellular microRNA detection with miRacles: microRNA-activated conditional looping of engineered switches. Science Advances. 5, eaau9443 (2019)) was shown using this approach. Applied here to viral RNA detection, many challenges of detecting a long viral RNA (>10,000 nucleotides) are solved in clinically relevant samples. In embodiments, an RNA fragmentation strategy has been developed, a novel signal multiplication strategy, a custom algorithm for choosing target sequences, and new workflows for measuring viral loads in biological and mock clinical samples with or without RNA pre-amplification. Using this approach, multiplexing can be used to detect multiple viruses simultaneously from a single sample and demonstrate high specificity even between closely related strains of Zika. In response to the COVID-19 pandemic, a process was developed and validated DNA nanoswitches (FIG. 1B) for the detection of SARS-CoV-2 RNA spiked into human saliva. Embodiments of the present disclosure are inherently non-enzymatic, but can optionally be combined with an isothermal amplification step, allowing use in low resource areas (FIG. 1C). This work enables direct detection of viral RNA without amplification and paves the way toward a low-cost assay for detection of RNA viruses.

In some embodiments, an example of test conditions may include: preparing and purifying nanoswitches; using 1-2 uL of nanoswitch at 250-1000 pM in a 10 uL reaction with test reagent, final buffer of 20 mM Tris-HCl pH 8 and 30 mM MgCl2; reacting the mixture at 40° C. for 20 minutes-2 hours; adding 1 uL of 300 mM EDTA; adding 1 uL of a 10× GelRed dye solution and 2 uL of a 6× loading dye; loading 10 uL of solution into a 0.8% unstained agarose gel; and running the gel electrophoresis at 75V (for a OWL B1A minigel) for 20-45 minutes.

### Results

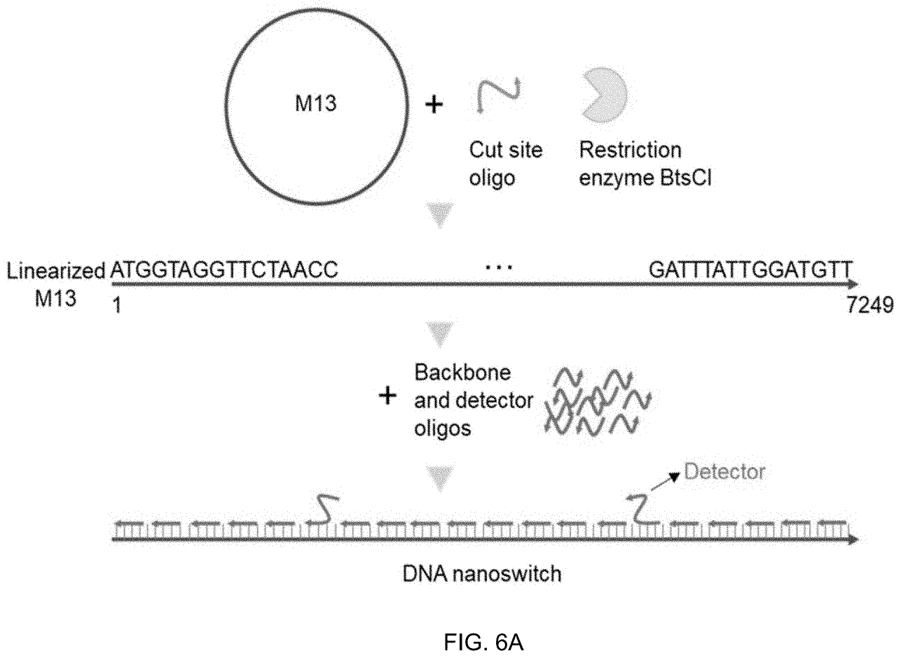

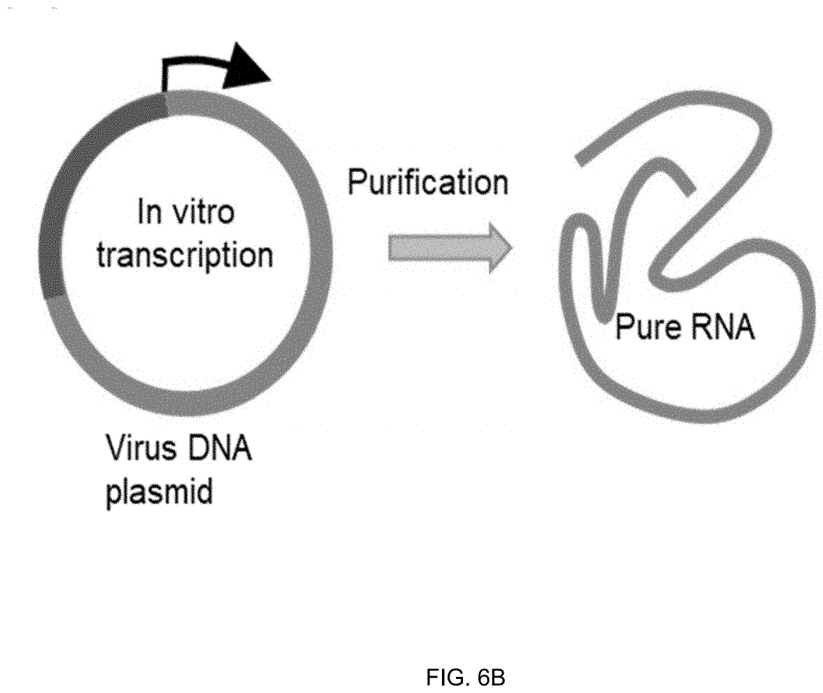

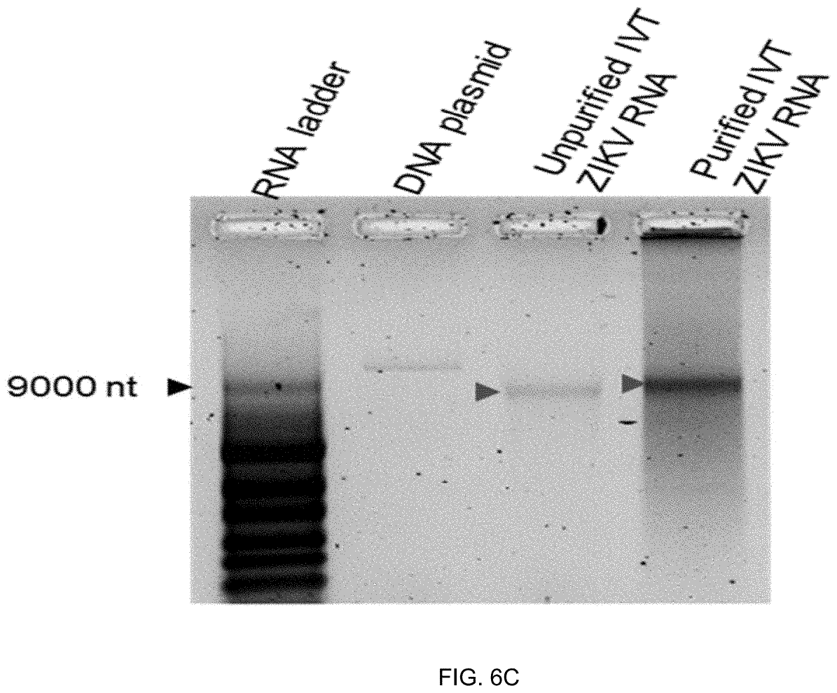

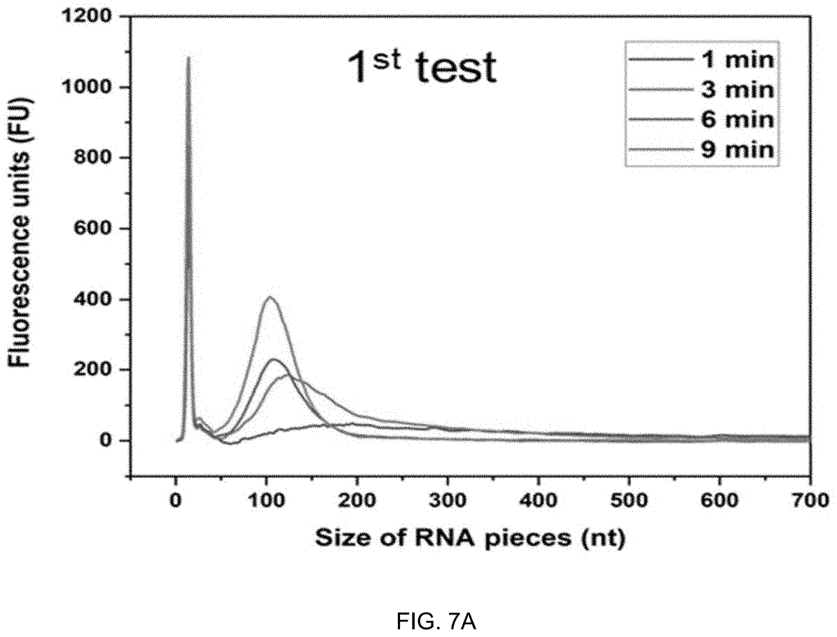

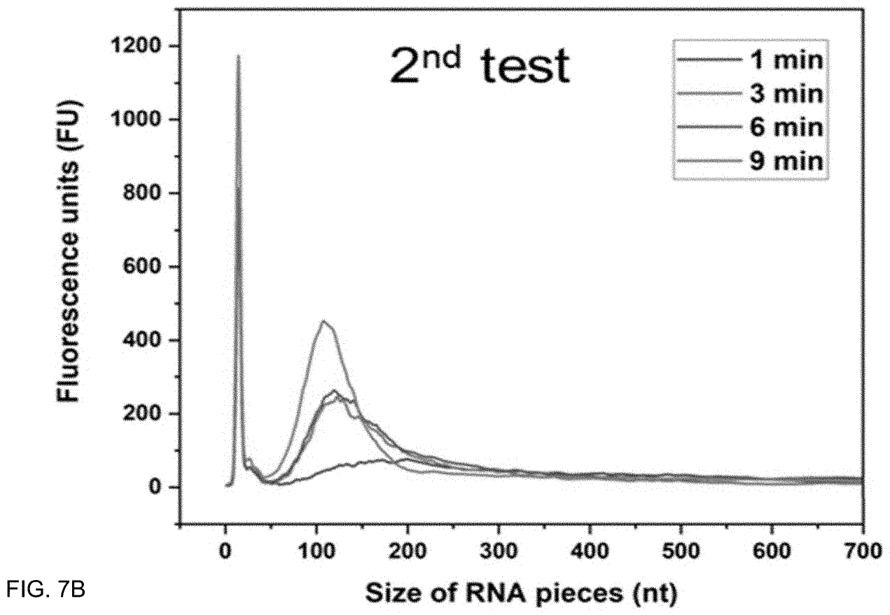

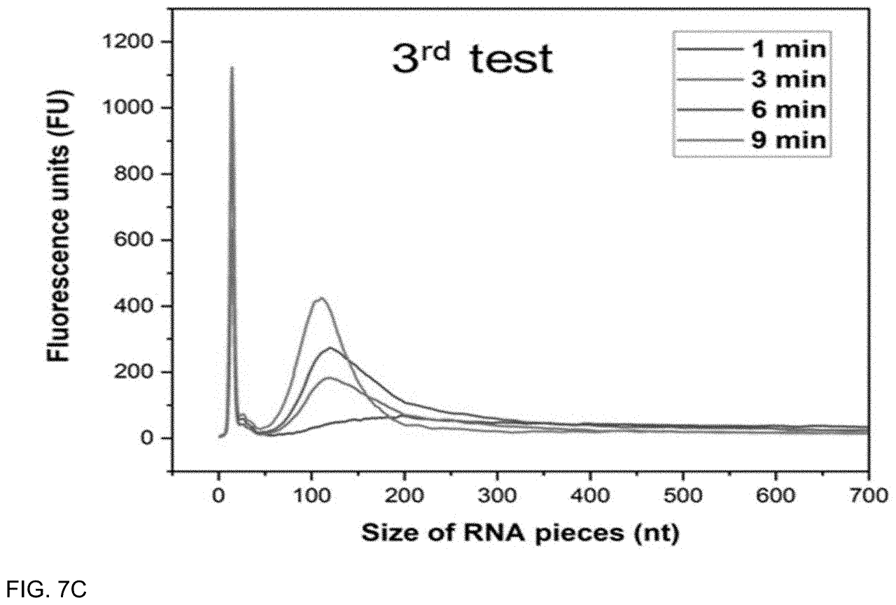

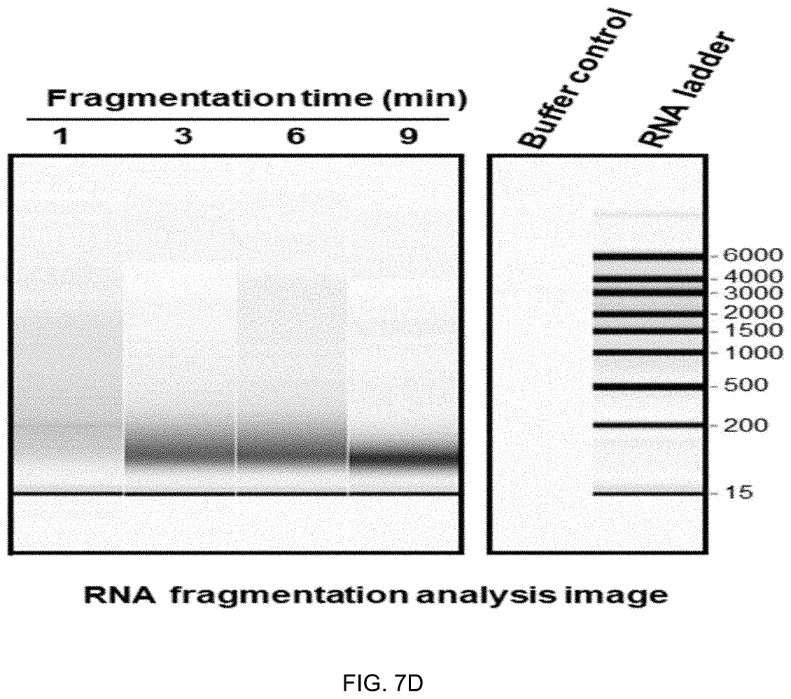

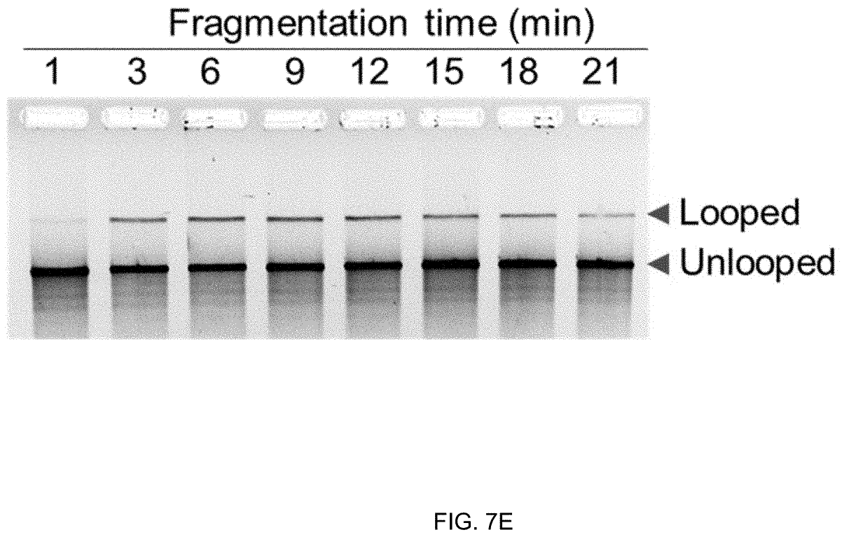

As a first proof-of-concept for detecting ZIKV, DNA nanoswitches were designed to target an already validated sequence in the ZIKV genome that has been used to bind primers in qPCR (22) (all oligo sequences are specified in Tables 1 to 10). DNA nanoswitches were made by hybridizing single-stranded DNA (ssDNA) oligos to linearized single-stranded M13mp18 (M13) genomic DNA in a thermal annealing ramp for 1 hour (See M. A. Koussa, K. Halvorsen, A. Ward, W. P. Wong, DNA nanoswitches: a quantitative platform for gel-based biomolecular interaction analysis. Nature Methods. 12, 123 (2015)) and purified them by high-performance liquid chromatography (HPLC) (23). For an initial detection target, in vitro transcribed RNA from the pFLZIKV infectious plasmid containing the full length genome of the Cambodia ZIKV isolate (FSS13025) was used (see e.g., FIG. 6A-C) (See also, C. Shan, X. Xie, A. E. Muruato, S. L. Rossi, C. M. Roundy, S. R. Azar, Y. Yang, R. B. Tesh, N. Bourne, A. D. Barrett, N. Vasilakis, S. C. Weaver, P.-Y. Shi, An Infectious cDNA Clone of Zika Virus to Study Viral Virulence, Mosquito Transmission, and Antiviral Inhibitors. Cell Host & Microbe. 19, 891-900 (2016)). Previous results have shown robust nanoswitch detection of small DNA and RNA sequences (20-30 nucleotides), but the long viral RNA is expected to have strong secondary structures that may interfere with detection (25). To overcome this, a chemical fragmentation method was used to segment the RNA into small pieces that are mostly shorter than 200 nucleotides (FIGS. 2A-B and FIGS. 7A-7E). By incubating with the nanoswitch in an annealing temperature ramp, successful detection of the fragmented viral RNAs by gel electrophoresis was shown, thus validating the approach of the present disclosure (see e.g., FIG. 2C).

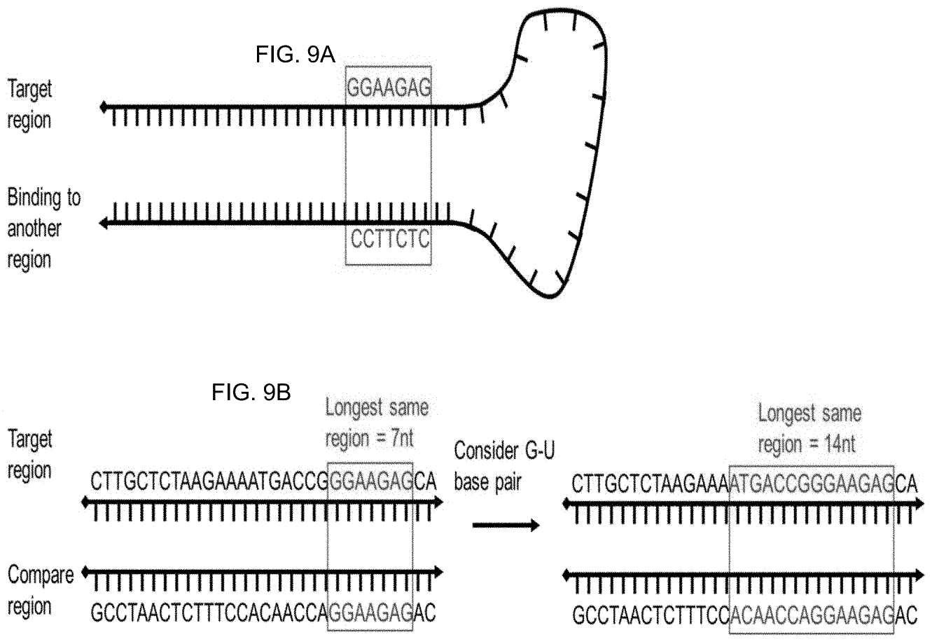

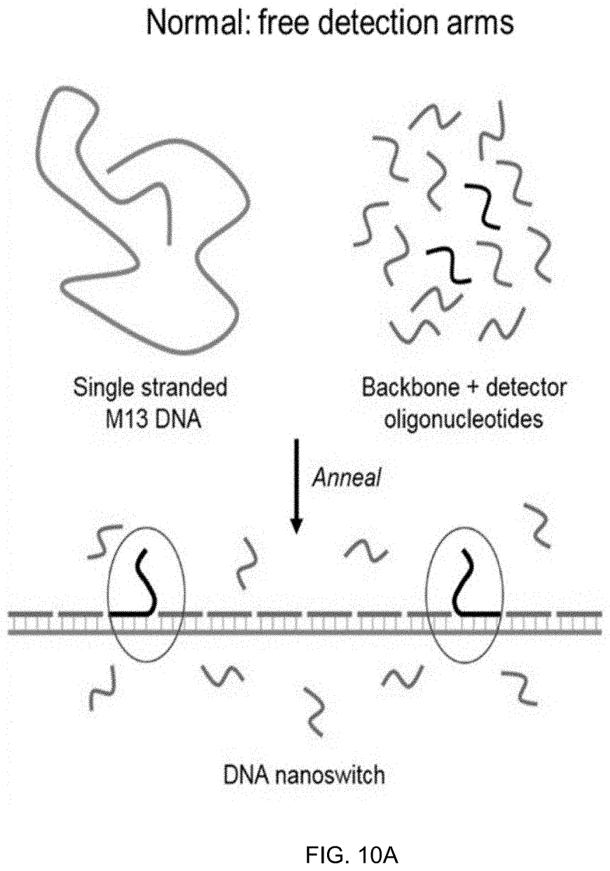

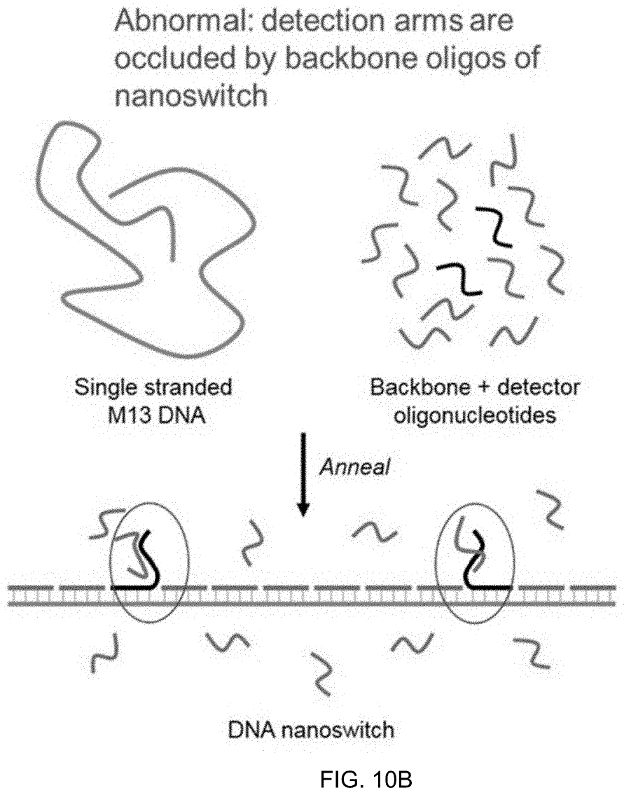

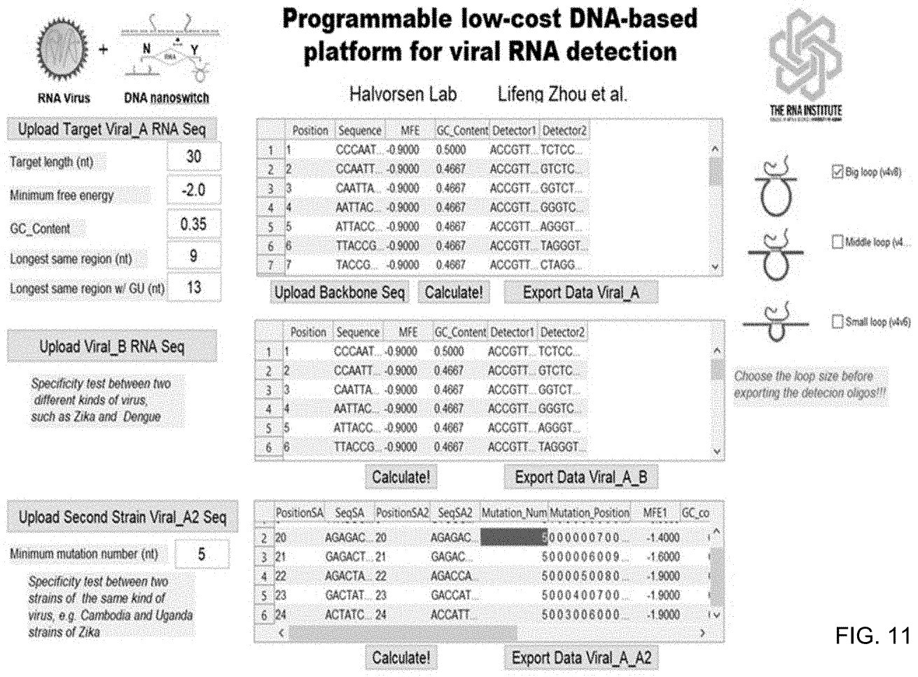

Having shown successful detection of ZIKV RNA using a single target sequence, it is possible to exploit the large genome size (˜11,000 nucleotides) to increase detection signal through multiple targets. Once the long viral RNA is fragmented, the number of available target sequences increases dramatically. Since the detection signal is proportional to the number of looped nanoswitches, a nanoswitch mixture for different target sequences within the viral genome is expected to provide an increased signal. To test this, an algorithm for choosing multiple sequence regions in the viral genome was developed that can be targeted by the nanoswitches. First, the default target length as 30 nucleotides was selected based on results from screening nanoswitches with different detection arm lengths (FIGS. 8A and 8B). Then, the algorithm selectively excluded target sequences that could form stable secondary structures (FIGS. 9A and 9B) and cross-binding with nanoswitch backbone oligos (FIGS. 10A and 10B), and enforced GC content and uniqueness of sequences. Based on these criteria, 18 target regions along the entire ZIKV RNA were chosen for testing and designed the nanoswitches. To facilitate use of the Matlab-based software, a graphical user interface (FIG. 11) was built.

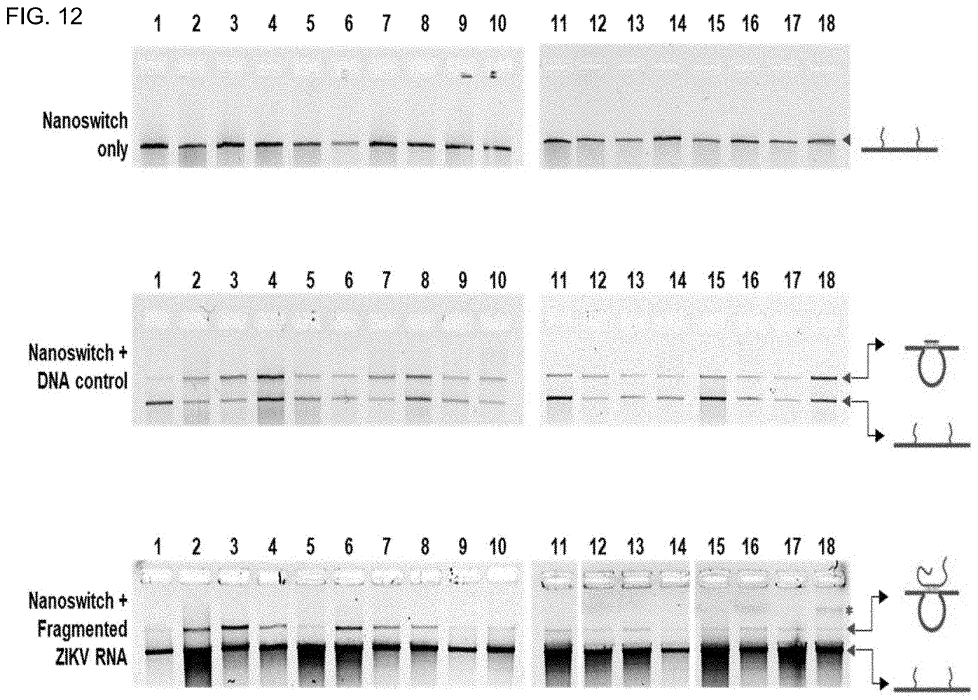

The quality and function of each nanoswitch in the panel of 18 nanoswitches was validated. All nanoswitches performed well with a molar excess of positive DNA controls (20:1 DNA control to nanoswitch), although they showed more signal variation with fragmented ZIKV RNA (FIG. 12). The nanoswitches were ranked from strongest to weakest signal and made a series of equimolar nanoswitch mixtures. Using these mixtures, the inherent signal multiplication strategy was validated using a low concentration pool of equimolar DNA fragments to mimic the fragmented RNA. It was observed that detection signal increased steadily up to around 12 different nanoswitches (FIGS. 2D-E), and then plateaued above that value. This plateau was not unexpected considering that the largest mixtures added lower performing nanoswitches that may contribute less to the overall sample. Since there was no significant change in performance between 12 and 18, the 18 nanoswitches mix was used for the follow up experiments.

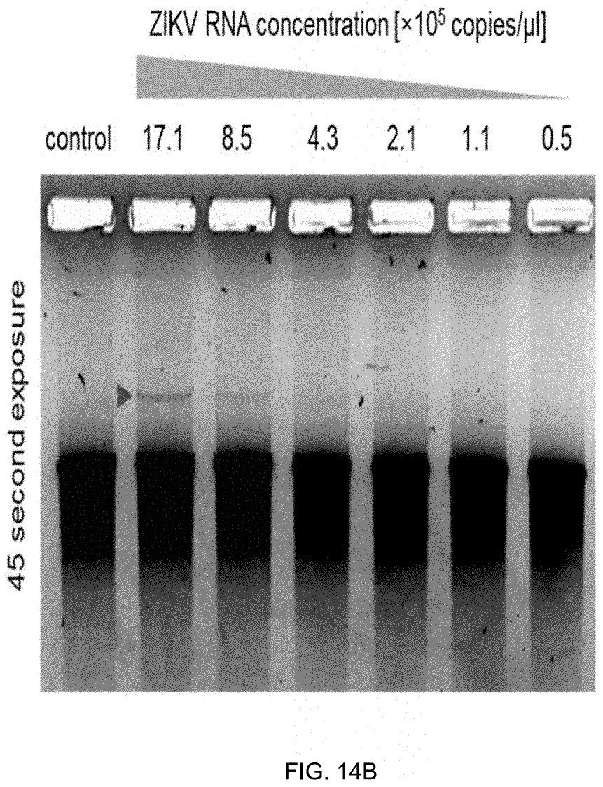

In embodiments, high sensitivity is one of the key requirements for virus detection. Clinical levels of ZIKV RNA in body fluids of infected patients are often in the femtomolar range (See e.g., D. Musso, D. J. Gubler, Zika Virus. Clinical Microbiology Reviews. 29, 487-524 (2016); K. Pardee, et al., Rapid, Low-Cost Detection of Zika Virus Using Programmable Biomolecular Components. Cell. 165, 1255-1266 (2016); and A.-C. Gourinat, O. O'Connor, E. Calvez, C. Goarant, M. Dupont-Rouzeyrol, Detection of Zika Virus in Urine. Emerg Infect Dis. 21, 84-86 (2015)) making amplification a prerequisite for most detection approaches. Based on observation that DNA nanoswitches can detect microRNAs (˜22 nucleotides) in the sub-picomolar scale (See A. R. Chandrasekaran, M. MacIsaac, P. Dey, O. Levchenko, L. Zhou, M. Andres, B. K. Dey, K. Halvorsen, Cellular microRNA detection with miRacles: microRNA-activated conditional looping of engineered switches. Science Advances. 5, eaau9443 (2019)) without amplification, it was desired to assess the sensitivity of the methods of the present disclosure for ZIKV RNA detection. The DNA nanoswitch mixture was reacted with different amounts of fragmented RNA in a 12-hour annealing temperature ramp from 40° C. to 25° C. The results showed visible detection for ZIKV RNA as low as 12.5 pg (˜3.5 attomole or ˜2.1×10⁶ copies) in a 10 μl reaction volume (FIG. 2F and FIG. 13). Consistent with FIG. 2E, the approach based on using a nanoswitch mix outperformed the highest performing nanoswitch used as a single agent, which had visible detection to about 50 pg (˜14 attomole) (FIGS. 14A and 14B).

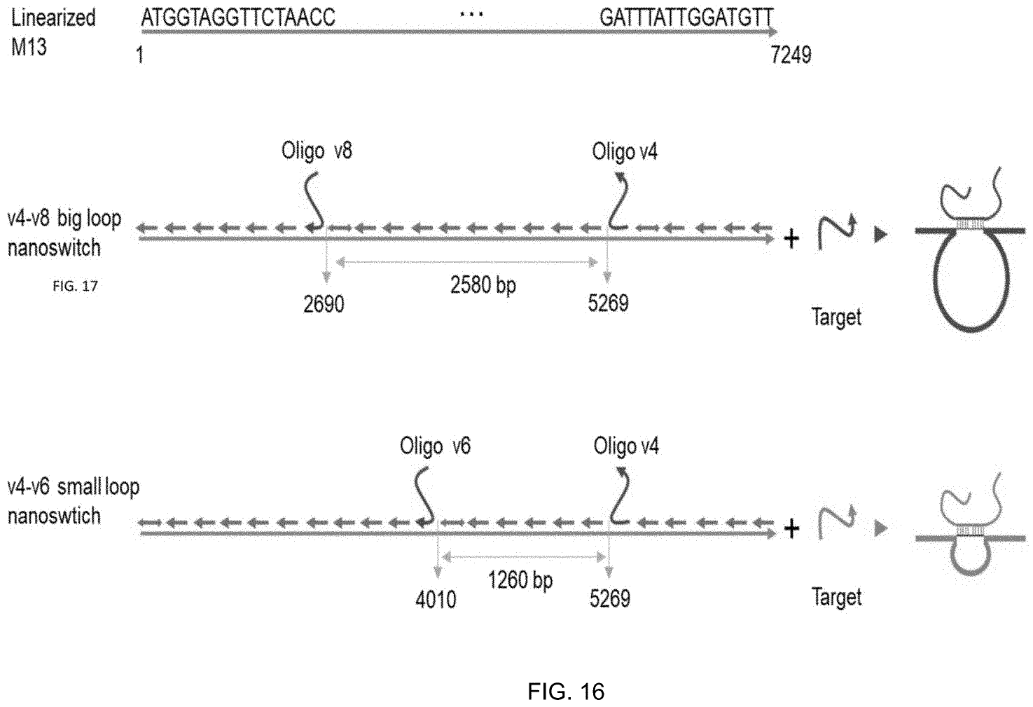

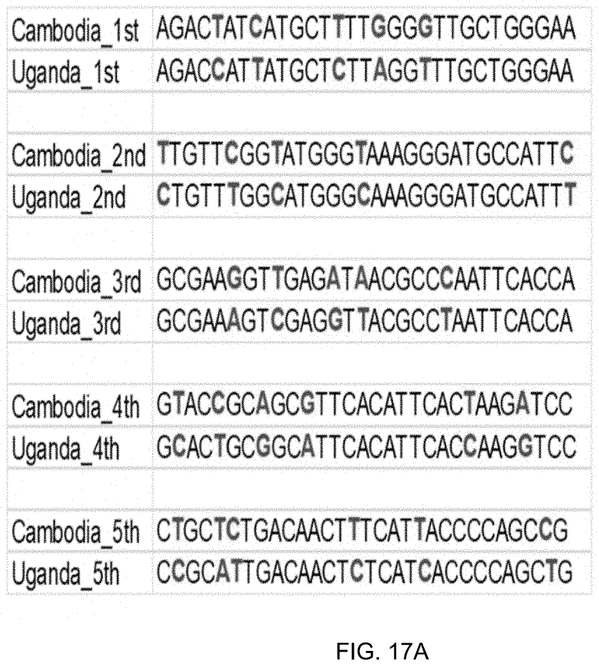

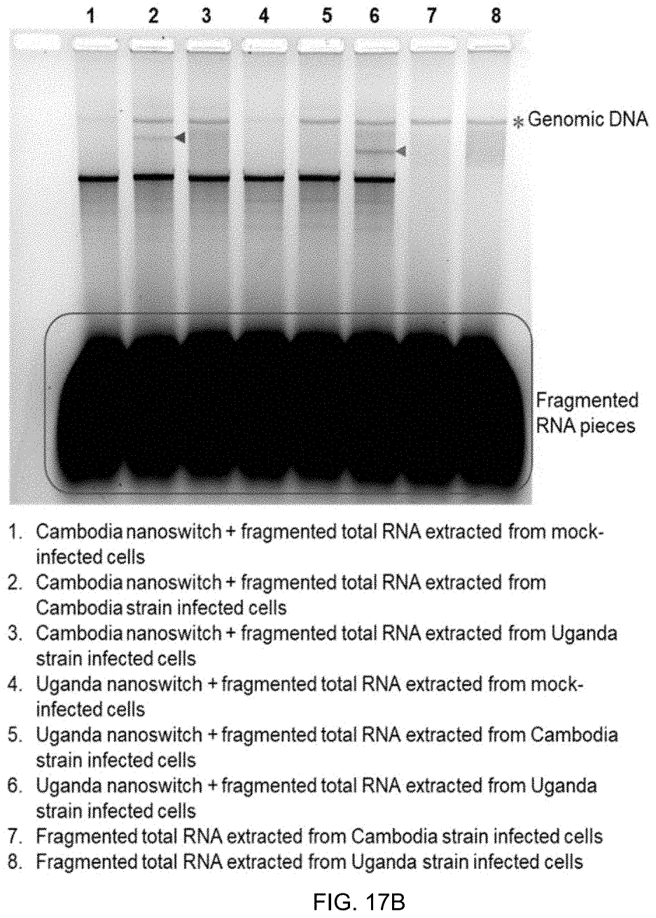

Another key requirement for a clinical virus detection assay is specificity. Since ZIKV and Dengue virus (DENV) have overlapping geographical distributions and clinical symptoms, infection with either virus may result in clinical misdiagnosis (See E. S. Paixão, M. G. Teixeira, L. C. Rodrigues, Zika, chikungunya and dengue: the causes and threats of new and re-emerging arboviral diseases. BMJ Global Health. 3, e000530 (2018)). Serological diagnostic assays are known to show antibody cross-reactivity between the two viruses, and DENV has some similarity to ZIKV in its envelope protein (See W. Dejnirattisai, P. Supasa, W. Wongwiwat, A. Rouvinski, G. Barba-Spaeth, T. Duangchinda, A. Sakuntabhai, V.-M. Cao-Lormeau, P. Malasit, F. A. Rey, J. Mongkolsapaya, G. R. Screaton, Dengue virus sero-cross-reactivity drives antibody-dependent enhancement of infection with zika virus. Nature Immunology. 17, 1102-1108 (2016)) and genome sequence (See e.g., K. Pardee, et al., Rapid, Low-Cost Detection of Zika Virus Using Programmable Biomolecular Components. Cell. 165, 1255-1266 (2016); and R. G. Huber, et al., Structure mapping of dengue and Zika viruses reveals functional long-range interactions. Nat Commun. 10, 1-13 (2019)). To test the specificity of this approach, a similar panel of nanoswitches was designed to detect DENV (FIG. 15). Using the pooled nanoswitches specific for ZIKV and DENV, each set was mixed with in vitro transcribed RNA from each virus and found perfect specificity, with each assay only detecting its correct target RNA (FIG. 3A). Using the programmability of the nanoswitch, a multiplexed system for simultaneous detection of ZIKV and DENV was demonstrated. In this case the DENV responsive nanoswitches were modified to form a smaller loop size (FIG. 16), causing two distinct detection bands to migrate to different positions in the gel. Specifically, ZIKV RNA-nanoswitch complex migrated slower/higher in the gel, while the complex of DENV RNA and the nanoswitch migrated faster/lower in the gel (FIG. 3B). Therefore, in a single reaction the nanoswitch of the present disclosure showed differential and specific detection of ZIKV and DENV RNA. By programming different loop sizes for different targets, the assay of the present disclosure can be expanded to multiple targets, such as for up to five viral targets (See e.g., A. R. Chandrasekaran, M. MacIsaac, P. Dey, O. Levchenko, L. Zhou, M. Andres, B. K. Dey, K. Halvorsen, Cellular microRNA detection with miRacles: microRNA-activated conditional looping of engineered switches. Science Advances. 5, eaau9443 (2019)). In addition to possible misdiagnosis between different viruses, there is an additional challenge in determining the specific strain of a virus. For example, in Latin America four different DENV serotypes are known to be present and co-circulate, where misdiagnosis of the infecting strain can have significant implications for treatment options (See e.g., J. Ramos-Castañeda, et al., Dengue in Latin America: Systematic Review of Molecular Epidemiological Trends. PLoS Negl Trop Dis. 11, e0005224 (2017)). Thus being able to accurately identify a circulating strain of virus broadly impacts medical care, surveillance and vector control (See e.g., A. D. Haddow, A. J. Schuh, C. Y. Yasuda, M. R. Kasper, V. Heang, R. Huy, H. Guzman, R. B. Tesh, S. C. Weaver, Genetic Characterization of Zika Virus Strains: Geographic Expansion of the Asian Lineage. PLoS Negl Trop Dis. 6, e1477 (2012)). ZIKV was first identified in Uganda in 1947 before spreading to Asia and the Americas, and ZIKV strains (classified within African or Asian lineage) share significant sequence homology (See e.g., O. Faye, et al., Molecular Evolution of Zika Virus during Its Emergence in the 20th Century. PLoS Negl Trop Dis. 8, e2636 (2014)). To investigate if the assays of the present disclosure can distinguish between the Asian and African lineages, nanoswitches were tested against two ZIKV strains which have ˜89% sequence homology, namely the FSS13025 isolated from Cambodia and the MR766 strain isolated from Uganda. In designing the ZIKV strain-specific nanoswitches, five target regions were identified that each have a 5-6 nucleotide difference (FIG. 3C and FIGS. 17A and 17B). To achieve better discernment of the detection signal, the nanoswitches for the Uganda strain were designed to form a smaller loop-size than those designed for Cambodia. Next, a human hepatocellular carcinoma cell line (Huh7) was infected with either the Cambodian or Ugandan ZIKV strain. Infected cells were processed to extract total RNA, which was then fragmented and incubated with nanoswitches to probe for viral RNA from either the ZIKV Cambodia or ZIKV Uganda infected cells. The results showed that the assay was able to discriminate between two strains of the same virus even with high genetic similarities (FIG. 3D and FIGS. 17A and 17B).

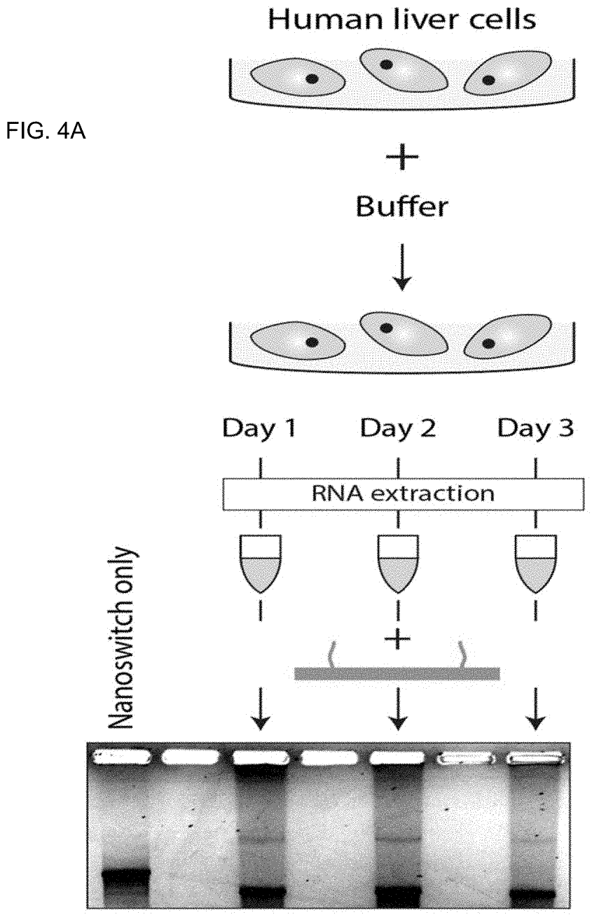

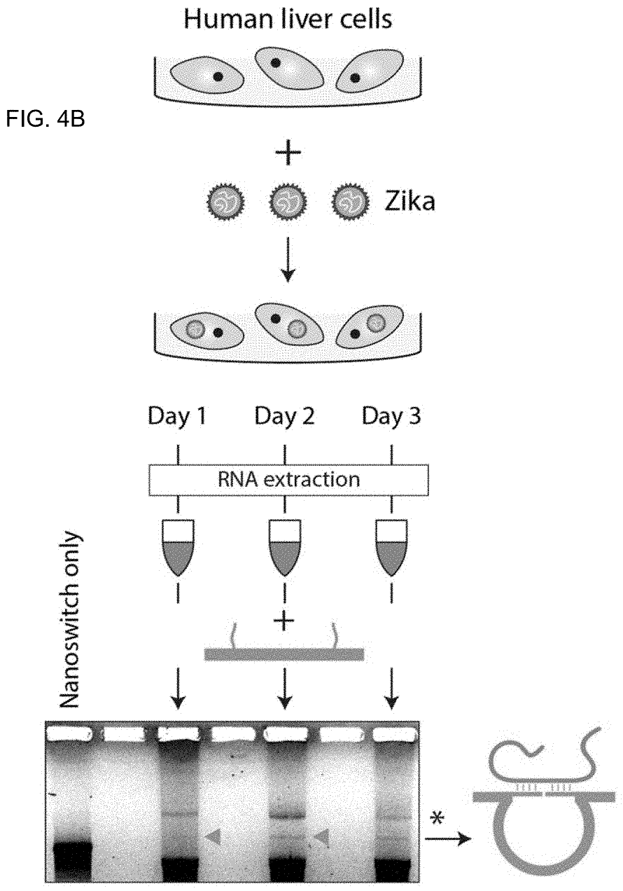

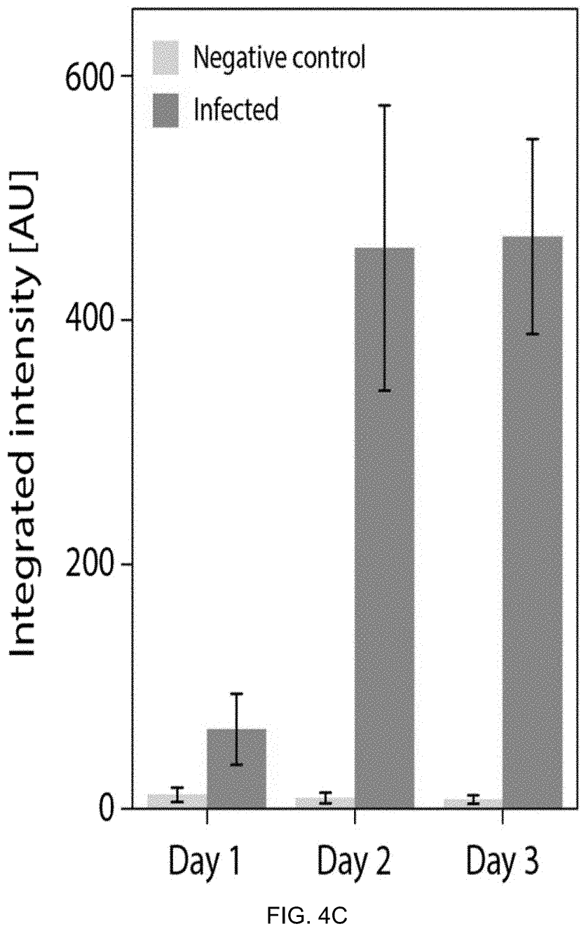

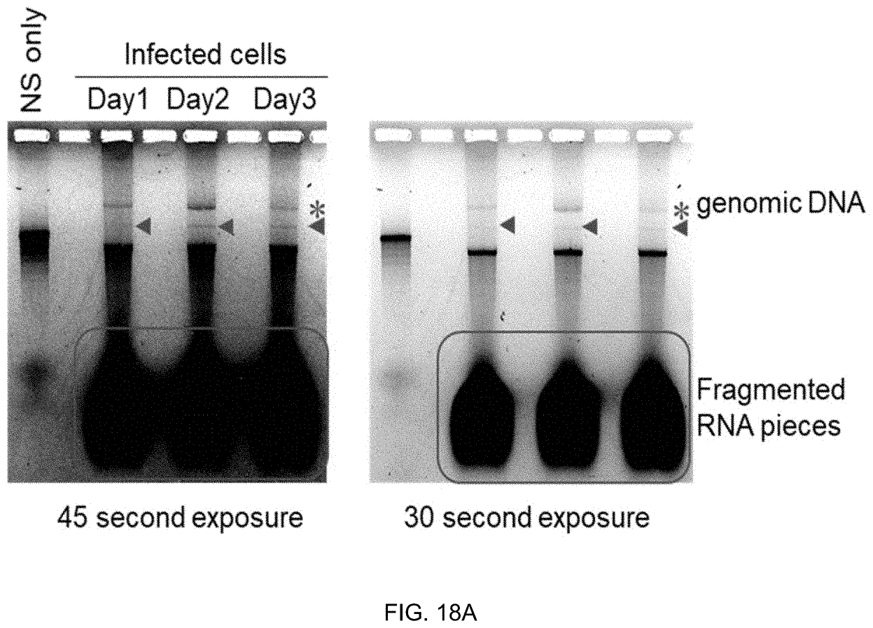

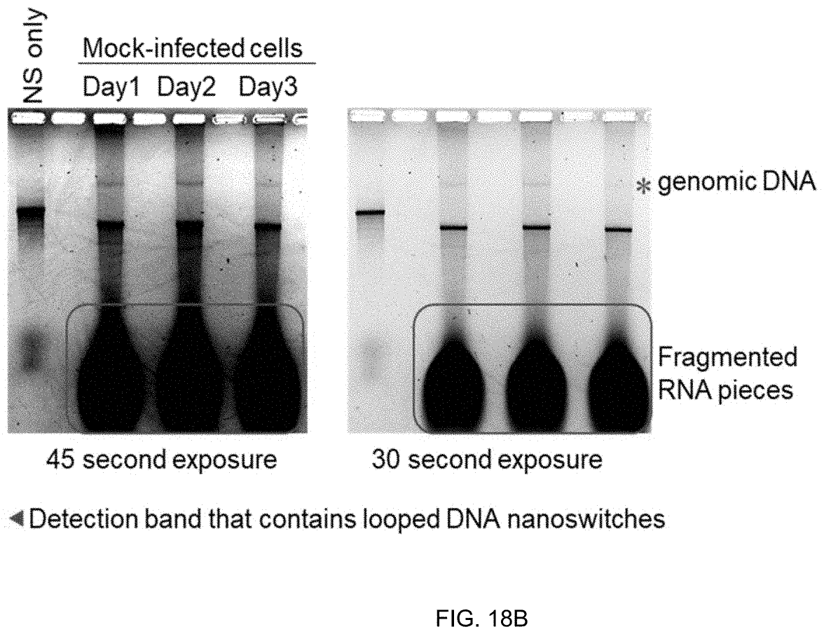

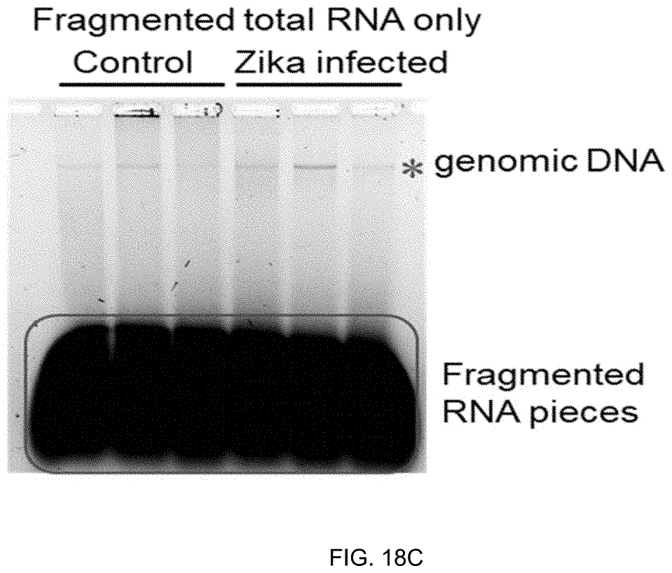

Further applying the techniques of the present disclosure to detect ZIKV RNA in biological samples, Huh7 cells were either mock-infected or infected with the Cambodia ZIKV strain at a multiplicity of infection of 1 and extracted RNA from the ZIKV infected cells at 1-, 2- and 3-days post-infection (See e.g., G. Bonenfant, N. Williams, R. Netzband, M. C. Schwarz, M. J. Evans, C. T. Pager, Zika Virus Subverts Stress Granules to Promote and Restrict Viral Gene Expression. Journal of Virology, 93, e00520-19 (2019)). The nanoswitch assays of the present disclosure detected ZIKV viral RNA from the infected cells but not the mock infected cells (See e.g., FIGS. 4A-B and FIG. 18A-18C). Detection results showed that the copies of ZIKV RNA within infected cells steadily increased upon the infection and plateaued at 2- and 3-days post-infection (FIG. 4C). These data demonstrate that assays of the present disclosure can detect ZIKV RNA in infected cell lines, and in contrast to typical RT-PCR assays without amplification of the viral RNA.

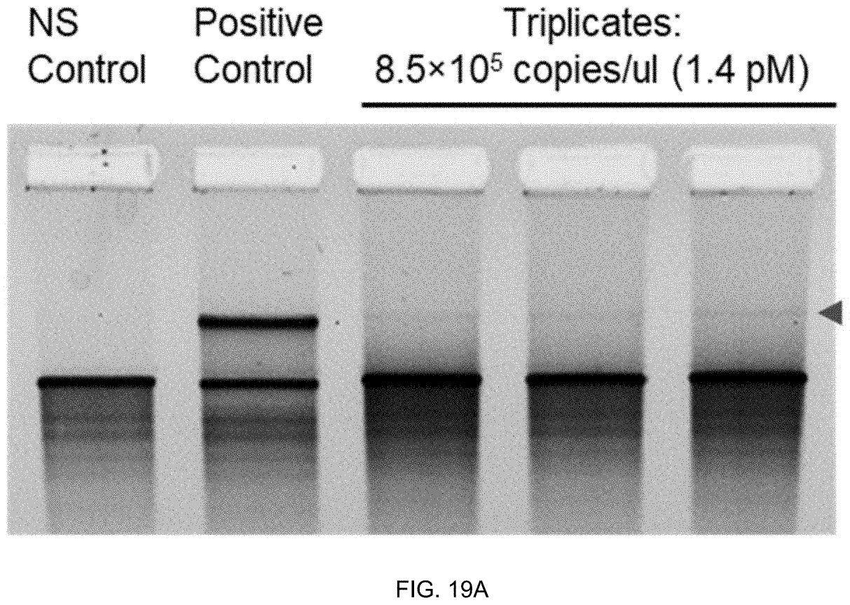

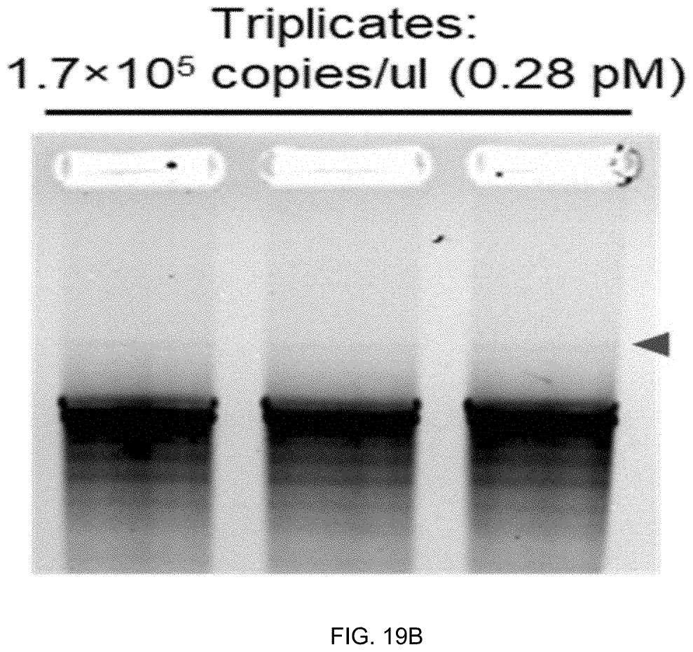

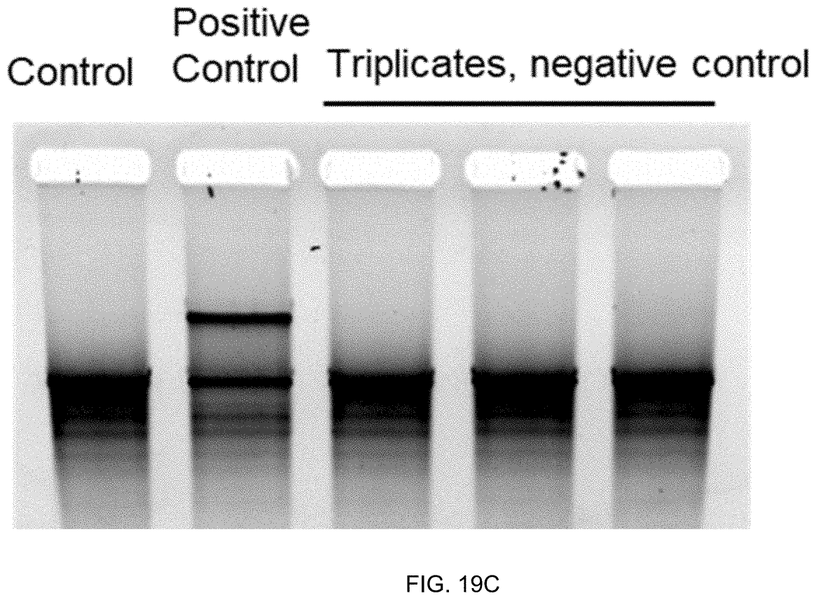

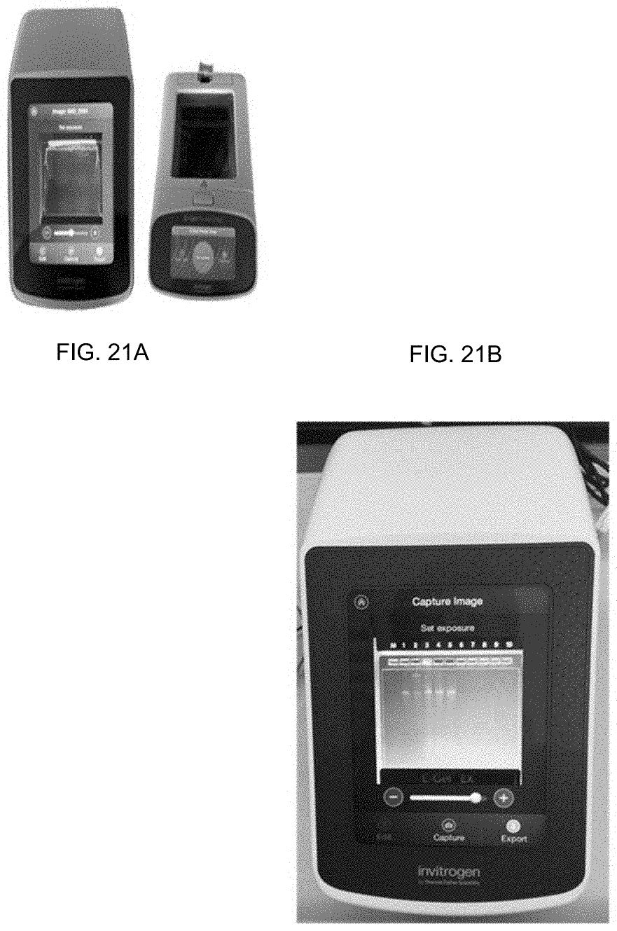

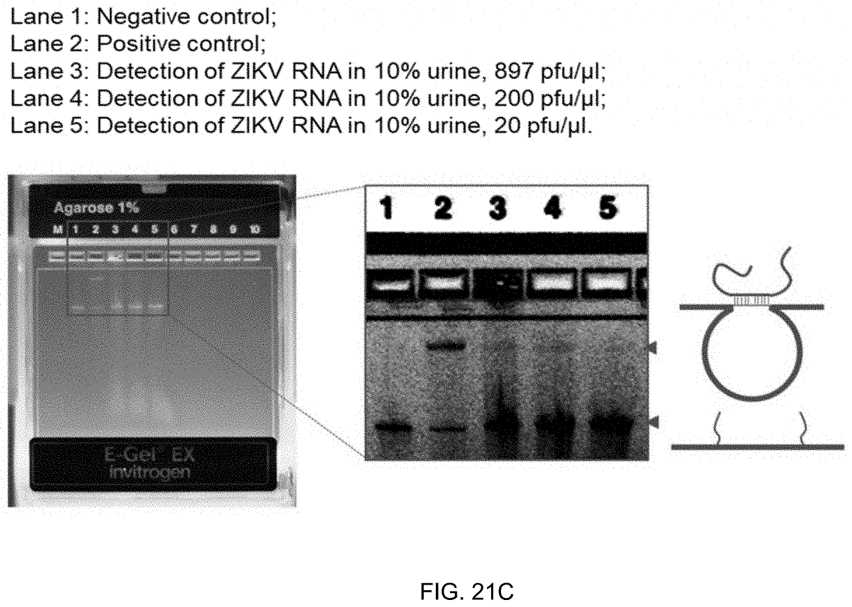

Moving toward clinical applications, detection of relevant levels of ZIKV RNA from biological fluids is demonstrated. ZIKV is present in the serum, urine, and other body fluids of infected patients (See e.g., G. Paz-Bailey, et al., Persistence of Zika Virus in Body Fluids—Final Report. New England Journal of Medicine, 379, 1234-1243 (2018). The viral loads can vary dramatically between individuals, body fluid, and post infection time (See V. M. Corman, et al., Assay optimization for molecular detection of Zika virus. Bull World Health Organ. 94, 880-892 (2016); and D. Musso, D. J. Gubler, Zika Virus. Clinical Microbiology Reviews. 29, 487-524) (2016)), but are frequently in the sub-femtomolar to femtomolar range, with ZIKV in human urine reported as high as 220×10⁶ copies/ml (365 fM) (26). While the nanoswitch sensitivity for in vitro transcribed viral RNA in buffer approaches clinically relevant concentrations, detection from body fluids is further challenged by varying viral loads and by body fluids that can reduce the performance of the nanoswitches due to physiological conditions and nuclease activity (See e.g., A. R. Chandrasekaran, J. Zavala, K. Halvorsen, Programmable DNA Nanoswitches for Detection of Nucleic Acid Sequences. ACS Sens. 1, 120-123 (2016); and C. H. Hansen, et al., Nanoswitch-linked immunosorbent assay (NLISA) for fast, sensitive, and specific protein detection. PNAS. 114, 10367-10372 (2017)). To overcome these potential difficulties, two independent solutions were investigated: 1) adding a pre-processing step to extract RNA from body fluids such as urine, or 2) adding an isothermal pre-amplification step. In the first approach, a clinically relevant amount of in vitro transcribed ZIKV RNA was spiked into human urine and processed viral RNA extraction using a commercial RNA extraction kit. RNase inhibitors were included to minimize RNA degradation in urine. The extracted RNA was then mixed with the nanoswitches and demonstrated non-enzymatic, clinical level detection of the RNA at 1.7×10⁵ copies/μl (0.28 pM) (See e.g., FIG. 5A and FIGS. 19A-C). In the second approach, it was demonstrated that detection can be coupled with other amplification approaches such as nucleic acid sequence-based amplification (NASBA) (See e.g., M. E. Gabrielle, V. et al., Nucleic acid sequence-based amplification (NASBA) for the identification of mycobacteria. Microbiology, 139, 2423-2429 (1993)). NASBA combines multiple enzymes and primers to achieve RNA amplification in a one-pot isothermal reaction (FIG. 20A). First, feasibility of the amplification of ZIKV RNA was shown by NASBA in water, followed by nanoswitch detection (FIGS. 20A-E). To mimic clinical samples, infectious ZIKV particles were spiked into either phosphate-buffered saline (PBS) or 10% human urine at clinical-levels (897 pfu/μl to 20 pfu/μl). From these samples the assay detected ZIKV RNA in ˜5 hours (FIG. 5B and FIGS. 20A-E). Further, it was shown that the assay can be performed using a commercially available buffer-less gel cartridge (ThermoFisher E-gel) and imaged on a small and potentially portable gel reader (FIGS. 21A-C). With the help of NASBA amplification, the detection ability of our method has about 1,000-fold increase, from sub-pM (˜10⁵ copies/μl) (FIG. 5A) to sub-fM (˜10² copies/μl) (FIGS. 20A-E) and the detection time was reduced from ˜13 hours to ˜5 hours.

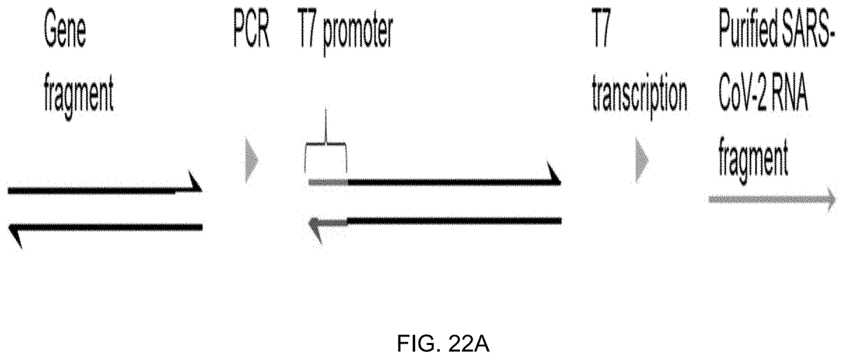

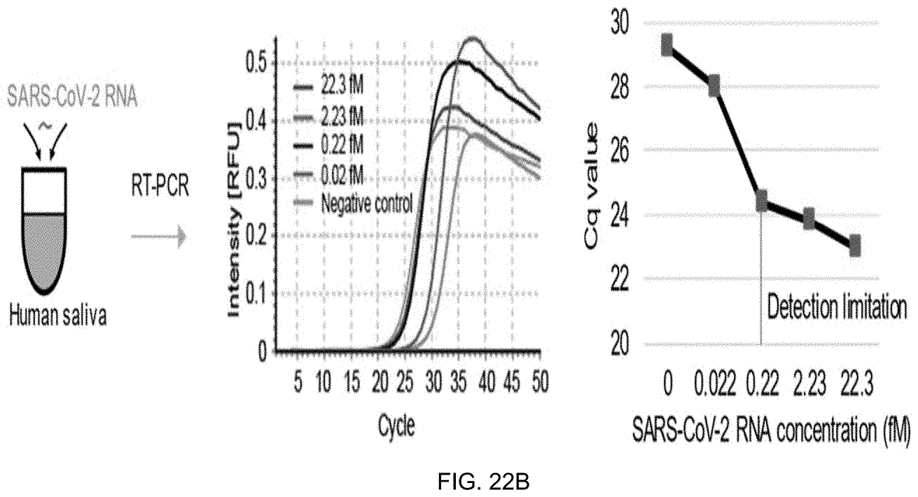

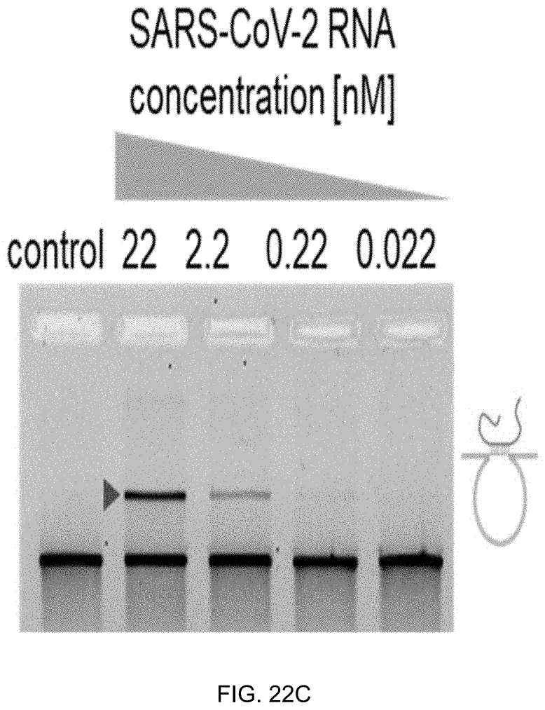

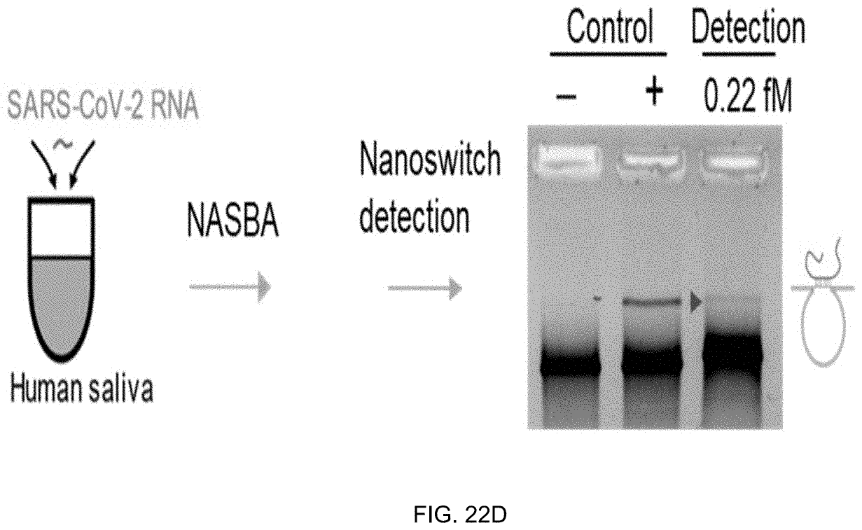

With the emerging outbreak of SARS-CoV-2 in January 2020, the DNA nanoswitches were tested against the new virus. Following a similar strategy as for ZIKV, a target region was identified, nanoswitches were developed, and used the NASBA strategy to detect a SARS-CoV-2 RNA in 10% human saliva. Following the ZIKV protocol detection was validated for an in vitro transcript of a short segment of SARS-CoV-2 RNA in ˜5 hours, and cross validated with RT-PCR (FIGS. 22A-D). Further optimizing the protocol times, detection of SARS-CoV-2 positive control RNA was achieved at a concentration as low as 200 copies/μl (around the clinical median (35-37)) in about 2 hours (1 hour NASBA, 40 minute nanoswitch incubation, 25 minute gel) (FIG. 5C and FIG. 23A-D).

Taken together, programmable DNA nanoswitches are suitable for a robust viral RNA detection platform, that is readily adaptable as shown in the detection of SARS-CoV-2. The platform has key advantages over existing methodologies in terms of selectivity and specificity, as shown in the experiments with ZIKV and closely related DENV, as well as two closely related ZIKV strains. Moreover, DNA nanoswitch viral RNA detection strategy has femtomolar detection limit without an RNA amplification step, and attomolar detection limit when used with amplification. These limits are within a clinically relevant range and therefore the DNA nanoswitch assay together with the bufferless gel cartridge presents a putative diagnostic assay for clinical detection of RNA viruses in low resource areas without significant laboratory infrastructure.

### Discussion

The functionality of the DNA nanoswitches of the present disclosure is largely enabled by DNA nanotechnology, which has become a well-established field that uses DNA as a functional material to fabricate nanostructures (See e.g., N. C. Seeman, DNA in a material world. Nature. 421, 427-431 (2003)). Biosensing is a particularly promising application of DNA nanotechnology (See e.g., M. Xiao, W. Lai, T. Man, B. Chang, L. Li, A. R. Chandrasekaran, H. Pei, Rationally Engineered Nucleic Acid Architectures for Biosensing Applications. Chem. Rev. 119, 11631-11717 (2019)), and reconfigurable DNA devices (See e.g., L. Zhou, A. E. Marras, C.-M. Huang, C. E. Castro, H.-J. Su, Paper Origami-Inspired Design and Actuation of DNA Nanomachines with Complex Motions. Small. 14, e1802580 (2018)) have been demonstrated for the detection of DNA, RNA, proteins, and pH. (See, e.g., L. Zhou, et al., Paper Origami-Inspired Design and Actuation of DNA Nanomachines with Complex Motions. Small. 14, e1802580 (2018); T. Funck, et al., Sensing Picomolar Concentrations of RNA Using Switchable Plasmonic Chirality. Angewandte Chemie. 130, 13683-13686 (2018); S. M. Douglas, et al., A Logic-Gated Nanorobot for Targeted Transport of Molecular Payloads. Science. 335, 831-834 (2012); and S. Modi, et al., A DNA nanomachine that maps spatial and temporal pH changes inside living cells. Nature Nanotechnology. 4, 325 (2009)). However, most designs are complex and require laborious readout with advanced microscopy that reduces their practicality. A few approaches have overcome this practicality hurdle to provide widely useful solutions to problems in biological imaging (e.g. DNA-PAINT in super-resolution microscopy (See e.g., R. Jungmann, et al., Multiplexed 3D cellular super-resolution imaging with DNA-PAINT and Exchange-PAINT. Nature methods. 11, 313-318 (2014)) and DNA scaffolds for NMR and cryo-EM and biosensing (e.g. detection of lysosomal disorders and mapping cellular endocytic pathways). (See e.g., M. J. Berardi, et al., Mitochondrial uncoupling protein 2 structure determined by NMR molecular fragment searching. Nature. 476, 109-113 (2011); T. G. Martin, et al., Design of a molecular support for cryo-EM structure determination. PNAS. 113, E7456-E7463 (2016); K. Leung, et al., A DNA nanomachine chemically resolves lysosomes in live cells. Nature Nanotechnology, 1 (2018); and S. Modi, et al., Two DNA nanomachines map pH changes along intersecting endocytic pathways inside the same cell. Nature Nanotech. 8, 459-467 (2013)). The DNA nanoswitches take a reductionist approach, resulting in assays that are robust and sensitive, yet simple to adapt and do not require multiple steps or expensive equipment. With this work, RNA virus detection is added to the existing suite of DNA nanoswitch assays that already includes protein (See e.g., C. H. Hansen, et al., Nanoswitch-linked immunosorbent assay (NLISA) for fast, sensitive, and specific protein detection. PNAS. 114, 10367-10372 (2017)) and microRNA (See e.g., A. R. Chandrasekaran, et al., Cellular microRNA detection with miRacles: microRNA-activated conditional looping of engineered switches. Science Advances. 5, eaau9443 (2019)) detection.

The simple DNA nanoswitch-based assay for detection of viral RNA overcomes some limitations of currently available methods for clinical detection of RNA viruses in resource-limited areas. These include 1) robust detection without enzymes or equipment, 2) maintaining low-cost and simplicity, and 3) providing specificity and versatility. Surprisingly, the current COVID-19 pandemic has shown us that these problems can affect rich countries as well, with many struggling to have testing outpace viral spread.

The intrinsically high signal of the nanoswitches is enhanced here with a new “target multiplication” strategy where viral RNA fragmentation is used to multiply the number of targets, and thus increase the signal intensity. Using this approach, excellent levels of detection in urine were reached without the use of enzyme-mediated amplification strategies. This is of significance because enzymes can be key drivers of assay cost and complexity due to requirements including cold storage/transportation, special buffers and reagents, and strict operating temperatures. These factors make enzymatic assays difficult for field use or for use in low resource areas without modern lab infrastructure. Despite these challenges, most currently available techniques rely on enzyme triggered amplification. (See e.g., D. Musso, D. J. Gubler, Zika Virus. Clinical Microbiology Reviews. 29, 487-524 (2016); K. Pardee, et al., Rapid, Low-Cost Detection of Zika Virus Using Programmable Biomolecular Components. Cell. 165, 1255-1266 (2016); J. S. Gootenberg, et al., Multiplexed and portable nucleic acid detection platform with Cas13, Cas12a, and Csm6. Science. 360, 439-444 (2018)). For the assays of the present disclosure, compatibility and dramatic signal improvement have been demonstrated with an optional enzymatic pre-amplification step (FIG. 5B-C). However, it is believed that further improvements should enable complete coverage of the clinical range without enzymes. A 30 ml sample of urine from a ZIKV-infected patient would contain from 10⁵ to 10⁹ copies of viral RNA (See e.g., E. S. Theel, D. J. Hata, Diagnostic Testing for Zika Virus: a Postoutbreak Update. Journal of Clinical Microbiology. 56, e01972-17 (2018), theoretically surpassing a current detection limit. Efficient sample preparation using a viral RNA extraction kit (FIG. 5A), for example, could facilitate use with the DNA nanoswitch assay.

Two key features of the approach are simplicity and low cost. The DNA nanoswitches align with the goals of “frugal science” movement, where cost and accessibility to new technologies are valued alongside typical performance metrics (See e.g., G. Whitesides, The frugal way: The promise of cost-conscious science. The Economist: The World in 2012 (2011), p. 154; and S. Reardon, Frugal science gets DIY diagnostics to world's poorest. New Scientist. 219, 20-21 (2013)).

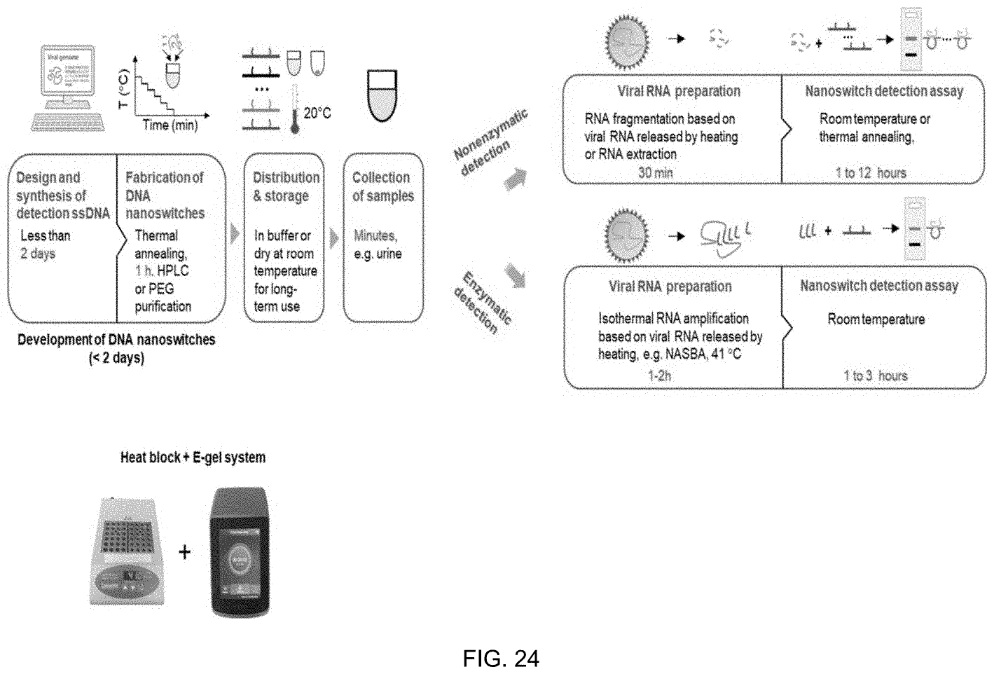

In embodiments, the nanoswitches cost around 1 penny per reaction and can be stored dry at room temperature for at least a month, and could be delivered globally without transportation or biosafety concerns. The assay consists of few steps and can be performed in a matter of hours with limited laboratory needs (FIG. 24). The assay uses a readout by gel electrophoresis, which is relatively inexpensive and already part of the workflow in many labs, which is comparatively simpler than many nanotechnology-based assays involving multiple incubation and wash steps. Improvements to the signal readout could potentially help make this approach even more lab independent. Successful detection with a commercially available buffer-less gel system (FIGS. 21A-C) takes us a step closer to enabling field deployment of the assay, and sample preparation could be aided by other frugal science approaches such as the “paperfuge” and low-cost thermal cycler. (See e.g., M. S. Bhamla, et al., Hand-powered ultralow-cost paper centrifuge. Nature Biomedical Engineering. 1, 0009 (2017); and G. Wong, et al., A Rapid and Low-Cost PCR Thermal Cycler for Low Resource Settings. PLOS ONE. 10, e0131701 (2015)). If purified viral RNA is used in NASBA, the entire detection could be shortened to two hours with a 30 min NASBA step (See e.g., K. Pardee, et al., Rapid, Low-Cost Detection of Zika Virus Using Programmable Biomolecular Components. Cell. 165, 1255-1266 (2016)) and a 1 hour nanoswitch detection assay (A. R. Chandrasekaran, et al., Cellular microRNA detection with miRacles: microRNA-activated conditional looping of engineered switches. Science Advances. 5, eaau9443 (2019)). The programmability of the system makes it versatile for a wide variety of viruses including ZIKV, DENV, and SARS-CoV-2 as shown herein. These can be detected with high specificity as shown for ZIKV and DENV (FIGS. 3A-B) and for different strains of ZIKV (FIG. 3C-D), even in a multiplexed fashion. Here single-stranded RNA viruses are investigated but assays for other RNA or DNA viruses could likely be developed similarly. The fast construction and purification processes can facilitate rapid production of DNA nanoswitches to detect an emerging viral threat, potentially in as little as 1-2 days from knowledge of the target sequences, limited mostly by oligo synthesis turnaround time (FIG. 1B). Due to the low cost of the test, the assay is useful for monitoring viral progression over time in patients, or for testing potentially infected insects or animals. Therefore, with future application towards point-of-care clinical applications in resource-limiting environments, embodiments of the platform described herein has a potential to improve accuracy and ease of diagnosis in humans, non-human vectors, and other animals. Ultimately this can enhance the ability to control spread of infection and more rapidly respond to emerging viral threats including the COVID-19 pandemic, and work toward a reduced death toll and economic burden.

### Example II

### A Rapid Non-Enzymatic Test for SARS-CoV-2 RNA Using DNA Nanoswitches.

The emergence of a highly contagious novel coronavirus in 2019 led to an unprecedented need for large scale diagnostic testing. This increased testing also created problematic reagent bottlenecks for routinely used assays, necessitating the development of alternate diagnostic tests that are low-cost and sensitive with rapid turnaround time. Here, a rapid diagnostic test for SARS-CoV-2 is demonstrated eliminating the need for costly enzymes. Embodiments include one or more DNA nanoswitches that respond to segments of the viral RNA by a change in shape that is readable by gel electrophoresis. Embodiments include a new multi-targeting approach and improve the limit of detection, and a new workflow that integrates with bufferless gel cartridges provides results in <1 hour without laboratory equipment. Embodiments of the present disclosure are applied to a cohort of clinical samples, and have excellent specificity and sensitivity. Since the method embodiments directly detects viral RNA, it is less likely to report false positives and also directly shows the level of virus. The healthcare battle against the COVID-19 pandemic will benefit from the embodiments of the present disclosure, both for rapid onsite testing as well as for monitoring viral loads in recovering patients.

### Introduction

A novel coronavirus, SARS-CoV-2 (severe acute respiratory syndrome coronavirus 2), has caused an outbreak of the disease COVID-19, (See e.g., Wang, C et al., A novel coronavirus outbreak of global health concern. The Lancet 395, 470-473 (2020)| and Zhu, N. et al. A Novel Coronavirus from Patients with Pneumonia in China, 2019. New England Journal of Medicine 382, 727-733 (2020)) with over 270 million reported cases and 5.3 million deaths globally since the start of the pandemic. (See e.g., WHO Coronavirus (COVID-19) Dashboard. https://covid19.who.int).

The pandemic highlights the need for development of rapid and low-cost alternate detection strategies for large scale testing. (See e.g., Udugama, B. et al. Diagnosing COVID-19: The Disease and Tools for Detection. ACS Nano 14, 3822-3835 (2020)). The gold-standard method for viral detection in clinical samples is RT-PCR (real-time polymerase chain reaction), which involves nucleic acid amplification and its monitoring via fluorescence. PCR-based tests, however, require days for diagnosing patients with suspected SARS-CoV-2, thus creating a gap in COVID positive individuals transmitting the virus before the results are obtained. Antigen tests can also be used to detect the spike protein on SARS-CoV-2 virions using lateral flow assay technology. These tests have been developed and marketed as rapid home-based tests, but are substantially less sensitive than PCR tests and still costly enough to limit their frequent use among much of the population. Serology tests are rapid and require minimal equipment, but these tests do not contribute to curtailing the spread of the disease as it can take several days to weeks following symptom onset for a patient to accumulate detectable levels of antibodies. (See e.g., Zhang, W. et al. Molecular and serological investigation of 2019-nCoV infected patients: implication of multiple shedding routes. Emerging Microbes & Infections 9, 386-389 (2020)). At various points in the pandemic, large scale global testing has strained supply chains and created bottlenecks of the reagents required for the PCR assay (See e.g., Esbin, M. N. et al. Overcoming the bottleneck to widespread testing: A rapid review of nucleic acid testing approaches for COVID-19 detection. RNA rna.076232.120 (2020) doi:10.1261/rna.076232.120), or general scarcity of rapid antigen tests. These limitations and bottlenecks in testing have negatively affected the ability to control the pandemic. Thus, the focus of new diagnostic assays has shifted from solely high sensitivity to also include aspects of cost, turnaround time, and operation outside a lab by non-specialists. (See e.g., Mina, M. J. et al., Rethinking Covid-19 Test Sensitivity—A Strategy for Containment. New England Journal of Medicine 0, null (2020)). These alternate testing strategies provide rapid results that aid in quick diagnosis and thus control of disease spread in contrast to highly sensitive tests with longer turnaround times. (See e.g., Toward COVID-19 Testing Any Time, Anywhere. The Scientist Magazine® www.the-scientist.com/news-opinion/toward-covid-19-testing-any-time-anywhere-67906; and Even imperfect Covid-19 tests can help control the pandemic. STAT www.statnews.com/2020/08/20/even-imperfect-covid-19-tests-can-help-control-the-pandemic/ (2020)). Over the past year, the frequency and speed of testing was found to be more practically relevant than test sensitivity for large scale COVID-19 testing. (See e.g., Larremore, D. B. et al. Test sensitivity is secondary to frequency and turnaround time for COVID-19 screening. Science Advances 7, eabd5393).

The challenges faced by traditional in vitro diagnostic techniques, and the need for more frequent testing, have accelerated the development of alternative COVID-19 testing strategies (See e.g., Udugama, B. et al. Diagnosing COVID-19: The Disease and Tools for Detection. ACS Nano 14, 3822-3835 (2020)). Several nanotechnology-based approaches have also been developed for detecting SARS-CoV-2, including those based on nanoparticles (See e.g., Moitra, P., et al., Selective Naked-Eye Detection of SARS-CoV-2 Mediated by N Gene Targeted Antisense Oligonucleotide Capped Plasmonic Nanoparticles. ACS Nano 14, 7617-7627 (2020), DNAzymes (See e.g., Sundah, N. R. et al. Catalytic amplification by transition-state molecular switches for direct and sensitive detection of SARS-CoV-2. Science Advances 7, eabe5940 (2021)), carbon nanotubes (See e.g., Pinals, R. L. et al. Rapid SARS-CoV-2 Spike Protein Detection by Carbon Nanotube-Based Near-Infrared Nanosensors. Nano Lett. 21, 2272-2280 (2021)), graphene (See e.g., Torrente-Rodriguez, R. M. et al. SARS-CoV-2 RapidPlex: A Graphene-Based Multiplexed Telemedicine Platform for Rapid and Low-Cost COVID-19 Diagnosis and Monitoring. Matter 3, 1981-1998 (2020)) and quantum dots (See e.g., Li, C. et al. Synthesis of polystyrene-based fluorescent quantum dots nanolabel and its performance in H5N1 virus and SARS-CoV-2 antibody sensing. Talanta 225, 122064 (2021)), with microfluidic (See e.g., Funari, R., Chu, K.-Y. & Shen, A. Q. Detection of antibodies against SARS-CoV-2 spike protein by gold nanospikes in an opto-microfluidic chip. Biosensors and Bioelectronics 169, 112578 (2020)), lateral flow (See e.g., Huang, C., Wen, T., Shi, F.-J., Zeng, X.-Y. & Jiao, Y.-J. Rapid Detection of IgM Antibodies against the SARS-CoV-2 Virus via Colloidal Gold Nanoparticle-Based Lateral-Flow Assay. ACS Omega 5, 12550-12556 (2020)), and optical (See e.g., Liu, T. et al. Quantification of antibody avidities and accurate detection of SARS-CoV-2 antibodies in serum and saliva on plasmonic substrates. Nature Biomedical Engineering 4, 1188-1196 (2020)), readout systems. Among new materials developed for biosensing, DNA-based nanostructures have been used to detect nucleic acids (See e.g., Chandrasekaran, A. R., Zavala, J. & Halvorsen, K. Programmable DNA Nanoswitches for Detection of Nucleic Acid Sequences. ACS Sens. 1, 120-123 (2016)), proteins (See e.g., Raveendran, M., Lee, A. J., Sharma, R., Wälti, C. & Actis, P. Rational design of DNA nanostructures for single molecule biosensing. Nature Communications 11, 4384 (2020), small molecules (See e.g., Jing, C. et al., An electrochemical aptasensor for ATP based on a configuration-switchable tetrahedral DNA nanostructure. Anal. Methods 12, 3285-3289 (2020), ions (See e.g., Narayanaswamy, N. et al. A pH-correctable, DNA-based fluorescent reporter for organellar calcium. Nature Methods 16, 95 (2019)), and changes in pH (See e.g., Idili, A., Vallée-Bélisle, A. & Ricci, F. Programmable pH-Triggered DNA Nanoswitches. J. Am. Chem. Soc. 136, 5836-5839 (2014)) or temperature. (See e.g., Gareau, D., Desrosiers, A. & Vallée-Bélisle, A. Programmable Quantitative DNA Nanothermometers. Nano Lett. 16, 3976-3981 (2016).

Recently, the precise spatial positioning and programmable control over shape of DNA nanostructures has been used to elucidate viral vaccine design principles (See e.g., Veneziano, R. et al. Role of nanoscale antigen organization on B-cell activation probed using DNA origami. Nature Nanotechnology 15, 716-723 (2020), and in viral diagnostics and therapeutics. (See e.g., Kwon, P. S. et al. Designer DNA architecture offers precise and multivalent spatial pattern-recognition for viral sensing and inhibition. Nature Chemistry 12, 26-35 (2020)). Here, a DNA nanotechnology based SARS-CoV-2 detection assay is provided based on programmable DNA nanoswitches. In embodiments, the DNA nanoswitch based assay is suitable for non-enzymatic, direct detection of SARS-CoV-2 by targeting multiple fragments of the SARS-CoV-2 genome. Assay embodiments have excellent sensitivity and specificity to discriminate single nucleotide variants, low cost (<$1) and has a gel-based readout that can be easily adapted for low-resource setting. Clinical utility is demonstrated herein by detecting the viral RNA in 10 clinically tested COVID-19 positive and 5 negative samples.

### Suitable Materials

Table 14 below shows suitable target RNA strands for detecting SARS-CoV-2 in accordance with the present disclosure.

TABLE 14
SARS-CoV-2 Target sequences

caaaccaaccaactttcgatctcttgtagatctgttctctaaacgaactttaaaatctgt (SEQ ID NO: 298)
gagccttgtccctggtttcaacgagaaaacacacgtccaactcagtttgcctgttttaca (SEQ ID NO: 299)
tcatttgacttaggcgacgagcttggcactgatccttatgaagattttcaagaaaactgg (SEQ ID NO: 300)
aatgtccaaattttgtatttcccttaaattccataatcaagactattcaaccaagggttg (SEQ ID NO: 301)
ttacttaccccaaaatgctgttgttaaaatttattgtccagcatgtcacaattcagaagt (SEQ ID NO: 302)
ggtcttaatgacaaccttcttgaaatactccaaaaagagaaagtcaacatcaatattgtt (SEQ ID NO: 303)
aattttaaagttacaaaaggaaaagctaaaaaaggtgcctggaatattggtgaacagaaa (SEQ ID NO: 304)
taactaacatctttggcactgtttatgaaaaactcaaacccgtccttgattggcttgaag (SEQ ID NO: 305)
gttaaatccagagaagaaactggcctactcatgcctctaaaagccccaaaagaaattatc (SEQ ID NO: 306)
agagtgtgaatatcacttttgaacttgatgaaaggattgataaagtacttaatgagaagt (SEQ ID NO: 307)
gaggatgaagaagaaggtgattgtgaagaagaagagtttgagccatcaactcaatatgag (SEQ ID NO: 308)
acaagacggcagtgaggacaatcagacaactactattcaaacaattgttgaggttcaacc (SEQ ID NO: 309)
actaacaatgccatgcaagttgaatctgatgattacatagctactaatggaccacttaaa (SEQ ID NO: 310)
gcttatgaaaattttaatcagcacgaagttctacttgcaccattattatcagctggtatt (SEQ ID NO: 311)
ttttggaaatgaagagtgaaaagcaagttgaacaaaagatcgctgagattcctaaagagg (SEQ ID NO: 312)
aagttcctcacagaaaacttgttactttatattgacattaatggcaatcttcatccagat (SEQ ID NO: 313)
gaaatgctagcgaaagctttgagaaaagtgccaacagacaattatataaccacttacccg (SEQ ID NO: 314)
aaactaaagccatagtttcaactatacagcgtaaatataagggtattaaaatacaagagg (SEQ ID NO: 315)
tatcttacttcttcttctaaaacacctgaagaacattttattgaaaccatctcacttgct (SEQ ID NO: 316)
gatggagctgatgttactaaaataaaacctcataattcacatgaaggtaaaacattttat (SEQ ID NO: 317)
taacactccaacaaatagagttgaagtttaatccacctgctctacaagatgcttattaca (SEQ ID NO: 318)
tgcaaaagagtcttgaacgtggtgtgtaaaacttgtggacaacagcagacaacccttaag (SEQ ID NO: 319)
ttaccagtgtggtcactataaacatataacttctaaagaaactttgtattgcatagacgg (SEQ ID NO: 320)
attcttatttcacagagcaaccaattgatcttgtaccaaaccaaccatatccaaacgcaa (SEQ ID NO: 321)
tgattataaacactacacaccctcttttaagaaaggagctaaattgttacataaacctat (SEQ ID NO: 322)
acttaaaccagcaaataatagtttaaaaattacagaagaggttggccacacagatctaat (SEQ ID NO: 323)
aaaccgtgtttgtactaattatatgccttatttctttactttattgctacaattgtgtac (SEQ ID NO: 324)
aaactgataaatattataatttggtttttactattaagtgtttgcctaggttctttaatc (SEQ ID NO: 325)
ttctttagacacctatccttctttagaaactatacaaattaccatttcatcttttaaatg (SEQ ID NO: 326)
ggccccgatttcagctatggttagaatgtacatcttctttgcatcattttattatgtatg (SEQ ID NO: 327)
agtttaaaagaccaataaatcctactgaccagtcttcttacatcgttgatagtgttacag (SEQ ID NO: 328)
tattaatgttatagtttttgatggtaaatcaaaatgtgaagaatcatctgcaaaatcagc (SEQ ID NO: 329)
aatacgttttcatcaacttttaacgtaccaatggaaaaactcaaaacactagttgcaact (SEQ ID NO: 330)
ttgtaataactatatgctcacctataacaaagttgaaaacatgacaccccgtgaccttgg (SEQ ID NO: 331)
gttcctttttgttgctgctattttctatttaataacacctgttcatgtcatgtctaaaca (SEQ ID NO: 332)
aacatctgttacacaccatcaaaacttatagagtacactgactttgcaacatcagcttgt (SEQ ID NO: 333)
tgacacacgttatgtgctcatggatggctctattattcaatttcctaacacctaccttga (SEQ ID NO: 334)
tcatgtagttgcctttaatactttactattccttatgtcattcactgtactctgtttaac (SEQ ID NO: 335)
gttatgttcacacctttagtacctttctggataacaattgcttatatcatttgtatttcc (SEQ ID NO: 336)
acttcagtaactcaggttctgatgttctttaccaaccaccacaaacctctatcacctcag (SEQ ID NO: 337)
ttgatacagccaatcctaagacacctaagtataagtttgttcgcattcaaccaggacaga (SEQ ID NO: 338)
aatggttcatgtggtagtgttggttttaacatagattatgactgtgtctctttttgttac (SEQ ID NO: 339)
ctatgaagtacaattatgaacctctaacacaagaccatgttgacatactaggacctcttt (SEQ ID NO: 340)
gtactcaatggtctttgttcttttttttgtatgaaaatgcctttttaccttttgctatgg (SEQ ID NO: 341)
gttttaagctaaaagactgtgttatgtatgcatcagctgtagtgttactaatccttatga (SEQ ID NO: 342)
tcatgtttttggccagaggtattgtttttatgtgtgttgagtattgccctattttcttca (SEQ ID NO: 343)
tacagtctaaaatgtcagatgtaaagtgcacatcagtagtcttactctcagttttgcaac (SEQ ID NO: 344)
ggcaaccttacaagctatagcctcagagtttagttcccttccatcatatgcagcttttgc (SEQ ID NO: 345)
tatgcagacaatgcttttcactatgcttagaaagttggataatgatgcactcaacaacat (SEQ ID NO: 346)
tgaaattagtatggacaattcacctaatttagcatggcctcttattgtaacagctttaag (SEQ ID NO: 347)
tttacaggatttgaaatgggctagattccctaagagtgatggaactggtactatctatac (SEQ ID NO: 348)
gcctgccaattcaactgtattatctttctgtgcttttgctgtagatgctgctaaagctta (SEQ ID NO: 349)
acaaatacctacaacttgtgctaatgaccctgtgggttttacacttaaaaacacagtctg (SEQ ID NO: 350)
ataaagtagctggttttgctaaattcctaaaaactaattgttgtcgcttccaagaaaagg (SEQ ID NO: 351)
ccacatatatcacgtcaacgtcttactaaatacacaatggcagacctcgtctatgcttta (SEQ ID NO: 352)
tatttcaataaaaaggactggtatgattttgtagaaaacccagatatattacgcgtatac (SEQ ID NO: 353)
ggtagtggagttcctgttgtagattcttattattcattgttaatgcctatattaaccttg (SEQ ID NO: 354)
cattgtgcaaactttaatgttttattctctacagtgttcccacctacaagttttggacca (SEQ ID NO: 355)
ttactaacaatgttgcttttcaaactgtcaaacccggtaattttaacaaagacttctatg (SEQ ID NO: 356)
ctgtattaatgctaaccaagtcatcgtcaacaacctagacaaatcagctggttttccatt (SEQ ID NO: 357)
catccctactataactcaaatgaatcttaagtatgccattagtgcaaagaatagagctcg (SEQ ID NO: 358)
catgcctaacatgcttagaattatggcctcacttgttcttgctcgcaaacatacaacgtg (SEQ ID NO: 359)
atttgcgtaaacatttctcaatgatgatactctctgacgatgctgttgtgtgtttcaata (SEQ ID NO: 360)
tgattatgtgtaccttccttacccagatccatcaagaatcctaggggccggctgttttgt (SEQ ID NO: 361)
atttgtacttacaatacataagaaagctacatgatgagttaacaggacacatgttagaca (SEQ ID NO: 362)
aaatgctgttacgaccatgtcatatcaacatcacataaattagtcttgtctgttaatccg (SEQ ID NO: 363)
gtgactggacaaatgctggtgattacattttagctaacacctgtactgaaagactcaagc (SEQ ID NO: 364)
aacctagaccaccacttaaccgaaattatgtctttactggttatcgtgtaactaaaaaca (SEQ ID NO: 365)
tcattttgctattggcctagctctctactacccttctgctcgcatagtgtatacagcttg (SEQ ID NO: 366)
caattacctgcaccacgcacattgctaactaagggcacactagaaccagaatatttcaat (SEQ ID NO: 367)
aattccttacacgtaaccctgcttggagaaaagctgtctttatttcaccttataattcac (SEQ ID NO: 368)
gtaggaatgtggcaactttacaagctgaaaatgtaacaggactctttaaagattgtagta (SEQ ID NO: 369)
ttttaaaatgaattatcaagttaatggttaccctaacatgtttatcacccgcgaagaagc (SEQ ID NO: 370)
gctaaaccaccgcctggagatcaatttaaacacctcataccacttatgtacaaaggactt (SEQ ID NO: 371)
gttgacatctatgaagtattttgtgaaaataggacctgagcgcacctgttgtctatgtga (SEQ ID NO: 372)
tgttaagcgtgttgactggactattgaatatcctataattggtgatgaactgaagattaa (SEQ ID NO: 373)
acaaagcttataaaatagaagaattattctattcttatgccacacattctgacaaattca (SEQ ID NO: 374)
aagtgcttttgttaatttaaaacaattaccatttttctattactctgacagtccatgtga (SEQ ID NO: 375)
ttagcttgtgggtttacaaacaatttgatacttataacctctggaacacttttacaagac (SEQ ID NO: 376)
tgactgacatagccaagaaaccaactgaaacgatttgtgcaccactcactgtcttttttg (SEQ ID NO: 377)
ttgtccaacaattacctgaaacttactttactcagagtagaaatttacaagaatttaaac (SEQ ID NO: 378)
aattagaagattttattcctatggacagtacagttaaaaactatttcataacagatgcgc (SEQ ID NO: 379)
tggtgtaaagatggccatgtagaaacattttacccaaaattacaatctagtcaagcgtgg (SEQ ID NO: 380)
tcaatatttaaacacattaacattagctgtaccctataatatgagagttatacattttgg (SEQ ID NO: 381)
ttattagtgatatgtacgaccctaagactaaaaatgttacaaaagaaaatgactctaaag (SEQ ID NO: 382)
acgcgaacaaatagatggttatgtcatgcatgcaaattacatattttggaggaatacaaa (SEQ ID NO: 383)
tgttaatcttacaaccagaactcaattaccccctgcatacactaattctttcacacgtgg (SEQ ID NO: 384)
gtactactttagattcgaagacccagtccctacttattgttaataacgctactaatgttg (SEQ ID NO: 385)
tttaagaatattgatggttattttaaaatatattctaagcacacgcctattaatttagtg (SEQ ID NO: 386)
tcttcaacctaggacttttctattaaaatataatgaaaatggaaccattacagatgctgt (SEQ ID NO: 387)
taagtgttatggagtgtctcctactaaattaaatgatctctgctttactaatgtctatgc (SEQ ID NO: 388)
aggttttaattgttactttcctttacaatcatatggtttccaacccactaatggtgttgg (SEQ ID NO: 389)
ctttccaacaatttggcagagacattgctgacactactgatgctgtccgtgatccacaga (SEQ ID NO: 390)
tgcagatcaacttactcctacttggcgtgtttattctacaggttctaatgtttttcaaac (SEQ ID NO: 391)
taataactctattgccatacccacaaattttactattagtgttaccacagaaattctacc (SEQ ID NO: 392)
aacacccaagaagtttttgcacaagtcaaacaaatttacaaaacaccaccaattaaagat (SEQ ID NO: 393)
taacggccttactgttttgccacctttgctcacagatgaaatgattgctcaatacacttc (SEQ ID NO: 394)
ctccaattttggtgcaatttcaagtgttttaaatgatatcctttcacgtcttgacaaagt (SEQ ID NO: 395)
tatgtccctgcacaagaaaagaacttcacaactgctcctgccatttgtcatgatggaaaa (SEQ ID NO: 396)
taataggaattgtcaacaacacagtttatgatcctttgcaacctgaattagactcattca (SEQ ID NO: 397)
gaccgcctcaatgaggttgccaagaatttaaatgaatctctcatcgatctccaagaactt (SEQ ID NO: 398)
tttgtttatgagaatcttcacaattggaactgtaactttgaagcaaggtgaaatcaagga (SEQ ID NO: 399)
tttactcacaccttttgctcgttgctgctggccttgaagccccttttctctatctttatg (SEQ ID NO: 400)
cccattactttatgatgccaactattttctttgctggcatactaattgttacgactattg (SEQ ID NO: 401)
tcaattgagtacagacactggtgttgaacatgttaccttcttcatctacaataaaattgt (SEQ ID NO: 402)
ttaatagttaatagcgtacttctttttcttgctttcgtggtattcttgctagttacacta (SEQ ID NO: 403)
taaatattatattagtttttctgtttggaactttaattttagccatggcagattccaacg (SEQ ID NO: 404)
cgcgttccatgtggtcattcaatccagaaactaacattcttctcaacgtgccactccatg (SEQ ID NO: 405)
ggacctgcctaaagaaatcactgttgctacatcacgaacgctttcttattacaaattggg (SEQ ID NO: 406)
tggaatcttgattacatcataaacctcataattaaaaatttatctaagtcactaactgag (SEQ ID NO: 407)
ttcaccatttcatcctctagctgataacaaatttgcactgacttgctttagcactcaatt (SEQ ID NO: 408)
cttctatttgtgctttttagcctttctgctattccttgttttaattatgcttattatctt (SEQ ID NO: 409)
ctaaatcacccattcagtacatcgatatcggtaattatacagtttcctgtttacctttta (SEQ ID NO: 410)
ccccaaggtttacccaataatactgcgtcttggttcaccgctctcactcaacatggcaag (SEQ ID NO: 411)
cagacgaattcgtggtggtgacggtaaaatgaaagatctcagtccaagatggtatttcta (SEQ ID NO: 412)
tcaagcctcttctcgttcctcatcacgtagtcgcaacagttcaagaaattcaactccagg (SEQ ID NO: 413)
catacaatgtaacacaagctttcggcagacgtggtccagaacaaacccaaggaaattttg (SEQ ID NO: 414)
catattgacgcatacaaaacattcccaccaacagagcctaaaaaggacaaaaagaagaag (SEQ ID NO: 415)
taaacgttttcgcttttccgtttacgatatatagtctactcttgtgcagaatgaattctc (SEQ ID NO: 416)
gaagagccctaatgtgtaaaattaattttagtagtgctatccccatgtgattttaatagc (SEQ ID NO: 417)

Table 15 below shows suitable left or first detector strands for certain SARS-CoV-2 target strands above.

FIRST OR LEFT DETECTOR STRANDS

TCTGTCCATCACGCAAATTAACCGTTGTAGACAGATTTTAAAGTTCGTTT
AGAGAACAGA (SEQ ID NO: 418)
CAATACTTCTTTGATTAGTAATAACATCACTGTAAAACAGGCAAACTGAG
TTGGACGTGT (SEQ ID NO: 419)
TTGCCTGAGTAGAAGAACTCAAACTATCGGCCAGTTTTCTTGAAAATCTT
CATAAGGATC (SEQ ID NO: 420)
CCTTGCTGGTAATATCCAGAACAATATTACCAACCCTTGGTTGAATAGTC
TTGATTATGG (SEQ ID NO: 421)
CGCCAGCCATTGCAACAGGAAAAACGCTCAACTTCTGAATTGTGACATGC
TGGACAATAA (SEQ ID NO: 422)
TGGAAATACCTACATTTTGACGCTCAATCGAACAATATTGATGTTGACTT
TCTCTTTTTG (SEQ ID NO: 423)
TCTGAAATGGATTATTTACATTGGCAGATTTTTCTGTTCACCAATATTCC
AGGCACCTTT (SEQ ID NO: 424)
CACCAGTCACACGACCAGTAATAAAAGGGACTTCAAGCCAATCAAGGACG
GGTTTGAGTT (SEQ ID NO: 425)
CATTCTGGCCAACAGAGATAGAACCCTTCTGATAATTTCTTTTGGGGCTT
TTAGAGGCAT (SEQ ID NO: 426)
GACCTGAAAGCGTAAGAATACGTGGCACAGACTTCTCATTAAGTACTTTA
TCAATCCTTT (SEQ ID NO: 427)
ACAATATTTTTGAATGGCTATTAGTCTTTACTCATATTGAGTTGATGGCT
CAAACTCTTC (SEQ ID NO: 428)
ATGCGCGAACTGATAGCCCTAAAACATCGCGGTTGAACCTCAACAATTGT
TTGAATAGTA (SEQ ID NO: 429)
CATTAAAAATACCGAACGAACCACCAGCAGTTTAAGTGGTCCATTAGTAG
CTATGTAATC (SEQ ID NO: 430)
AAGATAAAACAGAGGTGAGGCGGTCAGTATAATACCAGCTGATAATAATG
GTGCAAGTAG (SEQ ID NO: 431)
TAACACCGCCTGCAACAGTGCCACGCTGAGCCTCTTTAGGAATCTCAGCG
ATCTTTTGTT (SEQ ID NO: 432)
AGCCAGCAGCAAATGAAAAATCTAAAGCATATCTGGATGAAGATTGCCAT
TAATGTCAAT (SEQ ID NO: 433)
CACCTTGCTGAACCTCAAATATCAAACCCTCGGGTAAGTGGTTATATAAT
TGTCTGTTGG (SEQ ID NO: 434)
CAATCAATATCTGGTCAGTTGGCAAATCAACCTCTTGTATTTTAATACCC
TTATATTTAC (SEQ ID NO: 435)
CAGTTGAAAGGAATTGAGGAAGGTTATCTAAGCAAGTGAGATGGTTTCAA
TAAAATGTTC (SEQ ID NO: 436)
AAATATCTTTAGGAGCACTAACAACTAATAATAAAATGTTTTACCTTCAT
GTGAATTATG (SEQ ID NO: 437)
GATTAGAGCCGTCAATAGATAATACATTTGTGTAATAAGCATCTTGTAGA
TGCAGGGGAT (SEQ ID NO: 438)
AGGATTTAGAAGTATTAGACTTTACAAACACTTAAGGGTTGTCTGCTGTT
GTCCACAAGT (SEQ ID NO: 439)
ATTCGACAACTCGTATTAAATCCTTTGCCCCCGTCTATGCAATACAAAGT
TTCTTTAGAA (SEQ ID NO: 440)
GAACGTTATTAATTTTAAAAGTTTGAGTAATTGCGTTTGGATATGGTTGG
TTTGGTACAA (SEQ ID NO: 441)
TCTGTCCATCACGCAAATTAACCGTTGTAGATAGGTTTATGTAACAATTT
AGCTCCTTTC (SEQ ID NO: 442)
CAATACTTCTTTGATTAGTAATAACATCACATTAGATCTGTGTGGCCAAC
CTCTTCTGTA (SEQ ID NO: 443)
TTGCCTGAGTAGAAGAACTCAAACTATCGGGTACACAATTGTAGCAATAA
AGTAAAGAAA (SEQ ID NO: 444)
CCTTGCTGGTAATATCCAGAACAATATTACGATTAAAGAACCTAGGCAAA
CACTTAATAG (SEQ ID NO: 445)
CGCCAGCCATTGCAACAGGAAAAACGCTCACATTTAAAAGATGAAATGGT
AATTTGTATA (SEQ ID NO: 446)
TGGAAATACCTACATTTTGACGCTCAATCGCATACATAATAAAATGATGC
AAAGAAGATG (SEQ ID NO: 447)

Table 16 below shows suitable right or second detector strands for certain SARS-CoV-2 target strands above. It is noted that Tables 15 and 16 show paired detector strands when compared against each other in ascending order.

SECOND OR RIGHT DETECTOR STRANDS

TCTACAAGAGATCGAAAGTTGGTTGGTTTGTCAACCGATTGAGGGAGGGA
AGGTAAATAT (SEQ ID NO: 448)
GTTTTCTCGTTGAAACCAGGGACAAGGCTCTGACGGAAATTATTCATTAA
AGGTGAATTA (SEQ ID NO: 449)
AGTGCCAAGCTCGTCGCCTAAGTCAAATGATCACCGTCACCGACTTGAGC
CATTTGGGAA (SEQ ID NO: 450)
AATTTAAGGGAAATACAAAATTTGGACATTTTAGAGCCAGCAAAATCACC
AGTAGCACCA (SEQ ID NO: 451)
ATTTTAACAACAGCATTTTGGGGTAAGTAATTACCATTAGCAAGGCCGGA
AACGTCACCA (SEQ ID NO: 452)
GAGTATTTCAAGAAGGTTGTCATTAAGACCATGAAACCATCGATAGCAGC
ACCGTAATCA (SEQ ID NO: 453)
TTTAGCTTTTCCTTTTGTAACTTTAAAATTGTAGCGACAGAATCAAGTTT
GCCTTTAGCG (SEQ ID NO: 454)
TTTCATAAACAGTGCCAAAGATGTTAGTTATCAGACTGTAGCGCGTTTTC
CATCGGATTT (SEQ ID NO: 455)
GAGTAGGCCAGTTTCTTCTCTGGATTTAACTCGGTCATAGCCCCCTTATT
AGCGTTTGCC (SEQ ID NO: 456)
CATCAAGTTCAAAAGTGATATTCACACTCTATCTTTTCATAATCAAAATC
ACCGGAACCA (SEQ ID NO: 457)
TTCTTCACAATCACCTTCTTCTTCATCCTCGAGCCACCACCGGAACCGCC
TCCCTCAGAG (SEQ ID NO: 458)
GTTGTCTGATTGTCCTCACTGCCGTCTTGTCCGCCACCCTCAGAACCGCC
ACCCTCAGAG (SEQ ID NO: 459)
ATCAGATTCAACTTGCATGGCATTGTTAGTCCACCACCCTCAGAGCCGCC
ACCAGAACCA (SEQ ID NO: 460)
AACTTCGTGCTGATTAAAATTTTCATAAGCCCACCAGAGCCGCCGCCAGC
ATTGACAGGA (SEQ ID NO: 461)
CAACTTGCTTTTCACTCTTCATTTCCAAAAGGTTGAGGCAGGTCAGACGA
TTGGCCTTGA (SEQ ID NO: 462)
ATAAAGTAACAAGTTTTCTGTGAGGAACTTTATTCACAAACAAATAAATC
CTCATTAAAG (SEQ ID NO: 463)
CACTTTTCTCAAAGCTTTCGCTAGCATTTCCCAGAATGGAAAGCGCAGTC
TCTGAATTTA (SEQ ID NO: 464)
GCTGTATAGTTGAAACTATGGCTTTAGTTTCCGTTCCAGTAAGCGTCATA
CATGGCTTTT (SEQ ID NO: 465)
TTCAGGTGTTTTAGAAGAAGAAGTAAGATAGATGATACAGGAGTGTACTG
GTAATAAGTT (SEQ ID NO: 466)
AGGTTTTATTTTAGTAACATCAGCTCCATCTTAACGGGGTCAGTGCCTTG
AGTAACAGTG (SEQ ID NO: 467)
TAAACTTCAACTCTATTTGTTGGAGTGTTACCCGTATAAACAGTTAATGC
CCCCTGCCTA (SEQ ID NO: 468)
TTTACACACCACGTTCAAGACTCTTTTGCATTTCGGAACCTATTATTCTG
AAACATGAAA (SEQ ID NO: 469)
GTTATATGTTTATAGTGACCACACTGGTAAGTATTAAGAGGCTGAGACTC
CTCAAGAGAA (SEQ ID NO: 470)
GATCAATTGGTTGCTCTGTGAAATAAGAATGGATTAGGATTAGCGGGGTT
TTGCTCAGTA (SEQ ID NO: 471)
TTAAAAGAGGGTGTGTAGTGTTTATAATCATCAACCGATTGAGGGAGGGA
AGGTAAATAT (SEQ ID NO: 472)
ATTTTTAAACTATTATTTGCTGGTTTAAGTTGACGGAAATTATTCATTAA
AGGTGAATTA (SEQ ID NO: 473)
TAAGGCATATAATTAGTACAAACACGGTTTTCACCGTCACCGACTTGAGC
CATTTGGGAA (SEQ ID NO: 474)
TAAAAACCAAATTATAATATTTATCAGTTTTTAGAGCCAGCAAAATCACC
AGTAGCACCA (SEQ ID NO: 475)
GTTTCTAAAGAAGGATAGGTGTCTAAAGAATTACCATTAGCAAGGCCGGA
AACGTCACCA (SEQ ID NO: 476)
TACATTCTAACCATAGCTGAAATCGGGGCCATGAAACCATCGATAGCAGC
ACCGTAATCA (SEQ ID NO: 477)

Results and Discussion

### DNA Nanoswitch Design and Operation

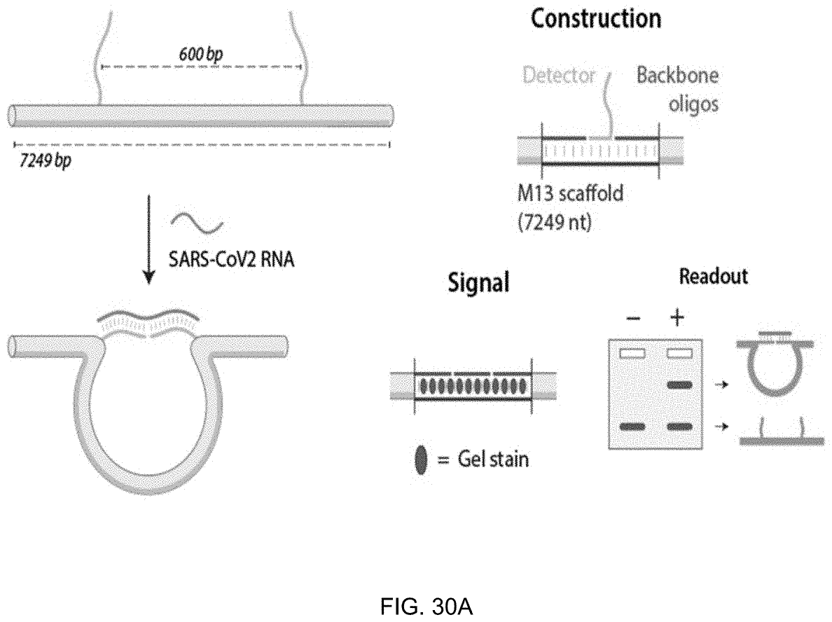

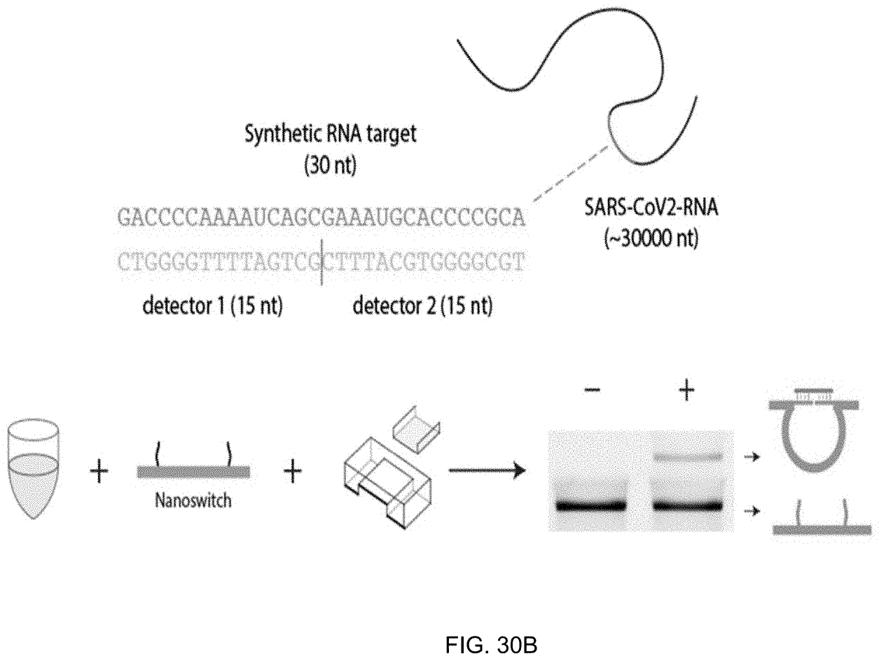

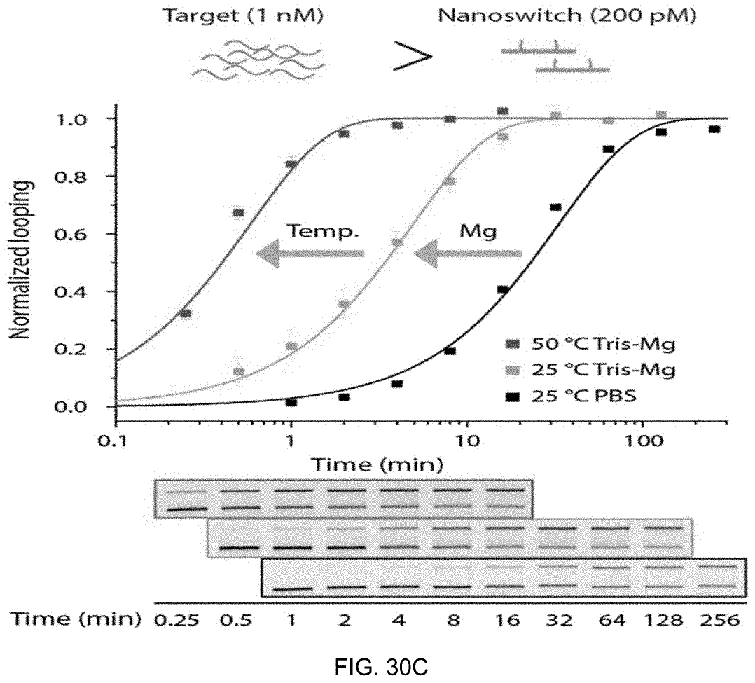

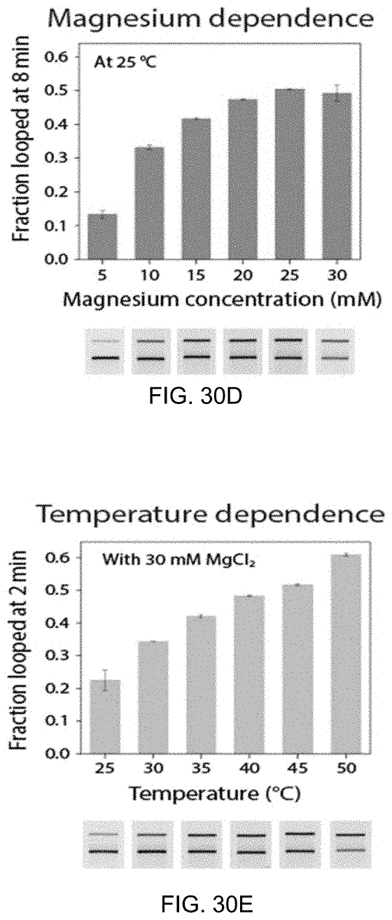

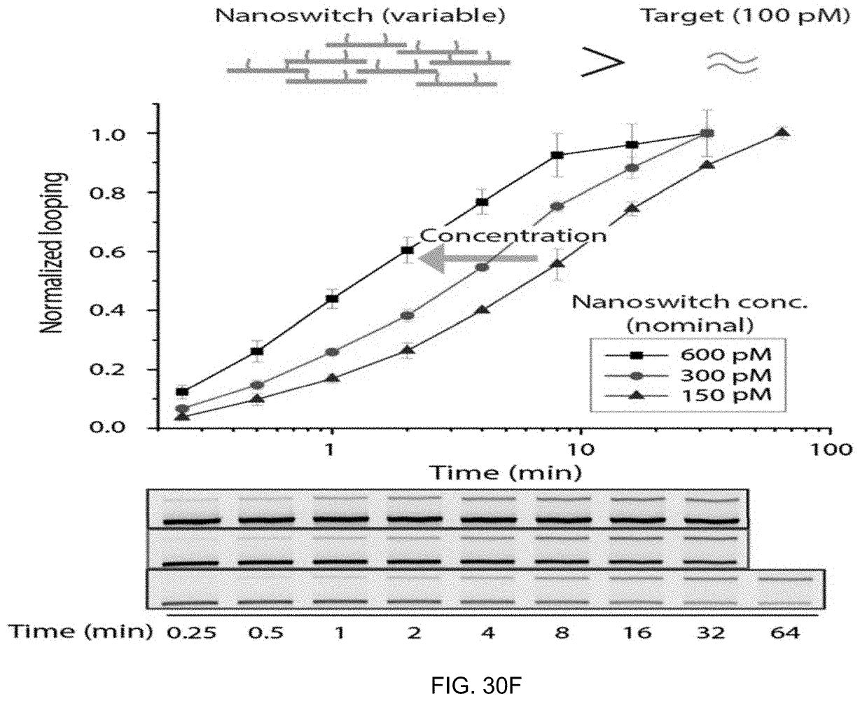

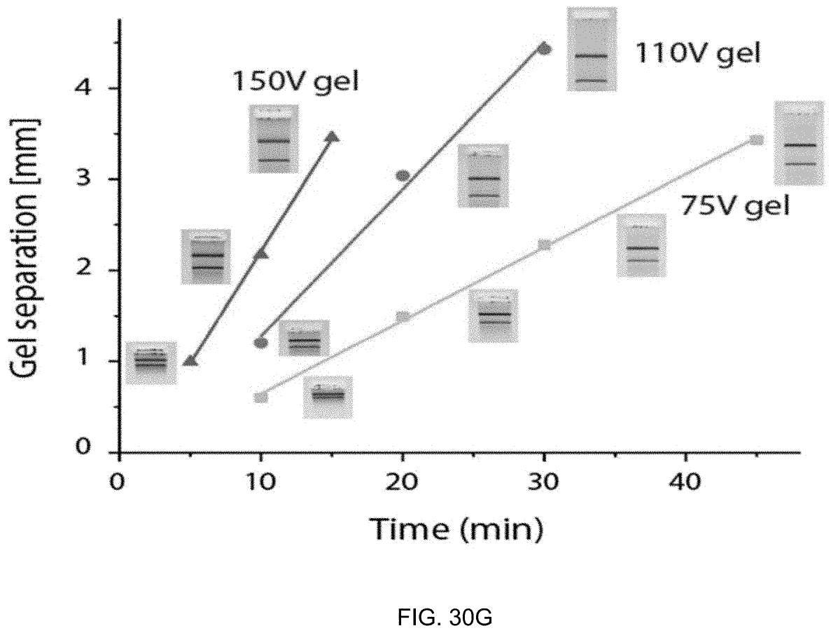

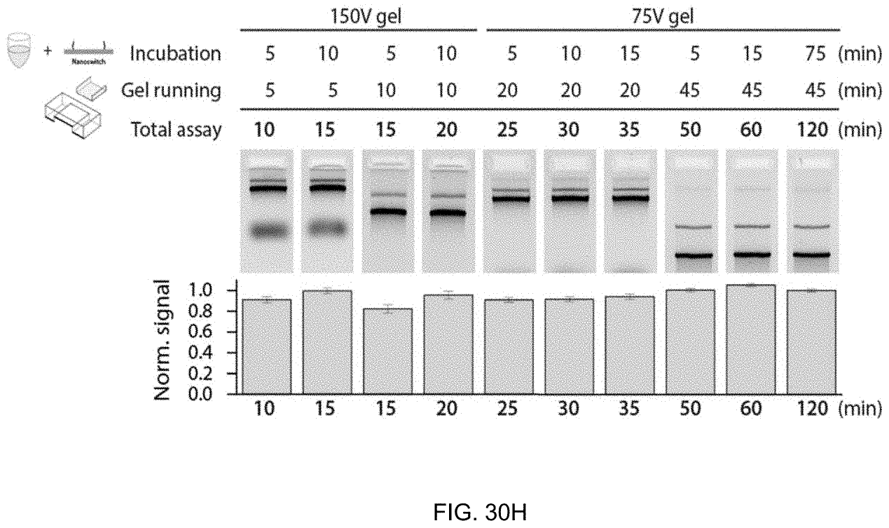

The DNA nanoswitch is constructed based on DNA origami principles. Typically, in DNA origami, a long single stranded DNA is folded into specific two- or three-dimensional shapes using short complementary strands. (See e.g., Rothemund, P. W. K. Folding DNA to create nanoscale shapes and patterns. Nature 440, 297-302 (2006)). Here, a simple linear duplex (the “off” state) was created using a 7249-nt long scaffold strand (commercially available M13 bacteriophage viral genome) tiled with short complementary backbone oligonucleotides. (See e.g., Chandrasekaran, A. R., Zavala, J. & Halvorsen, K. Programmable DNA Nanoswitches for Detection of Nucleic Acid Sequences. ACS Sens. 1, 120-123 (2016)). Two of the oligonucleotides contain single stranded extensions (detectors) that are complementary to parts of a target nucleic acid (FIG. 30A). On binding the target sequence, the nanoswitch is reconfigured from the linear “off” state to a looped “on” state. This conformational change is easily read out on an agarose gel where the on and off states of the nanoswitch migrate differently due to the topological difference (FIG. 30A, inset). Importantly, this approach requires no complex equipment or enzymatic amplification. The signal comes from the intercalation of thousands of dye molecules from regularly used DNA gel stains (GelRed in this case). In previous work, similar DNA nanoswitches were used for single molecule experiments, (See e.g., Halvorsen, et al., Nanoengineering a single-molecule mechanical switch using DNA self-assembly. Nanotechnology 22, 494005 (2011)) detection of microRNAs (See e.g., Chandrasekaran, A. R. et al. Cellular microRNA detection with miRacles: microRNA-activated conditional looping of engineered switches. Science Advances 5, eaau9443 (2019)), viral RNAs (see e.g., Zhou, L. et al. Programmable low-cost DNA-based platform for viral RNA detection. Science Advances eabc6246 (2020) doi:10.1126/sciadv.abc6246), antigens (see e.g., Hansen, C. H., Yang, D., Koussa, M. A. & Wong, W. P. Nanoswitch-linked immunosorbent assay (NLISA) for fast, sensitive, and specific protein detection. PNAS 114, 10367-10372 (2017)) and enzymes (See e.g., Chandrasekaran, A. R., Trivedi, R. & Halvorsen, K. Ribonuclease-Responsive DNA Nanoswitches. Cell Reports Physical Science 1, 100117 (2020)), as well as in molecular memory. (See e.g., Chandrasekaran, A. R., Levchenko, O., Patel, D. S., MacIsaac, M. & Halvorsen, K. Addressable configurations of DNA nanostructures for rewritable memory. Nucleic Acids Res 45, 11459-11465 (2017)).

Here, direct, non-enzymatic detection of SARS-CoV-2 viral RNA is provided. As a first step, a nanoswitch was designed with detectors that respond to a target sequence in SARS-CoV-2 that has been used as a PCR primer in other diagnostic tests. Using a synthetic RNA target, the nanoswitches could detect the SARS-CoV-2 fragment (FIG. 30B). Next a key challenge in detection of SARS-CoV-2 RNA was addressed: achieving a short detection time for a low concentration target. The goal was to achieve a less-than-30-minute reaction time, allowing for an additional 20-30 minute readout time for a total test time of less than 1 hour. Previous nanoswitch work with proteins had shown rapid detection with a 30-minute incubation (See e.g., Hansen, C. H., Yang, D., Koussa, M. A. & Wong, W. P. Nanoswitch-linked immunosorbent assay (NLISA) for fast, sensitive, and specific protein detection. PNAS 114, 10367-10372 (2017)), but this goal had not yet been achieved for nucleic acids, where secondary structures can slow reaction kinetics. To address this issue, the kinetics of the nanoswitch assay were screened for a variety of conditions using a synthetic RNA target that corresponds to a region in the SARS-CoV-2 genome N gene. (FIG. 30C). First, kinetic experiments were performed at room temperature in PBS, which took nearly 2 hours to reach completion. Addition of magnesium, which is known to facilitate nucleic acid hybridization was considered. Using a Tris-HCl buffer, different concentrations of magnesium were tested. It was found that achieving an excellent detection signal required at least 10 mM MgCl₂ and that kinetics were enhanced up to 30 mM MgCl₂ (See FIG. 30D). Choosing 30 mM MgCl₂, kinetics were measured at different temperatures, which showed a continued increase until ˜50 degrees Celsius (FIG. 30E). With a 1 nM target RNA concentration, the magnesium and elevated temperature enabled complete reactions in less than 2 minutes, nearly two orders of magnitude faster than our starting condition.

In a typical viral detection assay, the target concentration (˜fM level) is generally lower than the nanoswitch concentration (˜200-500 pM level). To assess more realistic conditions, the kinetics were evaluated for reactions with excess nanoswitch (FIG. 30F). First, it was confirmed that the rate of the signal accumulation relative to maximum signal is independent of the target concentration under these conditions. Next, the kinetics of the reactions were evaluated with varying nanoswitch concentrations. It was found that increasing the nanoswitch concentration similarly increased the reaction rate. At the highest concentration tested (˜600 pM), it took less than 2 minutes to reach half signal and less than 10 minutes to reach 90% signal (FIG. 30F). To ensure a short end-to-end assay time, suitable gel running conditions were also determined. In embodiments, band separation may be maximized at a given voltage by varying buffer height above the gel increasing separation. Then, nanoswitches may be imaged looped at different voltages and running times, reducing the gel running time to 5-20 minutes with minimal signal loss (FIG. 30G). These process conditions enabled detection of a 50 pM RNA target with an end-to-end assay time of as little as 10 minutes without sacrificing more than 20% of the signal (FIG. 30H).

More specifically, FIGS. 30A-30H depict DNA nanoswitch detection of SARS-CoV-2 RNA. FIG. 30A depicts the DNA nanoswitch is designed to form a loop upon interacting with a specific or preselected target sequence. The nanoswitch is stained and imaged on a gel for detection readout. FIG. 30B depicts the design and validation of a DNA nanoswitch targeting a 30 nt portion of the N-gene. FIG. 30C depicts reaction kinetics for a 30 nt RNA target in excess shows nearly two orders of magnitude improvement with optimal magnesium and temperature. FIG. 30D depicts the effect of various magnesium concentrations on room temperature kinetics. FIG. 30E depicts the effect of various temperatures on kinetics. FIG. 30F depicts the kinetics with limited target. FIG. 30G depicts the gel separation of nanoswitch with different times and voltages. FIG. 30H depicts the overall assay time for a 50 pM target.

### Targeting Multiple Viral RNA Fragments for Signal Enhancement

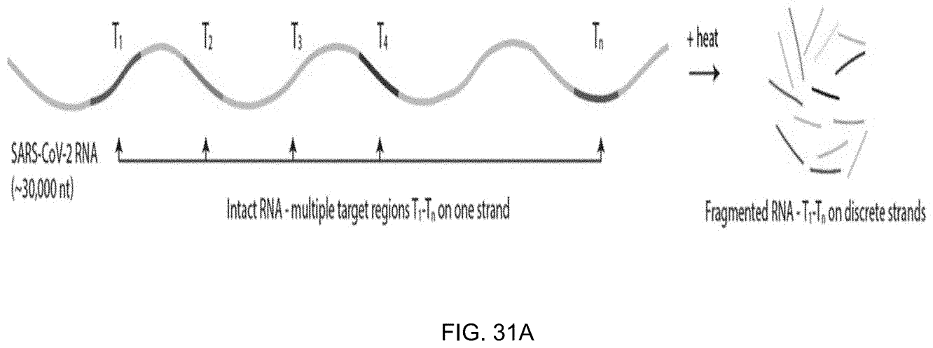

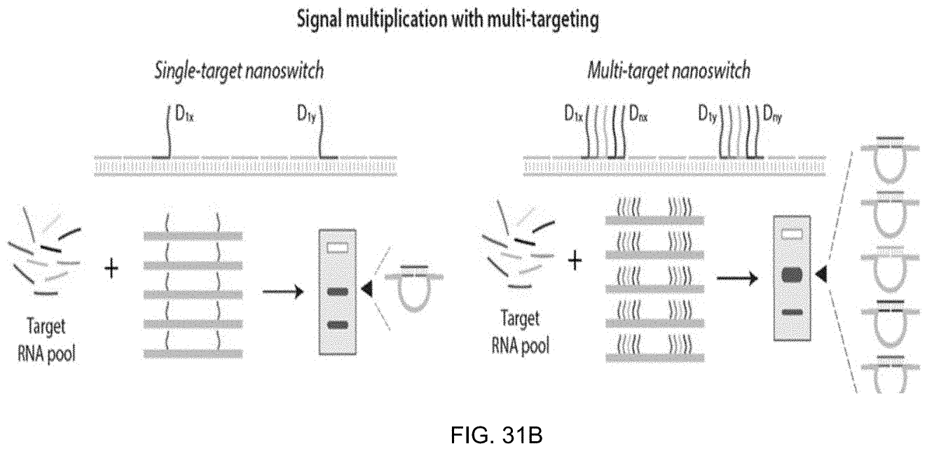

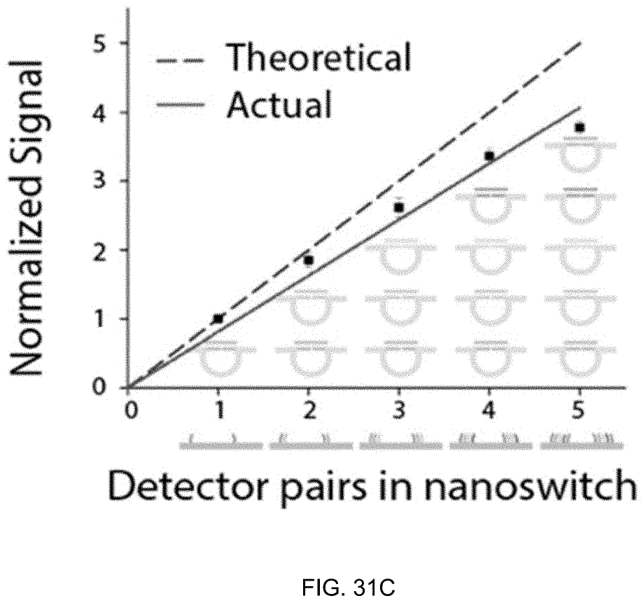

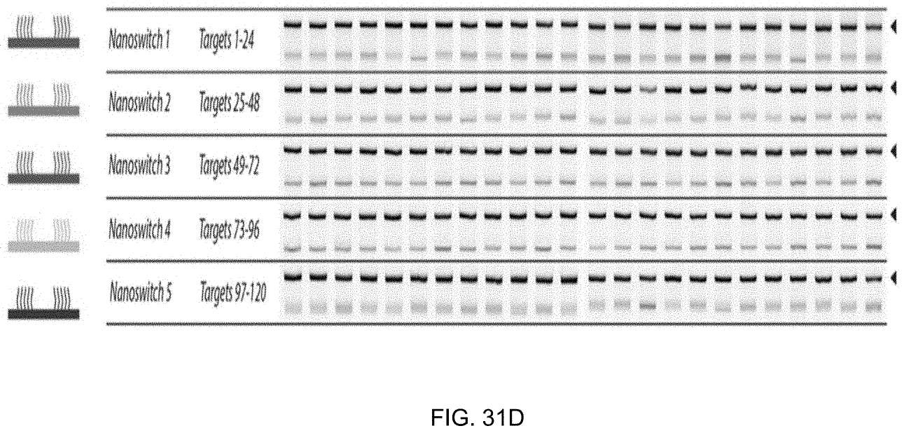

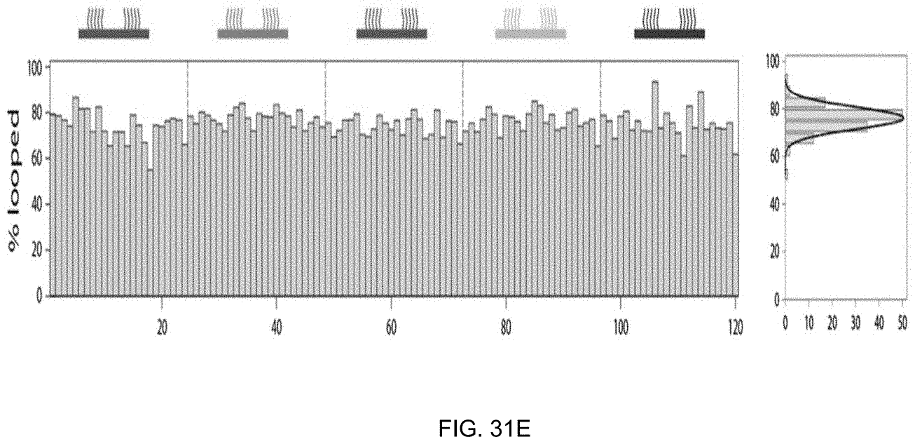

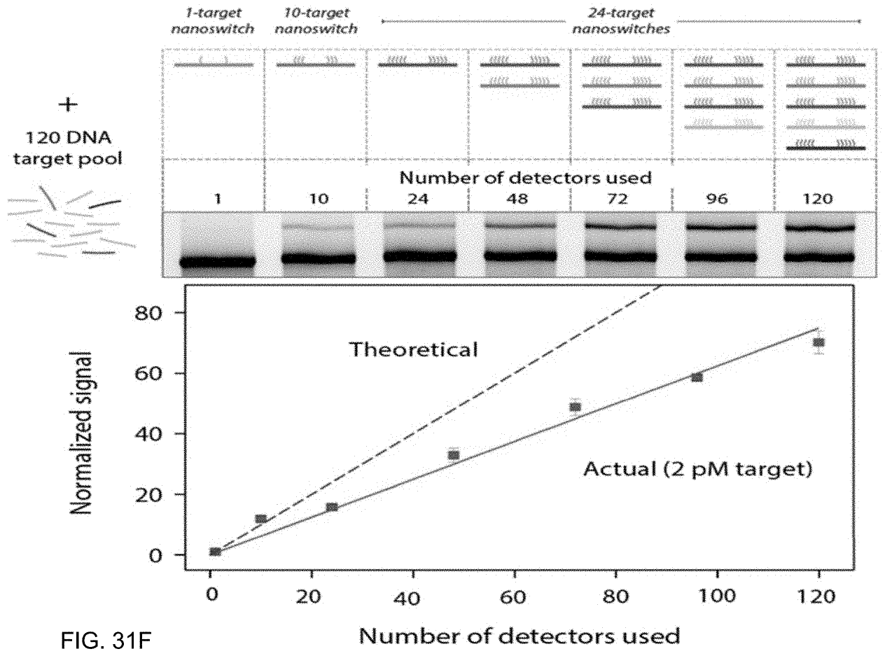

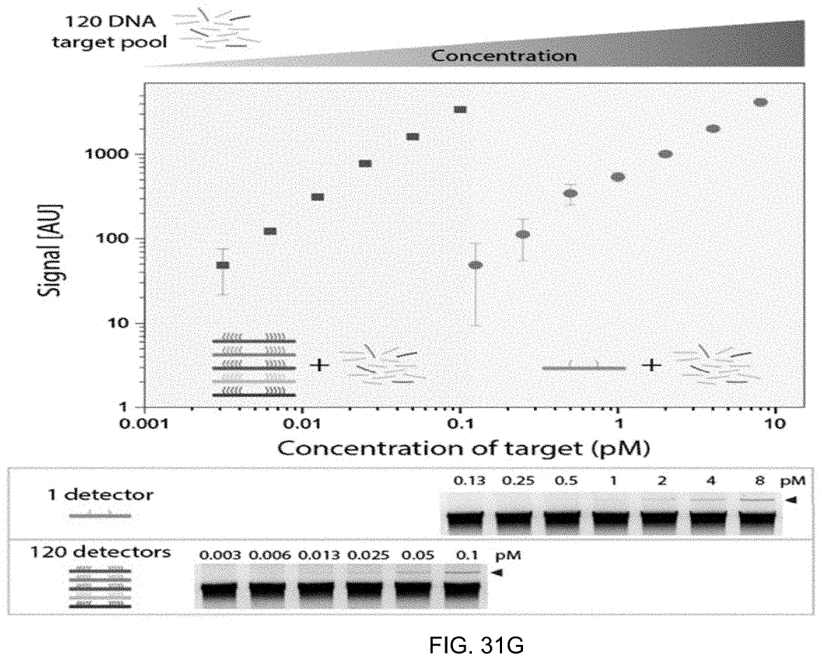

Having satisfied the rapid testing requirement, the analytical sensitivity of the assay for viral targets was determined. Sensitivity for RNA targets has been reported in the 10-100 fM range. Using the process condition parameters of the present disclosure, the sensitivity of the assay was tested for the single target sequence, and determined a limit of detection (LOD) of 122 fM or 0.76 amol. To improve sensitivity, a new strategy for multi-targeting of different fragments of viral RNA was provided. The concept is based on the idea that fragmenting the viral RNA produces many discrete targets from a single viral RNA genome (FIG. 31A). A typical assay with a single target sequence can capture one target RNA per viral RNA, but an assay with multiple target sequences can capture multiple target RNAs per viral RNA to increase signal. In a nanoswitch assay, this can be accomplished by targeting several different SARS-CoV-2 sequences with identical loop sizes to essentially add the signal from multiple targets (FIG. 31B). Here, embodiments include a new nanoswitch design where a plurality or multiple targeting regions can be integrated onto a single nanoswitch, building on previous ideas from combining individual single target nanoswitches. (See e.g., Zhou, L. et al. Programmable low-cost DNA-based platform for viral RNA detection. Science Advances eabc6246 (2020) doi:10.1126/sciadv.abc6246).

More specifically, FIGS. 31A-31G depict improving sensitivity with multi-targeting nanoswitches. FIG. 31A depicts a long viral RNA can have many targeting regions that end up on discrete strands after fragmentation. FIG. 31B depicts the concept of single-target and multi-target sensing in accordance with the present disclosure. FIG. 31C depicts validation of multi-target sensing by targeting one to five sequences in a five sequence pool. FIG. 31D depicts the development of five 24 target nanoswitches to enable 120 different target regions. Gels demonstrate detection of each individual target. FIG. 31E depicts the quantified looped % of each nanoswitch target, with a histogram showing the distribution. FIG. 31F depicts the detection of a mock viral RNA using 120 equimolar targets and corresponding signal increase with increased targeting. FIG. 31G depicts the overall sensitivity of the 120 targeting nanoswitch approach compared with single targeting.

To design a multi-targeting nanoswitch, pairwise detectors were designed with a fixed distance apart with each pair offset along the length of the nanoswitch. Each pairwise detector detects a unique target to form a loop at a unique position on the nanoswitch but with a common loop size as other pairwise detectors. This causes individual loops to be indistinguishable when using gel electrophoresis as the readout. When the nanoswitch concentration>>target concentration (as with SARS-CoV-2 detection), this results in ability to detect multiple SARS-CoV-2 fragments with an additive signal (FIG. 31B). To implement this concept, loops were identified that could be repositioned along the length of the nanoswitch with similar migrations. Different loop sizes and positions were screened, and it was found that a nearly centered loop of ˜2500 bp provided a constant signal when shifted. The identified design provided two 720 nt regions to place detector pairs. As a first proof of concept, a five target nanoswitch was made to recognize five 30 nt regions of the SARS-CoV-2 genome. It was confirmed that the nanoswitch responded to each of the five targets individually with an indistinguishable signal. In embodiments, the kinetics of binding a single RNA target is identical between a multi-target nanoswitch and a single target nanoswitch. To demonstrate the signal multiplication concept, an equimolar pool of the 5 RNA targets was used representing a perfectly fragmented viral RNA and tested them against 5 different nanoswitches with different number of detectors that can target from 1 to all 5 of the targets. It was show that for a constant target pool, a nearly stoichiometric increase was obtained in the signal as we increase the targeting capability (FIG. 31C).

Having proven the concept, the number of targets was expanded. Considering the long length of viral RNAs such as SARS-CoV-2 (˜30,000 nt) and the short length required for detection (˜20-60 nt), this idea can in principle be expanded to hundreds of targets and achieve a corresponding level of signal amplification. The 720 nt nanoswitch regions were dived into 30 nt segments to enable development of 24-target nanoswitches. Five such 24-target nanoswitches were provided to detect of up to 120 fragments that span the SARS-CoV-2 viral genome. The positive detection of each of the 120 individual targets was confirmed using DNA oligonucleotides (FIG. 31D), and showed that maximum detection efficiency appears randomly distributed with a mean ˜75% (FIG. 31E). To measure the signal enhancement, an equimolar pool was used of the 120 targets and measured the detection signal using single nanoswitches with 1, 10, or 24 detector pairs and mixtures of 1, 2, 3, 4, and 5 24-target nanoswitches. It was found that the signal increases nearly linearly with the number of detectors in the mixture (FIG. 31F) Using serial dilutions of the 120-target mixture, it was additionally confirmed a ˜65 fold improvement in the LOD between the 120 targeting nanoswitch mixture and a single target nanoswitch (FIG. 31G). An LOD of 1.9 fM or 0.01 Amol was determined, which would correspond to detection of ˜6,000 genome copies.

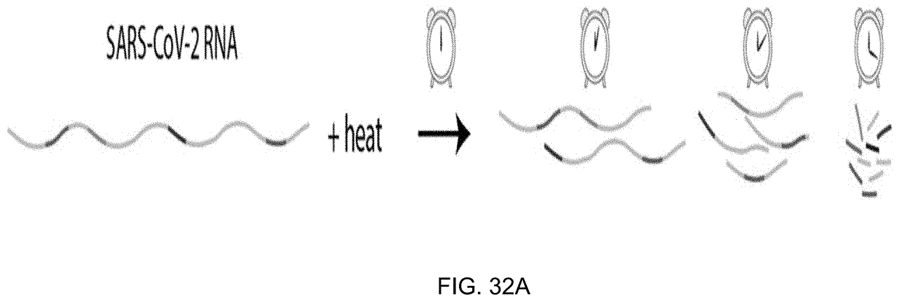

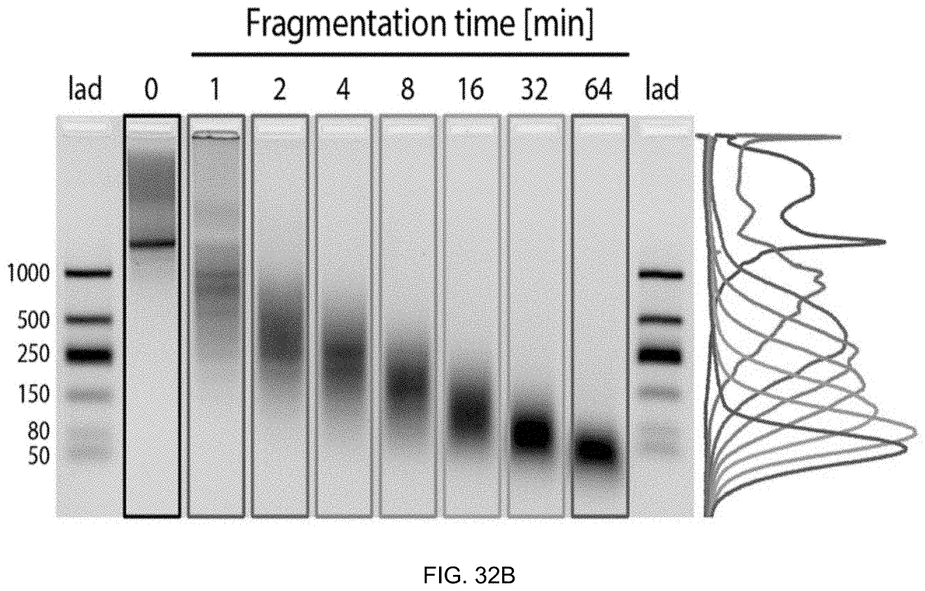

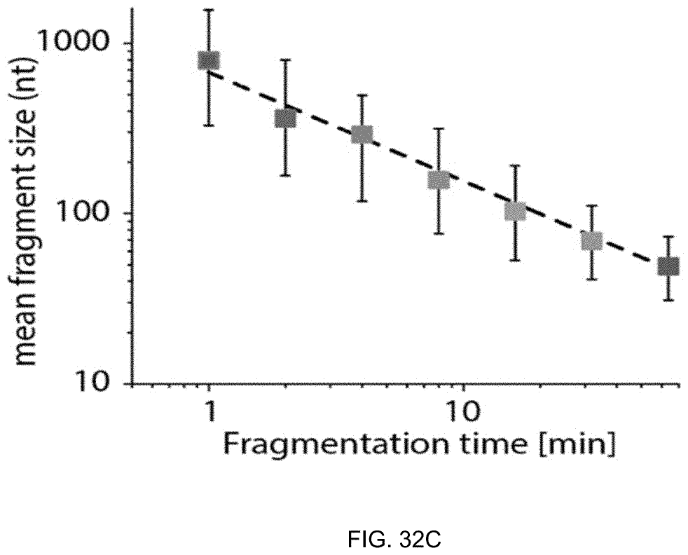

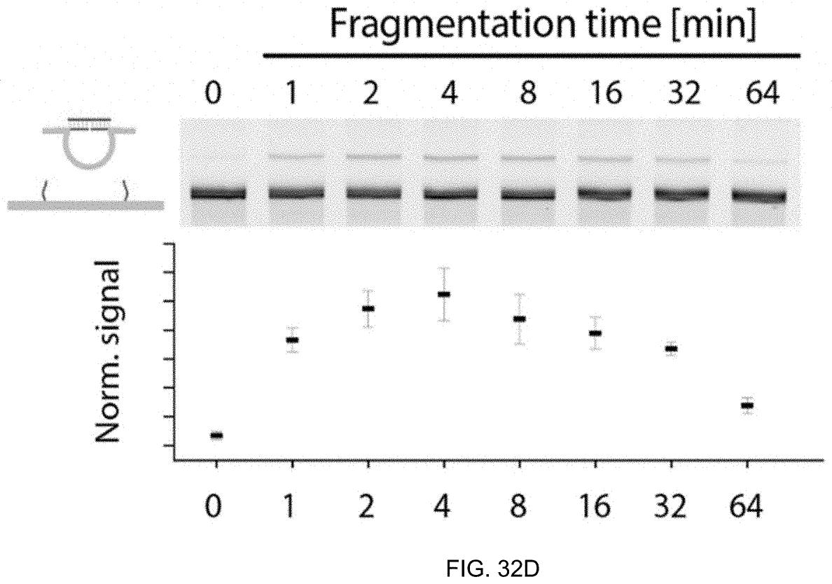

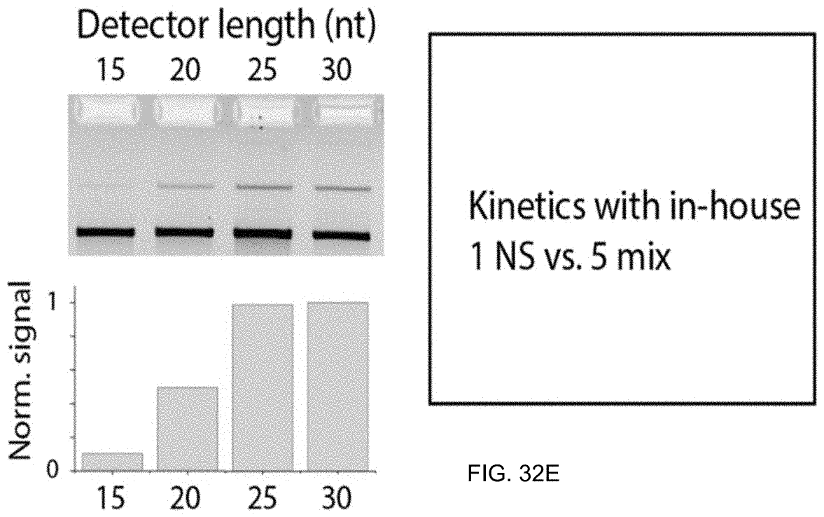

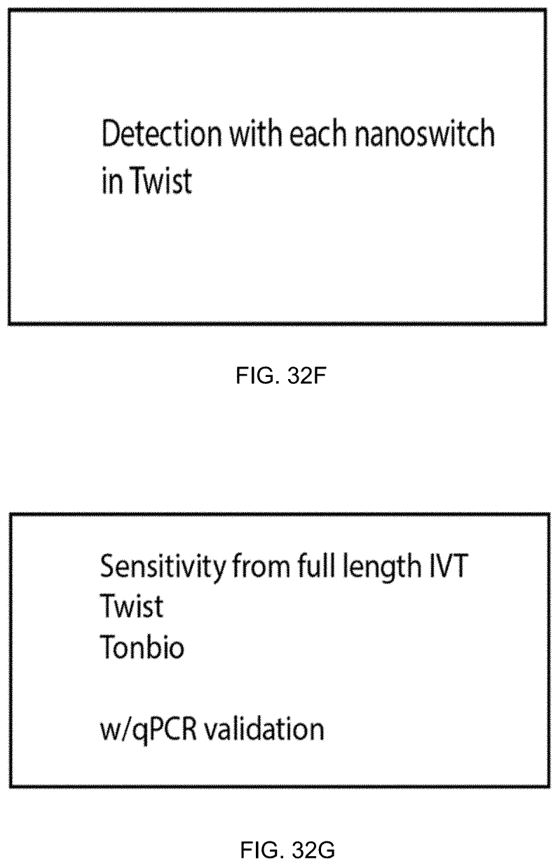

In embodiments, the signal enhancement strategy includes robustly fragmenting the long viral RNA. To develop and test fragmentation protocols, an IVT RNA of the SARS-CoV-2 N-gene (1260 nt) was made and purified. Three fragmentation reagents were tested, a commercial buffer and two homemade buffers and settled on a homemade Tris-Magnesium reagent with 3 mM MgCl2. Using the homemade fragmentation buffer, the N-gene RNA was fragmented for various times from 1 minute to ˜1 hour. The products ran on a 2% agarose gel (FIG. 32B) and estimated fragment sizes with a reference ladder. Mean fragment sizes were found decreasing from ˜800 nt for 1 minute to ˜50 nt for 64 minutes and widths of fragment size distributions narrowing from >1000 nt for 1 minute to ˜42 nt for 64 minutes (FIG. 32C). The products were tested from different fragmentation times individually with nanoswitches containing a single pair of detectors for a sequence in the IVT product. It was found that detection increased as RNA was fragmented but was inhibited as fragments became too small (FIG. 32D). From these results, an optimal fragmentation time of ˜8 minutes was found, balancing the quality of signal with our time requirements for a short test.

Next a nanoswitch design was obtained for the long IVT RNA. Long RNAs including viral RNAs are known to contain strong secondary structures, some of which may persist even as the RNA is fragmented into smaller pieces in the 200 nt range. Such structured regions could slow or otherwise impede binding to our DNA nanoswitch. Recent evidence in the lab suggested that longer detector regions enhanced capture of a 401 nt RNA. To see if that result applies to our fragmented viral RNA, nanoswitches were made and tested with detection regions of 15-30 nt such as 15, 20, 25 and 30 nt. It was found that overall detection signal clearly increased with longer detector lengths (FIG. 32E). Moving forward with 30 nt detectors, reaction kinetics were measured for the fragmented N-gene RNA and found complete reaction in a short period of time such as minutes. Through this process it was also confirmed that multi-targeting nanoswitches provide an enhanced signal that is roughly proportional to the number of targeting regions.

More specifically, FIG. 32A-32G depict detection of SARS-CoV-2 RNA. FIG. 32A depicts a scheme of SARS-CoV-2 detection. FIG. 32B depicts N-gene RNA in vitro transcribed is fragmented using heat at various times. FIG. 32C depicts distribution of apparent fragment sizes as a function of fragmentation time. FIG. 32D depicts nanoswitch-based detection of fragmented N-gene RNA shows optimal performance in the 2-16 minute range. FIG. 32E depicts a prophetic example of a process sequence for detecting clinical positives in the overall process sequence. For in vitro transcribed RNA that spans the whole SARS-CoV-2 genome, detection from each 24 target nanoswitch is similar. FIG. 32G depicts analytical sensitivity of the assay.

Choosing a short fragmentation time such as minutes, the IVT detection was expanded to a more realistic target RNA, spanning the full genome across 6 strands (Twist Biosciences). Using the process conditions of the present disclosure, the RNA with each of the 5 multi-targeting nanoswitches were detected in roughly equal proportion. Using a mixture of five 24-target nanoswitches that can detect 120 different SARS-CoV-2 fragments, an LOD for this full SARS-CoV-2 genome equivalent in aM is achieved.

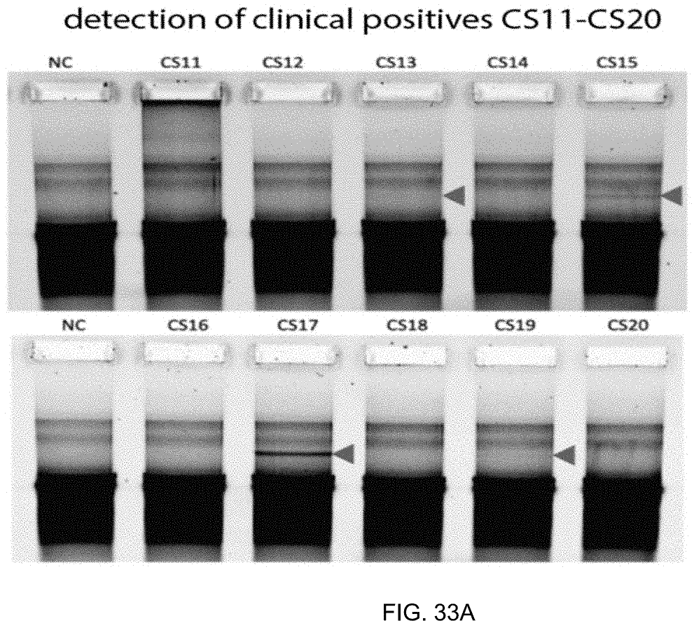

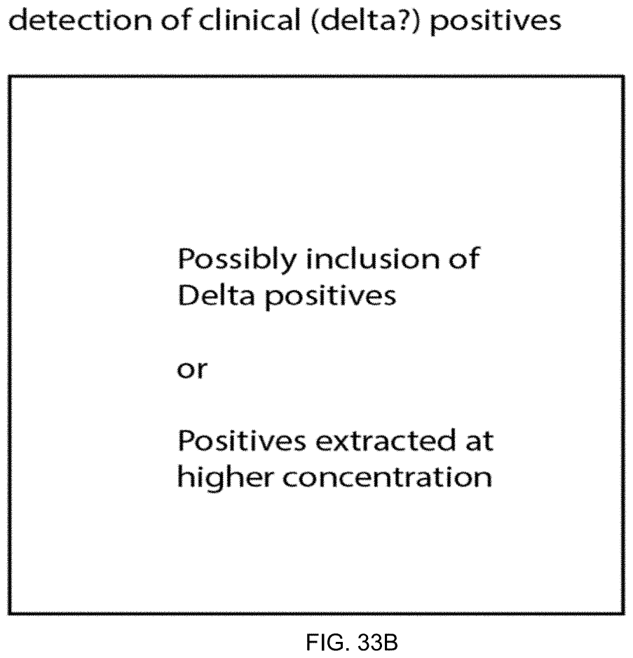

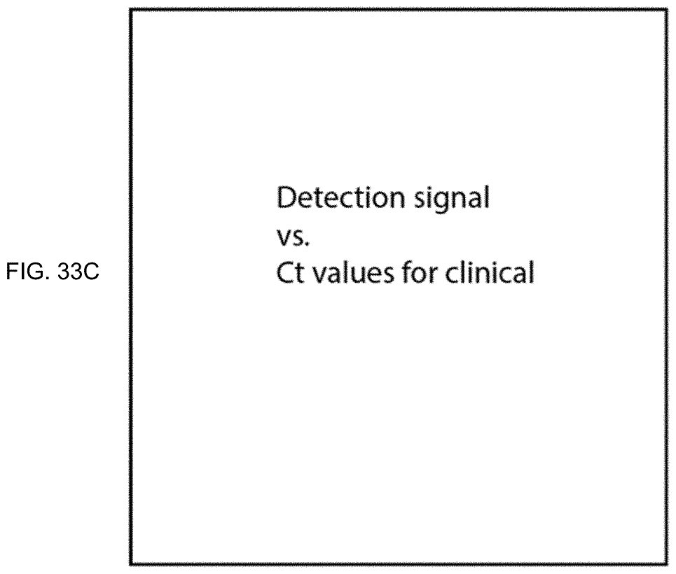

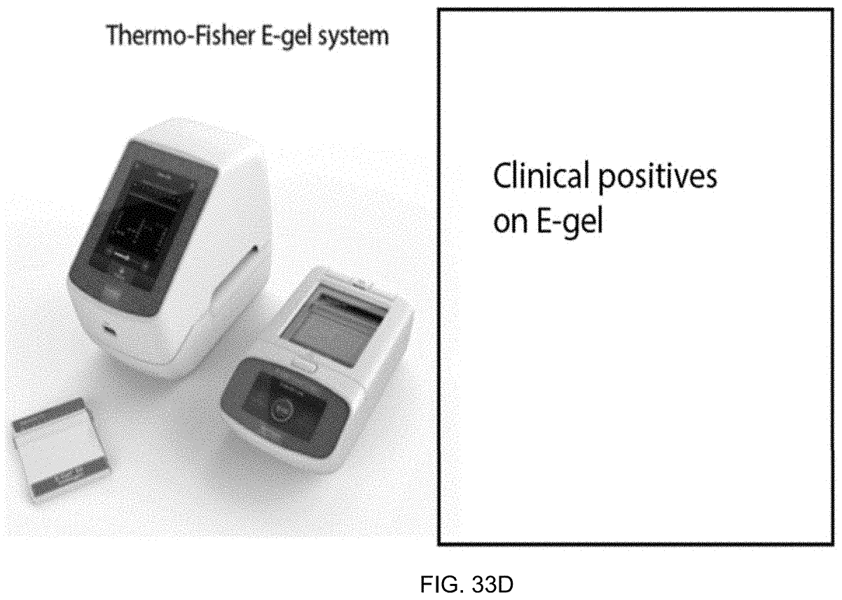

To test our assay on clinical samples, 10 positive and 5 negative samples were obtained of RNA extracts from nasopharangeal swabs (previously confirmed by RT-qPCR). The assay was able to confirm all 5 negatives and registered positive in the samples of FIGS. 33A-D. To further expand the range of detection, the fact that the large majority of viral test fluid is not used was taken advantage of Standard protocols for nasopharangeal swabs use 1-2 mL of transport fluid, and RNA extraction is usually processed in a 1:1 ratio with 50-100 uL collected and ultimately 5-10 uL tested. This provides up to 2 orders of magnitude in unused sensitivity, much of which could be utilized by processing different input and output volumes in RNA extraction. To simulate this, the positive clinical samples were retested with 10× concentration in samples and expanded the detection range.

More specifically, FIGS. 33A-33E depict the detection of clinical SARS-CoV-2. FIG. 33A depicts a nanoswitch based detection of RNA extracts from clinical negative and clinical positive samples. FIG. 33B depicts a prophetic example portion of the process sequence as shown. FIG. 33C depicts detection signal from nanoswitch assay compared to qPCR. FIG. 33D depicts proof of concept for using the E-gel to detect clinical SARS-CoV-2.

Overall, assay embodiments, provide a unique non-enzymatic approach for direct detection of SARS-CoV-2 RNA. The method is shown to be sensitive enough to capture an excellent percentage of clinical positives in <1 hour of end-to-end assay time. The DNA nanoswitch approach has many advantages. The direct detection without enzymes eliminates high cost and complexity of enzymes including cold chain, and also provides a direct linear response that is proportional to viral load, which has been shown to be related to infectivity (See e.g., He, X. et al. Temporal dynamics in viral shedding and transmissibility of COVID-19. Nature Medicine 26, 672-675 (2020)) and severity of the disease (See e.g., Pujadas, E. et al. SARS-CoV-2 viral load predicts COVID-19 mortality. The Lancet Respiratory Medicine 8, e70 (2020)). A related advantage that became obvious during the pandemic is that assay embodiments of the present disclosure use different reagents than almost all other approaches, helping to diversify against supply shortages or bottlenecks.

In embodiments, the multi-targeting approach provides coverage over large portions of the genome, which can enable detection even while variants continue to evolve. A robust detection of the reference strain from Wuhan was shown as well as more recent Delta and Omicron variants. This can be seen as a particular advantage against PCR based methods, where several variants have caused problems in some standard diagnostic assays. Alternatively, the flexible design of the nanoswitch and the detectors does allow options for designing for specificity between variants, especially in those cases where there are numerous mutations. This same versatility also enables the method to be quickly adapted in response to new diseases.

As it stands, embodiments of the present disclosure fill a niche somewhere between diagnostic RT-qPCR and rapid antigen tests. In comparison with RT-qPCR, test embodiments of the present disclosure are likely to be substantially less expensive and faster turnaround, but with the downside of lower analytical sensitivity. Regarding sensitivity, it has been argued that preventing SARS-CoV-2 spread is more about frequent testing than sensitivity. (See e.g., Larremore, D. B. et al. Test sensitivity is secondary to frequency and turnaround time for COVID-19 screening. Science Advances 7, eabd5393). Compared to RT-qPCR, method embodiments, have another advantage of being more tolerant to minor contaminants and being operable without the intense cleanliness that is required of RT-qPCR. Some RT-qPCR has been noted to be affected by false positives due to sample contamination. (See e.g., Braunstein, G. D., Schwartz, L., Hymel, P. & Fielding, J. False Positive Results With SARS-CoV-2 RT-PCR Tests and How to Evaluate a RT-PCR-Positive Test for the Possibility of a False Positive Result. Journal of Occupational and Environmental Medicine 63, e159 (2021)). Compared to rapid antigen tests, the method embodiments provide similar time and equal or superior analytical sensitivity and likely similar or lower cost if developed into a commercial product.

This work demonstrates DNA nanoswitches with clinical samples and represents the first step in development for clinical use. Gel electrophoresis provides a low-cost and simple readout, but is commonly seen as messy and crude with a process not too dissimilar to making molded Jello. Cartridge-based bufferless gels may be a simple way to improve the process, and it has been demonstrated that the test can be ported with some loss of sensitivity to the Invitrogen E-gel system with a cell phone camera. It is believed that slight product variations of gel percentage and dye type could recover the lost sensitivity and make this a compelling solution. Other systems such as automated electrophoresis could be also adapted for this purpose, or possibly capillary electrophoresis.

Overall, embodiments of the present disclosure provide an entirely new way to detect SARS-CoV-2 RNA that is in a class of its own for direct detection of RNA without enzymes. The COVID-19 pandemic has illustrated how over-reliance on single types of tests can cause reagent shortages and workflow bottlenecks that can negatively impact disease control. (See e.g., Esbin, M. N. et al. Overcoming the bottleneck to widespread testing: A rapid review of nucleic acid testing approaches for COVID-19 detection. RNA rna.076232.120 (2020) doi:10.1261/rna.076232.120). The minimalist nature of embodiments of the present disclosure requires relatively few and low cost reagents, enabling the assay to be substantially lower than $1/test at scale. This type of test is adaptable for frequent home testing or surveillance testing. The programmability of the nanoswitches ensure that this method can be readily applied to other RNA viruses and perhaps make an impact on this or future disease outbreaks.

The entire disclosure of all applications, patents, and publications cited herein are herein incorporated by reference in their entirety. While the foregoing is directed to embodiments of the present disclosure, other and further embodiments of the disclosure may be devised without departing from the basic scope thereof.

What is claimed:

1. A method of detecting an RNA virus by reconfiguring a nucleic acid comprising:
contacting a deoxyribonucleic acid (DNA) nanoswitch having a first conformation characterized as open with a biological specimen to form a mixture, wherein when the mixture comprises a ribonucleic acid-of-interest, the first conformation changes to a second conformation characterized as closed;
processing the mixture under conditions sufficient to separate the first conformation, and when present, the second conformation; and
reacting the first conformation, and when present, the second conformation with an indicator under conditions sufficient to form a signal.

2. The method of claim 1, wherein the ribonucleic acid-of-interest is viral ribonucleic acid (viral RNA) or a fragment thereof.

3. The method of claim 1, wherein the ribonucleic acid-of-interest binds to the deoxyribonucleic acid (DNA) nanoswitch to form a second conformation comprising a loop.

4. The method of claim 1, wherein the biological specimen comprises one or more ribonucleic acids-of-interest.

5. The method of claim 4, wherein the one or more ribonucleic acid-of-interest comprises RNA or one or more fragments thereof from ZIKA, EBOLA, SARS, or SARS-CoV-2.

6. The method of claim 1, wherein the ribonucleic acid-of-interest is SEQ ID NO:1, or a fragment thereof.

7. The method of claim 1, wherein processing the mixture comprises electrophoresing the mixture under conditions sufficient to separate the first conformation and the second conformation.

8. The method of claim 1, wherein a formation of the second conformation signals a presence of one or more ribonucleic acids.

9. The method of claim 1, wherein the second conformation has an altered functionality compared to the first conformation.

10. The method of claim 1, wherein the first conformation is configured to change to a second conformation when contacted with ribonucleic acid-of-interest, and wherein the second conformation is configured to report a ribonucleic acid-of-interest.

11. A method of reconfiguring a nucleic acid comprising:
contacting a deoxyribonucleic acid (DNA) nanoswitch having a first conformation, with a ribonucleic acid to form a DNA nanoswitch-nucleic acid complex having a second conformation, wherein the second conformation is characterized as locked;
processing a mixture under conditions sufficient to separate the first conformation and the second conformation; and
contacting the first conformation and second conformation with an indicator under conditions sufficient to form a signal.

12. The method of claim 11, wherein the signal is predetermined to show a presence or absence of ribonucleic acid.

13. A nucleic acid, comprising:
a DNA nanoswitch-nucleic acid complex comprising a deoxyribonucleic acid (DNA) nanoswitch and one or more ribonucleic acid binding sites, wherein the DNA nanoswitch has a first conformation characterized as open, and a second conformation characterized as closed when in a presence of ribonucleic acid-of-interest.

14. A DNA nanoswitch suitable for forming a DNA nanoswitch-ribonucleic acid complex, comprising:
a scaffold comprising a plurality of nucleotides or a polynucleotide sequence;
a plurality of backbone oligonucleotides hybridized to the scaffold to form a backbone polynucleotide;
a first detector strand comprising a nucleic acid sequence having a first segment hybridized to the scaffold or the backbone polynucleotide, and a second segment characterized as an overhang, wherein the second segment is hybridizable to, when present a first segment of a preselected target RNA nucleotide sequence or target RNA polynucleotide-of-interest; and
a second detector strand comprising a nucleic acid sequence having a first segment hybridized to the scaffold or the backbone polynucleotide, and a second segment characterized as an overhang, wherein the second segment is hybridizable to, when present, a second segment of the preselected target RNA nucleotide sequence or the target RNA polynucleotide-of-interest.

15. The DNA nanoswitch of claim 14, wherein the first detector strand comprises a nucleic acid sequence having a first segment hybridized to the scaffold, and a second segment characterized as an overhang, wherein the length of the first segment is about 5 to 30 nucleotides, and the length of the second segment is about 5 to 30 nucleotides.

16. The DNA nanoswitch of claim 14, wherein the second detector strand comprises a nucleic acid sequence having a first segment hybridized to the scaffold, and a second segment characterized as an overhang, wherein the length of the first segment is about 5 to 30 nucleotides, and the length of the second segment is about 5-30 nucleotides.

17. The DNA nanoswitch of claim 14, wherein first detector and the second detector form a pair of detectors.

18. The DNA nanoswitch of claim 17, wherein the DNA nanoswitch further comprises one or more additional pair of detectors, wherein the additional pair of detectors are hybridizable to one or more different segments of the preselected target RNA nucleotide sequence or the target RNA polynucleotide-of-interest.

19. A kit, comprising:
one or more DNA nanoswitches of claim 17, wherein, when present, the DNA nanoswitch hybridizes a viral RNA or a fragment thereof; and
a separation medium.

20. The kit of claim 19, further comprising: a buffer.


SEQUENCE LISTING (447 sequences)

SEQ ID NO: 1 - DNA, 30 bp, Artificial
  Synthetic
agcctaccttgacaagcaatcagacactca

SEQ ID NO: 2 - DNA, 55 bp, Artificial
  synthetic
accgttgtagcaatacttctttgattagtaataacatcactgagtgtctgattgc

SEQ ID NO: 3 - DNA, 55 bp, artificial
  synthetic
ttgtcaaggtaggcttcaaccgattgagggagggaaggtaaatattgacggaaat

SEQ ID NO: 4 - DNA, 30 bp, Artificial
  synthetic
aacgcccaattcaccaagagccgaagccac

SEQ ID NO: 5 - DNA, 45 bp, artificial
  synthetic
caatacttctttgattagtaataacatcacgtggcttcggctctt

SEQ ID NO: 6 - DNA, 45 bp, artificial
  synthetic
ggtgaattgggcgtttcaaccgattgagggagggaaggtaaatat

SEQ ID NO: 7 - DNA, 44 bp, artificial
  synthetic
caatacttctttgattagtaataacatcactggcttcggctctt

SEQ ID NO: 8 - DNA, 44 bp, artificial
  synthetic
ggtgaattgggcgttcaaccgattgagggagggaaggtaaatat

SEQ ID NO: 9 - DNA, 43 bp, artificial
  synthetic
caatacttctttgattagtaataacatcacggcttcggctctt

SEQ ID NO: 10 - DNA, 43 bp, artificial
  synthetic
ggtgaattgggcgtcaaccgattgagggagggaaggtaaatat

SEQ ID NO: 11 - DNA, 42 bp, artificial
  synthetic
caatacttctttgattagtaataacatcacgcttcggctctt

SEQ ID NO: 12 - DNA, 42 bp, artificial
  synthetic
ggtgaattgggctcaaccgattgagggagggaaggtaaatat

SEQ ID NO: 13 - DNA, 41 bp, artificial
  synthetic
caatacttctttgattagtaataacatcaccttcggctctt

SEQ ID NO: 14 - DNA, 41 bp, artificial
  synthetic
ggtgaattgggtcaaccgattgagggagggaaggtaaatat

SEQ ID NO: 15 - DNA, 40 bp, artificial
  synthetic
caatacttctttgattagtaataacatcacttcggctctt

SEQ ID NO: 16 - DNA, 40 bp, artificial
  synthetic
ggtgaattggtcaaccgattgagggagggaaggtaaatat

SEQ ID NO: 17 - DNA, 30 bp, artificial
  synthetic
gtgtgatgccaccatgagctatgaatgccc

SEQ ID NO: 18 - DNA, 55 bp, artificial
  synthetic
accgttgtagcaatacttctttgattagtaataacatcacgggcattcatagctc

SEQ ID NO: 19 - DNA, 55 bp, artificial
  synthetic
atggtggcatcacactcaaccgattgagggagggaaggtaaatattgacggaaat

SEQ ID NO: 20 - DNA, 30 bp, artificial
  synthetic
agtggacagaggctggggaaatggatgtgg

SEQ ID NO: 21 - DNA, 55 bp, Artificial
  synthetic
accgttgtagcaatacttctttgattagtaataacatcacccacatccatttccc

SEQ ID NO: 22 - DNA, 55 bp, artificial
  synthetic
cagcctctgtccacttcaaccgattgagggagggaaggtaaatattgacggaaat

SEQ ID NO: 23 - DNA, 30 bp, artificial; identical to SEQ ID NO: 4
  synthetic

SEQ ID NO: 24 - DNA, 55 bp, artificial
  synthetic
accgttgtagcaatacttctttgattagtaataacatcacgtggcttcggctctt

SEQ ID NO: 25 - DNA, 55 bp, artificial
  synthetic
ggtgaattgggcgtttcaaccgattgagggagggaaggtaaatattgacggaaat

SEQ ID NO: 26 - DNA, 30 bp, artificial
  synthetic
agggagtcaagaaggagcagttcacacggc

SEQ ID NO: 27 - DNA, 55 bp, artificial
  synthetic
accgttgtagcaatacttctttgattagtaataacatcacgccgtgtgaactgct

SEQ ID NO: 28 - DNA, 55 bp, artificial
  synthetic
ccttcttgactcccttcaaccgattgagggagggaaggtaaatattgacggaaat

SEQ ID NO: 29 - DNA, 30 bp, artificial
  synthetic
gtaccatcctgactcccctcgtagattggc

SEQ ID NO: 30 - DNA, 55 bp, artificial
  synthetic
accgttgtagcaatacttctttgattagtaataacatcacgccaatctacgaggg

SEQ ID NO: 31 - DNA, 55 bp, artificial
  synthetic
gagtcaggatggtactcaaccgattgagggagggaaggtaaatattgacggaaat

SEQ ID NO: 32 - DNA, 30 bp, artificial
  synthetic
acatcatgtggagatcagtagaaggggagc

SEQ ID NO: 33 - DNA, 55 bp, artificial
  synthetic
accgttgtagcaatacttctttgattagtaataacatcacgctccccttctactg

SEQ ID NO: 34 - DNA, 55 bp, artificial sequence
  synthetic
atctccacatgatgttcaaccgattgagggagggaaggtaaatattgacggaaat

SEQ ID NO: 35 - DNA, 30 bp, artificial sequence
  synthetic
gaagaacgacacatggaggctgaagagggc

SEQ ID NO: 36 - DNA, 55 bp, artificial sequence
  synthetic
accgttgtagcaatacttctttgattagtaataacatcacgccctcttcagcctc

SEQ ID NO: 37 - DNA, 55 bp, artificial sequence
  synthetic
catgtgtcgttcttctcaaccgattgagggagggaaggtaaatattgacggaaat

SEQ ID NO: 38 - DNA, 30 bp, artificial sequence
  synthetic
ctaattggacaccccgtgagagcatgctgc

SEQ ID NO: 39 - DNA, 55 bp, artificial sequence
  synthetic
accgttgtagcaatacttctttgattagtaataacatcacgcagcatgctctcac

SEQ ID NO: 40 - DNA, 55 bp, artificial sequence
  synthetic
ggggtgtccaattagtcaaccgattgagggagggaaggtaaatattgacggaaat

SEQ ID NO: 41 - DNA, 30 bp, artificial sequence
  synthetic
aaacagtccccggctcgatgtggcactaga

SEQ ID NO: 42 - DNA, 55 bp, artificial sequence
  synthetic
accgttgtagcaatacttctttgattagtaataacatcactctagtgccacatcg

SEQ ID NO: 43 - DNA, 55 bp, artificial sequence
  synthetic
agccggggactgttttcaaccgattgagggagggaaggtaaatattgacggaaat

SEQ ID NO: 44 - DNA, 30 bp, artificial sequence
  synthetic
cccggagagagagcgaggaacatccagact

SEQ ID NO: 45 - DNA, 55 bp, artificial sequence
  synthetic
accgttgtagcaatacttctttgattagtaataacatcacagtctggatgttcct

SEQ ID NO: 46 - DNA, 55 bp, artificial sequence
  synthetic
cgctctctctccgggtcaaccgattgagggagggaaggtaaatattgacggaaat

SEQ ID NO: 47 - DNA, 30 bp, artificial sequence
  synthetic
ggactacccagcaggaacttcaggatctcc

SEQ ID NO: 48 - DNA, 55 bp, artificial sequence
  synthetic
accgttgtagcaatacttctttgattagtaataacatcacggagatcctgaagtt

SEQ ID NO: 49 - DNA, 55 bp, artificial sequence
  synthetic
cctgctgggtagtcctcaaccgattgagggagggaaggtaaatattgacggaaat

SEQ ID NO: 50 - DNA, 30 bp, artificial sequence
  synthetic
gtgacgcattcccggactccaactcaccaa

SEQ ID NO: 51 - DNA, 55 bp, artificial sequence
  synthetic
accgttgtagcaatacttctttgattagtaataacatcacttggtgagttggagt

SEQ ID NO: 52 - DNA, 55 bp, artificial sequence
  synthetic
ccgggaatgcgtcactcaaccgattgagggagggaaggtaaatattgacggaaat

SEQ ID NO: 53 - DNA, 30 bp, artificial sequence
  synthetic
gagttccagaaaacaaaacatcaagagtgg

SEQ ID NO: 54 - DNA, 55 bp, artificial sequence
  synthetic
accgttgtagcaatacttctttgattagtaataacatcacccactcttgatgttt

SEQ ID NO: 55 - DNA, 55 bp, artificial sequence
  synthetic
tgttttctggaactctcaaccgattgagggagggaaggtaaatattgacggaaat

SEQ ID NO: 56 - DNA, 30 bp, artificial sequence
  synthetic
catctaatgggaaggagagaggagggggca

SEQ ID NO: 57 - DNA, 55 bp, artificial sequence
  synthetic
accgttgtagcaatacttctttgattagtaataacatcactgccccctcctctct

SEQ ID NO: 58 - DNA, 55 bp, artificial sequence
  synthetic
ccttcccattagatgtcaaccgattgagggagggaaggtaaatattgacggaaat

SEQ ID NO: 59 - DNA, 30 bp, artificial sequence
  synthetic
cacaggaatagccatgaccgacaccacacc

SEQ ID NO: 60 - DNA, 55 bp, artificial sequence
  synthetic
accgttgtagcaatacttctttgattagtaataacatcacggtgtggtgtcggtc

SEQ ID NO: 61 - DNA, 55 bp, artificial sequence
  synthetic
atggctattcctgtgtcaaccgattgagggagggaaggtaaatattgacggaaat

SEQ ID NO: 62 - DNA, 30 bp, artificial sequence
  synthetic
ggatggggagagagaattcaggaggtggtg

SEQ ID NO: 63 - DNA, 55 bp, artificial sequence
  synthetic
accgttgtagcaatacttctttgattagtaataacatcaccaccacctcctgaat

SEQ ID NO: 64 - DNA, 55 bp, artificial sequence
  synthetic
tctctctccccatcctcaaccgattgagggagggaaggtaaatattgacggaaat

SEQ ID NO: 65 - DNA, 30 bp, artificial sequence
  synthetic
gaggaagttctagagatgcaagacttgtgg

SEQ ID NO: 66 - DNA, 55 bp, artificial sequence
  synthetic
accgttgtagcaatacttctttgattagtaataacatcacccacaagtcttgcat

SEQ ID NO: 67 - DNA, 55 bp, artificial sequence
  synthetic
ctctagaacttcctctcaaccgattgagggagggaaggtaaatattgacggaaat

SEQ ID NO: 68 - DNA, 30 bp, artificial sequence
  synthetic
ctgagtcaaaaaaccccacgcgcttggagg

SEQ ID NO: 69 - DNA, 55 bp, artificial sequence
  synthetic
accgttgtagcaatacttctttgattagtaataacatcaccctccaagcgcgtgg

SEQ ID NO: 70 - DNA, 55 bp, artificial sequence
  synthetic
ggttttttgactcagtcaaccgattgagggagggaaggtaaatattgacggaaat

SEQ ID NO: 71 - DNA, 30 bp, artificial sequence
  synthetic
gtgactgaggactgcggaaatagaggaccc

SEQ ID NO: 72 - DNA, 55 bp, artificial sequence
  synthetic
accgttgtagcaatacttctttgattagtaataacatcacgggtcctctatttcc

SEQ ID NO: 73 - DNA, 55 bp, artificial sequence
  synthetic
gcagtcctcagtcactcaaccgattgagggagggaaggtaaatattgacggaaat

SEQ ID NO: 74 - DNA, 30 bp, artificial sequence
  synthetic
ctctcctcccagagcactataccagagacc

SEQ ID NO: 75 - DNA, 55 bp, artificial sequence
  synthetic
accgttgtagcaatacttctttgattagtaataacatcacggtctctggtatagt

SEQ ID NO: 76 - DNA, 55 bp, artificial sequence
  synthetic
gctctgggaggagagtcaaccgattgagggagggaaggtaaatattgacggaaat

SEQ ID NO: 77 - DNA, 30 bp, artificial sequence
  synthetic
tgctcactggacgatcggccgatttggaac

SEQ ID NO: 78 - DNA, 55 bp, artificial sequence
  synthetic
accgttgtagcaatacttctttgattagtaataacatcacgttccaaatcggccg

SEQ ID NO: 79 - DNA, 55 bp, artificial sequence
  synthetic
atcgtccagtgagcatcaaccgattgagggagggaaggtaaatattgacggaaat

SEQ ID NO: 80 - DNA, 30 bp, artificial sequence
  synthetic
ggccagcactccaagcaaaagcatccagag

SEQ ID NO: 81 - DNA, 55 bp, artificial sequence
  synthetic
accgttgtagcaatacttctttgattagtaataacatcacctctggatgcttttg

SEQ ID NO: 82 - DNA, 55 bp, artificial sequence
  synthetic
cttggagtgctggcctcaaccgattgagggagggaaggtaaatattgacggaaat

SEQ ID NO: 83 - DNA, 30 bp, artificial sequence
  synthetic
cacaccagaagggaaagtagtggacctcgg

SEQ ID NO: 84 - DNA, 55 bp, artificial sequence
  synthetic
accgttgtagcaatacttctttgattagtaataacatcacccgaggtccactact

SEQ ID NO: 85 - DNA, 55 bp, artificial sequence
  synthetic
ttcccttctggtgtgtcaaccgattgagggagggaaggtaaatattgacggaaat

SEQ ID NO: 86 - DNA, 30 bp, artificial sequence
  synthetic
aagccacttacgagccggatgttgacctcg

SEQ ID NO: 87 - DNA, 55 bp, artificial sequence
  synthetic
accgttgtagcaatacttctttgattagtaataacatcaccgaggtcaacatccg

SEQ ID NO: 88 - DNA, 55 bp, artificial sequence
  synthetic
gctcgtaagtggctttcaaccgattgagggagggaaggtaaatattgacggaaat

SEQ ID NO: 89 - DNA, 30 bp, artificial sequence
  synthetic
gcatggcgtagtggactagcggttagagga

SEQ ID NO: 90 - DNA, 55 bp, artificial sequence
  synthetic
accgttgtagcaatacttctttgattagtaataacatcactcctctaaccgctag

SEQ ID NO: 91 - DNA, 55 bp, artificial sequence
  synthetic
tccactacgccatgctcaaccgattgagggagggaaggtaaatattgacggaaat

SEQ ID NO: 92 - DNA, 30 bp, artificial sequence
  synthetic
caagctacagctcaaaggaatgtcatactc

SEQ ID NO: 93 - DNA, 55 bp, artificial sequence
  synthetic
accgttgtagcaatacttctttgattagtaataacatcacgagtatgacattcct

SEQ ID NO: 94 - DNA, 55 bp, artificial sequence
  synthetic
ttgagctgtagcttgtcaaccgattgagggagggaaggtaaatattgacggaaat

SEQ ID NO: 95 - DNA, 30 bp, artificial sequence
  synthetic
gacccatttcctcagagcaatgcaccaatc

SEQ ID NO: 96 - DNA, 55 bp, artificial sequence
  synthetic
accgttgtagcaatacttctttgattagtaataacatcacgattggtgcattgct

SEQ ID NO: 97 - DNA, 55 bp, artificial sequence
  synthetic
ctgaggaaatgggtctcaaccgattgagggagggaaggtaaatattgacggaaat

SEQ ID NO: 98 - DNA, 30 bp, artificial sequence
  synthetic
gaaggcaagaaacgcactggacaacttagc

SEQ ID NO: 99 - DNA, 55 bp, artificial sequence
  synthetic
accgttgtagcaatacttctttgattagtaataacatcacgctaagttgtccagt

SEQ ID NO: 100 - DNA, 55 bp, artificial sequence
  synthetic
gcgtttcttgccttctcaaccgattgagggagggaaggtaaatattgacggaaat

SEQ ID NO: 101 - DNA, 30 bp, artificial sequence
  synthetic
agaacccaagaaccgaaagaaggcacgaag

SEQ ID NO: 102 - DNA, 55 bp, artificial sequence
  synthetic
accgttgtagcaatacttctttgattagtaataacatcaccttcgtgccttcttt

SEQ ID NO: 103 - DNA, 55 bp, artificial sequence
  synthetic
cggttcttgggttcttcaaccgattgagggagggaaggtaaatattgacggaaat

SEQ ID NO: 104 - DNA, 30 bp, artificial sequence
  synthetic
agaccaacaccaagaggcacagtaatggac

SEQ ID NO: 105 - DNA, 55 bp, artificial sequence
  synthetic
accgttgtagcaatacttctttgattagtaataacatcacgtccattactgtgcc

SEQ ID NO: 106 - DNA, 55 bp, artificial sequence
  synthetic
tcttggtgttggtcttcaaccgattgagggagggaaggtaaatattgacggaaat

SEQ ID NO: 107 - DNA, 20 bp, artificial sequence
  synthetic
tctgtccatcacgcaaatta

SEQ ID NO: 108 - DNA, 20 bp, artificial sequence
  synthetic
tattcattaaaggtgaatta

SEQ ID NO: 109 - DNA, 20 bp, artificial sequence
  synthetic
tctgtccatcacgcaaatta

SEQ ID NO: 110 - DNA, 20 bp, artificial
  synthetic
tcgcaagacaaagaacgcga

SEQ ID NO: 111 - DNA, 20 bp, artificial sequence
  synthetic
tctgtccatcacgcaaatta

SEQ ID NO: 112 - DNA, 20 bp, artificial sequence
  synthetic
tcgcaagacaaagaacgcga

SEQ ID NO: 113 - DNA, 30 bp, artificial sequence; identical to SEQ ID NO: 4
  synthetic

SEQ ID NO: 114 - DNA, 55 bp, artificial sequence; identical to SEQ ID NO: 24
  synthetic

SEQ ID NO: 115 - DNA, 55 bp, artificial sequence; identical to SEQ ID NO: 25
  synthetic

SEQ ID NO: 116 - DNA, 30 bp, artificial sequence; identical to SEQ ID NO: 89
  synthetic

SEQ ID NO: 117 - DNA, 55 bp, artificial sequence; identical to SEQ ID NO: 90
  synthetic

SEQ ID NO: 118 - DNA, 55 bp, artificial sequence
  synthetic
tccactacgccatgctgggttatataactatatgtaaatgctgatgcaaatccaa

SEQ ID NO: 119 - DNA, 30 bp, artificial sequence
  synthetic
agactatcatgcttttggggttgctgggaa

SEQ ID NO: 120 - DNA, 55 bp, artificial sequence
  synthetic
accgttgtagcaatacttctttgattagtaataacatcacttcccagcaacccca

SEQ ID NO: 121 - DNA, 55 bp, artificial sequence
  synthetic
aaagcatgatagtcttcaaccgattgagggagggaaggtaaatattgacggaaat

SEQ ID NO: 122 - DNA, 30 bp, artificial sequence
  synthetic
ttgttcggtatgggtaaagggatgccattc

SEQ ID NO: 123 - DNA, 55 bp, artificial sequence
  synthetic
accgttgtagcaatacttctttgattagtaataacatcacgaatggcatcccttt

SEQ ID NO: 124 - DNA, 55 bp, artificial sequence
  synthetic
acccataccgaacaatcaaccgattgagggagggaaggtaaatattgacggaaat

SEQ ID NO: 125 - DNA, 30 bp, artificial
  synthetic
gcgaaggttgagataacgcccaattcacca

SEQ ID NO: 126 - DNA, 55 bp, artificial
  synthetic
accgttgtagcaatacttctttgattagtaataacatcactggtgaattgggcgt

SEQ ID NO: 127 - DNA, 55 bp, artificial sequence
  synthetic
tatctcaaccttcgctcaaccgattgagggagggaaggtaaatattgacggaaat

SEQ ID NO: 128 - DNA, 30 bp, artificial sequence
  synthetic
gtaccgcagcgttcacattcactaagatcc

SEQ ID NO: 129 - DNA, 55 bp, artificial sequence
  synthetic
accgttgtagcaatacttctttgattagtaataacatcacggatcttagtgaatg

SEQ ID NO: 130 - DNA, 55 bp, artificial sequence
  synthetic
tgaacgctgcggtactcaaccgattgagggagggaaggtaaatattgacggaaat

SEQ ID NO: 131 - DNA, 30 bp, artificial sequence
  synthetic
ctgctctgacaactttcattaccccagccg

SEQ ID NO: 132 - DNA, 55 bp, artificial sequence
  synthetic
accgttgtagcaatacttctttgattagtaataacatcaccggctggggtaatga

SEQ ID NO: 133 - DNA, 56 bp, artificial sequence
  synthetic
aagttgtcagagcagtcaaccgattgagggagggaaggtaaatattgacggaaata

SEQ ID NO: 134 - DNA, 30 bp, artificial sequence
  synthetic
agaccattatgctcttaggtttgctgggaa

SEQ ID NO: 135 - DNA, 55 bp, artificial sequence
  synthetic
accgttgtagcaatacttctttgattagtaataacatcacttcccagcaaaccta

SEQ ID NO: 136 - DNA, 55 bp, artificial sequence
  synthetic
agagcataatggtctgttttagcgaacctcccgacttgcgggaggttttgaagcc

SEQ ID NO: 137 - DNA, 30 bp, artificial
  synthetic
ctgtttggcatgggcaaagggatgccattt

SEQ ID NO: 138 - DNA, 55 bp, artificial
  synthetic
accgttgtagcaatacttctttgattagtaataacatcacaaatggcatcccttt

SEQ ID NO: 139 - DNA, 55 bp, artificial sequence
  synthetic
gcccatgccaaacaggttttagcgaacctcccgacttgcgggaggttttgaagcc

SEQ ID NO: 140 - DNA, 30 bp, artificial sequence
  synthetic
gcgaaagtcgaggttacgcctaattcacca

SEQ ID NO: 141 - DNA, 55 bp, artificial
  synthetic
accgttgtagcaatacttctttgattagtaataacatcactggtgaattaggcgt

SEQ ID NO: 142 - DNA, 55 bp, artificial
  synthetic
aacctcgactttcgcgttttagcgaacctcccgacttgcgggaggttttgaagcc

SEQ ID NO: 143 - DNA, 30 bp, artificial
  synthetic
gcactgcggcattcacattcaccaaggtcc

SEQ ID NO: 144 - DNA, 55 bp, artificial
  synthetic
accgttgtagcaatacttctttgattagtaataacatcacggaccttggtgaatg

SEQ ID NO: 145 - DNA, 55 bp, artificial
  synthetic
tgaatgccgcagtgcgttttagcgaacctcccgacttgcgggaggttttgaagcc

SEQ ID NO: 146 - DNA, 30 bp, artificial sequence
  synthetic
ccgcattgacaactctcatcaccccagctg

SEQ ID NO: 147 - DNA, 55 bp, artificial sequence
  synthetic
accgttgtagcaatacttctttgattagtaataacatcaccagctggggtgatga

SEQ ID NO: 148 - DNA, 55 bp, artificial sequence
  synthetic
gagttgtcaatgcgggttttagcgaacctcccgacttgcgggaggttttgaagcc

SEQ ID NO: 149 - DNA, 31 bp, artificial sequence
  synthetic
aattctaatacgactcactatagggagaagg

SEQ ID NO: 150 - DNA, 167 bp, artificial sequence
  synthetic
aatgctgtcagttcatggctcccagcacagtgggatgatcgttaatgatacaggacatgaaactgatgagaatagagcgaaggttgagataacgcccaattcaccaagagccgaagccaccctggggggttttggaagcctaggacttgattgtgaaccgaggacag

SEQ ID NO: 151 - DNA, 20 bp, artificial sequence
  synthetic
ctgtcctcggttcacaatca

SEQ ID NO: 152 - DNA, 55 bp, artificial sequence
  synthetic
aattctaatacgactcactatagggagaaggaatgctgtcagttcatggctccca

SEQ ID NO: 153 - DNA, 30 bp, artificial sequence; identical to SEQ ID NO: 4
  synthetic

SEQ ID NO: 154 - DNA, 55 bp, artificial sequence; identical to SEQ ID NO: 24
  synthetic

SEQ ID NO: 155 - DNA, 55 bp, artificial sequence; identical to SEQ ID NO: 25
  synthetic

SEQ ID NO: 156 - DNA, 30 bp, artificial sequence
  synthetic
gatgatcgttaatgatacaggacatgaaac

SEQ ID NO: 157 - DNA, 55 bp, artificial sequence
  synthetic
accgttgtagcaatacttctttgattagtaataacatcacgtttcatgtcctgta

SEQ ID NO: 158 - DNA, 55 bp, artificial sequence
  synthetic
tcattaacgatcatctcaaccgattgagggagggaaggtaaatattgacggaaat

SEQ ID NO: 159 - DNA, 132 bp, artificial sequence
  synthetic
tggggttttacaggtaacctacaaagcaaccatgatctgtattgtcaagtccatggtaatgcacatgtagctagttgtgatgcaatcatgactaggtgtctagctgtccacgagtgctttgttaagcgtgtt

SEQ ID NO: 160 - RNA, 132 nt, artificial sequence
  synthetic
ugggguuuuacagguaaccuacaaagcaaccaugaucuguauugucaaguccaugguaaugcacauguagcuaguugugaugcaaucaugacuaggugucuagcuguccacgagugcuuuguuaagcguguu

SEQ ID NO: 161 - DNA, 51 bp, artificial sequence
  synthetic
aattctaatacgactcactatagggagaaggtggggttttacrggtaacct

SEQ ID NO: 162 - DNA, 21 bp, artificial sequence
  synthetic
aacacgcttaacaaagcactc

SEQ ID NO: 163 - DNA, 30 bp, artificial sequence
  synthetic
ccatgatctgtattgtcaagtccatggtaa

SEQ ID NO: 164 - DNA, 55 bp, artificial sequence
  synthetic
accgttgtagcaatacttctttgattagtaataacatcacttaccatggacttga

SEQ ID NO: 165 - DNA, 55 bp, artificial sequence
  synthetic
caatacagatcatggtcaaccgattgagggagggaaggtaaatattgacggaaat

SEQ ID NO: 166 - DNA, 30 bp, artificial sequence
  synthetic
atgcaatcatgactaggtgtctagctgtcc

SEQ ID NO: 167 - DNA, 55 bp, artificial sequence
  synthetic
accgttgtagcaatacttctttgattagtaataacatcacggacagctagacacc

SEQ ID NO: 168 - DNA, 55 bp, artificial sequence
  synthetic
tagtcatgattgcattcaaccgattgagggagggaaggtaaatattgacggaaat

SEQ ID NO: 169 - DNA, 60 bp, artificial sequence
  synthetic
agagcataaagctaaatcggttgtaccaaaaacattatgaccctgtaatacttttgcggg

SEQ ID NO: 170 - DNA, 60 bp, artificial sequence
  synthetic
agaagcctttatttcaacgcaaggataaaaatttttagaaccctcatatattttaaatgc

SEQ ID NO: 171 - DNA, 60 bp, artificial sequence
  synthetic
aatgcctgagtaatgtgtaggtaaagattcaaaagggtgagaaaggccggagacagtcaa

SEQ ID NO: 172 - DNA, 60 bp, artificial sequence
  synthetic
atcaccatcaatatgatattcaaccgttctagctgataaattaatgccggagagggtagc

SEQ ID NO: 173 - DNA, 60 bp, artificial sequence
  synthetic
tatttttgagagatctacaaaggctatcaggtcattgcctgagagtctggagcaaacaag

SEQ ID NO: 174 - DNA, 60 bp, artificial sequence
  synthetic
agaatcgatgaacggtaatcgtaaaactagcatgtcaatcatatgtaccccggttgataa

SEQ ID NO: 175 - DNA, 60 bp, artificial sequence
  synthetic
tcagaaaagccccaaaaacaggaagattgtataagcaaatatttaaattgtaaacgttaa

SEQ ID NO: 176 - DNA, 60 bp, artificial sequence
  synthetic
tattttgttaaaattcgcattaaatttttgttaaatcagctcattttttaaccaatagga

SEQ ID NO: 177 - DNA, 60 bp, artificial sequence
  synthetic
acgccatcaaaaataattcgcgtctggccttcctgtagccagctttcatcaacattaaat

SEQ ID NO: 178 - DNA, 60 bp, artificial sequence
  synthetic
ggataggtcacgttggtgtagatgggcgcatcgtaaccgtgcatctgccagtttgagggg

SEQ ID NO: 179 - DNA, 60 bp, artificial sequence
  synthetic
acgacgacagtatcggcctcaggaagatcgcactccagccagctttccggcaccgcttct

SEQ ID NO: 180 - DNA, 60 bp, artificial sequence
  synthetic
ggtgccggaaaccaggcaaagcgccattcgccattcaggctgcgcaactgttgggaaggg

SEQ ID NO: 181 - DNA, 60 bp, artificial sequence
  synthetic
cgatcggtgcgggcctcttcgctattacgccagctggcgaaagggggatgtgctgcaagg

SEQ ID NO: 182 - DNA, 60 bp, artificial sequence
  synthetic
cgattaagttgggtaacgccagggttttcccagtcacgacgttgtaaaacgacggccagt

SEQ ID NO: 183 - DNA, 60 bp, artificial sequence
  synthetic
gccaagcttgcatgcctgcaggtcgactctagaggatccccgggtaccgagctcgaattc

SEQ ID NO: 184 - DNA, 60 bp, artificial
  synthetic
gtaatcatggtcatagctgtttcctgtgtgaaattgttatccgctcacaattccacacaa

SEQ ID NO: 185 - DNA, 60 bp, artificial sequence
  synthetic
catacgagccggaagcataaagtgtaaagcctggggtgcctaatgagtgagctaactcac

SEQ ID NO: 186 - DNA, 60 bp, artificial sequence
  synthetic
attaattgcgttgcgctcactgcccgctttccagtcgggaaacctgtcgtgccagctgca

SEQ ID NO: 187 - DNA, 60 bp, artificial sequence
  synthetic
ttaatgaatcggccaacgcgcggggagaggcggtttgcgtattgggcgccagggtggttt

SEQ ID NO: 188 - DNA, 60 bp, artificial sequence
  synthetic
gttgcagcaagcggtccacgctggtttgccccagcaggcgaaaatcctgtttgatggtgg

SEQ ID NO: 189 - DNA, 60 bp, artificial sequence
  synthetic
ttccgaaatcggcaaaatcccttataaatcaaaagaatagcccgagatagggttgagtgt

SEQ ID NO: 190 - DNA, 60 bp, artificial sequence
  synthetic
tgttccagtttggaacaagagtccactattaaagaacgtggactccaacgtcaaagggcg

SEQ ID NO: 191 - DNA, 60 bp, artificial sequence
  synthetic
aaaaaccgtctatcagggcgatggcccactacgtgaaccatcacccaaatcaagtttttt

SEQ ID NO: 192 - DNA, 60 bp, artificial sequence
  synthetic
ggggtcgaggtgccgtaaagcactaaatcggaaccctaaagggagcccccgatttagagc

SEQ ID NO: 193 - DNA, 60 bp, artificial sequence
  synthetic
ttgacggggaaagccggcgaacgtggcgagaaaggaagggaagaaagcgaaaggagcggg

SEQ ID NO: 194 - DNA, 60 bp, artificial sequence
  synthetic
cgctagggcgctggcaagtgtagcggtcacgctgcgcgtaaccaccacacccgccgcgct

SEQ ID NO: 195 - DNA, 60 bp, artificial sequence
  synthetic
taatgcgccgctacagggcgcgtactatggttgctttgacgagcacgtataacgtgcttt

SEQ ID NO: 196 - DNA, 60 bp, artificial sequence
  synthetic
cctcgttagaatcagagcgggagctaaacaggaggccgattaaagggattttagacagga

SEQ ID NO: 197 - DNA, 60 bp, artificial sequence
  synthetic
acggtacgccagaatcctgagaagtgtttttataatcagtgaggccaccgagtaaaagag

SEQ ID NO: 198 - DNA, 60 bp, artificial sequence
  synthetic
ttgcctgagtagaagaactcaaactatcggccttgctggtaatatccagaacaatattac

SEQ ID NO: 199 - DNA, 60 bp, artificial sequence
  synthetic
cgccagccattgcaacaggaaaaacgctcatggaaatacctacattttgacgctcaatcg

SEQ ID NO: 200 - DNA, 60 bp, artificial sequence
  synthetic
tctgaaatggattatttacattggcagattcaccagtcacacgaccagtaataaaaggga

SEQ ID NO: 201 - DNA, 60 bp, artificial sequence
  synthetic
cattctggccaacagagatagaacccttctgacctgaaagcgtaagaatacgtggcacag

SEQ ID NO: 202 - DNA, 60 bp, artificial sequence
  synthetic
acaatatttttgaatggctattagtctttaatgcgcgaactgatagccctaaaacatcgc

SEQ ID NO: 203 - DNA, 60 bp, artificial sequence
  synthetic
cattaaaaataccgaacgaaccaccagcagaagataaaacagaggtgaggcggtcagtat

SEQ ID NO: 204 - DNA, 60 bp, artificial sequence
  synthetic
taacaccgcctgcaacagtgccacgctgagagccagcagcaaatgaaaaatctaaagcat

SEQ ID NO: 205 - DNA, 60 bp, artificial sequence
  synthetic
caccttgctgaacctcaaatatcaaaccctcaatcaatatctggtcagttggcaaatcaa

SEQ ID NO: 206 - DNA, 60 bp, artificial sequence
  synthetic
cagttgaaaggaattgaggaaggttatctaaaatatctttaggagcactaacaactaata

SEQ ID NO: 207 - DNA, 60 bp, artificial sequence
  synthetic
gattagagccgtcaatagataatacatttgaggatttagaagtattagactttacaaaca

SEQ ID NO: 208 - DNA, 60 bp, artificial sequence
  synthetic
cattatcattttgcggaacaaagaaaccaccagaaggagcggaattatcatcatattcct

SEQ ID NO: 209 - DNA, 60 bp, artificial sequence
  synthetic
gattatcagatgatggcaattcatcaatataatcctgattgtttggattatacttctgaa

SEQ ID NO: 210 - DNA, 60 bp, artificial sequence
  synthetic
taatggaagggttagaacctaccatatcaaaattatttgcacgtaaaacagaaataaaga

SEQ ID NO: 211 - DNA, 60 bp, artificial sequence
  synthetic
aattgcgtagattttcaggtttaacgtcagatgaatatacagtaacagtaccttttacat

SEQ ID NO: 212 - DNA, 60 bp, artificial sequence
  synthetic
cgggagaaacaataacggattcgcctgattgctttgaataccaagttacaaaatcgcgca

SEQ ID NO: 213 - DNA, 60 bp, artificial sequence
  synthetic
gaggcgaattattcatttcaattacctgagcaaaagaagatgatgaaacaaacatcaaga

SEQ ID NO: 214 - DNA, 60 bp, artificial sequence
  synthetic
aaacaaaattaattacatttaacaatttcatttgaattaccttttttaatggaaacagta

SEQ ID NO: 215 - DNA, 60 bp, artificial sequence
  synthetic
cataaatcaatatatgtgagtgaataaccttgcttctgtaaatcgtcgctattaattaat

SEQ ID NO: 216 - DNA, 60 bp, artificial sequence
  synthetic
tttcccttagaatccttgaaaacatagcgatagcttagattaagacgctgagaagagtca

SEQ ID NO: 217 - DNA, 60 bp, artificial sequence
  synthetic
atagtgaatttatcaaaatcataggtctgagagactacctttttaacctccggcttaggt

SEQ ID NO: 218 - DNA, 60 bp, artificial sequence
  synthetic
gaaaactttttcaaatatattttagttaatttcatcttctgacctaaatttaatggtttg

SEQ ID NO: 219 - DNA, 60 bp, artificial sequence
  synthetic
aaataccgaccgtgtgataaataaggcgttaaataagaataaacaccggaatcataatta

SEQ ID NO: 220 - DNA, 60 bp, artificial sequence
  synthetic
ctagaaaaagcctgtttagtatcatatgcgttatacaaattcttaccagtataaagccaa

SEQ ID NO: 221 - DNA, 60 bp, artificial sequence
  synthetic
cgctcaacagtagggcttaattgagaatcgccatatttaacaacgccaacatgtaattta

SEQ ID NO: 222 - DNA, 60 bp, artificial sequence
  synthetic
ggcagaggcattttcgagccagtaataagagaatataaagtaccgacaaaaggtaaagta

SEQ ID NO: 223 - DNA, 60 bp, artificial sequence
  synthetic
attctgtccagacgacgacaataaacaacatgttcagctaatgcagaacgcgcctgttta

SEQ ID NO: 224 - DNA, 60 bp, artificial sequence
  synthetic
tcaacaatagataagtcctgaacaagaaaaataatatcccatcctaatttacgagcatgt

SEQ ID NO: 225 - DNA, 60 bp, artificial
  synthetic
agaaaccaatcaataatcggctgtctttccttatcattccaagaacgggtattaaaccaa

SEQ ID NO: 226 - DNA, 60 bp, artificial sequence
  synthetic
gtaccgcactcatcgagaacaagcaagccgtttttattttcatcgtaggaatcattaccg

SEQ ID NO: 227 - DNA, 60 bp, artificial sequence
  synthetic
cgcccaatagcaagcaaatcagatatagaaggcttatccggtattctaagaacgcgaggc

SEQ ID NO: 228 - DNA, 60 bp, artificial sequence
  synthetic
attttgcacccagctacaattttatcctgaatcttaccaacgctaacgagcgtctttcca

SEQ ID NO: 229 - DNA, 60 bp, artificial sequence
  synthetic
gagcctaatttgccagttacaaaataaacagccatattatttatcccaatccaaataaga

SEQ ID NO: 230 - DNA, 60 bp, artificial sequence
  synthetic
aacgattttttgtttaacgtcaaaaatgaaaatagcagcctttacagagagaataacata

SEQ ID NO: 231 - DNA, 60 bp, artificial sequence
  synthetic
aaaacagggaagcgcattagacgggagaattaactgaacaccctgaacaaagtcagaggg

SEQ ID NO: 232 - DNA, 60 bp, artificial sequence
  synthetic
taattgagcgctaatatcagagagataacccacaagaattgagttaagcccaataataag

SEQ ID NO: 233 - DNA, 60 bp, artificial sequence
  synthetic
agcaagaaacaatgaaatagcaatagctatcttaccgaagccctttttaagaaaagtaag

SEQ ID NO: 234 - DNA, 60 bp, artificial sequence
  synthetic
cagatagccgaacaaagttaccagaaggaaaccgaggaaacgcaataataacggaatacc

SEQ ID NO: 235 - DNA, 60 bp, artificial sequence
  synthetic
caaaagaactggcatgattaagactccttattacgcagtatgttagcaaacgtagaaaat

SEQ ID NO: 236 - DNA, 60 bp, artificial sequence
  synthetic
acatacataaaggtggcaacatataaaagaaacgcaaagacaccacggaataagtttatt

SEQ ID NO: 237 - DNA, 60 bp, artificial sequence
  synthetic
ttgtcacaatcaatagaaaattcatatggtttaccagcgccaaagacaaaagggcgacat

SEQ ID NO: 238 - DNA, 60 bp, artificial sequence
  synthetic
tcaccgtcaccgacttgagccatttgggaattagagccagcaaaatcaccagtagcacca

SEQ ID NO: 239 - DNA, 60 bp, artificial sequence
  synthetic
ttaccattagcaaggccggaaacgtcaccaatgaaaccatcgatagcagcaccgtaatca

SEQ ID NO: 240 - DNA, 60 bp, artificial sequence
  synthetic
gtagcgacagaatcaagtttgcctttagcgtcagactgtagcgcgttttcatcggcattt

SEQ ID NO: 241 - DNA, 60 bp, artificial sequecne
  synthetic
tcggtcatagcccccttattagcgtttgccatcttttcataatcaaaatcaccggaacca

SEQ ID NO: 242 - DNA, 60 bp, artificial sequence
  synthetic
gagccaccaccggaaccgcctccctcagagccgccaccctcagaaccgccaccctcagag

SEQ ID NO: 243 - DNA, 60 bp, artificial sequence
  synthetic
ccaccaccctcagagccgccaccagaaccaccaccagagccgccgccagcattgacagga

SEQ ID NO: 244 - DNA, 60 bp, artificial sequence
  synthetic
ggttgaggcaggtcagacgattggccttgatattcacaaacaaataaatcctcattaaag

SEQ ID NO: 245 - DNA, 60 bp, artificial sequence
  synthetic
ccagaatggaaagcgcagtctctgaatttaccgttccagtaagcgtcatacatggctttt

SEQ ID NO: 246 - DNA, 60 bp, artificial sequence
  synthetic
gatgatacaggagtgtactggtaataagttttaacggggtcagtgccttgagtaacagtg

SEQ ID NO: 247 - DNA, 60 bp, artificial sequence
  synthetic
cccgtataaacagttaatgccccctgcctatttcggaacctattattctgaaacatgaaa

SEQ ID NO: 248 - DNA, 60 bp, artificial sequence
  synthetic
ccaggcggataagtgccgtcgagagggttgatataagtatagcccggaataggtgtatca

SEQ ID NO: 249 - DNA, 60 bp, artificial sequence
  synthetic
ccgtactcaggaggtttagtaccgccaccctcagaaccgccaccctcagaaccgccaccc

SEQ ID NO: 250 - DNA, 60 bp, artificial sequence
  synthetic
tcagagccaccaccctcattttcagggatagcaagcccaataggaacccatgtaccgtaa

SEQ ID NO: 251 - DNA, 60 bp, artificial
  synthetic
cactgagtttcgtcaccagtacaaactacaacgcctgtagcattccacagacagccctca

SEQ ID NO: 252 - DNA, 60 bp, artificial sequence
  synthetic
tagttagcgtaacgatctaaagttttgtcgtctttccagacgttagtaaatgaattttct

SEQ ID NO: 253 - DNA, 60 bp, artificial sequence
  synthetic
gtatgggattttgctaaacaactttcaacagtttcagcggagtgagaatagaaaggaaca

SEQ ID NO: 254 - DNA, 60 bp, artificial sequence
  synthetic
actaaaggaattgcgaataataattttttcacgttgaaaatctccaaaaaaaaggctcca

SEQ ID NO: 255 - DNA, 60 bp, artificial sequence
  synthetic
aaaggagcctttaattgtatcggtttatcagcttgctttcgaggtgaatttcttaaacag

SEQ ID NO: 256 - DNA, 60 bp, artificial sequence
  synthetic
cttgataccgatagttgcgccgacaatgacaacaaccatcgcccacgcataaccgatata

SEQ ID NO: 257 - DNA, 60 bp, artificial sequence
  synthetic
ttcggtcgctgaggcttgcagggagttaaaggccgcttttgcgggatcgtcaccctcagc

SEQ ID NO: 258 - DNA, 60 bp, artificial sequecne
  synthetic
ctttttcatgaggaagtttccattaaacgggtaaaatacgtaatgccactacgaaggcac

SEQ ID NO: 259 - DNA, 60 bp, artificial sequence
  synthetic
caacctaaaacgaaagaggcaaaagaatacactaaaacactcatctttgacccccagcga

SEQ ID NO: 260 - DNA, 60 bp, artificial sequence
  synthetic
ttataccaagcgcgaaacaaagtacaacggagatttgtatcatcgcctgataaattgtgt

SEQ ID NO: 261 - DNA, 60 bp, artificial sequence
  synthetic
cgaaatccgcgacctgctccatgttacttagccggaacgaggcgcagacggtcaatcata

SEQ ID NO: 262 - DNA, 60 bp, artificial sequence
  synthetic
agggaaccgaactgaccaactttgaaagaggacagatgaacggtgtacagaccaggcgca

SEQ ID NO: 263 - DNA, 60 bp, artificial sequence
  synthetic
taggctggctgaccttcatcaagagtaatcttgacaagaaccggatattcattacccaaa

SEQ ID NO: 264 - DNA, 60 bp, artificial sequence
  synthetic
tcaacgtaacaaagctgctcattcagtgaataaggcttgccctgacgagaaacaccagaa

SEQ ID NO: 265 - DNA, 60 bp, artificial sequence
  synthetic
cgagtagtaaattgggcttgagatggtttaatttcaactttaatcattgtgaattacctt

SEQ ID NO: 266 - DNA, 60 bp, artificial sequence
  synthetic
atgcgattttaagaactggctcattataccagtcaggacgttgggaagaaaaatctacgt

SEQ ID NO: 267 - DNA, 60 bp, artificial sequence
  synthetic
taataaaacgaactaacggaacaacattattacaggtagaaagattcatcagttgagatt

SEQ ID NO: 268 - DNA, 60 bp, artificial sequence
  synthetic
taagagcaacactatcataaccctcgtttaccagacgacgataaaaaccaaaatagcgag

SEQ ID NO: 269 - DNA, 60 bp, Artificial sequence
  synthetic
aggcttttgcaaaagaagttttgccagagggggtaatagtaaaatgtttagactggatag

SEQ ID NO: 270 - DNA, 60 bp, artificial sequence
  synthetic
cgtccaatactgcggaatcgtcataaatattcattgaatccccctcaaatgctttaaaca

SEQ ID NO: 271 - DNA, 60 bp, artificial sequence
  synthetic
gttcagaaaacgagaatgaccataaatcaaaaatcaggtctttaccctgactattatagt

SEQ ID NO: 272 - DNA, 60 bp, artificial sequence
  synthetic
cagaagcaaagcggattgcatcaaaaagattaagaggaagcccgaaagacttcaaatatc

SEQ ID NO: 273 - DNA, 60 bp, artificial sequence
  synthetic
gcgttttaattcgagcttcaaagcgaaccagaccggaagcaaactccaacaggtcaggat

SEQ ID NO: 274 - DNA, 60 bp, artificial sequence
  synthetic
tagagagtacctttaattgctccttttgataagaggtcatttttgcggatggcttagagc

SEQ ID NO: 275 - DNA, 60 bp, artificial sequence
  synthetic
ttaattgctgaatataatgctgtagctcaacatgttttaaatatgcaactaaagtacggt

SEQ ID NO: 276 - DNA, 60 bp, artificial sequence
  synthetic
gtctggaagtttcattccatataacagttgattcccaattctgcgaacgagtagatttag

SEQ ID NO: 277 - DNA, 49 bp, artificial
  synthetic
tttgaccattagatacatttcgcaaatggtcaataacctgtttagctat

SEQ ID NO: 278 - DNA, 60 bp, artificial sequence
  synthetic
aacatccaataaatcatacaggcaaggcaaagaattagcaaaattaagcaataaagcctc

SEQ ID NO: 279 - DNA, 60 bp, artificial sequence
  synthetic
gtgagcgagtaacaacccgtcggattctccgtgggaacaaacggcggattgaccgtaatg

SEQ ID NO: 280 - DNA, 60 bp, artificial sequence
  synthetic
ttcttttcaccagtgagacgggcaacagctgattgcccttcaccgcctggccctgagaga

SEQ ID NO: 281 - DNA, 60 bp, artificial
  synthetic
tctgtccatcacgcaaattaaccgttgtagcaatacttctttgattagtaataacatcac

SEQ ID NO: 282 - DNA, 60 bp, artificial
  synthetic
attcgacaactcgtattaaatcctttgcccgaacgttattaattttaaaagtttgagtaa

SEQ ID NO: 283 - DNA, 60 bp, artificial sequence
  synthetic
tgggttatataactatatgtaaatgctgatgcaaatccaatcgcaagacaaagaacgcga

SEQ ID NO: 284 - DNA, 60 bp, artificial sequence
  synthetic
gttttagcgaacctcccgacttgcgggaggttttgaagccttaaatcaagattagttgct

SEQ ID NO: 285 - DNA, 60 bp, artificial sequence
  synthetic
tcaaccgattgagggagggaaggtaaatattgacggaaattattcattaaaggtgaatta

SEQ ID NO: 286 - DNA, 60 bp, artificial
  synthetic
gtattaagaggctgagactcctcaagagaaggattaggattagcggggttttgctcagta

SEQ ID NO: 287 - DNA, 60 bp, artificial sequence
  synthetic
agcgaaagacagcatcggaacgagggtagcaacggctacagaggctttgaggactaaaga

SEQ ID NO: 288 - DNA, 60 bp, artificial sequence
  synthetic
taggaataccacattcaactaatgcagatacataacgccaaaaggaattacgaggcatag

SEQ ID NO: 289 - DNA, 60 bp, artificial sequence
  synthetic
attttcatttggggcgcgagctgaaaaggtggcatcaattctactaatagtagtagcatt

SEQ ID NO: 290 - DNA, 20 bp, artificial sequence
  synthetic
tctgtccatcacgcaaatta

SEQ ID NO: 291 - DNA, 20 bp, artificial sequence
  synthetic
aattttaaaagtttgagtaa

SEQ ID NO: 292 - DNA, 20 bp, artificial sequence
  synthetic
tcgcaagacaaagaacgcga

SEQ ID NO: 293 - DNA, 20 bp, artificial sequence
  synthetic
tcgcaagacaaagaacgcga

SEQ ID NO: 294 - DNA, 20 bp, artificial sequence
  synthetic
tattcattaaaggtgaatta

SEQ ID NO: 295 - DNA, 20 bp, artificial sequence
  synthetic
tagcggggttttgctcagta

SEQ ID NO: 296 - DNA, 14 bp, artificial sequence
  synthetic
tctcatggcccttc

SEQ ID NO: 297 - DNA, 40 bp, artificial sequecne
  synthetic
ctactaatagtagtagcattaacatccaataaatcataca

SEQ ID NO: 298 - DNA, 60 bp, artificial
  synthetic
caaaccaaccaactttcgatctcttgtagatctgttctctaaacgaactttaaaatctgt

SEQ ID NO: 299 - DNA, 60 bp, artificial
  synthetic
gagccttgtccctggtttcaacgagaaaacacacgtccaactcagtttgcctgttttaca

SEQ ID NO: 300 - DNA, 60 bp, artificial
  synthetic
tcatttgacttaggcgacgagcttggcactgatccttatgaagattttcaagaaaactgg

SEQ ID NO: 301 - DNA, 60 bp, artificial
  synthetic
aatgtccaaattttgtatttcccttaaattccataatcaagactattcaaccaagggttg

SEQ ID NO: 302 - DNA, 60 bp, artificial
  synthetic
ttacttaccccaaaatgctgttgttaaaatttattgtccagcatgtcacaattcagaagt

SEQ ID NO: 303 - DNA, 60 bp, artificial
  synthetic
ggtcttaatgacaaccttcttgaaatactccaaaaagagaaagtcaacatcaatattgtt

SEQ ID NO: 304 - DNA, 60 bp, artificial
  synthetic
aattttaaagttacaaaaggaaaagctaaaaaaggtgcctggaatattggtgaacagaaa

SEQ ID NO: 305 - DNA, 60 bp, artificial
  synthetic
taactaacatctttggcactgtttatgaaaaactcaaacccgtccttgattggcttgaag

SEQ ID NO: 306 - DNA, 60 bp, artificial
  synthetic
gttaaatccagagaagaaactggcctactcatgcctctaaaagccccaaaagaaattatc

SEQ ID NO: 307 - DNA, 60 bp, artificial
  synthetic
agagtgtgaatatcacttttgaacttgatgaaaggattgataaagtacttaatgagaagt

SEQ ID NO: 308 - DNA, 60 bp, artificial
  synthetic
gaggatgaagaagaaggtgattgtgaagaagaagagtttgagccatcaactcaatatgag

SEQ ID NO: 309 - DNA, 60 bp, artificial
  synthetic
acaagacggcagtgaggacaatcagacaactactattcaaacaattgttgaggttcaacc

SEQ ID NO: 310 - DNA, 60 bp, artificial
  synthetic
actaacaatgccatgcaagttgaatctgatgattacatagctactaatggaccacttaaa

SEQ ID NO: 311 - DNA, 60 bp, artificial
  synthetic
gcttatgaaaattttaatcagcacgaagttctacttgcaccattattatcagctggtatt

SEQ ID NO: 312 - DNA, 60 bp, artificial
  synthetic
ttttggaaatgaagagtgaaaagcaagttgaacaaaagatcgctgagattcctaaagagg

SEQ ID NO: 313 - DNA, 60 bp, artificial
  synthetic
aagttcctcacagaaaacttgttactttatattgacattaatggcaatcttcatccagat

SEQ ID NO: 314 - DNA, 60 bp, artificial
  synthetic
gaaatgctagcgaaagctttgagaaaagtgccaacagacaattatataaccacttacccg

SEQ ID NO: 315 - DNA, 60 bp, artificial
  synthetic
aaactaaagccatagtttcaactatacagcgtaaatataagggtattaaaatacaagagg

SEQ ID NO: 316 - DNA, 60 bp, artificial
  synthetic
tatcttacttcttcttctaaaacacctgaagaacattttattgaaaccatctcacttgct

SEQ ID NO: 317 - DNA, 60 bp, artificial
  synthetic
gatggagctgatgttactaaaataaaacctcataattcacatgaaggtaaaacattttat

SEQ ID NO: 318 - DNA, 60 bp, artificial
  synthetic
taacactccaacaaatagagttgaagtttaatccacctgctctacaagatgcttattaca

SEQ ID NO: 319 - DNA, 60 bp, artificial
  synthetic
tgcaaaagagtcttgaacgtggtgtgtaaaacttgtggacaacagcagacaacccttaag

SEQ ID NO: 320 - DNA, 60 bp, artificial
  synthetic
ttaccagtgtggtcactataaacatataacttctaaagaaactttgtattgcatagacgg

SEQ ID NO: 321 - DNA, 60 bp, artificial
  synthetic
attcttatttcacagagcaaccaattgatcttgtaccaaaccaaccatatccaaacgcaa

SEQ ID NO: 322 - DNA, 60 bp, artificial
  synthetic
tgattataaacactacacaccctcttttaagaaaggagctaaattgttacataaacctat

SEQ ID NO: 323 - DNA, 60 bp, artificial
  synthetic
acttaaaccagcaaataatagtttaaaaattacagaagaggttggccacacagatctaat

SEQ ID NO: 324 - DNA, 60 bp, artificial
  synthetic
aaaccgtgtttgtactaattatatgccttatttctttactttattgctacaattgtgtac

SEQ ID NO: 325 - DNA, 60 bp, artificial
  synthetic
aaactgataaatattataatttggtttttactattaagtgtttgcctaggttctttaatc

SEQ ID NO: 326 - DNA, 60 bp, artificial
  synthetic
ttctttagacacctatccttctttagaaactatacaaattaccatttcatcttttaaatg

SEQ ID NO: 327 - DNA, 60 bp, artificial
  synthetic
ggccccgatttcagctatggttagaatgtacatcttctttgcatcattttattatgtatg

SEQ ID NO: 328 - DNA, 60 bp, artificial
  synthetic
agtttaaaagaccaataaatcctactgaccagtcttcttacatcgttgatagtgttacag

SEQ ID NO: 329 - DNA, 60 bp, artificial
  synthetic
tattaatgttatagtttttgatggtaaatcaaaatgtgaagaatcatctgcaaaatcagc

SEQ ID NO: 330 - DNA, 60 bp, artificial
  synthetic
aatacgttttcatcaacttttaacgtaccaatggaaaaactcaaaacactagttgcaact

SEQ ID NO: 331 - DNA, 60 bp, artificial
  synthetic
ttgtaataactatatgctcacctataacaaagttgaaaacatgacaccccgtgaccttgg

SEQ ID NO: 332 - DNA, 60 bp, artificial
  synthetic
gttcctttttgttgctgctattttctatttaataacacctgttcatgtcatgtctaaaca

SEQ ID NO: 333 - DNA, 60 bp, artificial
  synthetic
aacatctgttacacaccatcaaaacttatagagtacactgactttgcaacatcagcttgt

SEQ ID NO: 334 - DNA, 60 bp, artificial
  synthetic
tgacacacgttatgtgctcatggatggctctattattcaatttcctaacacctaccttga

SEQ ID NO: 335 - DNA, 60 bp, artificial
  synthetic
tcatgtagttgcctttaatactttactattccttatgtcattcactgtactctgtttaac

SEQ ID NO: 336 - DNA, 60 bp, artificial
  synthetic
gttatgttcacacctttagtacctttctggataacaattgcttatatcatttgtatttcc

SEQ ID NO: 337 - DNA, 60 bp, artificial
  synthetic
acttcagtaactcaggttctgatgttctttaccaaccaccacaaacctctatcacctcag

SEQ ID NO: 338 - DNA, 60 bp, artificial
  synthetic
ttgatacagccaatcctaagacacctaagtataagtttgttcgcattcaaccaggacaga

SEQ ID NO: 339 - DNA, 60 bp, artificial
  synthetic
aatggttcatgtggtagtgttggttttaacatagattatgactgtgtctctttttgttac

SEQ ID NO: 340 - DNA, 60 bp, artificial
  synthetic
ctatgaagtacaattatgaacctctaacacaagaccatgttgacatactaggacctcttt

SEQ ID NO: 341 - DNA, 60 bp, artificial
  synthetic
gtactcaatggtctttgttcttttttttgtatgaaaatgcctttttaccttttgctatgg

SEQ ID NO: 342 - DNA, 60 bp, artificial
  synthetic
gttttaagctaaaagactgtgttatgtatgcatcagctgtagtgttactaatccttatga

SEQ ID NO: 343 - DNA, 60 bp, artificial
  synthetic
tcatgtttttggccagaggtattgtttttatgtgtgttgagtattgccctattttcttca

SEQ ID NO: 344 - DNA, 60 bp, artificial
  synthetic
tacagtctaaaatgtcagatgtaaagtgcacatcagtagtcttactctcagttttgcaac

SEQ ID NO: 345 - DNA, 60 bp, artificial
  synthetic
ggcaaccttacaagctatagcctcagagtttagttcccttccatcatatgcagcttttgc

SEQ ID NO: 346 - DNA, 60 bp, artificial
  synthetic
tatgcagacaatgcttttcactatgcttagaaagttggataatgatgcactcaacaacat

SEQ ID NO: 347 - DNA, 60 bp, artificial
  synthetic
tgaaattagtatggacaattcacctaatttagcatggcctcttattgtaacagctttaag

SEQ ID NO: 348 - DNA, 60 bp, artificial
  synthetic
tttacaggatttgaaatgggctagattccctaagagtgatggaactggtactatctatac

SEQ ID NO: 349 - DNA, 60 bp, artificial
  synthetic
gcctgccaattcaactgtattatctttctgtgcttttgctgtagatgctgctaaagctta

SEQ ID NO: 350 - DNA, 60 bp, artificial
  synthetic
acaaatacctacaacttgtgctaatgaccctgtgggttttacacttaaaaacacagtctg

SEQ ID NO: 351 - DNA, 60 bp, artificial
  synthetic
ataaagtagctggttttgctaaattcctaaaaactaattgttgtcgcttccaagaaaagg

SEQ ID NO: 352 - DNA, 60 bp, artificial
  synthetic
ccacatatatcacgtcaacgtcttactaaatacacaatggcagacctcgtctatgcttta

SEQ ID NO: 353 - DNA, 60 bp, artificial
  synthetic
tatttcaataaaaaggactggtatgattttgtagaaaacccagatatattacgcgtatac

SEQ ID NO: 354 - DNA, 60 bp, artificial
  synthetic
ggtagtggagttcctgttgtagattcttattattcattgttaatgcctatattaaccttg

SEQ ID NO: 355 - DNA, 60 bp, artificial
  synthetic
cattgtgcaaactttaatgttttattctctacagtgttcccacctacaagttttggacca

SEQ ID NO: 356 - DNA, 60 bp, artificial
  synthetic
ttactaacaatgttgcttttcaaactgtcaaacccggtaattttaacaaagacttctatg

SEQ ID NO: 357 - DNA, 60 bp, artificial
  synthetic
ctgtattaatgctaaccaagtcatcgtcaacaacctagacaaatcagctggttttccatt

SEQ ID NO: 358 - DNA, 60 bp, artificial
  synthetic
catccctactataactcaaatgaatcttaagtatgccattagtgcaaagaatagagctcg

SEQ ID NO: 359 - DNA, 60 bp, artificial
  synthetic
catgcctaacatgcttagaattatggcctcacttgttcttgctcgcaaacatacaacgtg

SEQ ID NO: 360 - DNA, 60 bp, artificial
  synthetic
atttgcgtaaacatttctcaatgatgatactctctgacgatgctgttgtgtgtttcaata

SEQ ID NO: 361 - DNA, 60 bp, artificial
  synthetic
tgattatgtgtaccttccttacccagatccatcaagaatcctaggggccggctgttttgt

SEQ ID NO: 362 - DNA, 60 bp, artificial
  synthetic
atttgtacttacaatacataagaaagctacatgatgagttaacaggacacatgttagaca

SEQ ID NO: 363 - DNA, 60 bp, artificial
  synthetic
aaatgctgttacgaccatgtcatatcaacatcacataaattagtcttgtctgttaatccg

SEQ ID NO: 364 - DNA, 60 bp, artificial
  synthetic
gtgactggacaaatgctggtgattacattttagctaacacctgtactgaaagactcaagc

SEQ ID NO: 365 - DNA, 60 bp, artificial
  synthetic
aacctagaccaccacttaaccgaaattatgtctttactggttatcgtgtaactaaaaaca

SEQ ID NO: 366 - DNA, 60 bp, artificial
  synthetic
tcattttgctattggcctagctctctactacccttctgctcgcatagtgtatacagcttg

SEQ ID NO: 367 - DNA, 60 bp, artificial
  synthetic
caattacctgcaccacgcacattgctaactaagggcacactagaaccagaatatttcaat

SEQ ID NO: 368 - DNA, 60 bp, artificial
  synthetic
aattccttacacgtaaccctgcttggagaaaagctgtctttatttcaccttataattcac

SEQ ID NO: 369 - DNA, 60 bp, artificial
  synthetic
gtaggaatgtggcaactttacaagctgaaaatgtaacaggactctttaaagattgtagta

SEQ ID NO: 370 - DNA, 60 bp, artificial
  synthetic
ttttaaaatgaattatcaagttaatggttaccctaacatgtttatcacccgcgaagaagc

SEQ ID NO: 371 - DNA, 60 bp, artificial
  synthetic
gctaaaccaccgcctggagatcaatttaaacacctcataccacttatgtacaaaggactt

SEQ ID NO: 372 - DNA, 60 bp, artificial
  synthetic
gttgacatctatgaagtattttgtgaaaataggacctgagcgcacctgttgtctatgtga

SEQ ID NO: 373 - DNA, 60 bp, artificial
  synthetic
tgttaagcgtgttgactggactattgaatatcctataattggtgatgaactgaagattaa

SEQ ID NO: 374 - DNA, 60 bp, artificial
  synthetic
acaaagcttataaaatagaagaattattctattcttatgccacacattctgacaaattca

SEQ ID NO: 375 - DNA, 60 bp, artificial
  synthetic
aagtgcttttgttaatttaaaacaattaccatttttctattactctgacagtccatgtga

SEQ ID NO: 376 - DNA, 60 bp, artificial
  synthetic
ttagcttgtgggtttacaaacaatttgatacttataacctctggaacacttttacaagac

SEQ ID NO: 377 - DNA, 60 bp, artificial
  synthetic
tgactgacatagccaagaaaccaactgaaacgatttgtgcaccactcactgtcttttttg

SEQ ID NO: 378 - DNA, 60 bp, artificial
  synthetic
ttgtccaacaattacctgaaacttactttactcagagtagaaatttacaagaatttaaac

SEQ ID NO: 379 - DNA, 60 bp, artificial
  synthetic
aattagaagattttattcctatggacagtacagttaaaaactatttcataacagatgcgc

SEQ ID NO: 380 - DNA, 60 bp, artificial
  synthetic
tggtgtaaagatggccatgtagaaacattttacccaaaattacaatctagtcaagcgtgg

SEQ ID NO: 381 - DNA, 60 bp, artificial
  synthetic
tcaatatttaaacacattaacattagctgtaccctataatatgagagttatacattttgg

SEQ ID NO: 382 - DNA, 60 bp, artificial
  synthetic
ttattagtgatatgtacgaccctaagactaaaaatgttacaaaagaaaatgactctaaag

SEQ ID NO: 383 - DNA, 60 bp, artificial
  synthetic
acgcgaacaaatagatggttatgtcatgcatgcaaattacatattttggaggaatacaaa

SEQ ID NO: 384 - DNA, 60 bp, artificial
  synthetic
tgttaatcttacaaccagaactcaattaccccctgcatacactaattctttcacacgtgg

SEQ ID NO: 385 - DNA, 60 bp, artificial
  synthetic
gtactactttagattcgaagacccagtccctacttattgttaataacgctactaatgttg

SEQ ID NO: 386 - DNA, 60 bp, artificial
  synthetic
tttaagaatattgatggttattttaaaatatattctaagcacacgcctattaatttagtg

SEQ ID NO: 387 - DNA, 60 bp, artificial
  synthetic
tcttcaacctaggacttttctattaaaatataatgaaaatggaaccattacagatgctgt

SEQ ID NO: 388 - DNA, 60 bp, artificial
  synthetic
taagtgttatggagtgtctcctactaaattaaatgatctctgctttactaatgtctatgc

SEQ ID NO: 389 - DNA, 60 bp, artificial
  synthetic
aggttttaattgttactttcctttacaatcatatggtttccaacccactaatggtgttgg

SEQ ID NO: 390 - DNA, 60 bp, artificial
  synthetic
ctttccaacaatttggcagagacattgctgacactactgatgctgtccgtgatccacaga

SEQ ID NO: 391 - DNA, 60 bp, artificial
  synthetic
tgcagatcaacttactcctacttggcgtgtttattctacaggttctaatgtttttcaaac

SEQ ID NO: 392 - DNA, 60 bp, artificial
  synthetic
taataactctattgccatacccacaaattttactattagtgttaccacagaaattctacc

SEQ ID NO: 393 - DNA, 60 bp, artificial
  synthetic
aacacccaagaagtttttgcacaagtcaaacaaatttacaaaacaccaccaattaaagat

SEQ ID NO: 394 - DNA, 60 bp, artificial
  synthetic
taacggccttactgttttgccacctttgctcacagatgaaatgattgctcaatacacttc

SEQ ID NO: 395 - DNA, 60 bp, artificial
  synthetic
ctccaattttggtgcaatttcaagtgttttaaatgatatcctttcacgtcttgacaaagt

SEQ ID NO: 396 - DNA, 60 bp, artificial
  synthetic
tatgtccctgcacaagaaaagaacttcacaactgctcctgccatttgtcatgatggaaaa

SEQ ID NO: 397 - DNA, 60 bp, artificial
  synthetic
taataggaattgtcaacaacacagtttatgatcctttgcaacctgaattagactcattca

SEQ ID NO: 398 - DNA, 60 bp, artificial
  synthetic
gaccgcctcaatgaggttgccaagaatttaaatgaatctctcatcgatctccaagaactt

SEQ ID NO: 399 - DNA, 60 bp, artificial
  synthetic
tttgtttatgagaatcttcacaattggaactgtaactttgaagcaaggtgaaatcaagga

SEQ ID NO: 400 - DNA, 60 bp, artificial
  synthetic
tttactcacaccttttgctcgttgctgctggccttgaagccccttttctctatctttatg

SEQ ID NO: 401 - DNA, 60 bp, artificial
  synthetic
cccattactttatgatgccaactattttctttgctggcatactaattgttacgactattg

SEQ ID NO: 402 - DNA, 60 bp, artificial
  synthetic
tcaattgagtacagacactggtgttgaacatgttaccttcttcatctacaataaaattgt

SEQ ID NO: 403 - DNA, 60 bp, artificial
  synthetic
ttaatagttaatagcgtacttctttttcttgctttcgtggtattcttgctagttacacta

SEQ ID NO: 404 - DNA, 60 bp, artificial
  synthetic
taaatattatattagtttttctgtttggaactttaattttagccatggcagattccaacg

SEQ ID NO: 405 - DNA, 60 bp, artificial
  synthetic
cgcgttccatgtggtcattcaatccagaaactaacattcttctcaacgtgccactccatg

SEQ ID NO: 406 - DNA, 60 bp, artificial
  synthetic
ggacctgcctaaagaaatcactgttgctacatcacgaacgctttcttattacaaattggg

SEQ ID NO: 407 - DNA, 60 bp, artificial
  synthetic
tggaatcttgattacatcataaacctcataattaaaaatttatctaagtcactaactgag

SEQ ID NO: 408 - DNA, 60 bp, artificial
  synthetic
ttcaccatttcatcctctagctgataacaaatttgcactgacttgctttagcactcaatt

SEQ ID NO: 409 - DNA, 60 bp, artificial
  synthetic
cttctatttgtgctttttagcctttctgctattccttgttttaattatgcttattatctt

SEQ ID NO: 410 - DNA, 60 bp, artificial
  synthetic
ctaaatcacccattcagtacatcgatatcggtaattatacagtttcctgtttacctttta

SEQ ID NO: 411 - DNA, 60 bp, artificial
  synthetic
ccccaaggtttacccaataatactgcgtcttggttcaccgctctcactcaacatggcaag

SEQ ID NO: 412 - DNA, 60 bp, artificial
  synthetic
cagacgaattcgtggtggtgacggtaaaatgaaagatctcagtccaagatggtatttcta

SEQ ID NO: 413 - DNA, 60 bp, artificial
  synthetic
tcaagcctcttctcgttcctcatcacgtagtcgcaacagttcaagaaattcaactccagg

SEQ ID NO: 414 - DNA, 60 bp, artificial
  synthetic
catacaatgtaacacaagctttcggcagacgtggtccagaacaaacccaaggaaattttg

SEQ ID NO: 415 - DNA, 60 bp, artificial
  synthetic
catattgacgcatacaaaacattcccaccaacagagcctaaaaaggacaaaaagaagaag

SEQ ID NO: 416 - DNA, 60 bp, artificial
  synthetic
taaacgttttcgcttttccgtttacgatatatagtctactcttgtgcagaatgaattctc

SEQ ID NO: 417 - DNA, 60 bp, artificial
  synthetic
gaagagccctaatgtgtaaaattaattttagtagtgctatccccatgtgattttaatagc

SEQ ID NO: 418 - DNA, 60 bp, artificial
  synthetic
tctgtccatcacgcaaattaaccgttgtagacagattttaaagttcgtttagagaacaga

SEQ ID NO: 419 - DNA, 60 bp, artificial
  synthetic
caatacttctttgattagtaataacatcactgtaaaacaggcaaactgagttggacgtgt

SEQ ID NO: 420 - DNA, 60 bp, artificial
  synthetic
ttgcctgagtagaagaactcaaactatcggccagttttcttgaaaatcttcataaggatc

SEQ ID NO: 421 - DNA, 60 bp, artificial
  synthetic
ccttgctggtaatatccagaacaatattaccaacccttggttgaatagtcttgattatgg

SEQ ID NO: 422 - DNA, 60 bp, artificial
  synthetic
cgccagccattgcaacaggaaaaacgctcaacttctgaattgtgacatgctggacaataa

SEQ ID NO: 423 - DNA, 60 bp, artificial
  synthetic
tggaaatacctacattttgacgctcaatcgaacaatattgatgttgactttctctttttg

SEQ ID NO: 424 - DNA, 60 bp, artificial
  synthetic
tctgaaatggattatttacattggcagatttttctgttcaccaatattccaggcaccttt

SEQ ID NO: 425 - DNA, 60 bp, artificial
  synthetic
caccagtcacacgaccagtaataaaagggacttcaagccaatcaaggacgggtttgagtt

SEQ ID NO: 426 - DNA, 60 bp, artificial
  synthetic
cattctggccaacagagatagaacccttctgataatttcttttggggcttttagaggcat

SEQ ID NO: 427 - DNA, 60 bp, artificial
  synthetic
gacctgaaagcgtaagaatacgtggcacagacttctcattaagtactttatcaatccttt

SEQ ID NO: 428 - DNA, 60 bp, artificial
  synthetic
acaatatttttgaatggctattagtctttactcatattgagttgatggctcaaactcttc

SEQ ID NO: 429 - DNA, 60 bp, artificial
  synthetic
atgcgcgaactgatagccctaaaacatcgcggttgaacctcaacaattgtttgaatagta

SEQ ID NO: 430 - DNA, 60 bp, artificial
  synthetic
cattaaaaataccgaacgaaccaccagcagtttaagtggtccattagtagctatgtaatc

SEQ ID NO: 431 - DNA, 60 bp, artificial
  synthetic
aagataaaacagaggtgaggcggtcagtataataccagctgataataatggtgcaagtag

SEQ ID NO: 432 - DNA, 60 bp, artificial
  synthetic
taacaccgcctgcaacagtgccacgctgagcctctttaggaatctcagcgatcttttgtt

SEQ ID NO: 433 - DNA, 60 bp, artificial
  synthetic
agccagcagcaaatgaaaaatctaaagcatatctggatgaagattgccattaatgtcaat

SEQ ID NO: 434 - DNA, 60 bp, artificial
  synthetic
caccttgctgaacctcaaatatcaaaccctcgggtaagtggttatataattgtctgttgg

SEQ ID NO: 435 - DNA, 60 bp, artificial
  synthetic
caatcaatatctggtcagttggcaaatcaacctcttgtattttaatacccttatatttac

SEQ ID NO: 436 - DNA, 60 bp, artificial
  synthetic
cagttgaaaggaattgaggaaggttatctaagcaagtgagatggtttcaataaaatgttc

SEQ ID NO: 437 - DNA, 60 bp, artificial
  synthetic
aaatatctttaggagcactaacaactaataataaaatgttttaccttcatgtgaattatg

SEQ ID NO: 438 - DNA, 60 bp, artificial
  synthetic
gattagagccgtcaatagataatacatttgtgtaataagcatcttgtagagcaggtggat

SEQ ID NO: 439 - DNA, 60 bp, artificial
  synthetic
aggatttagaagtattagactttacaaacacttaagggttgtctgctgttgtccacaagt

SEQ ID NO: 440 - DNA, 60 bp, artificial
  synthetic
attcgacaactcgtattaaatcctttgcccccgtctatgcaatacaaagtttctttagaa

SEQ ID NO: 441 - DNA, 60 bp, artificial
  synthetic
gaacgttattaattttaaaagtttgagtaattgcgtttggatatggttggtttggtacaa

SEQ ID NO: 442 - DNA, 60 bp, artificial
  synthetic
tctgtccatcacgcaaattaaccgttgtagataggtttatgtaacaatttagctcctttc

SEQ ID NO: 443 - DNA, 60 bp, artificial
  synthetic
caatacttctttgattagtaataacatcacattagatctgtgtggccaacctcttctgta

SEQ ID NO: 444 - DNA, 60 bp, artificial
  synthetic
ttgcctgagtagaagaactcaaactatcgggtacacaattgtagcaataaagtaaagaaa

SEQ ID NO: 445 - DNA, 60 bp, artificial
  synthetic
ccttgctggtaatatccagaacaatattacgattaaagaacctaggcaaacacttaatag

SEQ ID NO: 446 - DNA, 60 bp, artificial
  synthetic
cgccagccattgcaacaggaaaaacgctcacatttaaaagatgaaatggtaatttgtata

SEQ ID NO: 447 - DNA, 60 bp, artificial
  synthetic
tggaaatacctacattttgacgctcaatcgcatacataataaaatgatgcaaagaagatg